(19) United States
(12) Patent Application Publication
(10) Pub. No.: US 2025/0163052 A1
(43) Pub. Date: May 22, 2025

(54) cGAS INHIBITORS AND USES THEREOF

(71) Applicant: Cornell University, Ithaca, NY (US)

(72) Inventors: Subhash Sinha et al., New York, NY (US); Li Gan, New York, NY (US); Ravi Kumar Nagiri, New York, NY (US); Sadaf Amin, New York, NY (US); Yige Huang, New York, NY (US)

(21) Appl. No.: 18/838,578

(22) PCT Filed: Feb. 14, 2023
(86) PCT No.: PCT/US2023/062593

§ 371 Date: Aug. 14, 2024

Related U.S. Application Data
(60) Provisional application No. 63/309,894, filed on Feb. 14, 2022.

(51) Int. Cl. C07D 471/04; A61K 31/4355; A61K 31/4365; A61K 31/437; A61K 31/444; A61K 31/506; A61P 25/28; C07D 471/14; C07D 491/048; C07D 495/04

(52) U.S. Cl. CPC C07D 471/04; A61K 31/4355; A61K 31/4365; A61K 31/437; A61K 31/444; A61K 31/506; A61P 25/28; C07D 471/14; C07D 491/048; C07D 495/04

(57) ABSTRACT

The disclosure relates generally novel inhibitors of human cGAS and methods of treating cGAS-related diseases or disorders in a patient.

### CROSS REFERENCE TO RELATED APPLICATIONS

This application claims priority to U.S. provisional patent applications No. 63/309,894, which was filed on Feb. 14, 2022, which is hereby incorporated by reference in its entirety.

### STATEMENT OF GOVERNMENT SUPPORT

This invention was made with government support under Contract No. 5250037401 (RO1AG051390) awarded by the National Institutes of Health. The government has certain rights in the invention.

### TECHNICAL FIELD

The present teachings relate generally to novel chemical compounds and methods useful for treating cGAS-related diseases or disorders.

### BACKGROUND

Innate immunity is considered a first line cellular stress response defending the host cell against invading pathogens and initiating signaling to the adaptive immunity system. These processes are triggered by conserved pathogen-associated molecular patterns (PAMPs) through sensing by diverse pattern recognition receptors (PRRs) and subsequent activation of cytokine and type I interferon gene expression. The major antigen-presenting cells, such as monocytes, macrophages, and dendritic cells produce interferons and are critical for eliciting adaptive T- and B-cell immune system responses. The major PRRs detect aberrant, i.e. mislocalized, immature, or unmodified nucleic acids on either the cell surface, the inside of lysosomal membranes, or the cytosol.

Cyclic GMP-AMP synthase (cGAS/MB21D1) is the predominant sensor for cytosolic dsDNA originating from pathogens or mislocalization of nuclear or mitochondrial self-dsDNA. Binding of dsDNA to cGAS activates the synthesis of c[G(2′,5′)pA(3′,5′)p], a diffusible cyclic dinucleotide referred to as cGAMP, which travels to and activates the endoplasmic reticulum membrane-anchored adaptor protein, Stimulator of interferon genes (STING/TMEM173).

Activated STING recruits and activates TANK binding kinase 1 (TBK1), which in turn phosphorylates the transcription factor family of interferon regulatory factors (IRFs) inducing cytokine and type I interferon mRNA expression. Type I interferons are expressed from over ten IFNA genes and one IFNB1 gene.

The critical role of cGAS in dsDNA sensing has been established in different pathogenic bacteria, viruses, and retroviruses. Additionally, cGAS is essential in various other biological processes such as cellular senescence and recognition of ruptured micronuclei in the surveillance of potential cancer cells.

While the cGAS pathway is important for host defense against invading pathogens, cellular stress and genetic factors may also cause accumulation of self-dsDNA in the cytosol, e.g. from nuclear or mitochondrial leakage. This can trigger autoinflammatory responses. Aicardi-Goutieres syndrome (AGS), a lupus-like severe autoinflammatory immune-mediated disorder, arises from loss-of-function mutation in TREX1, a primary DNA exonuclease responsible for degrading aberrant DNA in cytosol. Knockout of cGAS in TREX1-deficient mice prevented otherwise lethal autoimmune responses, supporting cGAS as a drug target and driver of interferonopathies. Likewise, embryonic lethality caused by deficiency of DNase II, an endonuclease responsible for degradation of excessive DNA in lysosomes during endocytosis, is completely rescued by additional knockout of STING or cGAS. Inhibition of cGAS, therefore, constitutes an important therapeutic strategy for preventing autoinflammatory diseases whose etiology involves anti-dsDNA antibodies. Systemic lupus erythematosus (SLE) may be one such disease [Pisetsky, Nat Rev Rheumatol 12, 102-110 (2016)].

In models of Parkinson's disease, the loss of dopaminergic neurons from the substantia nigra pars compacta, the motor defect, and inflammation observed in aged Parkin knockout mutator mice, which accumulate mutations in mitochondrial DNA, are rescued by loss of STING, suggesting that inflammation facilitates this phenotype (Sliter D, 2018). cGAS and STING have attracted the interest of structural biologists and medicinal chemists for identification of inhibitors and/or activators. An in silico screening effort using murine cGAS-DNA crystal structure led to the identification of a well-characterized small-molecule anti-malarial drug, quinacrine, as a potential cGAS inhibitor [An et ah, Immunol. 194, 4089-4093 (2015)]. However, quinacrine, a known DNA intercalator, was found to indirectly affect the cGAS activity through disruption of dsDNA conformation failing to activate the enzyme instead of directly binding and inhibiting the enzyme. Additionally, considerable off-target effect was observed through its interference with RIG-I pathway.

cGAS activation in microglia of tauopathy mice and human AD brains was investigated, and tau-induced cytosolic mtDNA leakage was identified as a potential mechanism to trigger cGAS-dependent type I interferon response in microglia. Using a combination of behavioral, electrophysiology and single nuclei (sn) RNA-seq, we demonstrated potent protective effects of cGAS inhibition against synaptic and cognitive deficits without affecting pathogenic tau load, and discovered a surprising mechanistic underpinning of the resilience induced by cGAS inhibition. Our findings link maladaptive immune responses with transcriptional network of Mef2c, an AD risk gene implicated in cognitive resilience against amyloid and tau pathology in AD patients, and support cGAS inhibitors as a novel therapeutic approach to enhance cognitive resilience in AD.

Prodrugs are inactive derivatives of a drug and are designed to undergo activation under physiologic conditions after the prodrugs have accumulated in target organs. Some prodrugs can also undergo enzyme-mediated activation and can be designed to undergo both passive and receptor-mediated active cellular uptake.

Small molecule inhibitors or prodrugs thereof that are specific for cGAS would be of great value in treating diseases that arise from inappropriate cGAS activity and the resulting undesired type I interferon activity. Examples of such autoimmune diseases include Aicardi-Goutieres syndrome (AGS) and systemic lupus erythematosus (SLE), a complex chronic systemic autoimmune disease that afflicts over 1.5 million Americans. Current treatments for SLE involve immuno-suppressive regimens associated with debilitating adverse side effects. Other possible utilities related to the suppression of undesired type I interferon activity would include treating inflammatory bowel disease (IBD) and neurodegenerative diseases.

There is a need for cGAS inhibitors that can circulate in tissue and plasma for extended periods or can enhance the brain concentration through the receptor mediated penetration across blood-brain barrier (BBB).

### SUMMARY

The instant disclosure relates to compounds of Formula (I), (II), or (III):

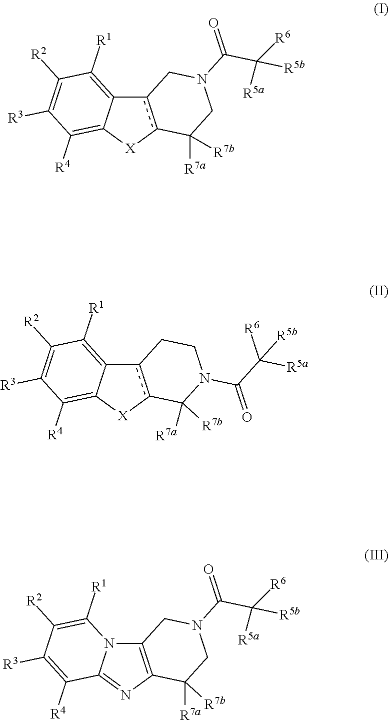

wherein:
R¹ is heteroaryl, halogen, aryl, cyclic amine, hydroxy, —OC(O)alkyl, —NH₂, —N(H)CO— alkyl, or alkoxy;
R² is H, alkyl, —CHF₂, —CF₃, —CN, —OR^{c}, halogen, or heterocyclyl;
R³ and R⁴ are independently H, halogen, —CHF₂, —CF₃, —CN, —OR^{c}, or —OCF₃;
R^{5a} and R^{5b} are independently H, alkyl, aryl, or cycloalkyl;
R⁶ is N(H)R^{a}, O-alkyl, OH, —CO₂R^{d};
or R^{5a} and R⁶ are taken together to form a 5-6 membered heterocyclyl;
R^{7a} and R^{7b} are independently H or alkyl, or, taken together with the carbon to which they are attached form a 3-membered aliphatic carbocyclic ring;
R^{a} is H, alkyl, —COR^{b}, —CON(H)R^{b}, or —CO₂R^{e};
R^{b} is alkyl, or aryl;
R^{c} is H, alkyl, or —C(O)-alkyl;
R^{d} is H or alkyl;
R^{e} is alkyl, or aryl;
X is NH, NMe, NEt, O, or S; and
[symbol] is a single or double bond;
   or a pharmaceutically acceptable salt thereof.

The disclosure also relates to pharmaceutical compositions comprising a compound of formula (I), (II), or (III).

This disclosure further provides a method of treating cGAS-related autoimmune diseases or disorders in a subject with a compound of the disclosure.

This disclosure further provides a method of inhibiting an inflammatory response in a subject with a compound of the disclosure.

This disclosure further provides a method of inhibiting dsDNA-triggered interferon expression in a subject with a compound of the disclosure.

This disclosure further provides a method of treating neurodegenerative diseases or disorders in a subject with a compound of the disclosure.

This disclosure further provides a method of treating epilepsy in a subject with a compound of the disclosure.

This disclosure further provides a method of treating viral infection-associated dementia in a subject with a compound of the disclosure.

This disclosure further provides a method of treating neurological disorders associated with COVID in a subject with a compound of the disclosure.

This disclosure further relates to pharmaceutical compositions comprising a pharmaceutically acceptable carrier and a compound of the disclosure.

These and other features, aspects and advantages of the present disclosure will become better understood with reference to the following figures, associated descriptions, and claims.

### BRIEF DESCRIPTION OF THE FIGURES

FIGS. 1A-1M show the cGAS-STING pathway is activated in hippocampi of tauopathy mice and human AD brains. FIG. 1A is a volcano plot of RNA-seq data from bulk hippocampal tissue from 8-9-month-old P301S and non-transgenic mice. Red dots represent genes with |log 2 fold change|>0.5. Wald test was used. All other genes are colored blue. Select upregulated interferon genes are labeled. n=7 non-transgenic, n=6 P301S; FIG. 1B is the gene set enrichment analysis showing hallmark pathways associated with top 500 DEGs upregulated in P301S compared to non-transgenic samples; FIG. 1C is the gene set enrichment analysis showing top transcription factors associated with top 500 DEGs upregulated in P301S compared to non-transgenic samples; FIG. 1D is the Ingenuity Pathway Analysis prediction of cGAS as an upstream regulator of upregulated DEGs, identified using Activation z-score>1 and p-value of overlap<0.05; FIG. 1E is the Western blots for phosphorylated Tank binding kinase (pTBK1), total Tank binding kinase 1 (TBK1) and GAPDH. Using hippocampal tissue lysates. Lanes 1-7: non-transgenic (ntg); Lanes 8-14: P301S; FIG. 1F is the ratio of pTBK1/TBK1 from (E) showing significantly higher phopho-TBK1 in P301S compared to non-transgenic hippocampi. ** p=0.0015 Student's two tailed t-test; FIG. 1G is the representative immunofluorescent images of non-transgenic and P301S hippocampi labeled with anti-Iba1 (green) and anti-STING antibodies. (Scale bar=50 μm); FIG. 1H is the quantification of Iba1 and Sting immunofluorescence intensities, showing increased Iba1 coverage and Iba1-Sting overlap in P301S hippocampi. Results presented as average intensity measurements from 3-4 section per animal. p-value<0.05. Student's two-tailed t-test. (n=5 Ntg, n=5 P301S); FIG. 1I is the schematic showing the patient number and brain region used for single-nuclei 10× genomics sequencing. (n=7, 4 males and 3 females); FIG. 1J is the UMAP plots showing expression of microglial marker genes INPP5D and CSF1R as well as STAT1 and cGAS (MB21D1) in snRNA-Seq of human microglial population; FIG. 1K is the Gene set enrichment analysis showing hallmark pathways associated enriched in cGAS expressing microglia; FIG. 1L is the representative western blots for pTBK1 and GAPDH using human frontal cortex brain lysates. Lanes 1-3: non-AD (Braak stage 0); Lanes 4-6: AD (Braak stage 6); FIG. 1M is the Ratio of pTBK1/GAPDH from (L) showing significantly higher phospho-TBK1 in human AD compared to non-AD brains. ** p<0.01, Student's two tailed t-test. (n=10 non-AD, n=8 AD)

FIGS. 2A-2J show interferon activation in tau-stimulated microglia is mediated by cGAS and mitochondrial DNA leakage; FIG. 2A is the Quantification of IFNB by ELISA and CXCL10 and CCL5 proteins by MagPix multiplex ELISA in culture media supernatants from untreated (Ctrl) and tau-treated (Tau) primary mouse microglia. IFNB: n=7, ** p=0.0016, Paired ttest. CXCL10 and CCL5, n=5, *** p=0.0004, ** p=0.0031, unpaired ttest; FIG. 2B is the epresentative western blots for phosphorylated Tank binding kinase (pTBK1), total Tank binding kinase 1 (TBK1) and GAPDH using mouse primary microglial cell lysates (Lane 1: untreated; Lane 2: treated with tau fibrils); FIG. 2C is the Ratio of pTBK1/TBK1 from FIG. 2A showing significantly higher phopho-TBK1 in tau fibril-treated primary microglia (Tau) compared to untreated (Ctrl). *, p<0.05, Student's paired t-test (n=3); FIG. 2D is the electron micrographs of primary mouse microglia treated with tau fibrils and immunogold labeled for antibody against tau. (L=lysosome, M=mitochondria); FIG. 2E is the ratio of mitochondrial DNA (Nd2) to genomic DNA (Tert) measured by RT-qPCR on DNA extracts of BV2 IfnB luciferase reporter cells treated for 7 days with ddC (40 or 80 μg/ml) or EtBr (50 or 100 ng/ml) to generate mtDNA-depleted (ρ°) cells. The values are normalized to the untreated sample (n=2), * p<0.0001, one-way ANOVA; FIG. 2F is the control and mtDNA-depleted (ρ°) IfnB luciferase-reporter BV2 cells were stimulated or not with tau fibrils. IfnB signal and viability were measured 16 h later. IfnB-luciferase signal is shown normalized to Cell TiterGlo signal to correct for viability/Cell count. (n=3), **** p<0.0001, two-way ANOVA; FIG. 2G is the bulk RNA-seq analysis for Cgas+/+ and Cgas−/− primary cultured microglia treated or not with tau fibrils or HT-stranded DNA. (n=3 per condition). Venn diagram showing overlap of genes upregulated by dsDNA and tau treatment in Cgas+/+ microglia. Log fold change>1 and FDR<0.05; FIG. 2H is the top 5 reactome pathways represented in upregulated DEGs common to dsDNA and tau treated Cgas+/+ microglia. FDR<0.05; FIG. 2I is the heatmap summary of interferon stimulated genes that are lower in Cgas−/− compared to Cgas+/+ microglia stimulated with HT-DNA or Tau; FIG. 2J is the string interaction plot of genes from (I) including interferon genes including Stat1, Sp100, and Ddx60.

FIGS. 3A-3J show partial or complete loss of Cgas mitigates tauopathy-associated microglial interferon signature. FIG. 3A is the Dot plot showing normalized cell type expression of Cgas (Mb21d1) and Sting (Tmem173) in single nuclei sequencing (snRNA-Seq) samples; FIG. 3B is the UMAP plots showing strong expression of marker genes P2ry12, Siglech, Sall1, and Csf1r in snRNA-Seq microglial population (n=6 per genotype); FIG. 3C is the UMAP plots colored according to microglial subclusters and split by genotype; FIG. 3D is the violin plots showing expression level of homeostatic (P2ry12, Siglech), disease associated (Apoe, Itgax) and interferon (Parp14, Stat1, Trim30a, Rnf213) genes in microglia clusters; FIG. 3E is the Dot plot showing interferon stimulated genes that are significantly lower in P301S Cgas+/− and P301S Cgas−/− microglia compared to P301S Cgas+/+ microglia; FIG. 3F is the representative 63× confocal images of immunostaining of phosphor-STAT1 in the CA1 stratum radiatum of mouse hippocampus. (Scale bar=10 μm); FIG. 3G is the Mean intensity of phosphor-STAT1 measured in CA1 striatum radiatum. Each circle represents the average intensity measurement of 3 images per animal. Statistical comparisons performed using Two-way ANOVA. Data are reported as mean±SEM (n=6 Cgas+/+, n=8 Cgas+/−, n=7 Cgas−/−, n=6 P301S Cgas+/+, n=9 P301S Cgas+/−, n=5 P301S Cgas−/−). (Cgas+/+vs P301S Cgas+/+: p=0.0002, P301S Cgas+/+vs P301S Cgas+/−: p=0.0002, P301S Cgas+/+vs P301S Cgas−/−: p=0.0415); FIG. 3H is the Heatmap showing association of gene modules with genotype; FIG. 3G is the Analysis of disease module 1 and 2 markers compared to disease associated, early response and late response microglia signatures.

FIG. 4A-4I show loss of Cgas rescues tauopathy-induced hippocampal synapse toxicity and memory deficits. FIG. 4A is the cumulative search distance during hidden trials (Session 1-12) in a Morris water maze (MVM) assessment of spatial learning and memory in 7-8-month-old P301S cGas+/+, P301S cGas+/−, and P301S cGas−/− and their non-transgenic littermates. Males and females were tested on separated days. Data presented here represents both sexes combined. n=12 cGas+/+, n=11 cGas+/−, n=11 cGas−/−, n=8 P301S cGas+/+, n=17 P301S cGas+/−, n=6 P301S cGas−/−. Two-way ANOVA. ****, p<0.0001; FIG. 4B is the percentage of time spent in the target or the average time spent in the nontarget (others) quadrants during the 24-hr probe in the MVM assessment. Paired two-tailed Student's ttest; FIG. 4C is the percentage of time spent in the target or the average time spent in the nontarget (others) quadrants during the 72-hr probe in the MVM assessment. Paired two-tailed Student's ttest; FIG. 4D is the field excitatory postsynaptic potentials (fEPSPs) were recorded in the dentate gyrus molecular layer and a TBS protocol was applied (arrow) to the perforant pathway to induce LTP. Representative traces show fEPSPs before and after LTP induction (top). Scale bars, 0.4 mV and 5 ms. The fEPSP slope measurements made up to 60 minutes after TBS were normalized to the mean baseline fEPSP slope before LTP induction (bottom, n=8-11 slices from three to four mice per group); FIG. 4E is the LTP magnitude was calculated from the normalized mean fEPSP slope 55-60 min after TBS was applied. (n=8-11 slices from three to four mice per group; *, p<0.05, **, p<0.01; one-way ANOVA, Bonferonni post-hoc analyses); FIG. 4F is the dot plot showing classification of excitatory neuron clusters by expression of granule, CA1 and CA2/3, CA2 markers; FIG. 4G is the Pie chart summarizing the proportion of DEGs from clusters pertaining to dentate gyrus (DG), CA1 and CA2/3 clusters; FIG. 4H is the representative confocal images of the hippocampal CA1 striatum radiatum labeled with PSD95 antibody. (Scale bar=10 μm); FIG. 4I is the mean intensity of PSD-95 puncta measured in CA1 striatum radiatum. Each circle represents the average intensity measurement of 3-5 images per animal. Statistical comparisons performed using one way or two-way ANOVA. (Cgas+/+vs P301S Cgas+/+: p=0.0275, P301S Cgas+/+vs P301S Cgas+/−: p=0.0002, P301S Cgas+/+vs P301S Cgas−/−: p=0.0415); Data are reported as mean±SEM

FIGS. 5A-5L show loss of cGAS rescues expression of Mef2c and its target genes in tauopathy neurons 905 500×3579. FIG. 5A is the Volcano plot showing representative differentially expressed genes that are upregulated in P301S Cgas−/− compared to P301S Cgas+/+ excitatory neurons. (log 2FC>0.1, FDR<0.05); FIG. 5B is the Representative 63× confocal images of immunostaining of NRG1 in the CA1 stratum radiatum of mouse hippocampus. (Scale bar=10 μm); FIG. 5C is the Mean intensity of NRG1 measured in CA1 striatum radiatum. Each circle represents the average intensity measurement of 3 images per animal. Statistical comparisons performed using Two-way ANOVA. Data are reported as mean±SEM (n=11 Cgas+/+, n=6 P301S Cgas+/+, n=8 P301S Cgas+/−, n=6 P301S Cgas^{-/-}. Cgas+/+ vs P301S Cgas+/+: p=0.0016, P301S Cgas+/+ vs P301S Cgas−/−: p=0.0234); FIG. 5D is the Volcano plot showing representative differentially expressed genes that are upregulated in P301S Cgas^{-/-} compared to P301S Cgas+/+ inhibitory neurons. (log 2FC>0.1, FDR<0.05); FIG. 5E is the Representative 25× confocal images of immunostaining of Mef2c and NeuN in the CA1 pyramidal layer of mouse hippocampus. (Scale bar=50 μm); FIG. 5F is the Mean intensity of Mef2c in Mef2c+, NeuN+ neurons in CA1 pyramidal layer. Each circle represents the average intensity measurement of 3 images per animal. *, p<0.05, unpaired t test. Data are reported as mean±SEM (n=4 P301S Cgas+/+, n=5 P301S Cgas−/−); FIG. 5G is the Venn diagram of the overlap between excitatory neuron DEGs, inhibitory neuron DEGs, and MEF2C target genes; FIG. 5H is the Heatmap showing the overlap between excitatory/inhibitory neuron DEGs and lists of transcription factor target genes (MEF2A, MEF2C, FOSL2, JUNB) and activity-induced differentially expressed genes (ARG and scARG). Numbers in each box represents the overlapping odds ratio; FIG. 5I is the Heatmap of the expression of significant DEGs (p.adj<0.5, log FC >=0.1 or <=−0.1) that are MEF2C targets in WT, P301S, and P301S Cgas−/− excitatory neuron clusters; FIG. 5J is the Heatmap of the expression of significant DEGs (p.adj <0.5, log FC >=0.1 or <=−0.1) that are MEF2C targets in WT, P301S, and P301S Cgas−/− inhibitory neuron clusters; FIG. 5K is the Dot plot showing the expression of significantly upregulated DEGs by Cgas deletion (p.adj <0.5, log FC >=0.1) that are positively correlated with human cognitive resilience in excitatory neuron clusters; FIG. 5L is the Dot plot showing the expression of significantly upregulated DEGs by Cgas deletion (p.adj <0.5, log FC >=0.1) that are positively correlated with human cognitive resilience in inhibitory neuron clusters.

FIGS. 6A-6I show brain permeable cGAS inhibitor elevates MEF2C target genes and protects against synaptic loss and spatial learning and memory deficits. FIG. 6A is the Venn diagram of the overlap between P301S TDI vs P301S Veh DEGs in excitatory neuron, inhibitory neuron, and MEF2C target genes; FIG. 6B is the Heatmap showing the overlap between excitatory/inhibitory neuron DEGs and lists of transcription factor target genes (MEF2A, MEF2C, FOSL2, JUNB) and activity-induced differentially expressed genes (ARG and scARG). Number in each box represents the overlapping odds ratio; FIG. 6C is the Dot plot showing the expression of significantly upregulated DEGs (p.adj <0.5, log FC >=0.1) that are MEF2C targets in Ntg Ctrl, Ntg TDI, P301S Ctrl, P301S TDI excitatory neuron clusters; FIG. 6D is the Dot plot showing the expression of significantly upregulated DEGs (p.adj <0.5, log FC >=0.1) that are MEF2C targets in Ntg Ctrl, Ntg TDI, P301S Ctrl, P301S TDI inhibitory neuron clusters; FIG. 6E is the Novel object recognition test for Ntg and P301S mice fed with 150 mg/kg TDI-6570 or control diet for three months. F: familiar object, N: novel object (n=9 Ntg control, n=6 Ntg TDI-6570, n=5 P301S control, n=12 P301S TDI6570). Statistical comparisons performed using two-way ANOVA. *, p<0.05, **, p<0.01; Data are reported as mean±SEM; FIG. 6F is the Representative confocal images of the hippocampal CA1 striatum radiatum labeled with PSD95 antibody. (Scale bar=10 μm); FIG. 6G is the Mean intensity of PSD95 puncta measured in CA1 striatum radiatum. Each circle represents the average intensity measurement of one image. 3-5 images were taken per animal. Statistical comparisons performed using mixed model (n=13 Ntg control, n=12 Ntg TDI-6570, n=9 P30IS control, n=12 P307S TDI-6570. Ntg control vs P301S control: p=0.0302, P30IS control vs P301S TDI-6570: p=0.0427); FIG. 6H is the Representative confocal images of the hippocampal CA1 striatum radiatum labeled with vGAT antibody. (Scale bar=10 μm); FIG. 6I is the Mean intensity of vGAT puncta measured in CA1 striatum radiatum. Each circle represents the average intensity measurement of one image. 3-5 images were taken per animal. Statistical comparisons performed using mixed model. (n=12 Ntg control, n=11 Ntg TDI-6570, n=8 P301S control, n=13 P301S TDI-6570. Ntg control vs P301S control: p=0.0453, P301S control vs P301S TDI-6570: p=0.0318).

FIG. 7 show Cgas deletion also modified transcriptomes of inhibitory neurons in tauopathy. Subclustering of pan-interneuron marker GAD1 and GAD2 positive neuron populations identified 9 inhibitory neuron subpopulations (FIG. 6A) (Arneson et al., 2018; Cembrowski et al., 2016). We found that Cgas deletion rescued tauopathy-induced downregulation of a subset of interneuron markers such as Pvalb, Vip, Reln, Lhx6, but not Sst or Cck. In addition, analyses of DEGs from interneurons revealed that Cgas deletion led to upregulation of genes involved GABA signaling, including GABA receptor Gabbr2 and GABA transporter Slc6a, supporting restoration of interneuron function by Cgas deletion (FIG. 6B). Interestingly, among the upregulated genes by Cgas deletion, we found many are involved in regulating neuronal excitability and seizure activity, including potassium channels and interacting subunits, Kcnc1, Kcnc2, Kacnip1, and sodium channel Scn1a (FIG. 6C). Indeed, Cgas deletion resulted in a striking upregulation of anti-seizure genes that were diminished in the interneurons of P301S mice (FIG. 6D), including Scn1a. Deficiency of Scn1a leads to Dravet syndrome, an intractable childhood epilepsy with generalized tonic-clonic seizures.

FIG. 8A-8D shows a working model illustrating cGAS-IFN-MEF2c axis in tauopathy. Under disease/vulnerable condition, pathogenic tau activates cGAS-dependent interferon response via mtDNA leakage in microglia, and reduction of MEF2c transcriptional network in excitatory and inhibitory neurons, resulting in cognitive dysfunction. Loss of cGAS reduces interferon response in microglia and enhances Mef2c transcriptional network, resulting in cognitive resilience.

### DETAILED DESCRIPTION

While the concepts of the present disclosure are illustrated and described in detail in the figures and descriptions herein, results in the figures and their description are to be considered as examples and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

The entire contents of each and every patent publication, non-patent publication, and reference text cited herein are hereby incorporated by reference, except that in the event of any inconsistent disclosure or definition from the present specification, the disclosure or definition herein shall be deemed to prevail.

In each of the foregoing and each of the following embodiments, it is to be understood that the formulae include and represent not only all pharmaceutically acceptable salts of the conjugate formulae. It is appreciated that certain functional groups, such as the hydroxy, amino, and like groups form complexes and/or coordination conjugates with water and/or various solvents, in the various physical forms of the compound of formula (I) or (II). It is understood that the formulae depicted throughout the disclosure are include and represent hydrates and/or solvates of compounds of formula (I), (II), or (III). It is also to be understood that the non-hydrates and/or non-solvates of compounds of formula (I), (II), or (III) are described by such formula, as well as the hydrates and/or solvates of the compounds of formula (I), (II), or (III).

### Definitions

For convenience, before further description of the present disclosure, some terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present disclosure to be more readily understood, some terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, to A only (optionally including elements other than B); or to B only (optionally including elements other than A); or yet, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, to at least one, optionally including more than one, A, with no B present (and optionally including elements other than B); or to at least one, optionally including more than one, B, with no A present (and optionally including elements other than A); or yet, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

Various compounds contained in compositions of the present disclosure may exist in particular geometric or stereoisomeric forms. In addition, polymers of the present disclosure may also be optically active. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

If, for instance, a particular enantiomer of compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate opticallyactive acid or base, followed by resolution of the diastereomers thus formed by fractional crystallization or chromatographic means well known in the art, and subsequent recovery of the pure enantiomers.

Structures depicted herein are also meant to include compounds that differ only in the presence of one or more isotopically enriched atoms. For example, compounds produced by the replacement of a hydrogen with deuterium or tritium, or of a carbon with a 13C- or 14C-enriched carbon are within the scope of this disclosure.

The term “prodrug” as used herein encompasses compounds that, under physiological conditions, are converted into therapeutically active agents. A common method for making a prodrug is to include selected moieties that are hydrolyzed under physiological conditions to reveal the desired molecule. The prodrug can be converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Pharmaceutical compositions of the present disclosure are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present disclosure may contain one or more acidic functional groups and, thus, can form pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the patient of one or more compound of the disclosure. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal suffering of a disease, disorder, or condition. A patient or subject can be a primate, canine, feline, or equine. A patient can ne subject is a bird. The bird can be a domesticated bird, such as chicken. The bird can be a fowl. A patient or subject can be a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl, tetracosyl, pentacosyl, hexacosyl, heptacosyl, octacosyl, nonacosyl, trocontanyl, pentacosenyl, and hexacosenyl. A straight chain or branched chain alkyl can have 30 or fewer carbon atoms in its backbone (e.g., C1-C30 for straight chains, C3-C30 for branched chains), or 20 or fewer. Alkyl groups may be substituted or unsubstituted.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH2)-, ethylene —(CH2CH2)-, n-propylene —(CH2CH2CH2)-, isopropylene —(CH2CH(CH3))-, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. In various aspects, cycloalkyls have from 3-10 carbon atoms in their ring structure, or 3-6 carbons in the ring structure. Cycloalkyl groups may be substituted or unsubstituted.

Unless the number of carbons is otherwise specified, “lower alkyl,” as used herein, means an alkyl group, as defined above, but having from one to ten carbons, or from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. A substituent designated herein as alkyl can be a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl but having one or more triple bonds in the moiety.

The term “alkylthio” refers to an alkyl group, as defined above, having a sulfur moiety attached thereto. The “alkylthio” moiety can be represented by one of —(S)-alkyl, —(S)-alkenyl, —(S)-alkynyl, and —(S)-(CH2)m-R1, wherein m and R1 are defined below. Representative alkylthio groups include methylthio, ethylthio, and the like. The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH2)m-R10, where m and R10 are described below.

The terms “amine” and “amino” are art-recognized and refer to both unsubstituted and substituted amines, e.g., a moiety that can be represented by the formulae:

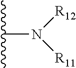

wherein R11 and R12 each independently represent a hydrogen, an alkyl, an alkenyl, —(CH2)m-R10, or R11 and R12 taken together with the N atom to which they are attached complete a heterocycle having from 4 to 8 atoms in the ring structure; R10 represents an alkenyl, aryl, cycloalkyl, a cycloalkenyl, a heterocyclyl, or a polycyclyl; and m is zero or an integer in the range of 1 to 8. In some instances, only one of R11 or R12 can be a carbonyl, e.g., R11, R12, and the nitrogen together do not form an imide. R11 and R12 each independently can represent a hydrogen, an alkyl, an alkenyl, or —(CH2)m- R10. Thus, the term “alkylamine” as used herein means an amine group, as defined above, having a substituted or unsubstituted alkyl attached thereto, i.e., at least one of R11 and R12 is an alkyl group. An amino group or an alkylamine is basic, meaning it has a conjugate acid with a pKa >7.00, i.e., the protonated forms of these functional groups have pKas relative to water above about 7.00.

The term “amide”, as used herein, refers to a group

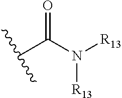

wherein each R13 independently represent a hydrogen or hydrocarbyl group, or two R13 are taken together with the N atom to which they are attached complete a heterocycle having from 4 to 8 atoms in the ring structure.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). In various aspects, aryl groups include 5- to 12-membered rings, or 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carbocyclic aryl groups include benzene, naphthalene, phenanthrene, phenol, aniline, and the like. Heteroaryl groups include substituted or unsubstituted aromatic 3- to 12-membered ring structures, 5- to 12-membered rings, or 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic. Each instance of an aryl group may be independently optionally substituted, i.e., unsubstituted (an “unsubstituted aryl”) or substituted (a “substituted aryl”) with one or more substituents; e.g., for instance from 1 to 5 substituents, 1 to 4 substituents, 1 to 3 substituents, 1 to 2 substituents or just 1 substituent. The aromatic ring may be substituted at one or more ring positions with one or more substituents, such as halogen, azide, alkyl, aryl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. For example, the aryl group can be an unsubstituted C5-C12 aryl or the aryl group can be a substituted C5-C10 aryl.

The term “halo”, “halide”, or “halogen” as used herein means halogen and includes, for example, and without being limited thereto, fluoro, chloro, bromo, iodo and the like, in both radioactive and non-radioactive forms. Halo can be selected from the group consisting of fluoro, chloro and bromo.

The terms “heterocyclyl” or “heterocyclic group” refer to 3- to 12-membered ring structures, 5- to 12-membered rings, or 5- to 10-membered rings, whose ring structures include one to four heteroatoms. Heterocycles can be monocyclic, bicyclic, spirocyclic, or polycyclic. Heterocycles can be saturated or unsaturated. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aryl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

A heteroaryl ring is an embodiment of a heterocyclyl group. The phrase “heterocyclyl group” includes fused ring species including those that include fused aromatic and non-aromatic groups. Representative heterocyclyl groups include, but are not limited to, piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

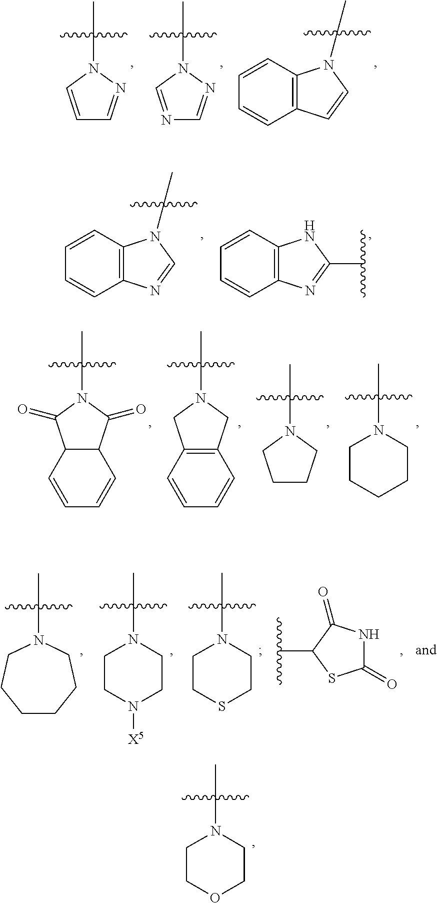

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “carbonyl” is art-recognized and includes such moieties as can be represented by the formula:

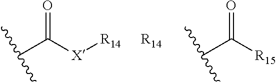

wherein X′ is a bond or represents an oxygen, a nitrogen, or a sulfur, and R14 represents a hydrogen, an alkyl, an alkenyl, —(CH2)m-R10 or a pharmaceutically acceptable salt, R15 represents a hydrogen, an alkyl, an alkenyl or —(CH2)m-R10, where m and R10 are as defined above. Where X′ is an oxygen and R14 or R15 is not hydrogen, the formula represents an “ester.” Where X′ is an oxygen, and R14 is as defined above, the moiety is referred to herein as a carboxyl group, and particularly when R14 is a hydrogen, the formula represents a “carboxylic acid”. Where X′ is an oxygen, and R15 is a hydrogen, the formula represents a “formate.” In general, where the oxygen atom of the above formula is replaced by a sulfur, the formula represents a “thiocarbonyl” group. Where X′ is a sulfur and R14 or R15 is not hydrogen, the formula represents a “thioester” group. Where X′ is a sulfur and R14 is a hydrogen, the formula represents a “thiocarboxylic acid” group. Where X′ is a sulfur and R15 is a hydrogen, the formula represents a “thioformate” group. On the other hand, where X′ is a bond, and R14 is not hydrogen, the above formula represents a “ketone” group. Where X′ is a bond, and R14 is a hydrogen, the above formula represents an “aldehyde” group.

The term “amido” as used herein refers to a group having the formula C(O)NRR, wherein R is defined herein and can each independently be, e.g., hydrogen, alkyl, aryl or each R, together with the nitrogen atom to which they are attached, form a heterocyclyl group.

As used herein, the term “nitro” means —NO₂; the term “halogen” designates-F, —Cl, —Br, or —I; the term “sulfhydryl” means —SH; the term “hydroxyl” means-OH; the term “sulfonyl” means —SO₂—; the term “azido” means —N3; the term “cyano” means-CN; the term “isocyanato” means-NCO; the term “thiocyanato” means —SCN; the term “isothiocyanato” means —NCS; and the term “cyanato” means —OCN.

As used herein, the definition of each expression, e.g., alkyl, m, n, etc., when it occurs more than once in any structure, is intended to be independent of its definition elsewhere in the same structure.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl, or an aromatic or heteroaromatic moiety. The substituents on substituted alkyls can be selected from C1-6 alkyl, C3-6 cycloalkyl, halogen, carbonyl, cyano, or hydroxyl. The substituents on substituted alkyls can be selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “substituted” as used herein also refers to a group that is substituted with one or more groups including, but not limited to, the following groups: halogen (e.g., F, Cl, Br, and I), R, OR, ROH (e.g., CH₂OH), OC(O)N(R)₂, CN, NO, NO₂, ONO₂, azido, CF₃, OCF₃, methylenedioxy, ethylenedioxy, (C₃-C₂₀) heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl, (C₆-C₂₀) aryl, heterocyclyl or polyalkylene oxide groups, such as polyalkylene oxide groups of the formula (CH₂CH₂O)_{f}—R—OR, —(CH₂CH₂CH₂O)_{g}—R—OR, —(CH₂CH₂O)_{f}(CH₂CH₂CH₂O)_{g}—R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

For purposes of this disclosure, the chemical elements are identified in accordance with the Periodic Table of the Elements, CAS version, Handbook of Chemistry and Physics, 67th Ed., 1986-87, inside cover.

### Compounds of the Disclosure

The disclosure relates compounds of Formula (I) or (II):

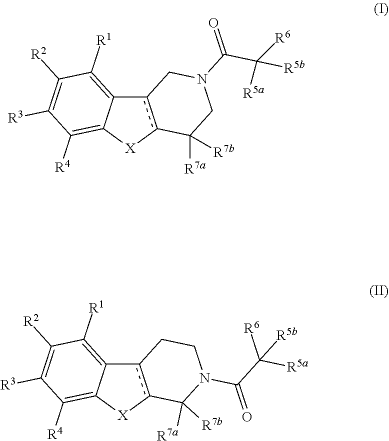

wherein:
R¹ is heteroaryl, halogen, aryl, cyclic amine, hydroxy, —OC(O)alkyl, —NH₂, —N(H)CO— alkyl, or alkoxy;
R² is H, alkyl, —CHF₂, —CF₃, —CN, —OR^{c}, halogen, or heterocyclyl;
R³ and R⁴ are independently H, halogen, —CHF₂, —CF₃, —CN, —OR^{c}, or —OCF₃;
R^{5a} and R^{5b} are independently H, alkyl, aryl, or cycloalkyl;
R⁶ is N(H)R^{a}, O-alkyl, OH, —CO₂R^{d};
or R^{5a} and R⁶ are taken together to form a 5-6 membered heterocyclyl;
R^{7a} and R^{7b} are independently H or alkyl, or, taken together with the carbon to which they are attached form a 3-membered aliphatic carbocyclic ring;
R^{a} is H, alkyl, —COR^{b}, —CON(H)R^{b}, or —CO₂R^{e};
R^{b} is alkyl, or aryl;
R^{c} is H, alkyl, or —C(O)-alkyl;
R^{d} is H or alkyl;
R^{e} is alkyl, or aryl;
X is NH, NMe, NEt, O, or S; and
[symbol] is a single or double bond;
or a pharmaceutically acceptable salt thereof.

The invention relates to compounds of formula (I). The invention relates to compounds of formula (II). The invention also relates to prodrugs of compounds of formula (I), or (II) or pharmaceutically acceptable salt thereof.

R¹ can be substituted or unsubstituted heteroaryl. R′ can be a N-containing heteroaryl. R¹ can be substituted or unsubstituted C—C or C—N linked monocyclic or bicyclic heteroaryl. R′ can be substituted or unsubstituted aryl. R′ can be ortho, meta or para aminophenyl, methoxyphenyl, or fluorophenyl. R¹ can be substituted or unsubstituted aryl. R¹ can be halogen. R¹ can be substituted or unsubstituted cyclic amine. R′ can be functionalized and unfunctionalized azacyclobutane, azacyclobutanone, azacyclopentane, azacyclopemtanone. R¹ can be hydroxy. R¹ can be substituted or unsubstituted —OC(O)alkyl. R¹ can be —NH₂. R′ can be substituted or unsubstituted —N(H)COalkyl. R¹ can be alkoxy. R¹ can be C₁-C₄alkoxy.

R¹ can be

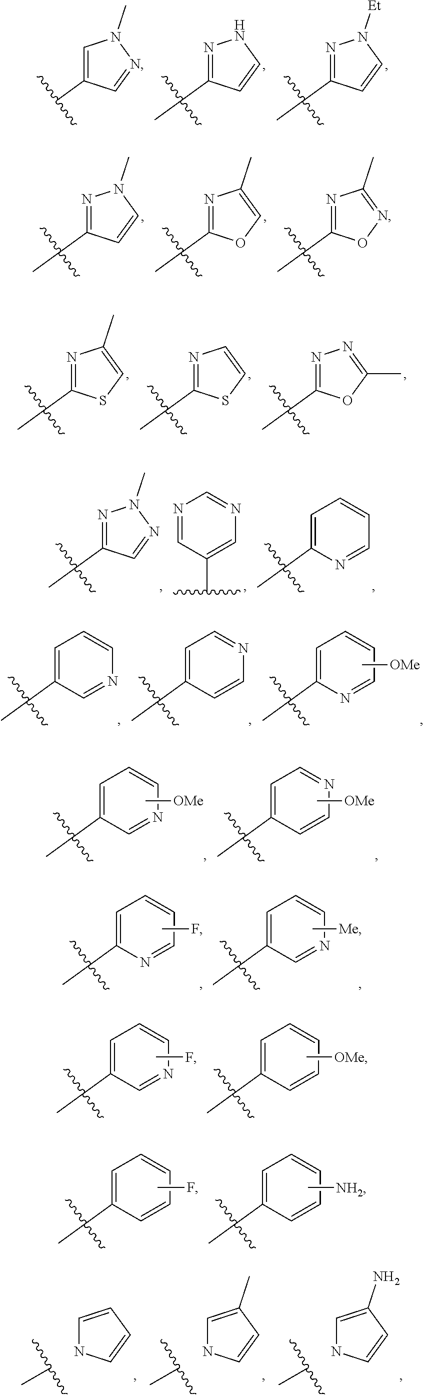

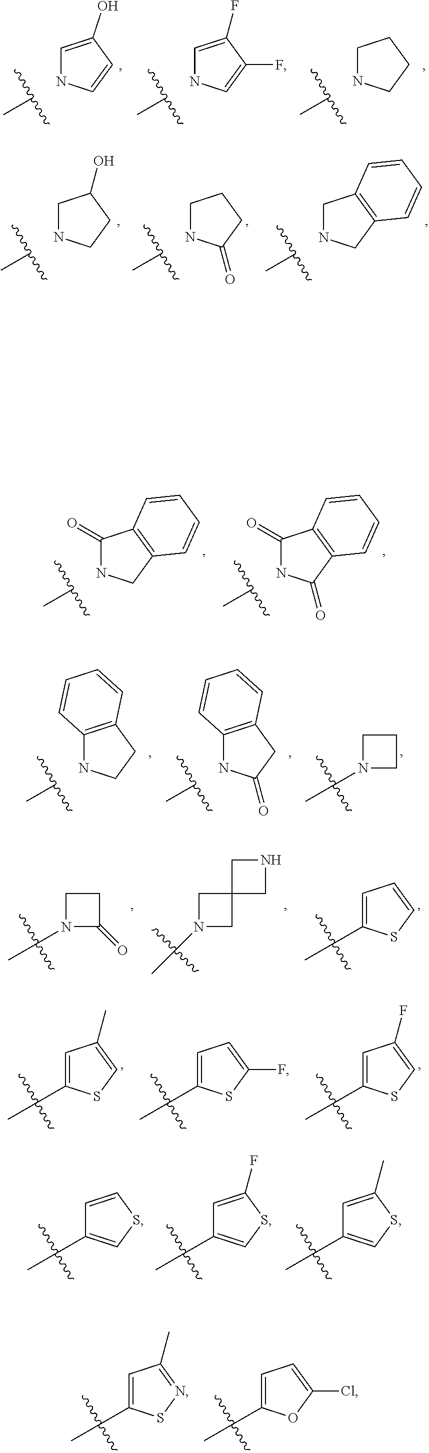

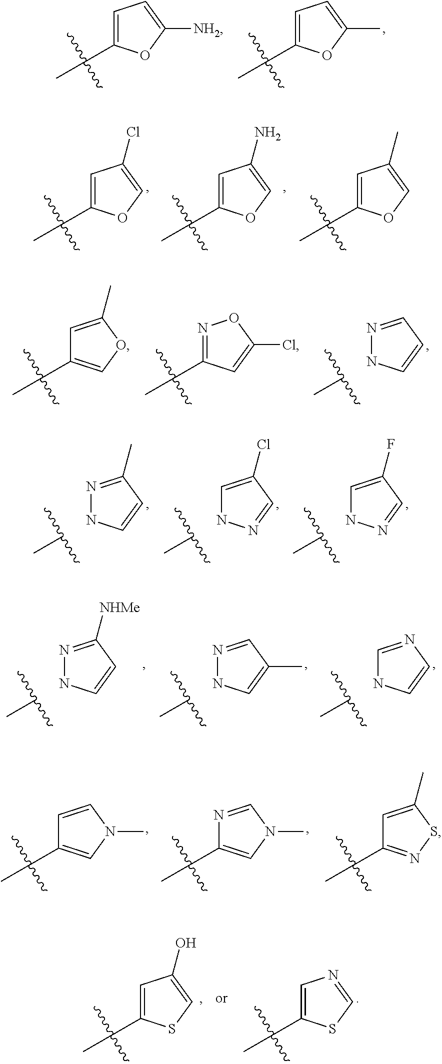

R² can be H. R² can be substituted or unsubstituted alkyl. R² can be C₁₋₄-alkyl. R² can be CHF₂. R² can be —CF₃. R² can be —CN. R² can be —ORC. R² can be —OH. R² can be OMe. R² can be halogen. R² can be Cl or F. R² can be substituted or unsubstituted heterocyclyl.
R² can be substituted or unsubstituted 5-membered heterocyclyl.
R² can be substituted or unsubstituted 6-membered heterocyclyl.

R³ can be H, R³ can be halogen. R³ can be Cl or F. R³ can be —CHF₂. R³ can be —CF₃. R³ can be —CN. R³ can be —ORC. R³ can be —OCF₃.

R⁴ can be H, R⁴ can be halogen. R⁴ can be Cl or F. R⁴ can be —CHF₂. R⁴ can be —CF₃. R⁴ can be —CN. R⁴ can be —ORC. R⁴ can be —OCF₃.

R³ and R⁴ can be the same, for example, R³ and R⁴ can both be halogen, such as Cl.

R³ and R⁴ can be the different, for example, R³ and R⁴ can both be halogen, but R³ is Cl and R⁴ is F, or R³ is H and R⁴ is halogen.

R^{5a} can be H. R^{5a} can be substituted or unsubstituted alkyl. R^{5a} can be methyl. R^{5a} can be substituted and unsubstituted aryl. R^{5a} can be substituted or unsubstituted cycloalkyl.

R^{5b} can be H. R^{5b} can be substituted or unsubstituted alkyl. R^{5b} can be methyl. R^{5b} can be substituted or unsubstituted aryl. R^{5b} can be substituted or unsubstituted cycloalkyl.

R^{5a} and R^{5b} can be the same, for example R^{5a} and R$$ can both be methyl. R⁵³ and R⁵⁰ can be the different, for example R^{5a} can be H. and R^{5b} can be methyl.

R⁶ can be N(H)R^{a}. R⁶ can be NH₂. R⁶ can be N(H)C(O)OtBu. R⁶ can be O-alkyl. R⁶ can be OH. R⁶ can be —CO₂R^{d}. R⁶ can be —OCH₃, —OC₂H₅, —OC₃H₇, —OC₄H₉, —OCH₁₁, —OC₆H₁₃, —OC₇H₁₅, —OC₈H₁₇, —OC₁₀H₂₁, —OC₁₂H₂₅, —OC₁₄H₂₉ or —OC₁₆H₃₃. R⁶ can be —NHCH₃, —NHC₂H₅, —NHC₃H₇, —NHC₄H₉, —NHC₆H₁₃, —NHC—H₁₅, —NHC₈H₁₇, —NHC₁₀H₂₁, —NHC₁₄H₂₉ or —NHC₁₆H₃₃.

R^{5a} and R⁶ can be taken together to form a substituted or unsubstituted 5-membered heterocyclyl. R^{5a} and R⁶ can be taken together to form a substituted or unsubstituted 6-membered heterocyclyl.

R^{7a} can be H. R^{7a} can be alkyl.

R^{7b} can be H. R^{7b} can be alkyl.

R^{7a} and R^{7b} can be the same, for example R^{7a} and R^{7b} can both be methyl. R^{7a} and R^{7b} can be the different, for example R^{7a} can be H, and R^{7b} can be methyl.

R^{7a} and R⁷⁸ can be taken together with the carbon to which they are attached to form a 3-membered aliphatic carbocyclic ring.

R^{a} can be H. R^{a} can be substituted or unsubstituted alkyl. R^{a} can be —COR^{b}. R^{a} can be —CON(H)R^{b}. R^{a} can be —CO₂R^{e}. R^{a} can be —CO₂tBu.

R^{b} can be substituted or unsubstituted alkyl. R^{b} can be substituted or unsubstituted aryl.

R^{e} can be H. R^{c} can be substituted or unsubstituted alkyl. R^{e} can be substituted or unsubstituted-C(O)-alkyl,

R^{d} can be H. R^{d} can be substituted or unsubstituted alkyl.

R^{e} can be substituted or unsubstituted alkyl. R^{e} can be substituted or unsubstituted aryl.

X can be NH. X can be NMe. X can be NEt. X can be O. X can be S.

[symbol] can be a single bond. [symbol] can be a double bond.

A compound of formula (I) can be a compound selected from:

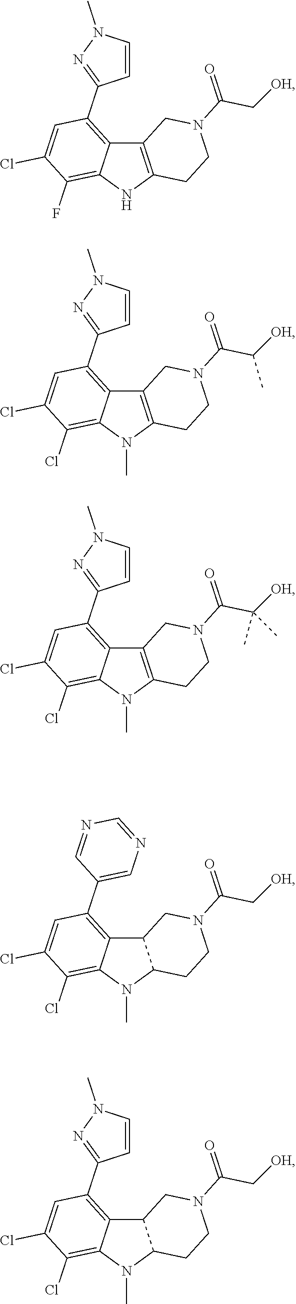

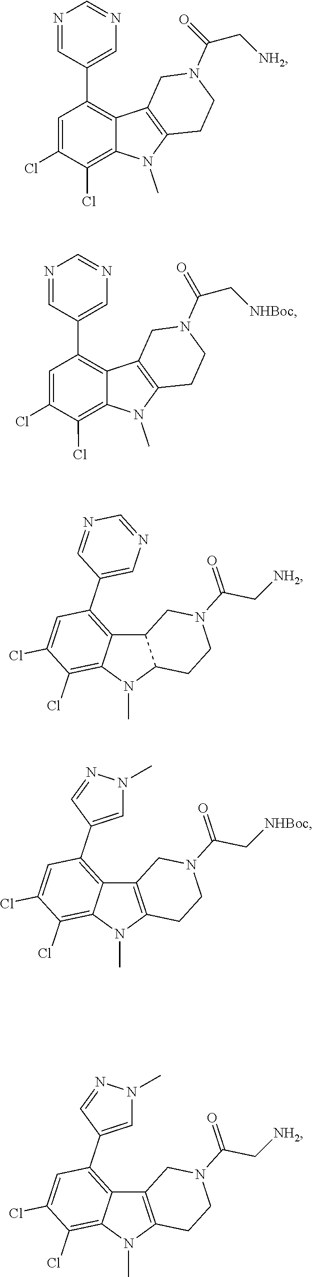

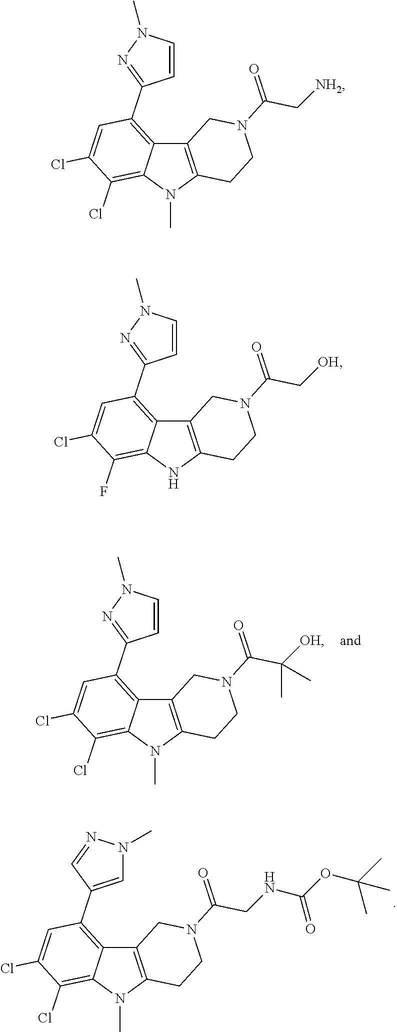

A compound of formula (I) can be a compound selected from:

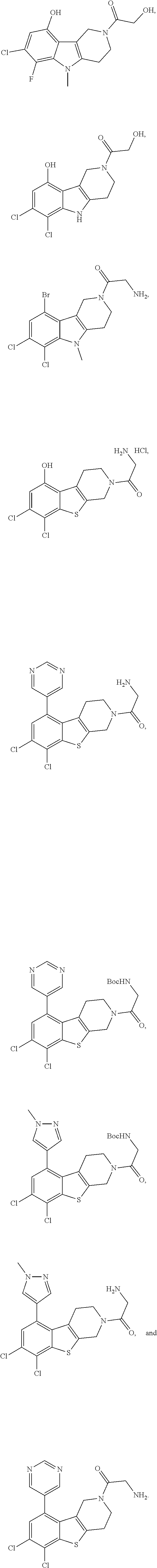

A compound of formula (II) can be a compound selected from:

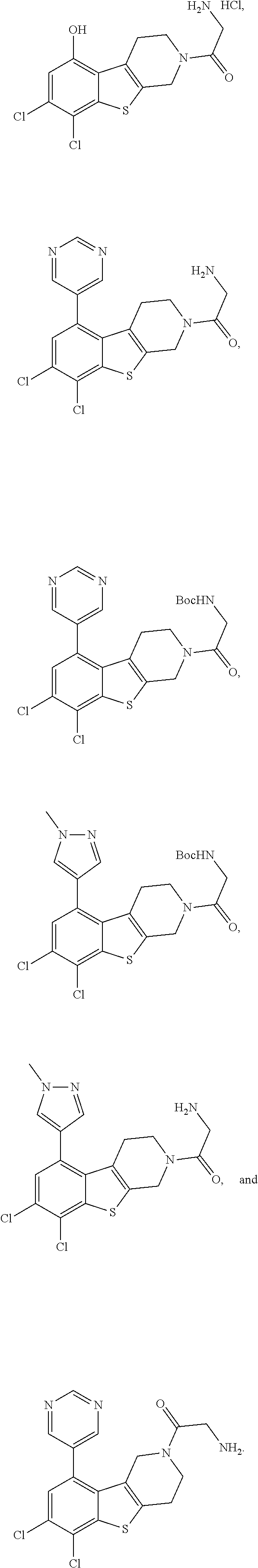

A compound of formula (I), or (II) can be a compound from Table 1.

All diastereomers of the compounds of the formulae (I)—(II) are contemplated herein.

Also contemplated herein are isotopomers, which are compounds where one or more atoms in the compound has been replaced with an isotope of that atom. Thus, for example, the disclosure relates to compounds wherein one or more hydrogen atoms is replaced with a deuterium or wherein a fluorine atom is replaced with an ¹⁹F atom.

Compounds of the disclosure can be synthesized by any method well known in the art.

### Methods of Treatment

The disclosure relates to a method of treating cGAS-related diseases or disorders comprising the step of administering to a subject in need thereof a therapeutically effective amount of any one of the aforementioned compounds.

The disclosure relates to a method of inhibiting an inflammatory response comprising the step of administering to a subject in need thereof a therapeutically effective amount of any one of the aforementioned compounds

The disclosure relates to a method of inhibiting dsDNA-triggered interferon expression comprising the step of administering to a subject in need thereof a therapeutically effective amount of any one of the aforementioned compounds.

This disclosure further provides a method of treating a neurodegenerative disease or disorder comprising the step of administering to a subject in need thereof a therapeutically effective amount of any one of the aforementioned compounds.

The neurodegenerative disease or disorder can be selected from the group consisting of AIDS dementia complex, Alzheimer's disease, amyotrophic lateral sclerosis, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Huntington's disease, Kennedy's disease, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome. In one embodiment, the neurodegenerative disease is selected from the group consisting of Alzheimer's disease, amyotrophic lateral sclerosis, Huntington's disease, and Parkinson's disease.

The neurodegenerative disease or disorder can be selected from the group consisting of Alzheimer's Disease, Parkinson's disease, Tauopathies, or Frontotemporal dementia.

This disclosure further provides a method of treating epilepsy, including Dravet syndrome and other drug-resistant seizure.

This disclosure further provides a method of treating viral infection-associated dementia, such as HIV dementia.

This disclosure further provides a method of treating neurological disorders associated with COVID.

### Pharmaceutical Compositions, Routes of Administration, and Dosing

In certain embodiments, the disclosure is directed to a pharmaceutical composition, comprising a compound of the disclosure and a pharmaceutically acceptable carrier. In certain embodiments, the pharmaceutical composition comprises a plurality of compounds of the disclosure and a pharmaceutically acceptable carrier.

In certain embodiments, a pharmaceutical composition of the disclosure further comprises at least one additional pharmaceutically active agent other than a compound of the disclosure. The at least one additional pharmaceutically active agent can be an agent useful in the treatment of ischemia-reperfusion injury.

Pharmaceutical compositions of the disclosure can be prepared by combining one or more compounds of the disclosure with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the disclosure being administered, the size of the subject, or the severity of the disease or condition. One of ordinary skill in the art can empirically determine the effective amount of a particular compound of the disclosure and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1000 milligrams/kg per day. Oral doses in the range of 0.5 to 50 milligrams/kg, in one or more administrations per day, can yield therapeutic results. Dosage may be adjusted appropriately to achieve desired drug levels, local or systemic, depending upon the mode of administration. For example, intravenous administration may vary from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound of the disclosure can be administered in an amount equal or equivalent to 0.2-2000 milligram (mg) of compound per kilogram (kg) of body weight of the subject per day. The compounds of the disclosure can be administered in a dose equal or equivalent to 2-2000 mg of compound per kg body weight of the subject per day. The compounds of the disclosure can be administered in a dose equal or equivalent to 20-2000 mg of compound per kg body weight of the subject per day. The compounds of the disclosure can be administered in a dose equal or equivalent to 50-2000 mg of compound per kg body weight of the subject per day. The compounds of the disclosure can be administered in a dose equal or equivalent to 100-2000 mg of compound per kg body weight of the subject per day. The compounds of the disclosure can be administered in a dose equal or equivalent to 200-2000 mg of compound per kg body weight of the subject per day. Where a precursor or prodrug of the compounds of the disclosure is to be administered rather than the compound itself, it is administered in an amount that is equivalent to, i.e., sufficient to deliver, the above-stated amounts of the compounds of the invention.

The formulations of the compounds of the disclosure can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

The compounds of the disclosure can be administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed by the present invention.

The compounds of the disclosure can be administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds of the disclosure can be administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound or precursor thereof can be administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed by the present invention.

The compound or precursor thereof can be administered at concentrations that range from 0.10 microgram/mL to 500.0 microgram/mL. For example, the concentration may be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed by the present invention.

The formulations of the disclosure can be administered in pharmaceutically acceptable solutions, which may routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the disclosure can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also contemplated are oral dosage forms of the compounds of the disclosure. The compounds of the disclosure may be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

The location of release of a compound of the disclosure may be the stomach, the small intestine (the duodenum, the jejunum, or the ileum), or the large intestine. One skilled in the art has available formulations which will not dissolve in the stomach, yet will release the material in the duodenum or elsewhere in the intestine. The release can avoid the deleterious effects of the stomach environment, either by protection of the compound of the disclosure or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the disclosure may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the disclosure or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (a1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this disclosure are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the disclosure. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the disclosure may also be prepared in different formulations depending on the type of chemical modification or the type of device employed.

Formulations suitable for use with a nebulizer, either jet or ultrasonic, will typically comprise a compound of the disclosure dissolved in water at a concentration of about 0.1 to 25 mg of biologically active compound of the disclosure per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the disclosure caused by atomization of the solution in forming the aerosol.

Formulations for use with a metered-dose inhaler device will generally comprise a finely divided powder containing the compound of the disclosure suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the disclosure and may also include a bulking agent, such as lactose, sorbitol, sucrose, or mannitol in amounts which facilitate dispersal of the powder from the device, e.g., 50 to 90% by weight of the formulation. The compound of the disclosure can be prepared in particulate form with an average particle size of less than 10 micrometers (mm), or 0.5 to 5 mm, for delivery to the deep lung.

Nasal delivery of a pharmaceutical composition of the present disclosure is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present disclosure solution into a chamber of defined volume, which chamber has an aperture dimensioned to aerosolize and aerosol formulation by forming a spray when a liquid in the chamber is compressed. The chamber is compressed to administer the pharmaceutical composition of the present disclosure. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. The nasal inhaler can provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the disclosure contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

The therapeutic agent(s), including specifically but not limited to a compound of the disclosure, may be provided in particles. Particles as used herein means nanoparticles or microparticles (or in some instances larger particles) which can consist in whole or in part of the compound of the disclosure or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the disclosure in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that can results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the disclosure contained herein in view of information known to the ordinarily skilled artisan, and may be made without departing from the scope of the disclosure or any embodiment thereof. Having now described the present disclosure in detail, the same will be more clearly understood by reference to the following examples, which are included herewith for purposes of illustration only and are not intended to be limiting of the disclosure.

### Examples

The invention now being generally described, it will be more readily understood by reference to the following examples, which are included merely for purposes of illustration of certain aspects and embodiments of the present invention and are not intended to limit the invention.

### Material and Methods

### Mice

P301S transgenic mice (https://www.jax.org/strain/008169) were crossed with cGas knockout mice (https://www.jax.org/strain/026554) to generate littermate P301S cGas+/+ mice, P301S cGas+/− and littermate P301S cGas−/− mice, as well as non-transgenic littermates. Mice of both sexes were used behavioral, histological and biochemical analyses. Mice underwent behavioral testing at 7 to 8 months of age and had not been used for any other experiments. At 9 to 10 months of age, the same mice were used for pathology and RNA-seq studies. For TDI-6570 in vivo treatment, P301S and non-transgenic littermate mice at 6-7 months were used for diet experiments and were assayed for behavior and histology at 9-10 months. Mice were housed under specific pathogen-free conditions under a 12 h light-dark cycle and all mouse protocols were approved by the Institutional Animal Care and Use Committee, University of California, San Francisco.

### RNA Isolation

Brains recovered from freshly perfused mice, were dissected to isolate hippocampi and cortices. Hippocampi were split in two and frozen at −80° C., until experimentation. To isolate RNA, hippocampi were thawed on ice for 30 mins and then homogenized. Briefly, hippocampi were passed through using a 21 G needle in solution of RLT buffer containing 1% β-mercaptoethanol. Following homogenization, samples were spun down briefly and then frozen at −80° C. overnight. The next day, samples were thawed on ice and then spun down at 4° C. for 6 min at 14000 rpm. RNA isolation was performed on hippocampal lysates according to manufacturer's protocol (RNeasy mini-kit, Qiagen). Isolated RNA was submitted to Weill Cornell Medicine Genomics Core for analysis of RNA quality and integrity. All samples passed QC and RNA sequencing libraries were prepared for sequencing using NovaSeq. For validation real-time qPCR experiments, cDNA was prepared using iScript Reverse Transcription supermix (BioRad). Rt-qPCR was performed on an ABI7900HT sequence detector (Applied biosystems) using SYBR green PCR master mix (Applied Biosystems) in triplicate. The change in Ct values between the transcript of interest and mouse GAPDH was calculated. The relative gene expression was then determined using 2^{ΔCt} then expressed as a relative fold change.

### Western Blot:

For mouse brain samples, half hippocampi were mechanically homogenized on ice in RIPA buffer containing protease and phosphatase inhibitors (Millipore Sigma). 50 ug of hippocampal lysates were used to analyze protein expression in the brain. Samples were loaded into on NuPage Bis-Tris gels (ThermoFisher) and run in SDS running buffer, at 150V for ˜2.5 hours. Gels were transferred on to methanol activated nitrocellulose membrane (BioRad) overnight in a cold room. Membranes were washed 3×10 min in TBS with 0.01% Triton X-100 (TBST) and blocked for 1 hr in 5% milk TBST. Appropriate primary antibodies were diluted in 1% milk TBST and incubated at 4° C. overnight. The following day, membranes were washed 3×10 min TBST then incubated with appropriate secondary antibodies in 1% milk TBST for 1 hr at room temperature. Membranes were washed again to minimize non-specific binding and then treated with ECL (BioRad) for 60 seconds and developed in a dark room. Blots were scanned at 300 DPI and quantified using ImageJ.

For human brain samples, frontal cortex lysates were prepared as described previously (Min et al., 2010). Briefly, human or mouse brain tissues were lysed in RIPA buffer containing protease inhibitor cocktail (Sigma), 1 mM phenylmethyl sulfonyl fluoride (Sigma), phosphatase inhibitor cocktail (Roche), and HDAC inhibitors, including 5 mM nicotinamide (Sigma) and 1 mM trichostatin A (Sigma). After sonication, lysates were centrifuged at 170,000 g at 4° C. for 15 min. Supernatants were collected and analyzed by Western blot. Protein concentration was measured by BCA assay (Thermo Scientific).

For cultured primary microglia, 1-2 million cells were lysed in M-PER™ Mammalian Protein Extraction Reagent (Thermo Scientific) supplemented with HALT protease and phosphatase inhibitor cocktail (Thermo Scientific) and 150 mM NaCl. Samples were rotated at 4° C. for 10 min. Lysates were cleared by centrifugation at 16000×g for 15 min at 4° C. Protein concentration was measured by BCA assay.

Nuclei Isolation from Frozen Mouse Hippocampi

Hippocampi isolation from frozen mouse hippocampi was adapted from a previous study with modifications (Grubman et al., 2019; Habib et al., 2017). All procedures were done on ice or at 4° C. In brief, post-mortem brain tissue was placed in 1500 μl of Sigma nuclei PURE lysis buffer (Sigma, NUC201-1KT) and homogenized with a Dounce tissue grinder (Sigma, D8938-1SET) with 20 strokes with pestle A and 15 strokes with pestle B. The homogenized tissue was filtered through a 35-μm cell strainer, then centrifuged at 600 g for 5 min at 4° C. and washed three times with 1 ml of PBS containing 1% BSA, 20 mM DTT and 0.2 U μl⁻¹ recombinant RNase inhibitor. Then the nuclei were centrifuged at 600 g for 5 min at 4° C. and re-suspended in 800 μl of PBS containing 0.04% BSA and 1× DAPI, followed by FACS sorting to remove cell debris. The FACS-sorted suspension of DAPI-stained nuclei were counted and diluted to a concentration of 1000 nuclei per microliter in PBS containing 0.04% BSA.

### Droplet-Based Single Nuclei RNA Sequencing

For droplet-based snRNA-seq, libraries were prepared with Chromium Single Cell 3′ Reagent Kits v3 (10× Genomics, PN-1000075) according to the manufacturer's protocol. The snRNA-seq libraries were sequenced on the NovaSeq 6000 sequencer (Illumina) with 100 cycles.

Analysis of Droplet-Based Single-Nuclei RNA-Seq Data from Human Brain Tissue

Gene counts were obtained by aligning reads to the mm10 genome with Cell Ranger software (v.3.1.0) (10× Genomics). To account for unspliced nuclear transcripts, reads mapping to pre-mRNA were counted. Cell Ranger 3.1.0 default parameters were used to call cell barcodes. We further removed genes expressed in no more than 2 cells, cells with unique gene counts over 4,000 or less than 200, and cells with high fraction of mitochondrial reads (>5%). Potential doublet cells were predicted using DoubletFinder (McGinnis et al., 2019) for each sample separately with high confidence doublets removed. Normalization and clustering were done with the Seurat package v3.2.2 (Stuart et al., 2019). In brief, counts for all nuclei were scaled by the total library size multiplied by a scale factor (10,000), and transformed to log space. A set of 2000 highly variable genes were identified with SCTransform from sctransform R package in the variable stabilization mode. This returned a corrected unique molecular identifiers (UMI) count matrix, a log-transformed data matrix, and Pearson residuals from the regularized negative binomial regression model. Principal component analysis (PCA) was done on all genes, and t-SNE was run on the top 20 PCs. Cell clusters were identified with the Seurat functions FindNeighbors (using the top 20 PCs) and FindClusters (resolution=0.02). In this analysis, the neighborhood size parameter pK was estimated using the mean-variance normalized bimodality coefficient (BCmvn) approach, with 20 PCs used and pN set as 0.25 by default. For each cluster, we assigned a cell-type label using statistical enrichment for sets of marker genes (Lake et al., 2018; Wang et al., 2018) and manual evaluation of gene expression for small sets of known marker genes. Differential gene expression analysis was done using the FindMarkers function and MAST (Finak et al., 2015). To identify gene ontology and pathways enriched in the differentially expressed genes (DEGs), DEGS were analysed using (MSigDB) gene annotation database (Liberzon et al., 2011; Subramanian et al., 2005). To control for multiple testing, we employed the Benjamini-Hochberg (BH) approach to constrain the false discovery rate (FDR). For trajectory analysis, Seurat objects were converted to cds objects and analysed using Monocle 3 (Cao et al., 2019; Qiu et al., 2017; Trapnell et al., 2014). Moran's I spatial autocorrelation analysis was performed to identify gene modules significantly associated with microglial trajectory. Enrichment analysis of target genes was performed with GeneOverlap package in R. Briefly, the MEF2C, MEF2A, JunB and FOSL2 target gene list, and human cognitive resilience gene lists (kindly shared by Dr. Li Huei Tsai) and P301S Cgas−/− vs P301S EN/IN DEG lists were used as the input for the comparison. The results include the overlapping p value and the odds ratio which examines the association between the 2 datasets.

### Antibodies

Antibodies used in immunofluorescence analysis were as follows: Secondary antibodies used were Alexa fluor donkey anti-rabbit/goat 488 and anti-mouse 555, and Jackson donkey anti-goat 555 at 1:500 (Invitrogen).

STING (D2P2F, Cell Signaling Technology, 1:300), anti-IBA1 (ab5076, Abcam, 1:500), anti-PSD-95 (1:500, Millipore MAB1596), anti-vGAT (Ab5062, Millipore, 1:500), anti-pSTAT1 (mAb9167, Cell Signaling, 1:500), anti-NRG1 (MA5-12896, Invitrogen, 1:100), MC1 (a generous gift from Dr. Peter Davis), anti-MEF2C (MAB6786, R&D systems1: 200)

Antibodies used in western blot were as follows: STING (as above), TBK1 (D1B4, Cell Signaling Technology, 1:1000), pTBK1 (D52C2, Cell Signaling Technology, 1:500), Caspase-3 (9661, Cell Signaling Technology, 1:1000), GAPDH (MAB374, Millipore, 1:10000 and GTX100118, GeneTex, 1:10000). Secondaries used were anti-rabbit HRP (401393, Calbiochem, 1:2000) or anti-mouse HRP (401253, Calbiochem, 1:2000), anti-NeuN (ABN78, Millipore, 1:500). For Immuno-gold labeling electron microscopy antibody against tau (A0024, Agilent Technologies, 1:1000) was used,

### Immunofluorescence:

Hemibrains from transcardially perfused mice were placed in 4% paraformaldehyde for 48 h, followed by 30% sucrose PBS for 48 h at 4° C. Sections were cut coronally at 40 μm using a freezing microtome (Leica) and placed in cryoprotective medium at −20° C. until use. 8-10 free floating sections per mouse. All washing steps were 3×5 min. Sections were washed in TBST (0.01% Triton X-100), permeabilized with TBST (0.5% Triton X-100) for 15 min, then washed again. Sections were then placed in antigen unmasking solution (citrate buffer, pH 6.0, h-3300) and placed in a 90° C. incubator for 30 min, when applicable. Sections were washed and then blocked in 10% normal donkey serum (NDS, Vector BMK-2202) in PBST for 2 hours at room temperature. Primary antibodies were diluted in 5% NDS PBST and incubated overnight at 4° C. The following day, sections were washed thoroughly and incubated in appropriate secondary antibodies (1:500, Invitrogen) for 1 hour. Sections were washed, mounted on slides and imaged using a Keyence BZ—X700 microscope. For pSTAT1 imaging, CA1 region of the mouse brain sections was imaged with Zeiss Apotome 20× objective (Carl Zeiss). Images were taken with Z stacks of 7 μm interval at 1 μm step size. For MEF2C, Images of the mouse brain CA1 pyramidal region were acquired using 25× objective of the Zeiss LSM880 confocal microscope (Carl Zeiss). A 4 by 1 tile scan and Z stack of 12 μm at a step size of 3 μm were used to generate maximum intensity projection, stitched images for each section. For PSD-95, vGAT and NRG1, slides were imaged using a Zeiss LSM 880 Confocal microscope. Quantification was done using ImageJ software (NIH) using percentage area based on thresholding determined using negative and positive controls. For higher resolution, images were acquired with LSM 880 confocal microscopy and Zen Black image acquisition software with 40× objective. The CA1 region of the hippocampus was imaged with 1 μm interval Z stack over a total distance of 15 μm per slice and a 2×2 tile scan. Final images were processed with maximum intensity projection.

### Behavioral Studies

cGas+/+ mice, P301S cGas+/− and littermate P301S cGas−/− mice were compared to their respective nontransgenic or P301S littermates. Experimenters were blinded to mouse genotypes throughout the experiments. Male and female mice were tested on separated days.

Cgas+/+, Cgas+/− and Cgas−/− mice were compared to their respective P301S transgenic littermates. Male and female mice were tested on separated days and experimenters were blinded to mouse genotypes throughout the experiments.

For experiments involving TDI-6570 treatment, male P301S and Non-transgenic littermates fed with TDI-6570 or control diet were used.

### Morris Water Maze

The water maze consists of a pool (122 cm in diameter) containing opaque water (20±1° C.) and a platform (10 cm in diameter) 1.5 cm below the surface. Three different images were posted on the walls of the room as spatial cues. Hidden platform training (days 1-7) consisted of 14 sessions (two per day, 2 hrs apart), each with two trials. The mouse was placed into the pool at alternating quadrants for each trial. A trial ended when the mouse located the platform or after 60 sec had elapsed. At 24 and 72 hrs after training, the mice were tested in probe trials, in which the hidden platform was removed, and mice were allowed to swim for 60 sec. Mice received 7 days of hidden platform training before the 24-hr and 72-hr probe trials. Visible platform testing was done 24 hrs after the last probe trial. Performance was measured with an Etho Vision video tracking system (Noldus Information Technology).

### Elevated Plus Maze

The maze consists of two 15×2-inch open arms without walls and two closed arms with walls 6.5 inches tall and is 30.5 inches above the ground. Mice were moved to the testing room 1 hr before testing to acclimate to the dim lighting. Mice were individually placed in the maze at the intersection of the open and closed arms and allowed to explore the maze for 10 min.

### Open Field

Mice were individually placed into brightly lit automated activity chambers equipped with rows of infrared photocells connected to a computer (San Diego Instruments). Open field activity was recorded for 5 min. Recorded beam breaks were used to calculate total time of activity.

### Novel Object Recognition Test

Mice were habituated to opaque open field arenas (40×40 cm) for two 10-minute trials spaced on the two days leading up to object recognition. Twenty-four hours after the second arena habituation trial, two identical objects (glass jars) were placed with the center of each arena. Mice were allowed to explore these objects for a single 15-minute trial. The subsequent day after object habituation, one of the identical objects were replaced with a novel object (DUPLO® block structure) for a 15-minute test period. Video recording and tracking (Ethovision v15, Noldus, Wageningen, the Netherlands) was used to determine total distance moved. An experimenter blind to the groups manually scored the time mice spent exploring each object. Preference was calculated based on the total time an individual mouse spent exploring both objects.

### Electrophysiology

The brain was quickly dissected from anesthetized mice and placed into ice-cold dissection solution containing (in mM): 210 sucrose, 2.5 KCl, 1.25 NaH2PO4, 25 NaHCO₃, 7 glucose, 2 MgSO4, and 0.5 CaCl2 (gassed with 95% 02-5% CO₂, PH ˜7.4). Horizontal slices (400 μm thickness) were made on a vibratome and then the slices were incubated for 30 minutes in artificial cerebral spinal fluid (ACSF) warmed to 35° C. containing (in mM): 119 NaCl, 2.5 KCl, 26.2 NaHCO₃, 1 NaH2PO4, 11 glucose, 1.3 MgSO4, and 2.5 CaCl2 (gassed with 95% 02-5% CO₂, pH ˜7.4). Slices were then kept at room temperature in oxygenated ACSF until recordings were performed.

Field recordings were performed in the dentate gyrus molecular layer of the acute horizontal brain slices placed in a recording chamber. Slices were submerged in oxygenated ACSF that was continuously perfused at 30° C. The glass recording electrode (˜3 M (2 pipette resistance) was filled with ACSF and lowered ˜50 μm into the molecular layer of the dorsal blade of the dentate gyrus. A bipolar tungsten electrode (FHC, Bowdoin, ME), located ˜150 μm from the recording electrode, was used to stimulate the perforant pathway inputs to the dentate gyrus. Stimulus pulses were generated by a Model 2100 Isolated Pulse Stimulator (A-M Systems). Responses were evoked every 30 s with stimulus intensities ranging from 5-40 μA with a 0.5-ms stimulus duration. After recording fEPSPs in response to 5-40 μA stimulation, the stimulus intensity was adjusted to evoke 30% of the maximal fEPSP slope to set the baseline for LTP recordings. LTP recordings were performed in the presence of picrotoxin (100 μM, Sigma). After recording baseline fEPSPs for 20 min, theta burst stimulation (TBS) was applied which included 10 theta bursts applied every 15 seconds and each theta burst consisted of 10 bursts (4 pulses, 100 Hz) every 200 ms. The stimulus intensity was raised to a level that was 60% of the maximal fEPSP slope only during TBS and then returned to the stimulus intensity used during the baseline recording following TBS. The fEPSP slope was normalized to the baseline responses before LTP induction. Recordings were performed using a Multiclamp 700B amplifier (Molecular Devices), digitized at 10 kHz, and acquired with WinLTP software (version 1.11b, University of Bristol) and analyzed using WinLTP software. Recordings and analyses were done blind to mouse genotype.

### Microglia Culture and Isolation

BV2 microglia culture. The BV2 microglia cell line was maintained in growth media-DMEM (Thermo Fisher) supplemented with 10% heat-inactivated fetal bovine serum (FBS, Hyclone) and 1% penicillin/streptomycin (Life Technologies) in HERAcell 150i incubators (Caisson Labs) at 37C with 5% CO₂. BV2 microglia were serially passaged once plates reached 80-90% confluency.

Isolation and culture of postnatal primary microglia. Primary microglial cells were harvested from mouse pups at postnatal day 1-3 (P1-P3). Briefly, the brain cortices were isolated and minced. Tissues were dissociated in 0.25% Trypsin-EDTA for 10 min at 37° C. and agitated every 5 min. Trypsin was neutralized with complete medium (DMEM (Thermo Fisher) supplemented with 10% heat-inactivated fetal bovine serum (FBS, Hyclone), and were filtered through 70 μm cell strainers (BD Falcon) and pelleted by centrifugation at 1500 rpm. Mixed glial cultures were maintained in growth medium at 37° C. and 5% CO₂ for 7-10 d in vitro. Once bright round cells began to appear in the mixed glial cultures, recombinant mouse granulocyte macrophage colony stimulating factor (1 ng/ml, Life Technologies) was added to promote microglia proliferation. Primary microglial cells were harvested by mechanical agitation after 48-72 hours and plated on poly-L lysine coated t-75 flasks (Corning) in growth media. Assays were performed 24-48 hours post microglia plating.

### Interferon Beta Reporter System

Molecular Cloning. BV2 microglia were transiently transfected with interferon beta reporter plasmid, pNiFty3-I-Lucia (Invivogen), using Lipofectamine 2000 (ThermoFisher). Transfection media was removed 6 hours post transfection and replaced with complete growth media. Selection with Zeocin was conducted for 3 weeks. Resistant cells were plated as single clones in 96-well plate (Corning). Genomic DNA was isolated from clones to confirm integration of luciferase reporter. Clones were then validated for induction of interferon in response to cGas agonists.

Luciferase reporter assays. BV2 IfnB reporter microglia were stimulated with 0 to 10 ug HT-DNA (Sigma) or cyclic GAMP (Invivogen). DNA was delivered to cells using Lipofectamine 2000 (ThermoFisher). Luciferase levels secreted in media was measured using Quanti-Luc (Invivogen). Luminescence was measured on a BioTek Synergy hybrid reader.

cGas Agonist Assays

Cell viability. BV2 microglia were treated with 0 to 100 μM TDI 6570 solubilized in DMSO. 24 hours post treatment, cell viability was measured using Cell Titer Glo assay (Promega). Briefly, cells in 96-well plate were equilibrated at room temperature for 30 minutes and then lysed. Luminescence was measure on a BioTek Synergy hybrid reader.

Functional assays. Primary postnatal microglia or BV2 IfnB reporter microglia were treated with 0-50 μM TDI6570, with or without HT-DNA transfection. RNA was isolated, cDNA prepared, and levels of cGas-related genes measured using the following primers:

Cxcl10
F: CCAAGTGCTGCCGTCATTTTC.
R: GGCTCGCAGGGATGATTTCAA
Gapdh
F: ACCACAGTCCATGCCATAC.
R: CACCACCCTGTTGCTGTAGCC
cGas
F: CATCTTCCCAGCCTGACATT.
R: CACGCTTCCTGCTATGATGA
IfnB
F: CAGCTCCAAGAAAGGACGAAC.
R: GGCAGTGTAACTCTCTGCAT

Alternatively, luciferase levels in media were measured as described above.

### Mitochondrial DNA Depletion Assay

BV2 IfnB reporter microglia were treated with 50-100 ng/ml Ethidium Bromide (EtBr, Sigma Aldrich) or 40-80 μg/ml Dideoxycytidine (ddC, Sigma Aldrich) in DMEM supplemented with 10% FBS (GIBCO), 100 units/mL penicillin, 100 μg/mL streptomycin for seven days. On day 7, cells were detached from the plate by scraping. A portion of the cells was saved for mtDNA quantification PCR assay. The rest of the cells were plated in DMEM F12 supplemented with 100 units/mL penicillin, 100 μg/mL streptomycin and let rest for 48 h. Cells were then treated with ON4R tau fibrils or ABT-737+Q-VD-Oph (ABT+QVD, 10 μM each, SelleckChem) for 24 h before being assayed for Luciferase reporter activity and cell viability.

For mtDNA depletion qPCR, DNA was extracted from cell pellets using DNeasy Blood and Tissue Kit (QIAGEN). The ratio of mtDNA (Nd2) versus genomic DNA (Tert) was measured by SybrGreen real-time PCR (Bio-rad Labratories) using the following primer pairs:

Nd2 Forward:
CCATCAACTCAATCTCACTTCTATG
Nd2 Reverse:
GAATCCTGTTAGTGGTGGAAGG
Tert Forward:
CTAGCTCATGTGTCAAGACCCTCTT
Tert Reverse:
GCCAGCACGTTTCTCTCGTT

### Enzyme-Linked Immunosorbent Assay (ELISA) and Multiplex Bead-Based Immunoassay

Cell culture media from cultured primary mouse microglia were collected 24 h after stimulation and cleared by centrifugation at 2000 rpm for 5 min. The supernatants were diluted 1:10 and assayed using VeriKine-HS™ Mouse IFN Beta Serum ELISA Kit (PBL Assay Science) according to the manufacturer's instructions. CXCL10 and CCL5 were measured with MILLIPLEX MAP Mouse Cytokine/Chemokine Magnetic Bead kit (Millipore) using a MagPix System.

### Electron Microscopy

Electron microscopy experiments were performed by the electron microscopy core facility at Weill Cornell Medicine. Cells were washed with serum-free media or appropriate buffer then fixed with a modified Karmovsky's fix of 2.5% glutaraldehyde, 4% parafomaldehye and 0.02% picric acid in 0.1M sodium cacodylate buffer at pH 7.2. Following a secondary fixation in 1% osmium tetroxide, 1.5% potassium ferricyanide, samples were dehydrated through a graded ethanol series, and embedded in situ in LX-112 resin (Ladd Research Industries). En face ultrathin sections were cut using a Diatome diamond knife (Diatome, USA, Hatfield, PA) on a Leica Ultracut S ultramicrotome (Leica, Vienna, Austria). Sections were collected on copper grids and further contrasted with lead citrate. For immunolabeling, the sections were collected on 200 mesh nickel grids. Briefly, sections were rehydrated in PBS. Unreacted aldehydes were quenched by 50 mM glycine in PBS followed by blocking for host of secondary AB (Aurion, EMS) for 15 min at RT. Primary AB incubation was done overnight at 4° C. in PBS-c (PBS+0.2% BSA-c [Aurion, EMS]). The next day, sections were washed 6× in PBS-c and incubated with secondary antibody (Aurion gold conjugate 1:100 in BB) for 1-2 h at RT. Washes were done with PBS-c and then water. Sections were then fixed in 2.5% glutaraldehye in 0.1 M buffer, PBS washed, contrasted with uranyl acetate and let air dry after final wash with water. Samples were viewed on a JEM 1400 electron microscope (JEOL, USA, Inc., Peabody, MA) operated at 120 kV. Digital images were captured on a Veleta 2K×2K CCD camera (Olympus-SIS, Germany).

### Pharmacokinetic Evaluation of TDI-6570

To test pharmacokinetics, TDI-6570 (50 mg/kg as a suspension in 0.5% methylcellulose solution in water containing 0.2% Tween 80) was administered to 8-weeks old CD-1 male mice (n=3 for each time point) intraperitoneally (IP) and both plasma (EDTA-K2) and brain tissues were collected at various time points (0.5, 2, 4, 8 and 24 hours post drug administration). Transcardial perfusion were performed with saline prior to brain collection. Bioanalysis of brain tissue and plasma extracts were performed by LC-MS/MS.

### Statistical Analysis

Statistical analysis was performed using GraphPad Prism 8. All data are expressed as the mean±SEM.

### Chemistry

All commercial chemicals and solvents were reagent grade and used without further purification. All air-sensitive reactions were performed under argon protection. Column chromatography was performed using Silica gel SNAP columns. Analytical thin layer chromatography was performed on Merck 250 μM silica gel F254 plates, and preparative thin layer chromatography on Merck 1000 μM silica gel F254 plates obtained from EMD Millipore corporation. The identity of each product was determined using mass spectrometry and NMR using CDCl₃ as solvents unless otherwise mentioned. Chemical shifts are reported in δ values in ppm downfield from TMS as the internal standard. 1H data are reported as follows: chemical shift, multiplicity (s=singlet, d=doublet, t=triplet, q=quartet, br=broad, m=multiplet), coupling constant (Hz), integration.

### Results

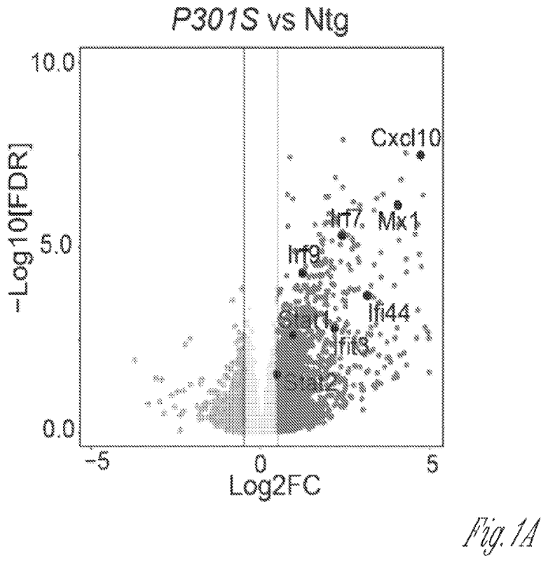

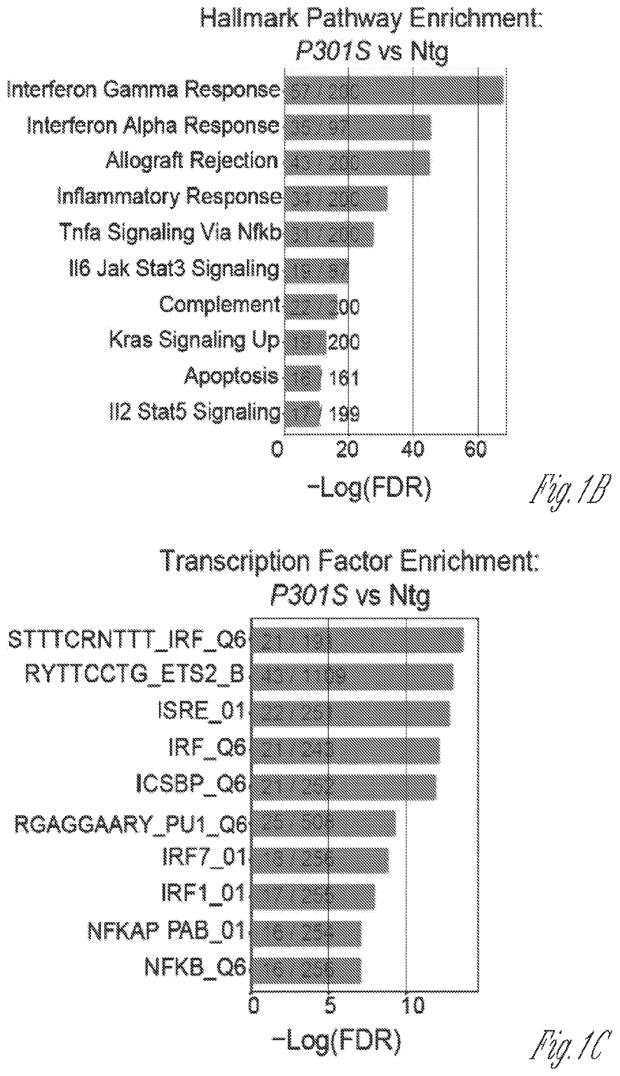

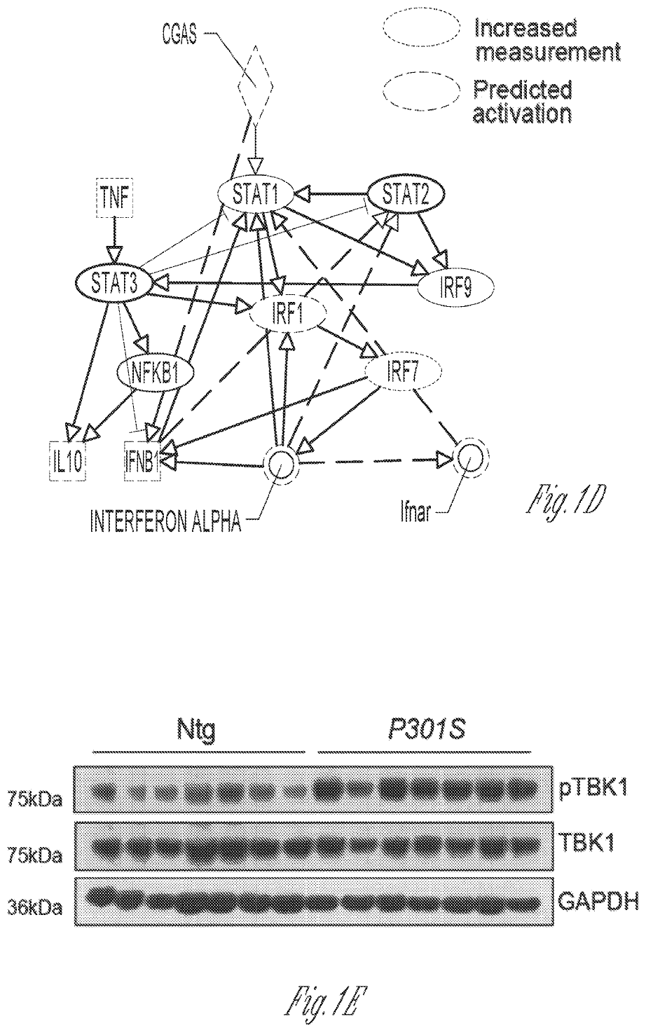

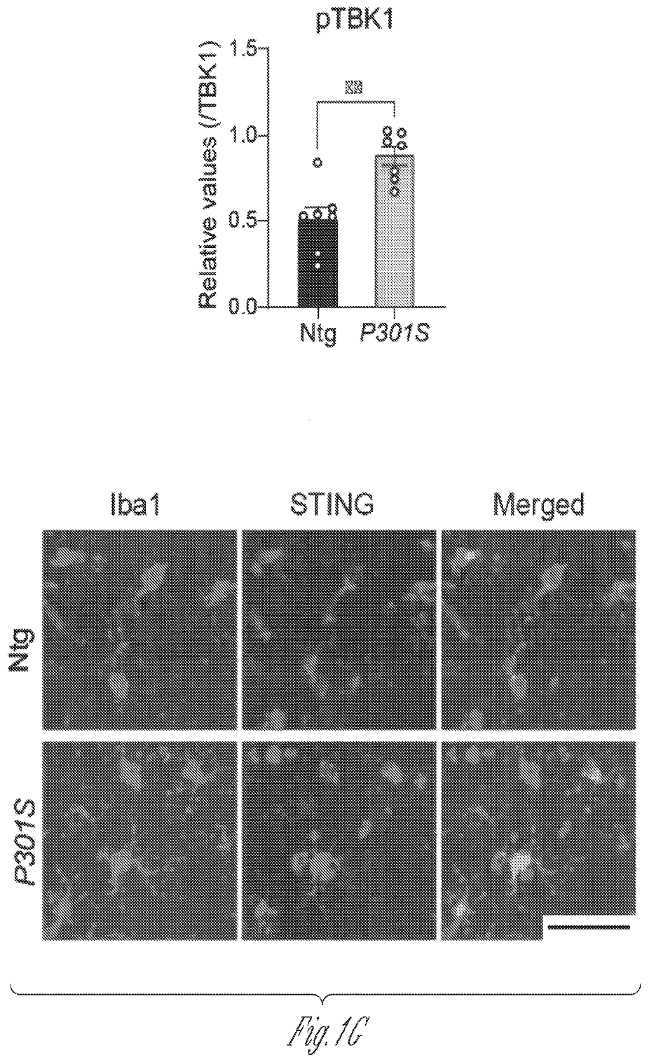

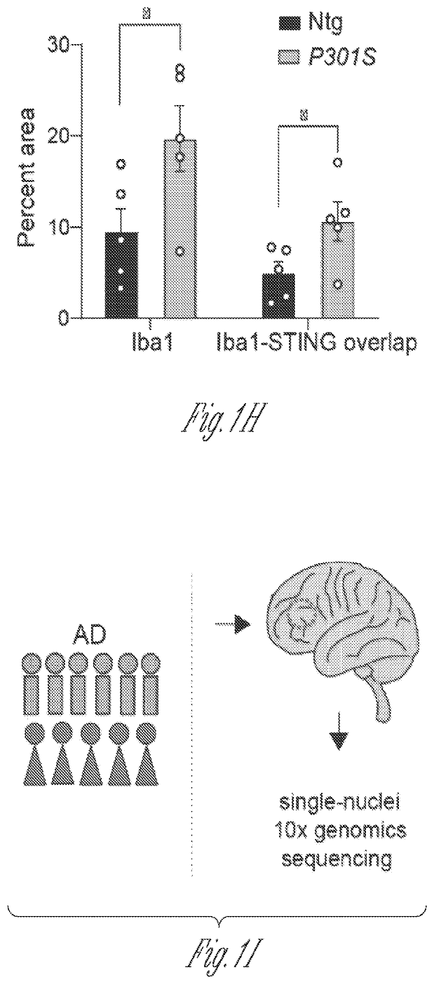

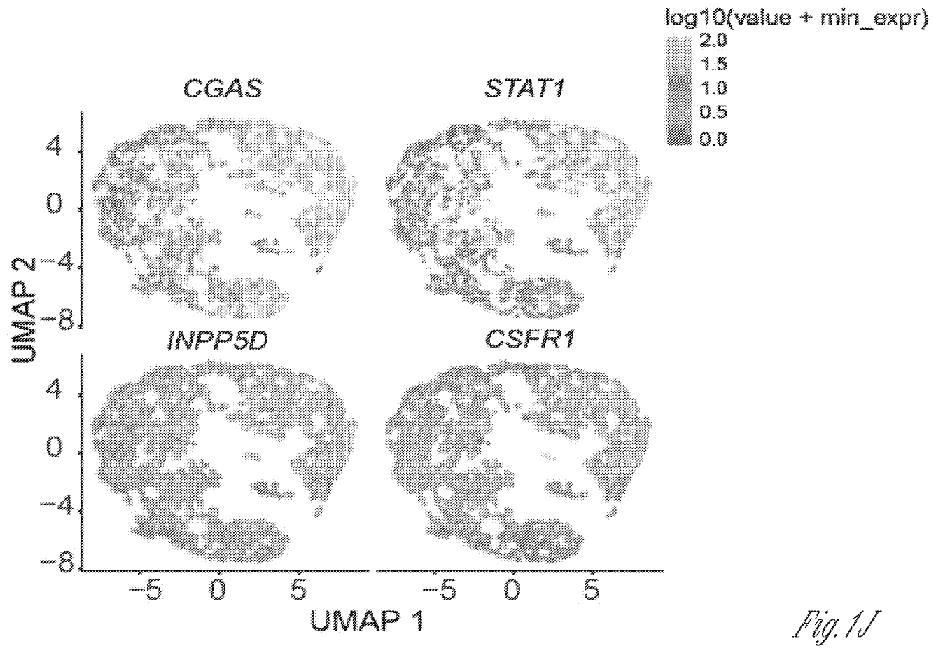

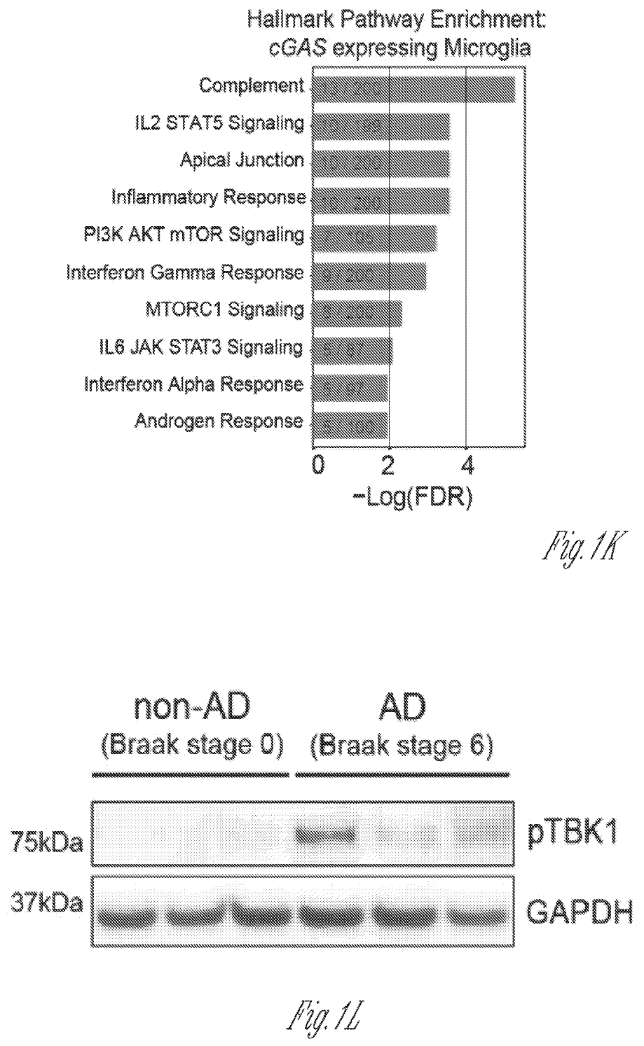

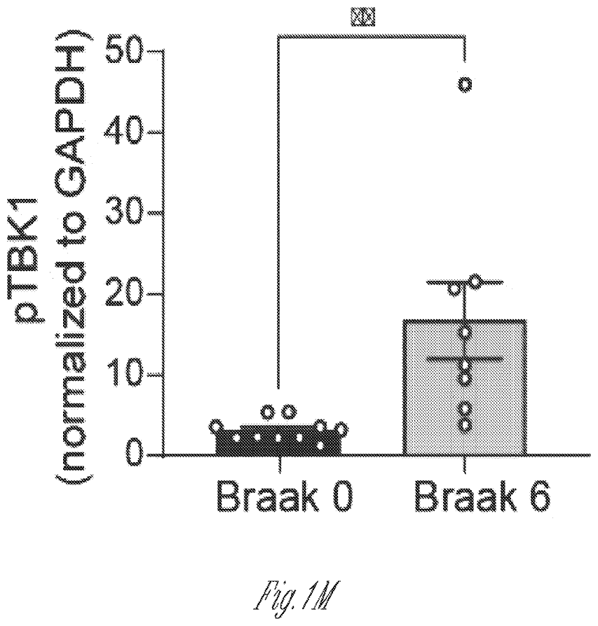

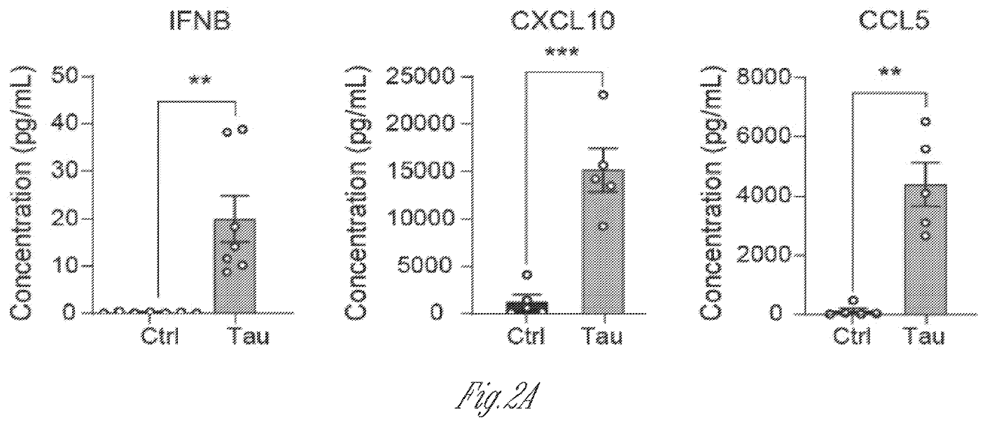

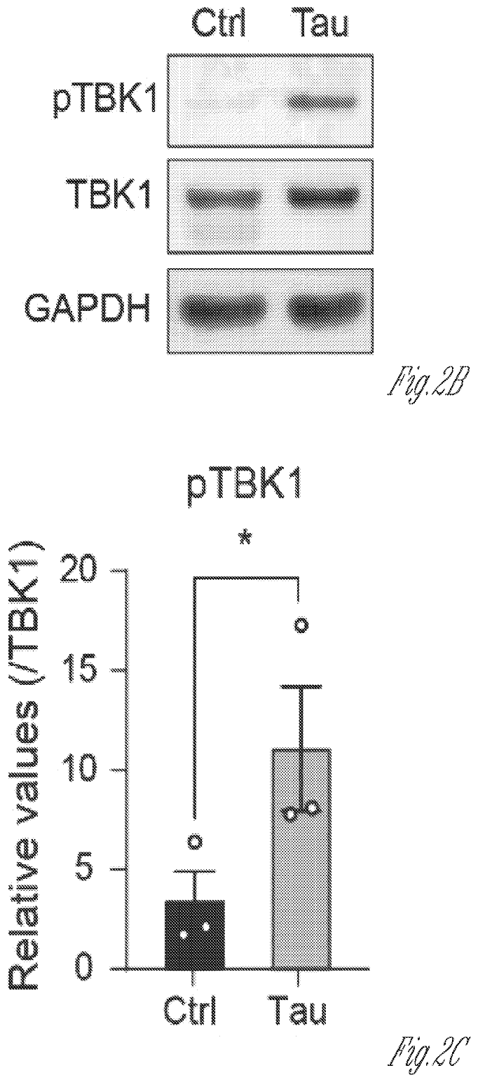

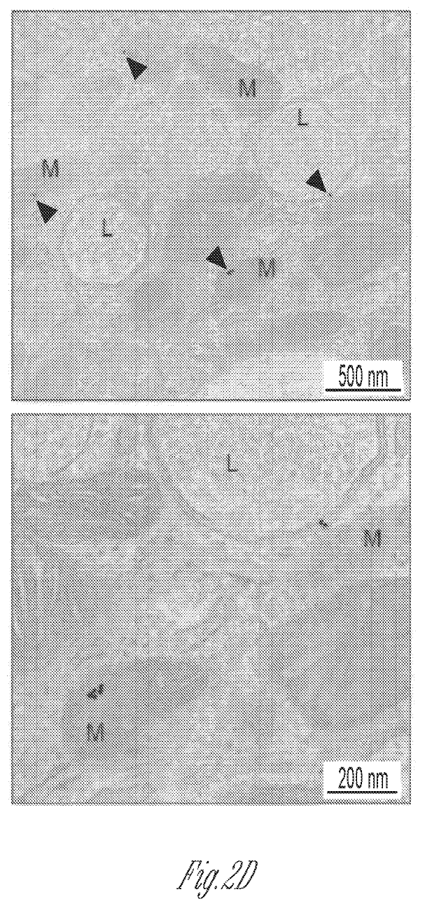

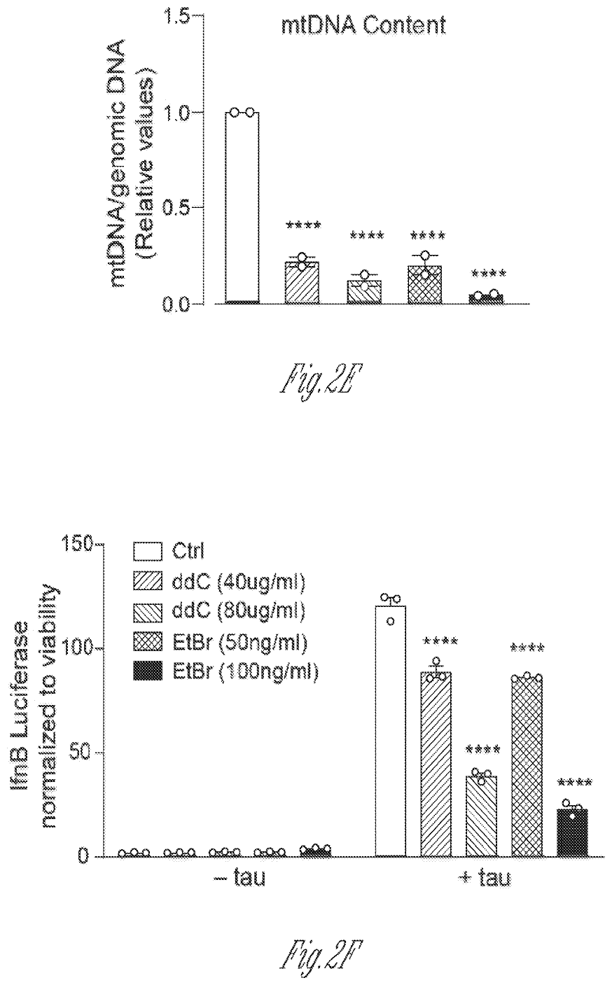

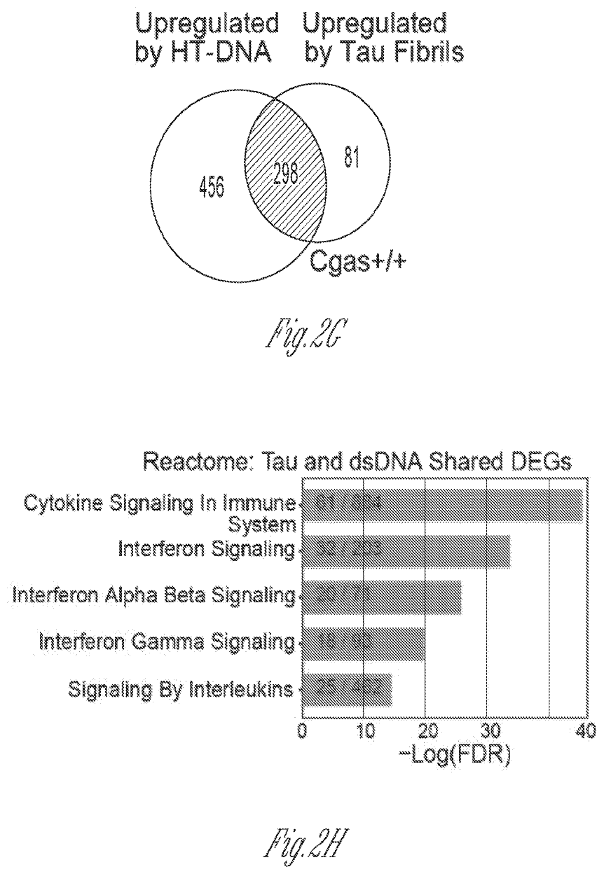

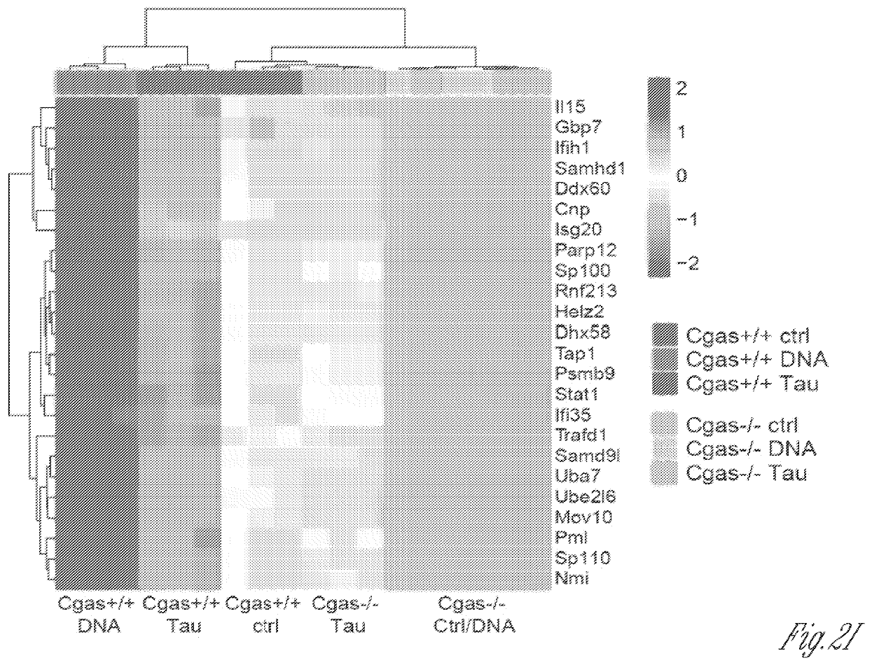

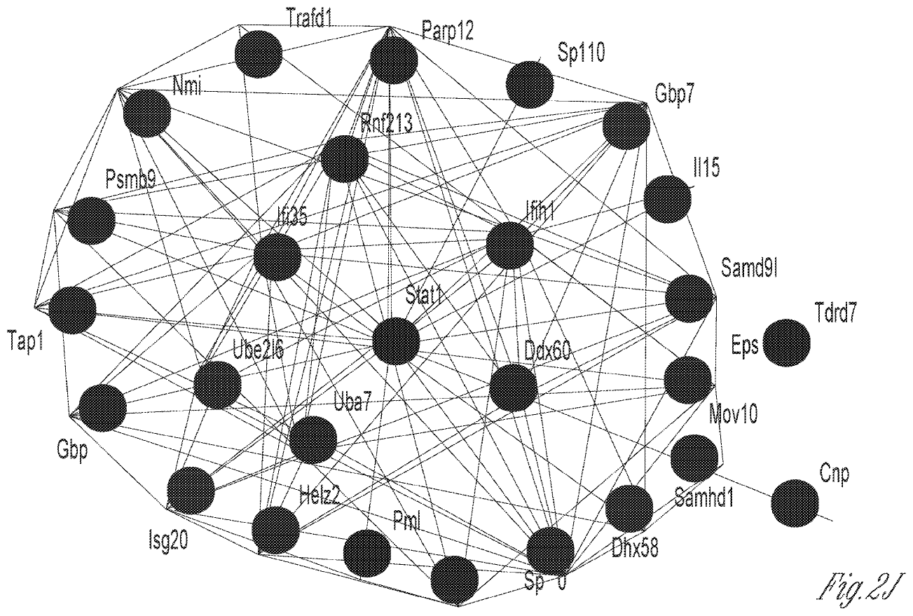

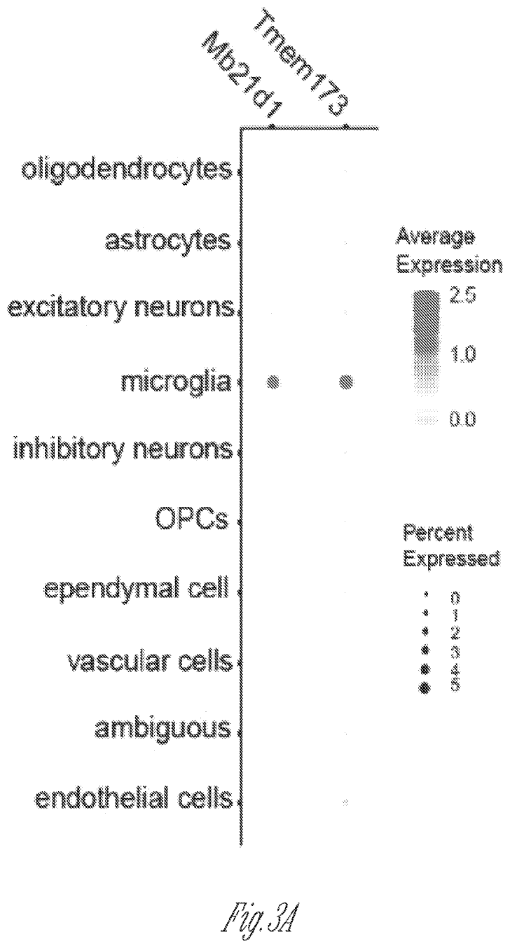

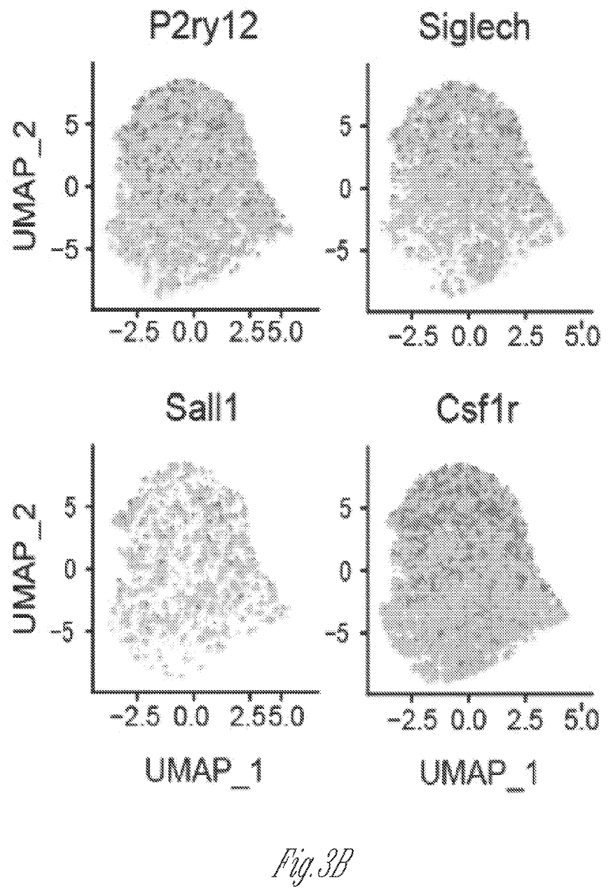

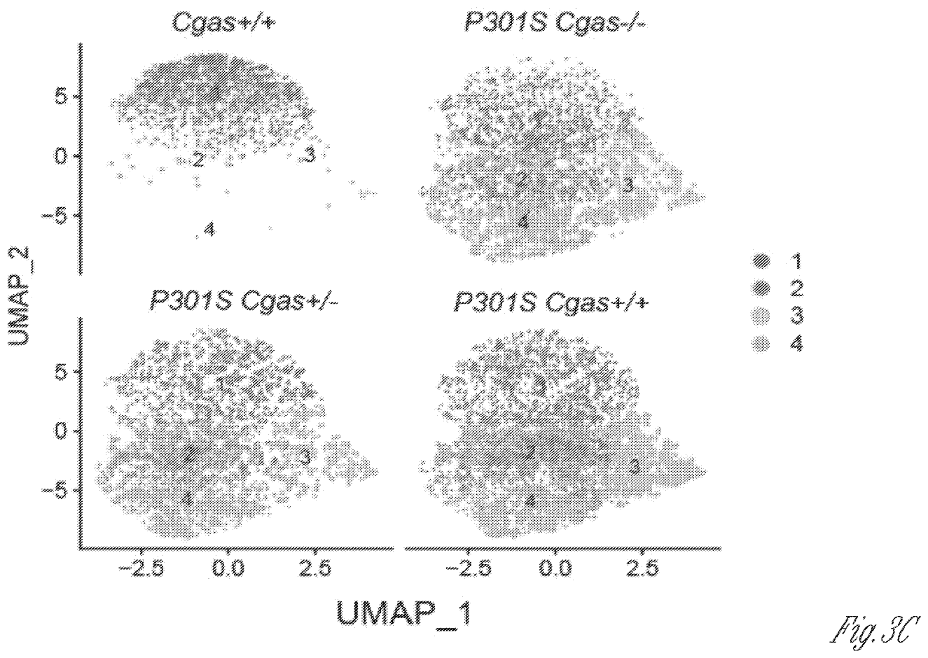

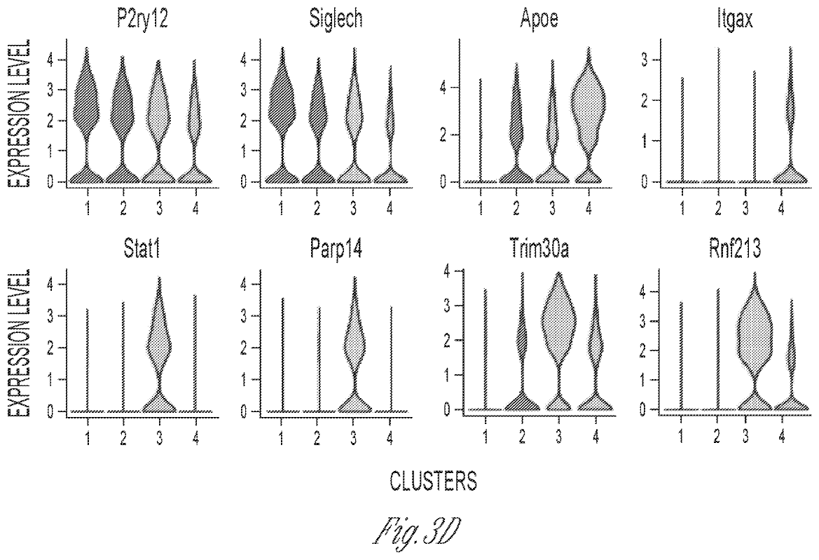

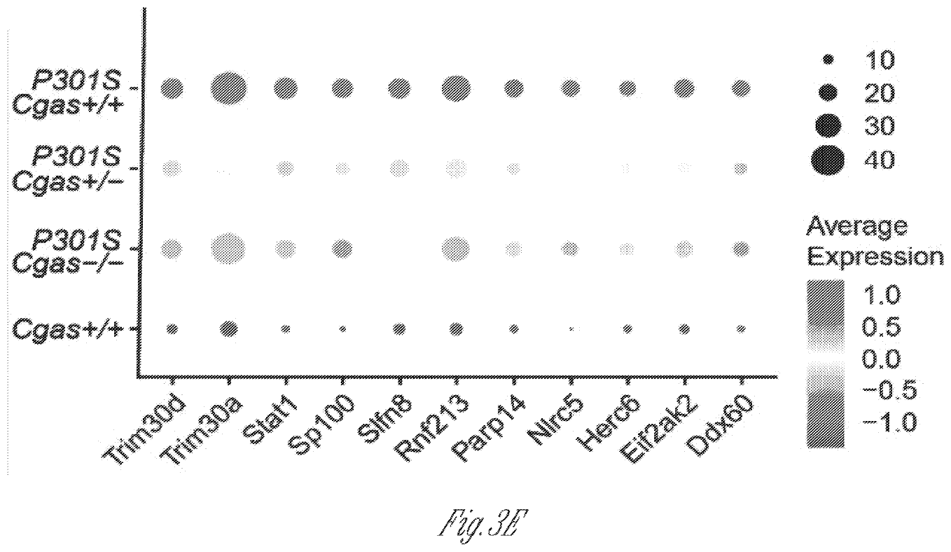

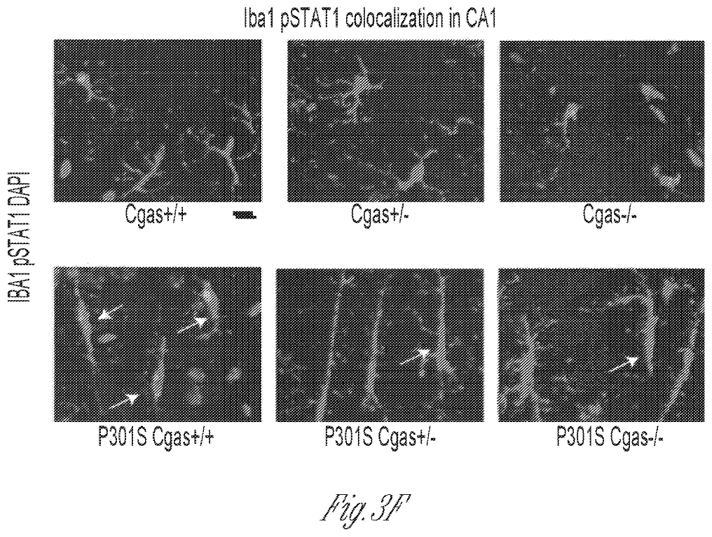

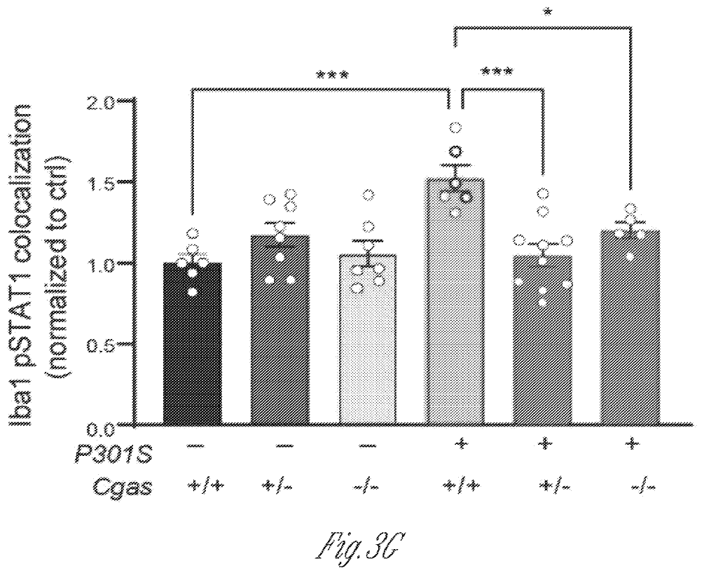

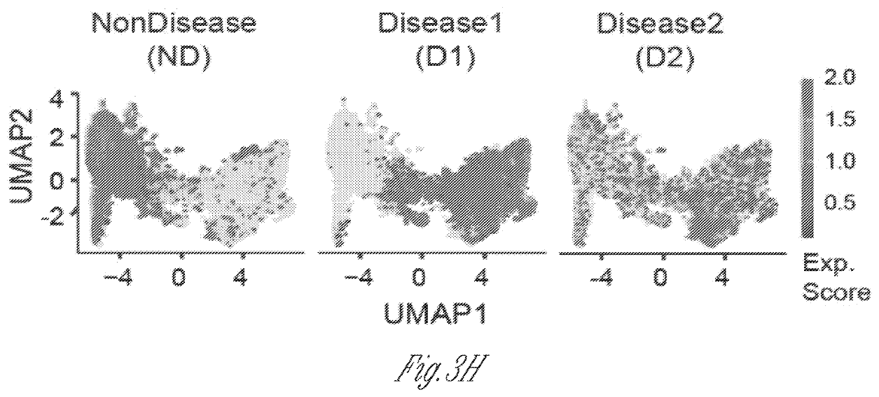

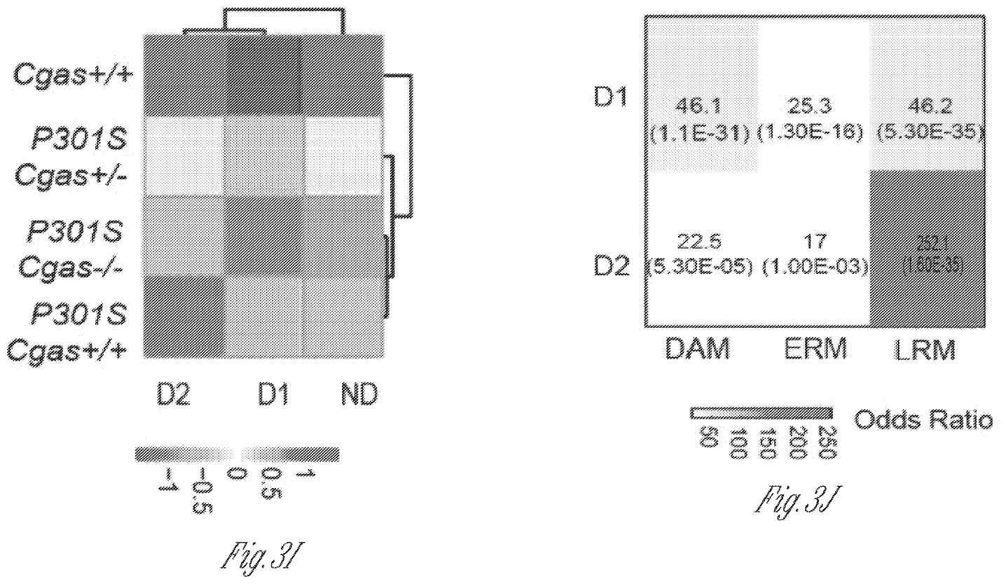

The cGas-STING Pathway is Activated in Hippocampi of Tauopathy Mice and Human AD Brains (FIG. 1).

FIG. 1A is a volcano plot of RNA-seq data from bulk hippocampal tissue from 8-9-month-old P301S and non-transgenic mice. Red dots represent genes with |log 2 fold change|>0.5. Wald test was used. All other genes are colored blue. Select upregulated interferon genes are labeled. n=7 non-transgenic, n=6 P301S; FIG. 1B is the gene set enrichment analysis showing hallmark pathways associated with top 500 DEGs upregulated in P301S compared to non-transgenic samples; FIG. 1C is the gene set enrichment analysis showing top transcription factors associated with top 500 DEGs upregulated in P301S compared to non-transgenic samples; FIG. 1D is the Ingenuity Pathway Analysis prediction of cGAS as an upstream regulator of upregulated DEGs, identified using Activation z-score>1 and p-value of overlap<0.05; FIG. 1E is the Western blots for phosphorylated Tank binding kinase (pTBK1), total Tank binding kinase 1 (TBK1) and GAPDH. Using hippocampal tissue lysates. Lanes 1-7: non-transgenic (ntg); Lanes 8-14: P301S; FIG. 1F is the ratio of pTBK1/TBK1 from (E) showing significantly higher phopho-TBK1 in P301S compared to non-transgenic hippocampi. ** p=0.0015 Student's two tailed t-test; FIG. 1G is the representative immunofluorescent images of non-transgenic and P301S hippocampi labeled with anti-Iba1 (green) and anti-STING antibodies. (Scale bar=50 μm); FIG. 1H is the quantification of Iba1 and Sting immunofluorescence intensities, showing increased Iba1 coverage and Iba1-Sting overlap in P301S hippocampi. Results presented as average intensity measurements from 3-4 section per animal. p-value <0.05. Student's two-tailed t-test. (n=5 Ntg, n=5 P301S); FIG. 1I is the schematic showing the patient number and brain region used for single-nuclei 10× genomics sequencing. (n=7, 4 males and 3 females); FIG. 1J is the UMAP plots showing expression of microglial marker genes INPP5D and CSF1R as well as STAT1 and cGAS (MB21D1) in snRNA-Seq of human microglial population; FIG. 1K is the Gene set enrichment analysis showing hallmark pathways associated enriched in cGAS expressing microglia; FIG. 1L is the representative western blots for pTBK1 and GAPDH using human frontal cortex brain lysates. Lanes 1-3: non-AD (Braak stage 0); Lanes 4-6: AD (Braak stage 6); FIG. 1M is the Ratio of pTBK1/GAPDH from (L) showing significantly higher phospho-TBK1 in human AD compared to non-AD brains. ** p<0.01, Student's two tailed t-test. (n=10 non-AD, n=8 AD)

Interferon Activation in Tau-Stimulated Microglia is Mediated by cGAS and Mitochondrial DNA Leakage (FIG. 2).

FIG. 2A is the Quantification of IFNB by ELISA and CXCL10 and CCL5 proteins by MagPix multiplex ELISA in culture media supernatants from untreated (Ctrl) and tau-treated (Tau) primary mouse microglia. IFNB: n=7, ** p=0.0016, Paired ttest. CXCL10 and CCL5, n=5, *** p=0.0004, ** p=0.0031, unpaired ttest; FIG. 2B is the epresentative western blots for phosphorylated Tank binding kinase (pTBK1), total Tank binding kinase 1 (TBK1) and GAPDH using mouse primary microglial cell lysates (Lane 1: untreated; Lane 2: treated with tau fibrils); FIG. 2C is the Ratio of pTBK1/TBK1 from FIG. 2A showing significantly higher phopho-TBK1 in tau fibril-treated primary microglia (Tau) compared to untreated (Ctrl). *, p<0.05, Student's paired t-test (n=3); FIG. 2D is the electron micrographs of primary mouse microglia treated with tau fibrils and immunogold labeled for antibody against tau. (L=lysosome, M=mitochondria); FIG. 2E is the ratio of mitochondrial DNA (Nd2) to genomic DNA (Tert) measured by RT-qPCR on DNA extracts of BV2 IfnB luciferase reporter cells treated for 7 days with ddC (40 or 80 μg/ml) or EtBr (50 or 100 ng/ml) to generate mtDNA-depleted (ρ°) cells. The values are normalized to the untreated sample (n=2), **** p<0.0001, one-way ANOVA; FIG. 2F is the control and mtDNA-depleted (ρ°) IfnB luciferase-reporter BV2 cells were stimulated or not with tau fibrils. IfnB signal and viability were measured 16 h later. IfnB-luciferase signal is shown normalized to Cell TiterGlo signal to correct for viability/Cell count. (n=3), **** p<0.0001, two-way ANOVA; FIG. 2G is the bulk RNA-seq analysis for Cgas+/+ and Cgas−/− primary cultured microglia treated or not with tau fibrils or HT-stranded DNA. (n=3 per condition). Venn diagram showing overlap of genes upregulated by dsDNA and tau treatment in Cgas+/+ microglia. Log fold change >1 and FDR <0.05; FIG. 2H is the top 5 reactome pathways represented in upregulated DEGs common to dsDNA and tau treated Cgas+/+ microglia. FDR <0.05; FIG. 2I is the heatmap summary of interferon stimulated genes that are lower in Cgas−/− compared to Cgas+/+ microglia stimulated with HT-DNA or Tau; FIG. 2J is the string interaction plot of genes from (I) including interferon genes including Stat1, Sp100, and Ddx60.

Partial or Complete Loss of cGas Mitigates Tauopathy-Associated Microglial Interferon Signature (FIG. 3).

FIG. 3A is the Dot plot showing normalized cell type expression of Cgas (Mb21d1) and Sting (Tmem173) in single nuclei sequencing (snRNA-Seq) samples; FIG. 3B is the UMAP plots showing strong expression of marker genes P2ry12, Siglech, Sall1, and Csf1r in snRNA-Seq microglial population (n=6 per genotype); FIG. 3C is the UMAP plots colored according to microglial subclusters and split by genotype; FIG. 3D is the violin plots showing expression level of homeostatic (P2ry12, Siglech), disease associated (Apoe, Itgax) and interferon (Parp14, Stat1, Trim30a, Rnf213) genes in microglia clusters; FIG. 3E is the Dot plot showing interferon stimulated genes that are significantly lower in P301S Cgas+/− and P301S Cgas−/− microglia compared to P301S Cgas+/+ microglia; FIG. 3F is the representative 63× confocal images of immunostaining of phosphor-STAT1 in the CA1 stratum radiatum of mouse hippocampus. (Scale bar=10 μm); FIG. 3G is the Mean intensity of phosphor-STAT1 measured in CA1 striatum radiatum. Each circle represents the average intensity measurement of 3 images per animal. Statistical comparisons performed using Two-way ANOVA. Data are reported as mean #SEM (n=6 Cgas+/+, n=8 Cgas+/−, n=7 Cgas−/−, n=6 P301S Cgas+/+, n=9 P301S Cgas+/−, n=5 P301S Cgas−/−). (Cgas+/+ vs P301S Cgas+/+: p=0.0002, P301S Cgas+/+ vs P301S Cgas+/−: p=0.0002, P301S Cgas+/+ vs P301S Cgas−/−: p=0.0415); FIG. 3H is the Heatmap showing association of gene modules with genotype; FIG. 3G is the Analysis of disease module 1 and 2 markers compared to disease associated, early response and late response microglia signatures.

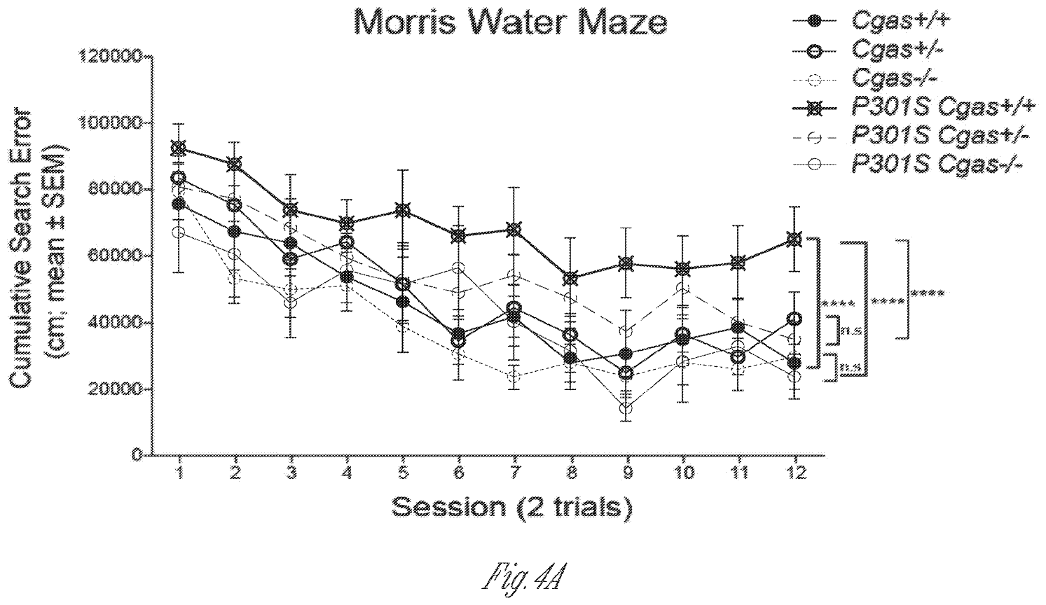

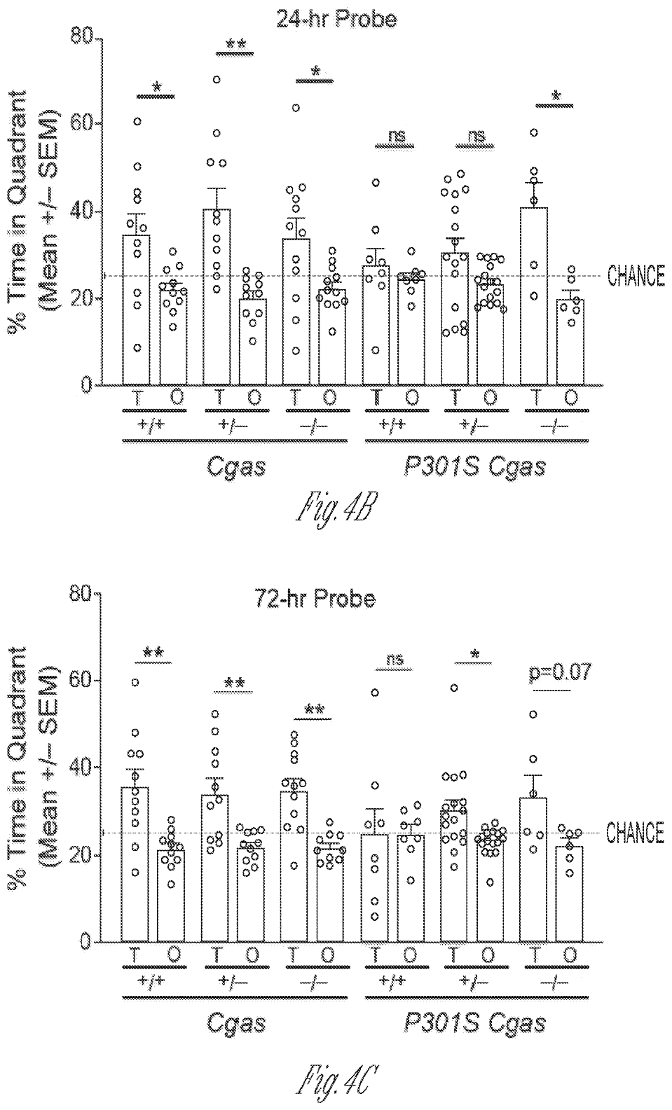

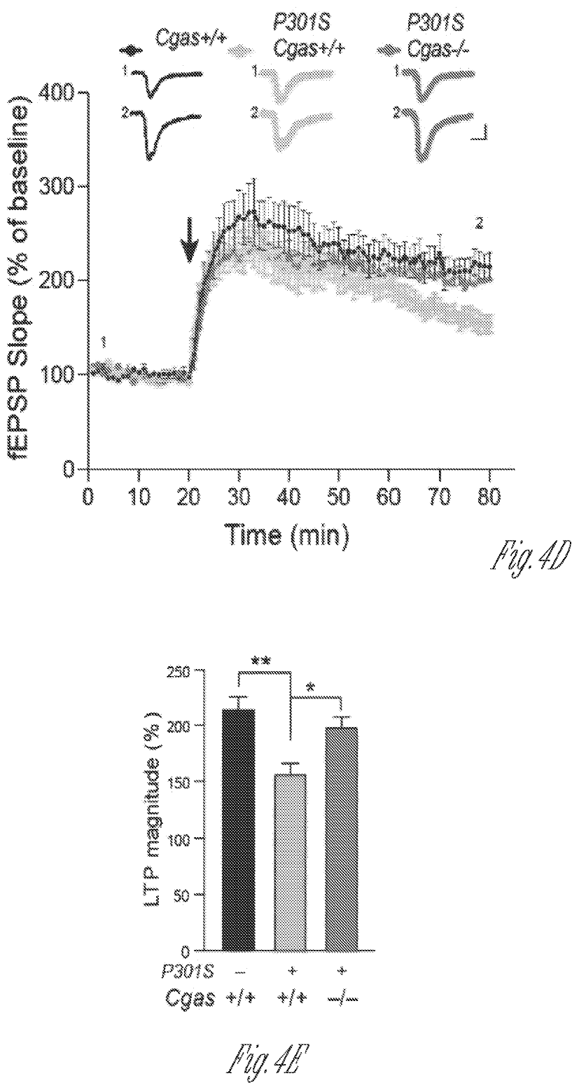

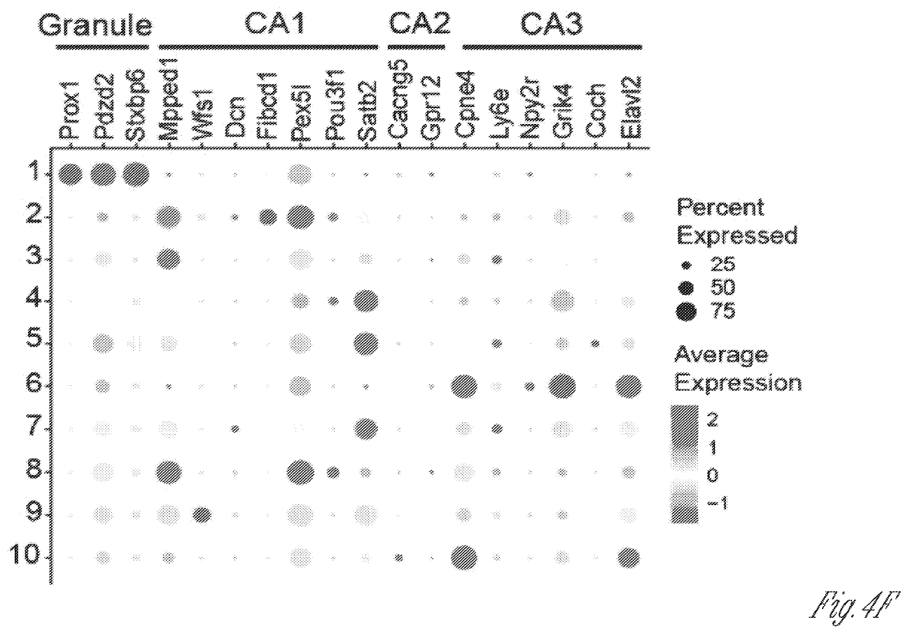

Partial or Complete Loss of cGAS Rescues Tauopathy-Induced Hippocampal Synapse Toxicity and Memory Deficits (FIG. 4).

FIG. 4A is the cumulative search distance during hidden trials (Session 1-12) in a Morris water maze (MVM) assessment of spatial learning and memory in 7-8-month-old P301S cGas+/+, P301S cGas+/−, and P301S cGas−/− and their non-transgenic littermates. Males and females were tested on separated days. Data presented here represents both sexes combined. n=12 cGas+/+, n=11 cGas+/−, n=11 cGas−/−, n=8 P301S cGas+/+, n=17 P301S cGas+/−, n=6 P301S cGas−/−. Two-way ANOVA. ****, p<0.0001; FIG. 4B is the percentage of time spent in the target or the average time spent in the nontarget (others) quadrants during the 24-hr probe in the MVM assessment. Paired two-tailed Student's ttest;

FIG. 4C is the percentage of time spent in the target or the average time spent in the nontarget (others) quadrants during the 72-hr probe in the MVM assessment. Paired two-tailed Student's ttest; FIG. 4D is the field excitatory postsynaptic potentials (fEPSPs) were recorded in the dentate gyrus molecular layer and a TBS protocol was applied (arrow) to the perforant pathway to induce LTP. Representative traces show fEPSPs before and after LTP induction (top). Scale bars, 0.4 mV and 5 ms. The fEPSP slope measurements made up to 60 minutes after TBS were normalized to the mean baseline fEPSP slope before LTP induction (bottom, n=8-11 slices from three to four mice per group); FIG. 4E is the LTP magnitude was calculated from the normalized mean fEPSP slope 55-60 min after TBS was applied. (n=8-11 slices from three to four mice per group; *, p<0.05, **, p<0.01; one-way ANOVA, Bonferonni post-hoc analyses);

FIG. 4F is the dot plot showing classification of excitatory neuron clusters by expression of granule, CA1 and CA2/3, CA2 markers; FIG. 4G is the Pie chart summarizing the proportion of DEGs from clusters pertaining to dentate gyrus (DG), CA1 and CA2/3 clusters; FIG. 4H is the representative confocal images of the hippocampal CA1 striatum radiatum labeled with PSD95 antibody. (Scale bar=10 μm); FIG. 4I is the mean intensity of PSD-95 puncta measured in CA1 striatum radiatum. Each circle represents the average intensity measurement of 3-5 images per animal. Statistical comparisons performed using one way or two-way ANOVA. (Cgas+/+vs P301S Cgas+/+: p=0.0275, P301S Cgas+/+vs P301S Cgas+/−: p=0.0002, P301S Cgas+/+vs P301S Cgas−/−: p=0.0415); Data are reported as mean±SEM.

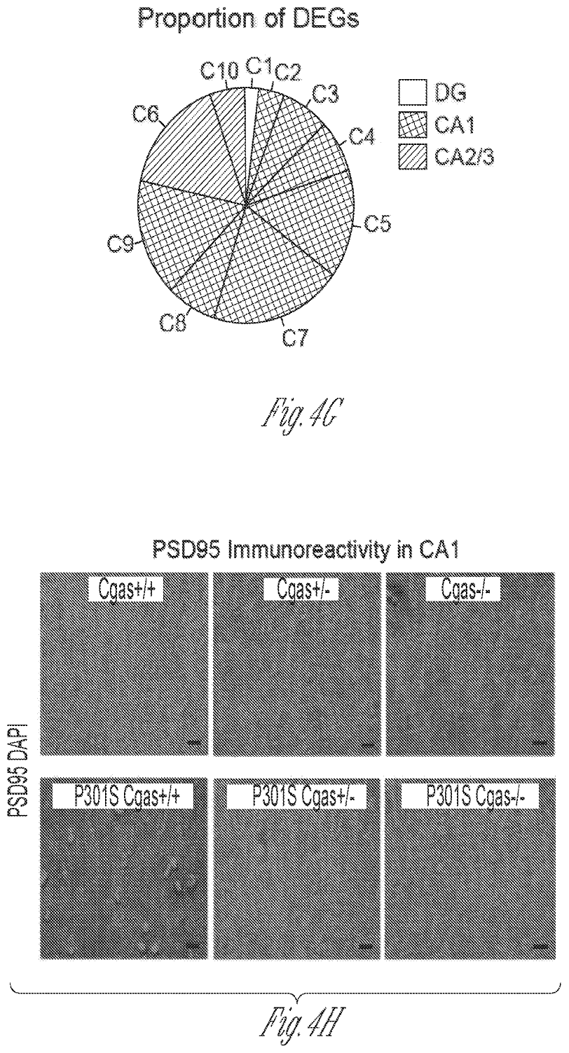

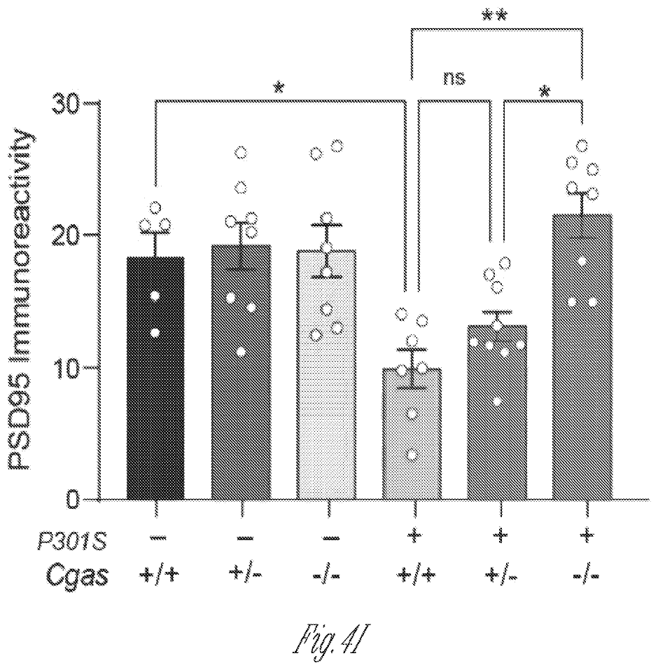

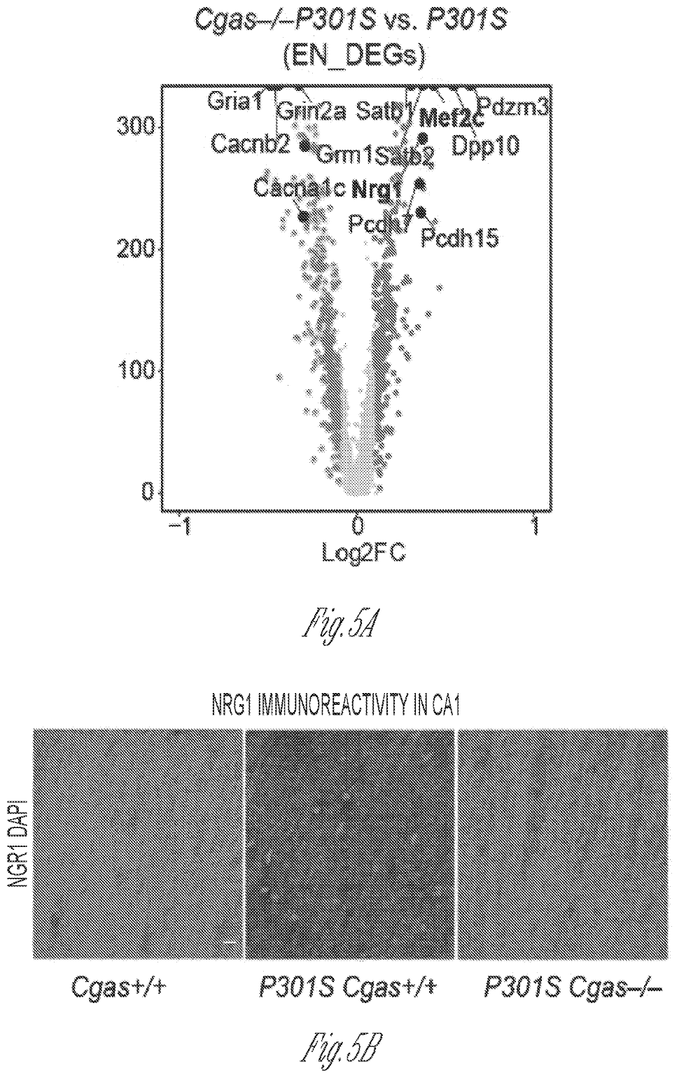

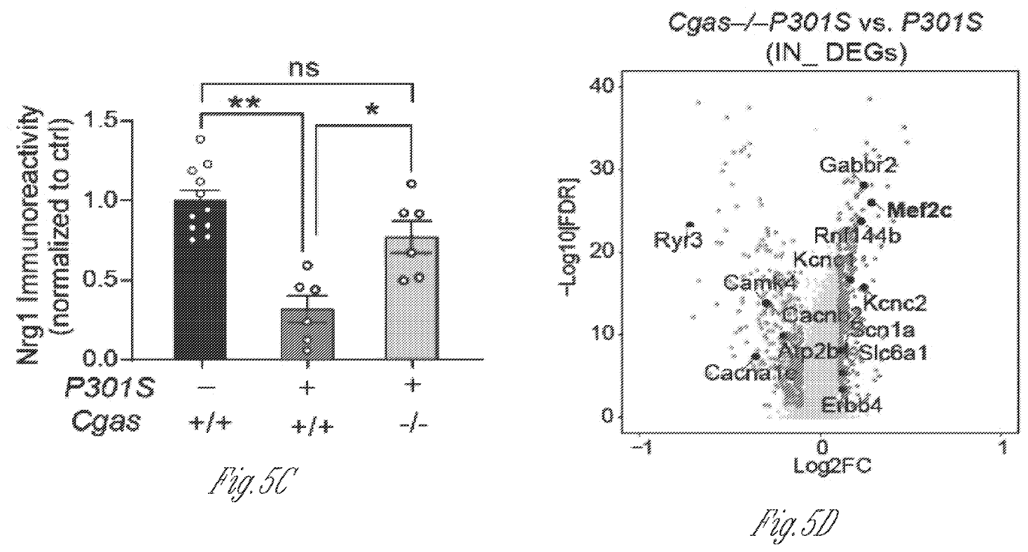

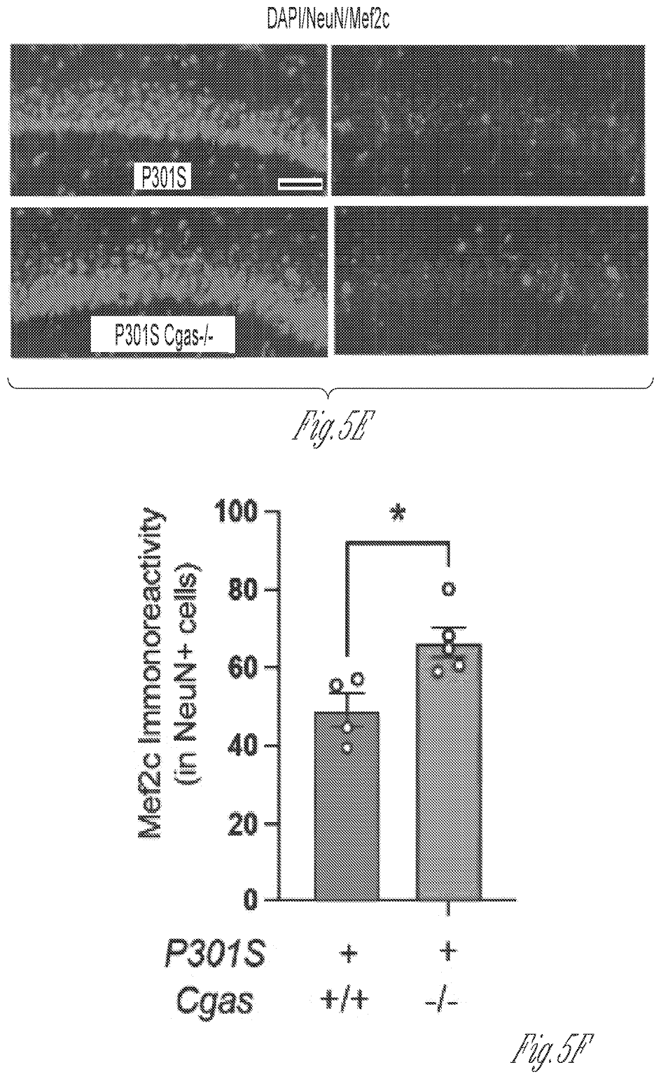

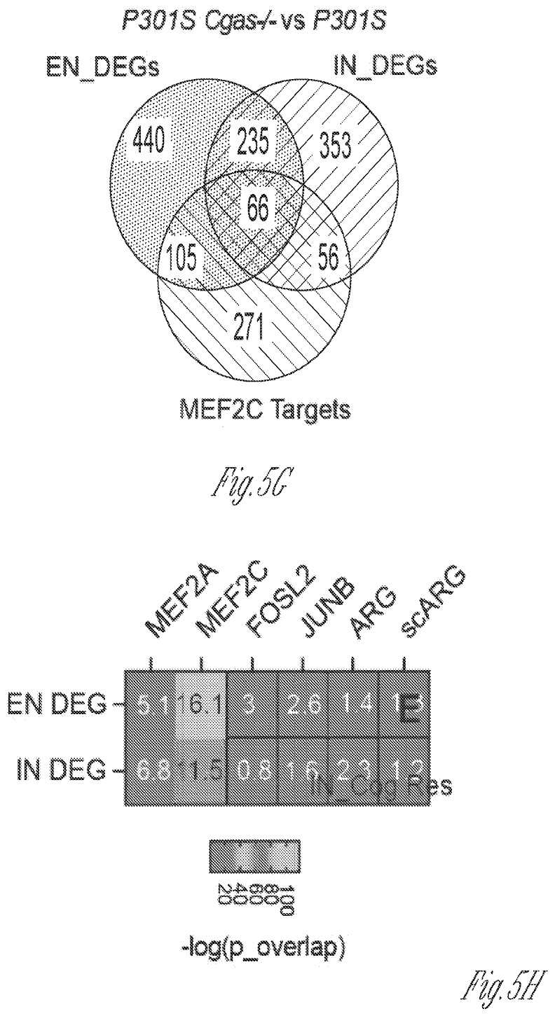

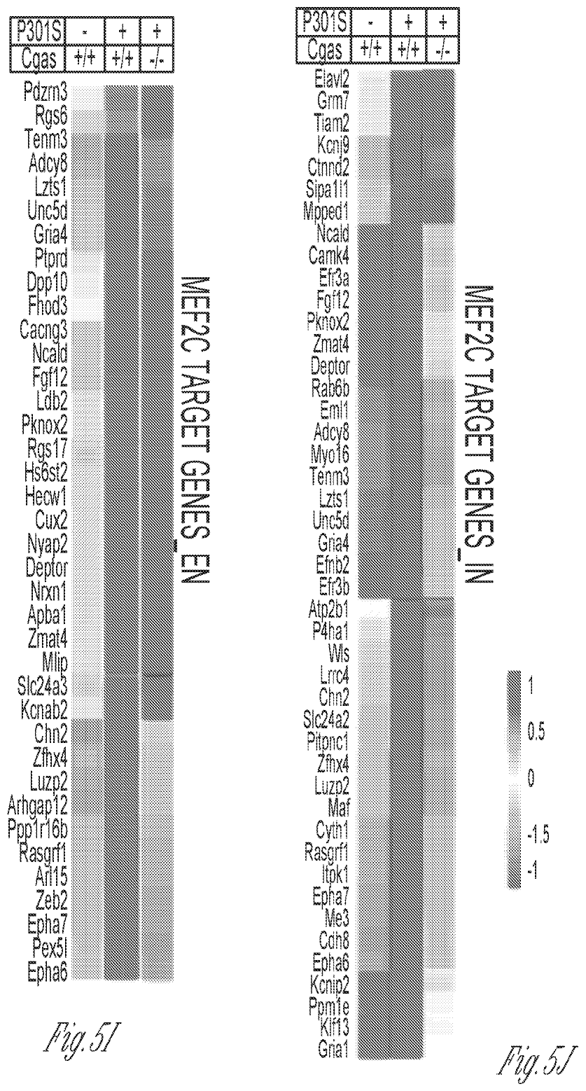

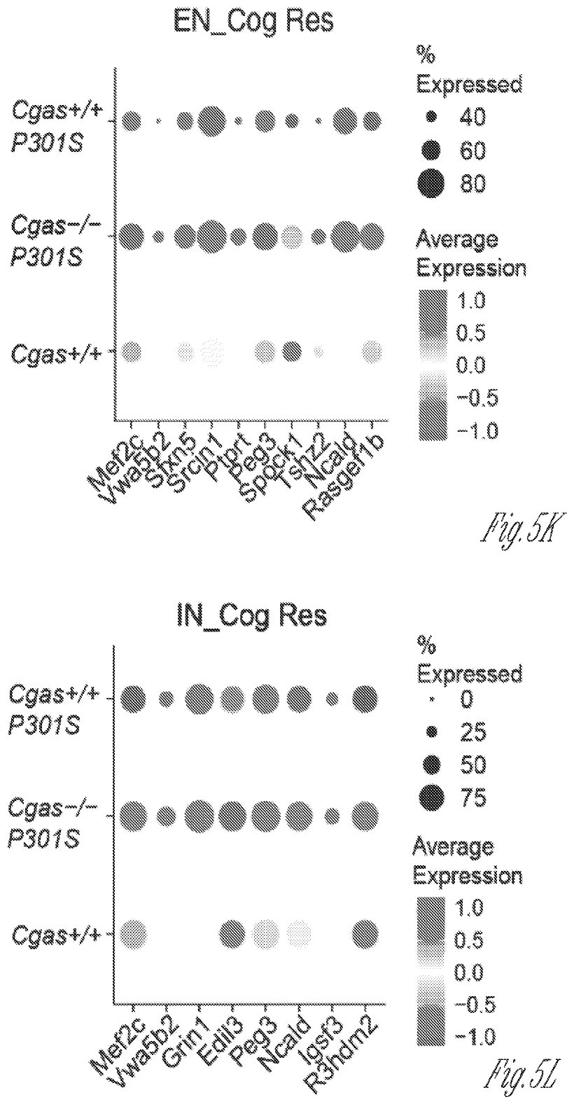

Loss of cGAS Rescues Expression of Mef2c and its Target Genes in Tauopathy Neurons 905 500×3579 (FIG. 5).

FIG. 5A is the Volcano plot showing representative differentially expressed genes that are upregulated in P301S Cgas−/− compared to P301S Cgas+/+ excitatory neurons. (log 2FC>0.1, FDR<0.05); FIG. 5B is the Representative 63× confocal images of immunostaining of NRG1 in the CA1 stratum radiatum of mouse hippocampus. (Scale bar=10 μm);

FIG. 5C is the Mean intensity of NRG1 measured in CA1 striatum radiatum. Each circle represents the average intensity measurement of 3 images per animal. Statistical comparisons performed using Two-way ANOVA. Data are reported as mean±SEM (n=11 Cgas+/+, n=6 P30IS Cgas++, n=8 P30IS Cgas+1, n=6 P30IS Cgas+. Cgas+/+ vs P30IS Cgas+/+: p=0.0016, P301S Cgas+/+ vs P301S Cgas−/−: p=0.0234); FIG. 5D is the Volcano plot showing representative differentially expressed genes that are upregulated in P301S Cgas' compared to P30IS Cgas+/+ inhibitory neurons. (log 2FC>0.1, FDR<0.05); FIG. 5E is the Representative 25× confocal images of immunostaining of Mef2c and NeuN in the CA1 pyramidal layer of mouse hippocampus. (Scale bar=50 μm); FIG. 5F is the Mean intensity of Mef2c in Mef2c+, NeuN+ neurons in CA1 pyramidal layer. Each circle represents the average intensity measurement of 3 images per animal. *, p<0.05, unpaired t test. Data are reported as mean±SEM (n=4 P30IS Cgas+/+, n=5 P301S Cgas−/−); FIG. 5G is the Venn diagram of the overlap between excitatory neuron DEGs, inhibitory neuron DEGs, and MEF2C target genes; FIG. 5H is the Heatmap showing the overlap between excitatory/inhibitory neuron DEGs and lists of transcription factor target genes (MEF2A, MEF2C, FOSL2, JUNB) and activity-induced differentially expressed genes (ARG and scARG). Numbers in each box represents the overlapping odds ratio; FIG. 5I is the Heatmap of the expression of significant DEGs (p.adj <0.5, log FC >=0.1 or <=−0.1) that are MEF2C targets in WT, P301S, and P301S Cgas−/− excitatory neuron clusters; FIG. 5J is the Heatmap of the expression of significant DEGs (p.adj <0.5, log FC >=0.1 or <=−0.1) that are MEF2C targets in WT, P301S, and P301S Cgas−/− inhibitory neuron clusters; FIG. 5K is the Dot plot showing the expression of significantly upregulated DEGs by Cgas deletion (p.adj <0.5, log FC >=0.1) that are positively correlated with human cognitive resilience in excitatory neuron clusters;

FIG. 5L is the Dot plot showing the expression of significantly upregulated DEGs by Cgas deletion (p.adj <0.5, log FC >=0.1) that are positively correlated with human cognitive resilience in inhibitory neuron clusters.

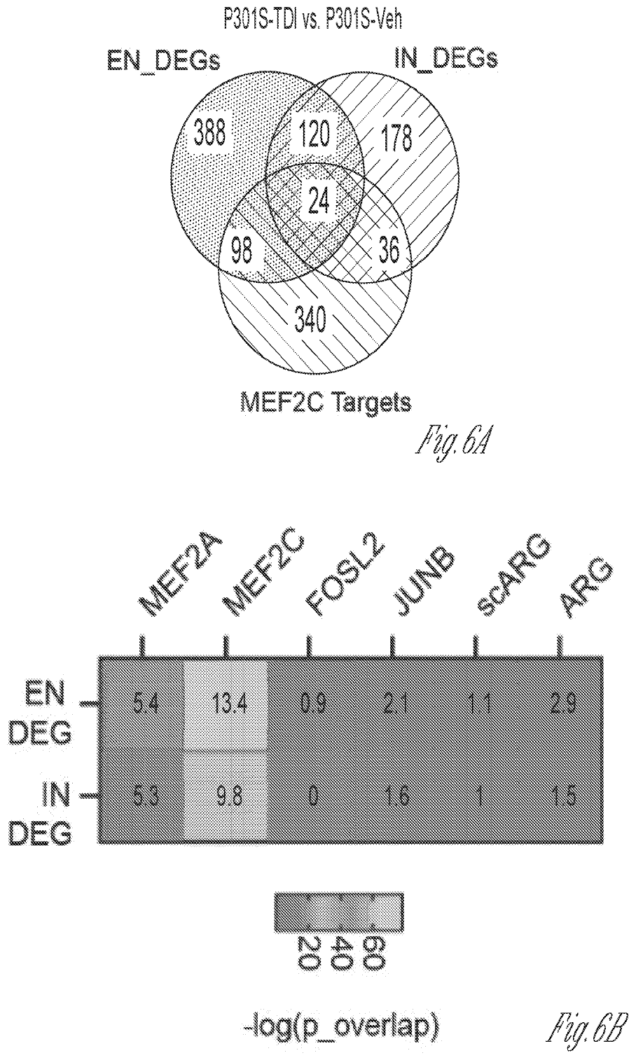

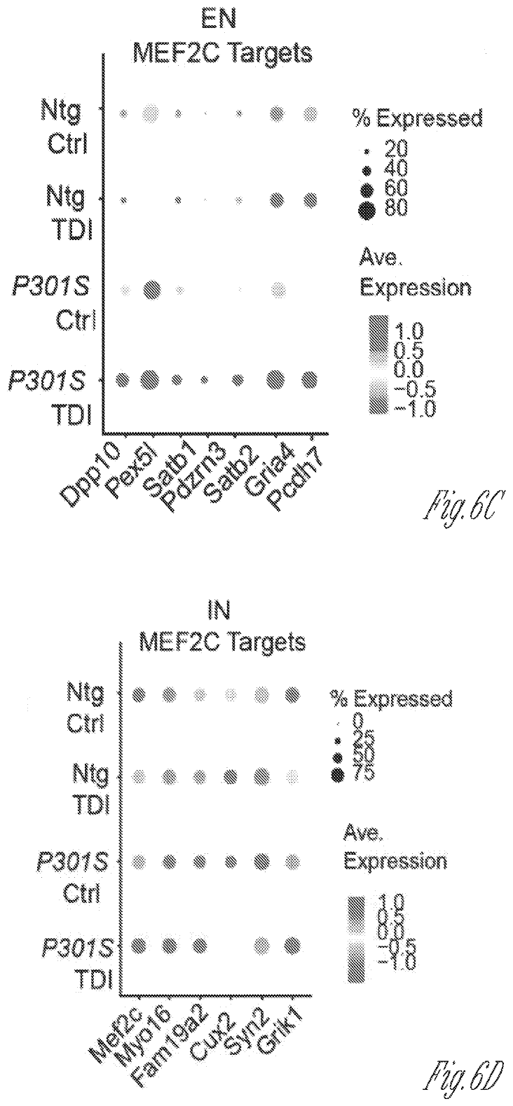

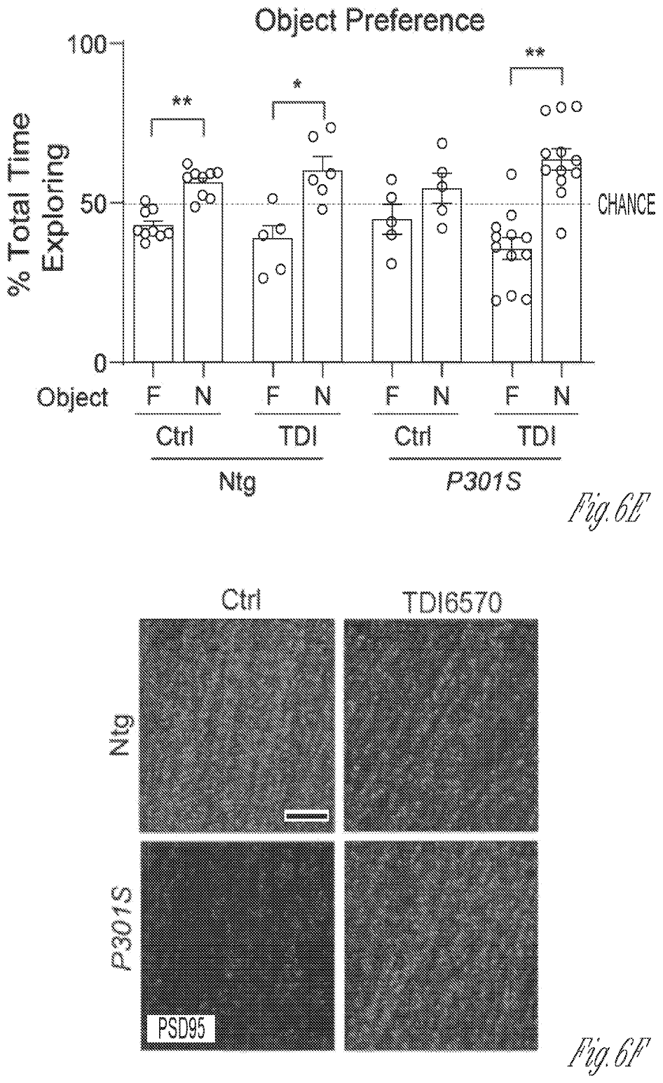

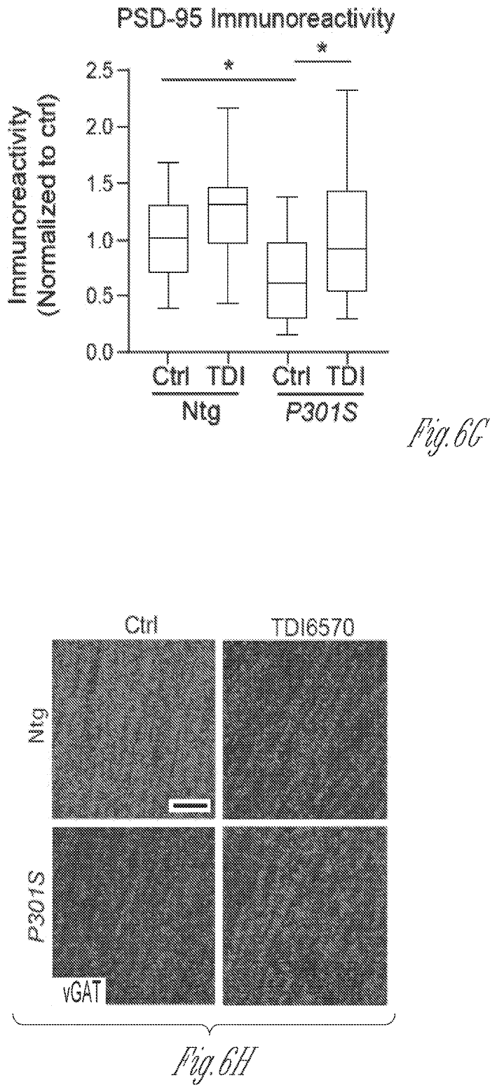

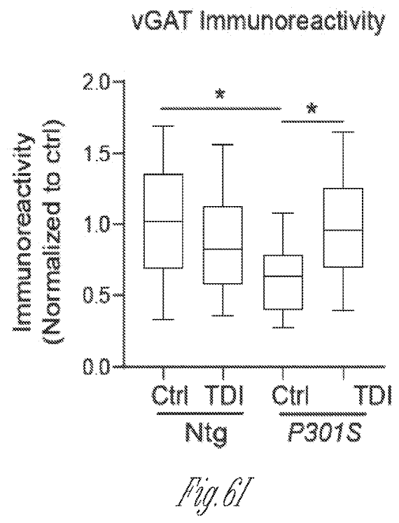

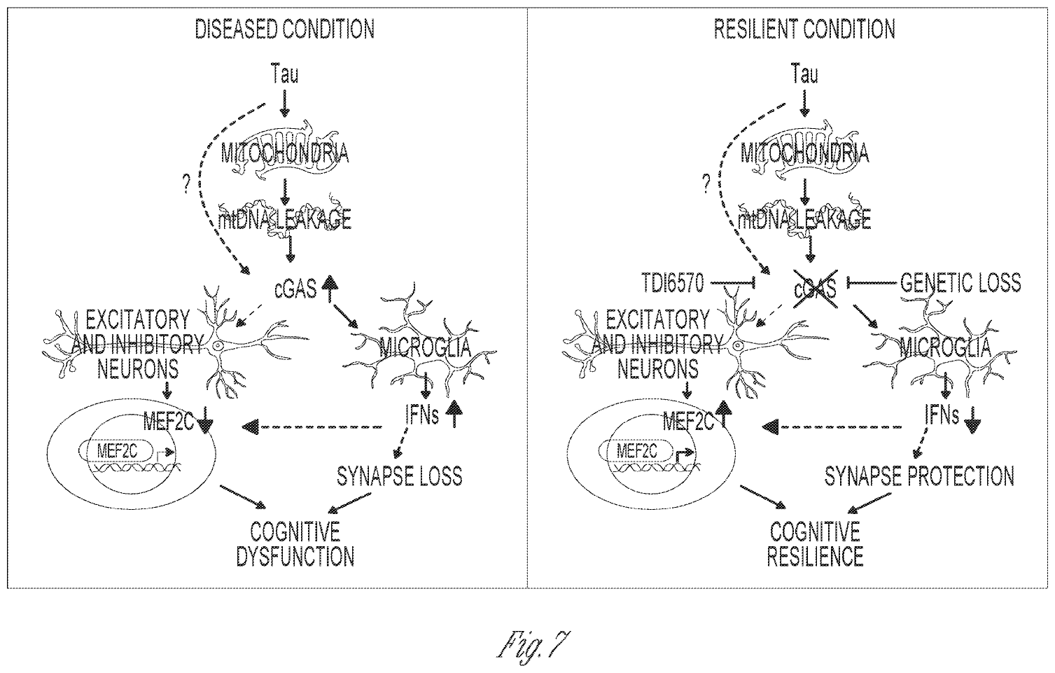

Brain Permeable cGAS Inhibitor Elevates MEF2C Target Genes and Protects Against Synaptic Loss and Spatial Learning and Memory Deficits (FIG. 6).

FIG. 6A is the Venn diagram of the overlap between P301S TDI vs P301S Veh DEGs in excitatory neuron, inhibitory neuron, and MEF2C target genes; FIG. 6B is the Heatmap showing the overlap between excitatory/inhibitory neuron DEGs and lists of transcription factor target genes (MEF2A, MEF2C, FOSL2, JUNB) and activity-induced differentially expressed genes (ARG and scARG). Number in each box represents the overlapping odds ratio; FIG. 6C is the Dot plot showing the expression of significantly upregulated DEGs (p.adj <0.5, log FC >=0.1) that are MEF2C targets in Ntg Ctrl, Ntg TDI, P301S Ctrl, P301S TDI excitatory neuron clusters; FIG. 6D is the Dot plot showing the expression of significantly upregulated DEGs (p.adj <0.5, log FC>=0.1) that are MEF2C targets in Ntg Ctrl, Ntg TDI, P301S Ctrl, P301S TDI inhibitory neuron clusters; FIG. 6E is the Novel object recognition test for Ntg and P301S mice fed with 150 mg/kg TDI-6570 or control diet for three months. F: familiar object, N: novel object (n=9 Ntg control, n=6 Ntg TDI-6570, n=5 P301S control, n=12 P301S TDI6570). Statistical comparisons performed using two-way ANOVA. *, p<0.05, **, p<0.01; Data are reported as mean±SEM; FIG. 6F is the Representative confocal images of the hippocampal CA1 striatum radiatum labeled with PSD95 antibody. (Scale bar=10 μm); FIG. 6G is the Mean intensity of PSD95 puncta measured in CA1 striatum radiatum. Each circle represents the average intensity measurement of one image. 3-5 images were taken per animal. Statistical comparisons performed using mixed model (n=13 Ntg control, n=12 Ntg TDI-6570, n=9 P301S control, n=12 P30IS TDI-6570. Ntg control vs P301S control: p=0.0302, P301S control vs P301S TDI-6570: p=0.0427); FIG. 6H is the Representative confocal images of the hippocampal CA1 striatum radiatum labeled with vGAT antibody. (Scale bar=10 μm); FIG. 6I is the Mean intensity of vGAT puncta measured in CA1 striatum radiatum. Each circle represents the average intensity measurement of one image. 3-5 images were taken per animal. Statistical comparisons performed using mixed model. (n=12 Ntg control, n=11 Ntg TDI-6570, n=8 P301S control, n=13 P301S TDI-6570. Ntg control vs P301S control: p=0.0453, P301S control vs P30IS TDI-6570: p=0.0318). Given the striking protective effect of cGas depletion on P301S behavioral impairment, small molecule cGas inhibitors were developed. The initial focus was on a known cGAS inhibitor, TDI-65701. TDI-6570 inhibits both mouse and human cGAS with a sub-micromolar activity (IC₅₀: 0.0128 μM and 0.138 μM, respectively). TDI-6570 was prepared in multigram scale using a 4-step process, earlier described by Lama, et al.¹

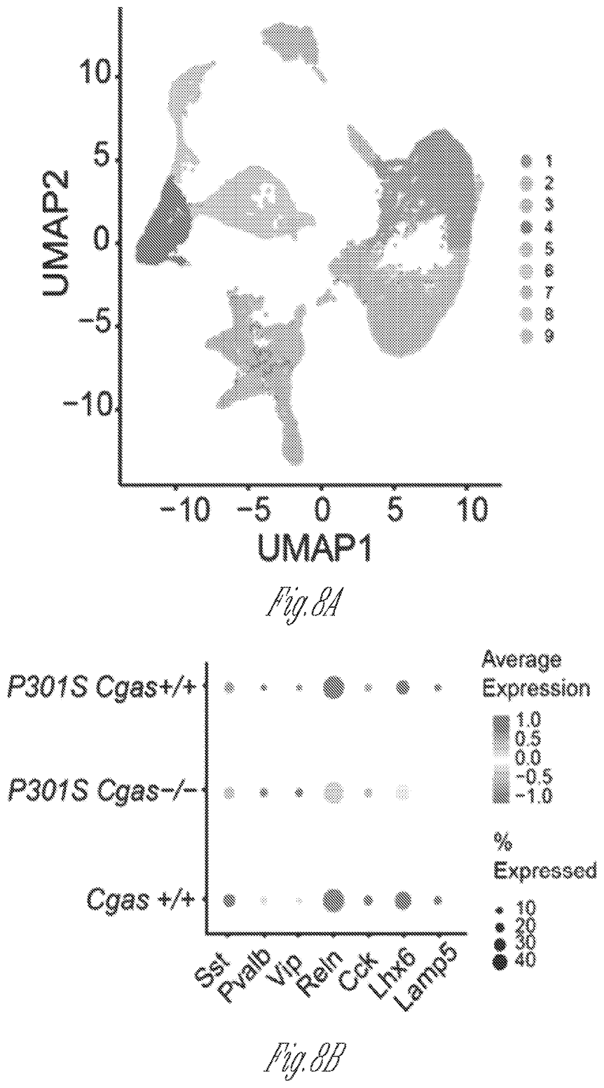

Working Model Illustrating cGAS-IFN-MEF2c Axis in Tauopathy (FIG. 7).

Under disease/vulnerable condition, pathogenic tau activates cGAS-dependent interferon response via mtDNA leakage in microglia, and reduction of MEF2c transcriptional network in excitatory and inhibitory neurons, resulting in cognitive dysfunction. Loss of cGAS reduces interferon response in microglia and enhances Mef2c transcriptional network, resulting in cognitive resilience.

### Cgas Deletion Modified Transcriptomes of Inhibitory Neurons in Tauopathy

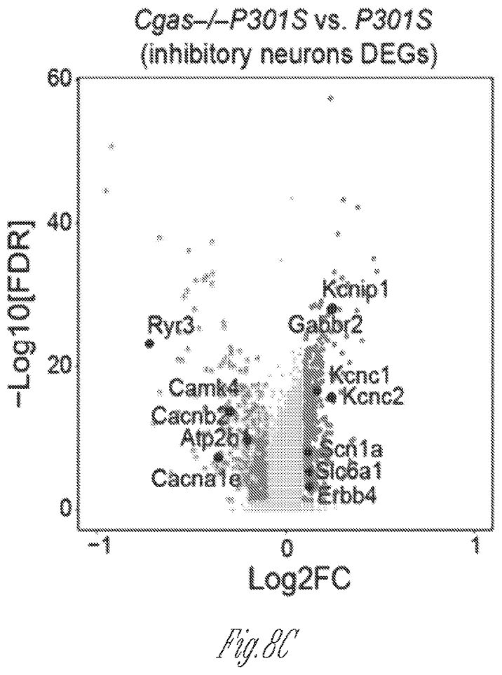

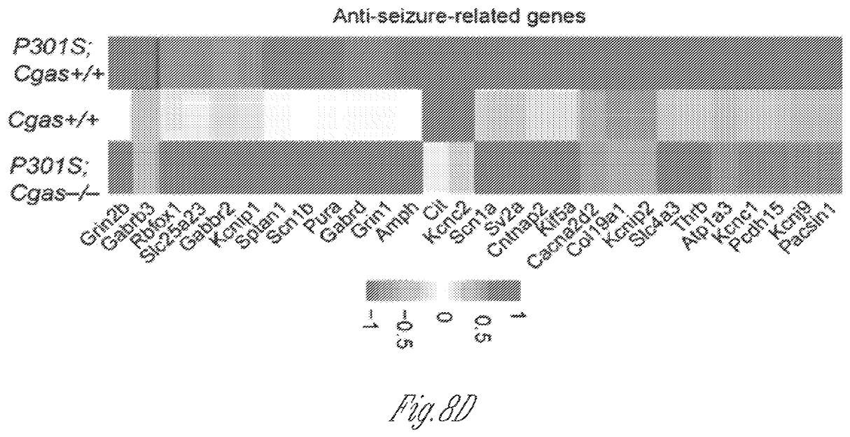

(FIG. 8). Subclustering of pan-interneuron marker GAD1 and GAD2 positive neuron populations identified 9 inhibitory neuron subpopulations (FIG. 8A) (Arneson et al., 2018; Cembrowski et al., 2016). We found that Cgas deletion rescued tauopathy-induced downregulation of a subset of interneuron markers such as Pvalb, Vip, Reln, Lhx6, but not Sst or Cck. In addition, analyses of DEGs from interneurons revealed that Cgas deletion led to upregulation of genes involved GABA signaling, including GABA receptor Gabbr2 and GABA transporter Slc6a, supporting restoration of interneuron function by Cgas deletion (FIG. 8B). Interestingly, among the upregulated genes by Cgas deletion, we found many are involved in regulating neuronal excitability and seizure activity, including potassium channels and interacting subunits, Kcnc1, Kcnc2, Kacnip1, and sodium channel Scn1a (FIG. 8C). Indeed, Cgas deletion resulted in a striking upregulation of anti-seizure genes that were diminished in the interneurons of P301S mice (FIG. 8D), including Scn1a. Deficiency of Scn1a leads to Dravet syndrome, an intractable childhood epilepsy with generalized tonic-clonic seizures.

cGAS-STING Pathway is Activated in Tau Transgenic Mice and in Human AD Brains

To characterize gene expression changes associated with tauopathy, bulk RNA sequencing of P301S tau transgenic and non-transgenic hippocampi (8-9 months) were performed. Differential gene expression (DEG) analysis revealed a striking upregulation of interferon genes in P301S compared to non-transgenic hippocampi, and enrichment of interferon response factor (IRF) and ISRE (interferon sensitive response elements) transcription factor motifs (FIG. 1A-C). Ingenuity pathway analysis of predicted upstream regulators of upregulated DEGs confirmed numerous components of interferon signaling, including Ifnar1, Stat1, and Irf3. Notably, components of the cGAS-STING pathway including cyclic GMP-AMP synthase (cGAS), stimulator of interferon genes (STING) and tank binding kinase 1 (TBK1), were also predicted activators of upregulated DEGs (FIG. 1D).

cGAS-STING pathway activation was tested by immunoblotting for TBK1 phosphorylation and observed increased TBK1 phosphorylation in P301S hippocampal lysates (FIG. 1E-F). Additionally, immunofluorescent labeling were performed and significantly increased microglial STING expression in P301S was observed compared to non-transgenic hippocampi (FIG. 1G-H). Together, these studies show that the cGAS-STING pathway is activated in the P301S tauopathy mice.

To investigate the involvement of cGAS in microglial interferon response in human AD, levels of TBK1 phosphorylation in a cohort of healthy versus AD (Braak stage 0 vs VI) post-mortem brain samples were assessed and showed that pTBK1 levels were significantly elevated in human AD brains (FIG. 1L-M). Additionally, an independent cohort of AD single-nuclei RNA-seq (snRNA-seq) of mid-frontal cortex of 11 AD patients with ApoE3/4 genotype (6 males and 5 females, FIG. 1I) were examined. SnRNA-seq experiments were performed and analyzed as described previously (Sayed et al., 2021). Sequencing reads obtained from multiplets were excluded using DoubletFinder (McGinnis et al., 2019) and reads from low-quality nuclei were removed by thresholding gene counts, UMI counts, and percent mitochondrial genes per nuclei (Figure S2). Analyses of the INPP5D- and CSF1R-positive microglia identified a subpopulation of STAT1-positive microglia, which overlapped with cells expressing CGAS (FIG. 1J). Hallmark Pathway analysis of the CGAS+ microglia revealed enrichment for complement, interferon gamma, and alpha responses (FIG. 1K). Taken together, the results indicate cGAS-STING signaling contribute to interferon activation in human AD.

Tau Fibrils Activate Microglial Interferon Signaling Through cGAS Activation and Mitochondrial DNA Leakage

To determine if pathogenic tau directly activates interferon signaling in microglia, levels of IFNB, CXCL10 and CCL5 proteins in culture media from primary microglia treated with tau fibrils using multiplex ELISA were measured and confirmed their induction in response to tau (FIG. 2A). Tau treatment also led to robust TBK1 phosphorylation indicating that tau directly induces activation of cGAS-STING signaling in microglia (FIG. 2B-C).

Next, the mechanism of interferon activation following tau treatment was investigated. To determine the subcellular localization of tau fibrils in microglia, electron microscopy of tau-treated primary microglia with immunogold labeling against tau at 24 hours after treatment was performed. Surprisingly, tau was found to be localized in mitochondria besides in lysosomes, their expected location after phagocytosis (FIG. 2D). We reasoned that tau in mitochondria may trigger mtDNA release which can activate cGAS and interferon signaling. To facilitate quantification of interferon responses, an IfnB luciferase reporter BV2 cell line was generated. These cells upregulate IfnB in response to known cGAS agonists such as cGAMP and herring testis DNA (HT-DNA) as well as tau fibrils. mtDNA-depleted cells (ρ° cells) by treating the IfnB BV2 cells with low dose Ethidium Bromide (EtBr) or Dideoxycytidine (ddC) (Hashiguchi and Zhang-Akiyama, 2009; Kaguni, 2004) were then generated. qPCR for mitochondrial and nuclear genes confirmed a dose-dependent depletion of mtDNA after ddC and EtBr treatment (FIG. 2E). To test if our system can reliably measure interferon responses induced by mtDNA leakage, we co-treated IfnB luciferase BV2 cells with Bcl2 inhibitor ABT-737 and caspase inhibitor Q-VD-OPH (QVD) to trigger mtDNA leakage without apoptosis induction (Rongvaux et al., 2014; White et al., 2014). ABT-737+ QVD treatment activated IfnB-dependent luciferase expression, which were markedly dampened with ddC- or EtBr-induced mtDNA depletion. Remarkably, both ddC and EtBr treatment significantly dampened microglial IfnB response to tau fibrils in a dose-dependent manner, strongly supporting the involvement of mtDNA as a part of tau-dependent IfnB responses in microglia (FIG. 2F).

To investigate if cGAS mediates tau induced interferon responses, Cgas and Cgas^{+/+} primary microglia were treated with tau fibrils or HT-DNA and performed RNA sequencing. Microglial response to HT-DNA depends on cGAS as evidenced by complete elimination of transcriptomic responses in Cgas/microglia. In comparison, Cgas deletion only reduced a subset of tau-stimulated inflammatory and cytokine expression. Nevertheless, further analyses revealed striking overlap of microglial responses induced by HT-DNA and Tau, with almost 80% of genes upregulated by tau being also upregulated by HT-DNA (FIG. 2G). Pathway enrichment terms such as interferon-alpha and -beta signaling and interferon-gamma signaling were overrepresented pathways in these shared DEGs, suggesting that HT-DNA and tau engage similar interferon response genes to promote microglial inflammation (FIG. 2H). Indeed, Cgas deletion diminished expression of a subset of interferon stimulated genes, including Stat1, Ddx60, Isg20, Rnf213, Parp12, Ifi35, and Sp100, in microglia treated with HT-DNA or tau fibrils (FIG. 2I-J). Thus, microglial cGAS promotes tau-induced interferon and inflammatory responses, overlapping with those induced by cytosolic DNA and at least partly mediated by mitochondrial DNA leakage.

cGAS Loss Mitigates Tauopathy-Associated Microglial Interferon Signature In Vivo

To directly assess the role of cGAS in tauopathy, we crossed P301S mice to Cgas^{-/-} mice to generate a cohort of transgenic and non-transgenic litters expressing two, one or no copy of functional Cgas. We performed single nuclei RNA sequencing (snRNA-Seq) of hippocampi from 8-9 months old Cgas^{+/+}, P301S Cgas^{+/+}, P301S Cgas^{+/-}, and P301S Cgas^{-/-} mice. Stringent quality control measures were implemented to exclude sequencing reads obtained from multiplets using DoubletFinder (McGinnis et al., 2019) and from low-quality nuclei by thresholding gene counts, UMI counts, and percent mitochondrial genes per nuclei. Unsupervised clustering was then performed to group the resultant 234,474 high quality nuclei into transcriptionally distinct clusters, which represented all of the major cell types of the brain.

In our snRNA-seq data, Cgas (Mb21d1) and Sting (Tmem173) were detected only in the microglial cluster characterized by strong expression of microglial markers like Csf1r, P2ry12, and Siglech (FIG. 3A-B). To dissect how Cgas reduction affects microglial responses to tauopathy, microglia were further subclustered into 4 microglial subpopulations, with Cgas+/+ samples comprised primarily of cluster 1 microglia, while all 4 clusters were found in tauopathy samples, confirming robust transformation of microglial states in tauopathy as reported previously (Sayed et al., 2021) (FIG. 3C). Cluster 1 expressed high levels of homeostatic genes, P2ry12 and Siglech, clusters 2, 3, and 4 showed reduced expression of these homeostatic genes and simultaneous upregulation of disease associated (Apoe, Itgax) and interferon stimulated genes (Stat1, Parp14, Trim30a and Rnf213) (FIG. 3D). Cluster 3 was enriched with interferon genes specifically, which is distinct from cluster 4 enriched with the well-established disease-associated microglial phenotype (DAM) reported in mouse amyloid model (Keren-Shaul et al., 2017) (FIG. 3D). Compared with Cgas+/+ microglia, expression of genes associated with interferon responses (Trim 30a, Trim 30b, Stat1, Ddx60, Rnf213, Parp14, and others) was much stronger in microglia of P301S Cgas+/+ mice (FIG. 3E). To validate the suppression of interferon responses in microglia by cGAS reduction, we performed immunofluorescent labeling of phospho-STAT1. The increase in the pSTAT1 signal in the microglia of hippocampal CA1 region of P301S Cgas+/+ was significantly diminished in the P301S Cgas+/− and P301S Cgas brains (FIG. 3F-G).

Using trajectory analysis to model microglial transformation from homeostatic to disease signatures (Trapnell et al., 2014), three gene modules that were significantly associated with microglial disease transformation (FIG. 3H) were identified. Gene ontology analysis was performed to identify overrepresented gene signatures associated with the two microglial disease modules. Disease module 1 (D1) markers were associated with enrichment terms such as cell activation, defense response and immune response system, and disease module 2 (D2) markers were enriched with genes involved in response to virus and response to type I interferon. D1 genes included DAM genes such as Apoe, Lyz2, and Itgax, while D2 was characterized by expression of interferon genes. Compared to P301S Cgas^{+/+} P301S Cgas^{+/-} and P301S Cgas^{-/-} microglia exhibited no change in D1 module, but diminished D2 (FIG. 3I). Moreover, D2 correlated most strongly with late response microglia (LRM) signature which was associated with synapse and neuron loss, and cognitive impairment in p25 induction model of neurodegeneration (FIG. 3J) (Mathys et al., 2017).

Loss of cGAS Rescues Tauopathy-Induced Memory Deficits and Hippocampal Synapse Loss and Plasticity

Our data showed that loss of cGAS fine-tunes microglial responses to suppress a subset of disease response modules. Cgas deletion affected tauopathy-induced spatial learning and memory was evaluated using the Morris Water Maze (MWM) behavioral test. To assess spatial learning, hidden platform trials over 6 consecutive days were performed and measured cumulative distance covered to locate the platform in each trial (FIG. 4A). Cgas^{+/-} and Cgas^{-/-} mice phenocopied Cgas^{+/+} mice, while P301S Cgas^{+/+} mice exhibited impaired learning compared to non-transgenic mice, denoted by significantly higher search distance on the last hidden platform trial (Session 12). Remarkably, P301S Cgas^{+/-} and P301S Cgas^{-/-} mice performed similarly to Cgas^{+/+} mice in the hidden platform trial, showing strong protection induced by cGAS loss (FIG. 4A). In the 24-hour probe trials to assess spatial memory, P301S Cgas/mice spent significantly more time exploring the target platform quadrant compared to other quadrants whereas P301S Cgas^{+/+} were not able to discriminate the target quadrant from others (FIG. 4B). The beneficial effects of cGAS loss on spatial memory persisted in the 72-hour probe trials, demonstrating a strong rescue of spatial memory deficits (FIG. 4C). Swim speeds, vision, overall activity and anxiety levels were unaltered across all genotypes, supporting specific effects of cGAS loss on spatial learning and memory in tauopathy mice.

Tau-induced deficits in hippocampal synaptic plasticity has been previously linked to tauopathy-related memory loss (Tracy et al., 2016). TBS induced long-term potentiation (LTP) to similar levels in P301S Cgas^{+/+} and P301S Cgas/slices at the early phase of LTP. However, LTP magnitude was significantly reduced in P301S Cgas^{+/+} compared to P301S Cgas/slices by 60 minutes post induction, indicating that the late phase LTP impairment in P301S Cgas^{+/+} hippocampus was rescued by Cgas deletion (FIG. 4D-E). Thus, cGAS loss protects against tau-mediated plasticity deficits in hippocampal circuit.

Evaluation of how the loss of cGAS affected neuronal changes in hippocampal circuit, subclustering analyses of excitatory neuron populations were performed and identified 10 transcriptionally distinct excitatory neuron subpopulations (FIG. 4F), a majority of which showed non-overlapping expression of dentate granule, CA1, and CA2/3 neuron specific subtype markers (Arneson et al., 2018; Cembrowski et al., 2016; Dong et al., 2009; Sarkar et al., 2018) (FIG. 4F). Comparing excitatory neurons of P301S Cgas^{-/-} vs P301S Cgas^{+/+}, an enrichment of DEGs in clusters expressing CA1 markers, and CA3 to lesser extent, was observed but not those expressing granule cell markers (FIG. 4G). The effects of cGAS on CA1 hippocampal synapses were further examined with immunofluorescent labeling and quantification of densities of PSD-95, a marker excitatory postsynaptic terminal, in striatum radiatum. cGAS loss resulted in a dose-dependent rescue of tauopathy-induced PSD-95 loss of CA1 pyramidal neurons (FIG. 4H-I). No differences in accumulation of insoluble tau aggregates in hippocampi and entorhinal cortices of transgenic mice was observed using a conformation-specific tau antibody, MC1. These findings support the notion that cGAS loss confers synaptic and cognitive resilience in the presence of tau pathology.

### Deleting Cgas Elevates Expression of Mef2c and its Target Genes in Excitatory and Inhibitory Neurons.

To further dissect how cGAS loss confers protection against tau toxicity, the DEGs in the hippocampal excitatory and inhibitory neurons were examined. Top DEGs in the excitatory neurons, included genes implicated in transcription regulation and chromatin remodeling (Mef2c, Satb1, Satb2), genes regulating excitability (a potassium channel regulator, Dpp10), and genes involved in synapse maintenance (Nrg1 Pcdh7, Pcdh5) (Jaitner et al., 2016; Li et al., 2008; Li et al., 2017; Wang et al., 2020) (FIG. 5A). Using immunofluorescent labelling for NRG1 in the CA1, we validated that NRG1 protein levels were indeed downregulated in tauopathy and rescued by deletion of Cgas (FIG. 5B-C). Cgas deletion also modified transcriptomes of inhibitory neurons in tauopathy. Subclustering of pan-interneuron marker GAD1 and GAD2 positive neuron populations identified 9 inhibitory neuron subpopulations (Arneson et al., 2018; Cembrowski et al., 2016). Genes upregulated by Cgas deletion in interneurons included genes involved in GABAergic signaling, a GABA transporter (Slc6a1), a GABA receptor (Gabbr2), ion channels regulating neuronal excitability and firing, such as shaw-type potassium channels (Kcnc1, Kcnc2). Genes downregulated by Cgas deletion also included calcium channels (Cacnb2, Cacna1e), and Ryanodine Receptor-Calcium Release Channel (Ryr3) (FIG. 5D).

Among the top DEGs upregulated in both excitatory and inhibitory neurons in P301S Cgas/mice is Mef2c, a transcription factor implicated in the late-onset AD, which was recently linked to cognitive resilience in AD brains (Barker et al., 2021). Using immunohistochemistry, elevated MEF2C expression in the hippocampi of P301S Cgas/mice was confirmed. To examine if MEF2C is a key regulator in the neuronal response to Cgas deletion, the DEGs (P301S Cgas^{-/-} vs. P301S) in both excitatory and inhibitory neurons with MEF2C target genes were compared, and observed a striking overrepresentation of the MEF2C target genes in the P301S Cgas^{-/-} excitatory and inhibitory neurons (FIG. 5G). These neuronal transcriptomic changes were specific to the MEF2C network but not to other MEF2 family members or transcription factors regulating neuronal activity, as no strong overlap between the DEGs and MEF2A, FOSL2, and JUNB target genes using two published data sets of transcriptional changes after neuronal activity (Barker et al., 2021) (FIG. 5G).

Cgas loss induced specific enhancement of MEF2C transcriptional network in P301S mice, both as a transcription activator and a repressor in excitatory and inhibitory neurons (FIG. 5I, J). In excitatory neurons (ENs), MEF2C target genes rescued by Cgas deletion in P301S mice included genes involved in axonal guidance, dendritic growth and synaptic maintenance (Tenm3, Unc5d, Nrxn1, Lzts1 Ptprd, Fhod3, Hs6st2), and those in regulating calcium signaling/homeostasis (Cacng3, Ncald, Slc24a3) (FIG. 5I). Similarly, Cgas deletion in inhibitory neurons (INs) also rescued MEF2C target genes involved in axonal guidance, growth and synaptic maintenance (Tenm3, Unc5d, Lzts1, Ctnnd2, Cdh8, Sipall1), and those regulating calcium signaling/homeostasis (Ncald, Camk4) (FIG. 5J). In agreement with the finding that MEF2C overexpression ameliorated hyperexcitability in P301S mice (Barker et al., 2021) we found that Cgas deletion also rescued genes involved in regulating neuronal excitability, such as potassium channel and regulatory subunits, Kcnab2, Dpp10 in ENs (FIG. 5I) and Kcnj9, Kcnip2 in INs (FIG. 5J). Among the downregulated MEF2C target genes by Cgas deletion in both ENs and INs were genes involved in Eph/Ephrin signaling (Eph6 and Eph7) (FIG. 5I-J), blocking of which was found to promote regeneration during injury (Teng et al., 2019). Further examination of Mef2C target genes upregulated in Cgas-ENs and INs revealed multiple overlaps with genes involved in cognitive resilience in AD brains, including Ncald, Rasgef1b, Igsf3, and R3hdm2 (FIG. 5K-L). Thus, the enhanced MEF2C transcription network could drive the protective mechanism underlying Cgas^{-/-} neurons in the presence of tau pathology.

Cgas deletion resulted in a striking upregulation of anti-seizure genes that were diminished in the interneurons of P301S mice (FIG. 6A-D), including Scn1a. Deficiency of Scn1a leads to Dravet syndrome, an intractable childhood epilepsy with generalized tonic-clonic seizures.

Pharmaceutical Inhibition of cGAS Protects Against Synapse Loss and Improves Cognition in Tauopathy Mice

To determine the efficacy of TDI-6570 against tau-mediated neurotoxicity, a cohort of 6-months old Ntg and P301S mice with was fed with 150 mg/kg TDI-6570 or control diet for three months. The effects of TDI-6570 on excitatory and inhibitory neuronal populations were first examined with single-nuclei RNA-seq. Consistent with the genetic deletion of Cgas, Mef2c was a top upregulated gene in the inhibitory neuron clusters. To assess if MEF2C transcription network was significantly enriched in neurons of TDI-6570 treated P301S mice, we again performed overlap analysis between the DEGs and MEF2C target genes in both excitatory and inhibitory neurons (FIG. 7A). Consistent with results from genetic deletion of Cgas, we observed strong enrichment of MEF2C target genes in the P301S TDI-6570 DEGs of both excitatory and inhibitory neurons, but not MEF2A target genes, neuron activity regulating transcription factors FOSL and JUNB target genes, nor activity-induced genes (ARG and scARG) (FIG. 7B). We also identified many MEF2C target genes among the top upregulated genes in P301S TDI-6570 vs P301S excitatory neuron and inhibitory neuron clusters (FIG. 7C-D)

Novel object recognition test paradigm was performed to evaluate the effect of cGAS inhibition on memory. The P301S mice fed with the control diet showed a defect in recognizing the novel (N) object among familiar (F) object (FIG. 7E). Remarkably, this defect was rescued in mice fed with TDI-6750 indicating that cGAS inhibition can ameliorate the memory deficits observed in tauopathies (FIG. 7E). Given the protective effects of Cgas deletion on synapses in tauopathy, we asked if cGAS inhibition can also alter the hippocampal synaptic density of P301S mice. Immunofluorescent staining for excitatory (PSD-95, FIG. 7F-G) and inhibitory (vGAT, FIG. 7H-I) synapses showed that chronic treatment with TDI-6570 rescued P301S-dependent synapse loss. Thus, pharmacological inhibition of cGAS protected against the synaptic loss and cognitive deficits in tauopathies, likely via enhancing MEF2C transcriptional network and associated cognitive resilience.

Our current study linked hyperactive cGAS-interferon antiviral response with diminished MEF2C-associated cognitive resilience. In diseased condition, tau-induced cGAS hyperactivation promotes microglial type I interferon response, reduces neuronal MEF2C transactivation, and renders neurons vulnerable to tau toxicity (FIG. 8). On the other hand, cGAS ablation diminishes microglial type I interferon response, enhances neuronal MEF2C transcription network, leading to cognitive resilience against tau pathology (FIG. 8). Pharmacological inhibition of cGAS with TDI-6570 also enhanced MEF2C target genes and restored synaptic integrity and memory, supporting the therapeutic potential of targeting cGAS-MEF2C axis to improve resilience to treat AD.

Our analyses of two independent datasets revealed that tau induced strong cGAS activation and type I interferon response in tauopathy mice, consistent with a most recent study performed in cultured microglia (Jin et al., 2021). Activation of interferon responses in AD brains and mouse models were reported in previous studies, which was found to be implicated in complement-associated synapse loss (Roy et al., 2020). Using snRNA-seq of human AD microglia, a subpopulation of microglia with overlapping expression of cGAS and STAT1 was identified, which exhibit enrichment in complement and interferon responses. Our finding that levels of pTBK1 were elevated in AD brains vs. normal brains further supports that cGAS-STING activation could underlie the interferon responses in AD. Activation of STING signaling has been linked with the pathogenesis of other neurodegenerative diseases and inhibiting STING activation has been shown to result in protection against deleterious effects of interferon responses in Parkinson's, Huntington's diseases, and ALS (Sharma et al., 2020; Sliter et al., 2018; Yu et al., 2020), supporting a converging deleterious role for cGAS-STING activation in neurodegeneration.

Expression of Cgas in the brain is most enriched in microglia, and our in vitro studies provide direct evidence that tau induces cGAS-dependent interferon signaling in microglia. As the sensor of cytosolic DNA, cGAS could be activated by leakage of DNA from mitochondria or nucleus (Rongvaux et al., 2014; White et al., 2014). We showed that following phagocytosis tau is found in mitochondria as well as lysosomes. It is possible that entry of tau into mitochondria could cause leakage of mitochondrial DNA into the cytosol which triggers cGAS-STING activation. Indeed, by depletion mtDNA using two independent methodologies, we showed that tau-induced interferon responses were reduced in a dose-dependent manner in mtDNA depleted microglia. These findings are consistent with the observations that TDP-43 overexpression leads to the release of mtDNA and cGAS-STING activation and results in neuroinflammation. Studies have shown TDP-43 may enter the mitochondrial matrix via the mitochondrial import inner membrane translocase TIM22, which leads to destabilization of mitochondria and mtDNA leakage (Yu et al., 2020). Whether TIM22 or other mitochondrial translocases are involved in the entry of tau into mitochondria is unknown. It is conceivable that mitochondrial stress induced by tau's entry into mitochondria directly or indirectly leads to the leakage of mtDNA. Despite the clear evidence of mtDNA in tau-induced microglial cGAS-interferon response, our study does not exclude possible contribution of cytosolic DNA of nuclear origin, such as cytosolic chromatin fragments elevated as a result of genomic instability, could also stimulate cGAS-STING activation.

Cgas deletion in tau-stimulated microglia mitigated interferon and inflammatory signaling in vitro and in vivo. Our snRNA-seq data showed that rather than abolishing tau-induced microglial responses to tauopathy, Cgas deletion fine-tunes the microglial responses with DAM responses largely unaffected while only interferon-related genes selectively reduced. Studies have shown that glial interferon signaling can drive synapse elimination by microglia via excessive complement activities in tauopathy (Dejanovic et al., 2018; Litvinchuk et al., 2018).

MEF2C is dramatically downregulated in neurons of HIV-associated dementia patients suggesting an interplay of antiviral signaling and MEF2C transcription network could exist in the brain (Yelamanchili et al., 2010). It remains to be determined if microglial interferon can downregulate neuronal Mef2c. Mouse models with cell type-specific deletion of Cgas are needed to further dissect the link between cGAS and regulators of cognitive resilience and neuronal excitability.

In AD brains, accumulation of amyloid plaques and neurofibrillary tangles precedes clinical symptoms by decades, indicative of a long period of cognitive resilience in healthy aging. Harnessing and promoting brain's intrinsic cognitive resilience mechanism could lead to effective treatment. Our revelation that cGAS inactivation induces striking protection in the presence of tau pathology supports an exciting new class of therapeutic strategy that could be efficacious even after the onset of plaque and tangle pathologies. This along with the fact that Cgas are healthy and fertile supports further evaluation of pharmacological inhibition of cGAS as a promising therapeutic strategy for AD.

Synthesis. All new compounds were confirmed using 1HNMR (Table 1) and mass spectral and/or LC-MS analysis.

1. Indole derivatives. We focused on new indole derivatives, previously not described and no cGAS activity known. These compounds were prepared using the readily available starting materials that are obtained in 3-4 steps from the commercially available compounds as described in Scheme 1.

1.1. Compounds 1 and 2 (Scheme 1A). Commercially available 2-fluoro-3-chloro nitrobenzene was converted to indole A-2 in 4 steps (Lama, et. al, WO2019153002A1). Intermediate A-2 reacted with 2 acetoxyacetyl chloride to give amide A-3 and the latter underwent Pd-catalyzed bromide-hydroxide conversion to afford compound 1 or Suzuki coupling with boronic acid to give compound 1 (Scheme 1A).

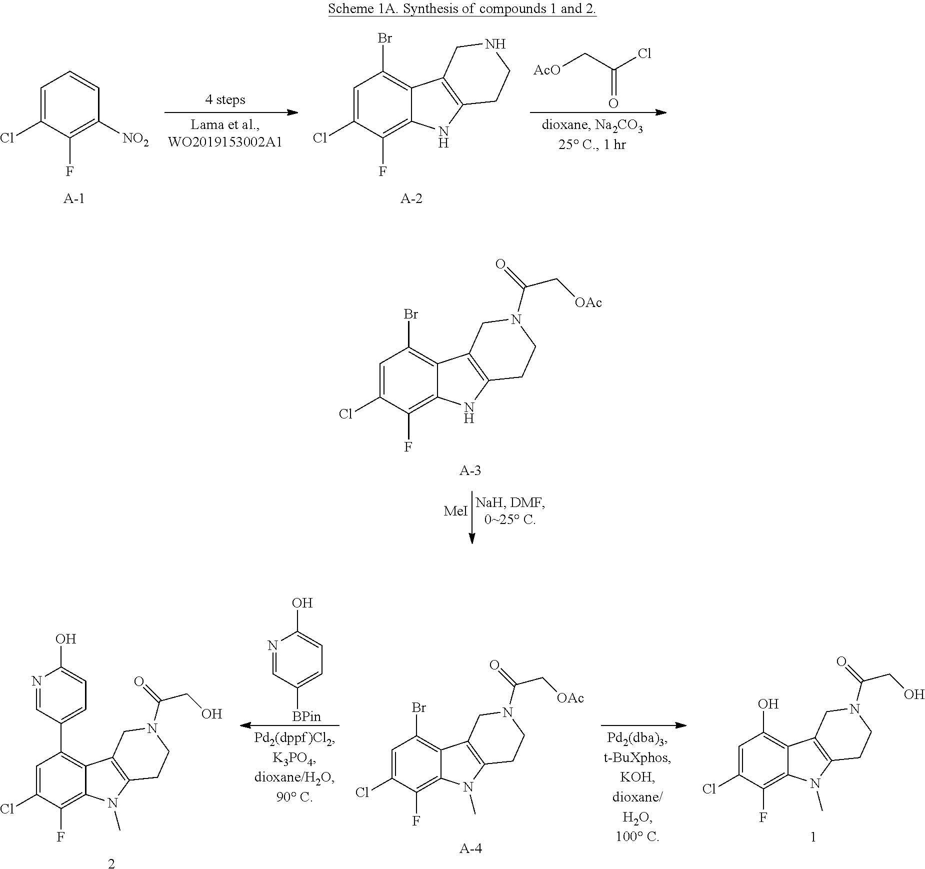

Compound A-2. Commercially available compound A-1 underwent 4 steps transformation, including bromination, nitro-reduction to amine, hydrazine formation and indole synthesis, as described previously (Lama, et. al, WO2019153002A1) to afford A-2 HCl salt (6.2 g, crude) as a white solid. ESI [M+H]=304.9/302.9

Compound A-3. A mixture of A-2, (1.5 g, 4.9 mmol, 1 eq.) in dioxane (20 mL) and Na₂CO₃ sat.aq. (10 mL) was added (2-chloro-2-oxo-ethyl) acetate (4.7 g, 34.4 mmol, 7.0 eq.), then the mixture was stirred at 25° C. for 1 hr. The mixture was quenched with H₂O (10 mL) and extracted with EtOAc (30 mL*3), washed with brine (15 mL*2), dried over Na₂SO₄, filtered, concentrated and purified by column chromatography (SiO₂, Pet. ether: THF, 1:0 to 1:1) to yield A-3, (1.2 g, 2.9 mmol, 57.7% yield) as a yellow solid. ESI [M+H]=404.9/402.9

Compound A-4. A mixture of A-3 (1.1 g, 2.7 mmol, 1 eq.) in DMF (30 mL) was added NaH (218 mg, 5.5 mmol, 60% purity, 2 eq.) and CH₃I (773.6 mg, 5.5 mmol, 2 eq.) at 0° C., then the mixture was stirred at 25° C. for 1 hr. The mixture was quenched by 1 N HCl (15 mL) and extracted with EtOAc (50 mL*3). The organic layer was washed with brine (10 mL*5), dried over Na₂SO₄, filtered, concentrated and purified by prep-HPLC (TFA condition; column: Phenomenex luna C18 250*50 mm*10 μm; mobile phase: [water (0.1% TFA)-ACN]; B %: 50%-70%, 10 min) to yield A-4, (370 mg, 885.9 umol, 32.5% yield) as a white solid. ESI [M+H]=419.0/417.0

Compound 1. A mixture of A-4, (100 mg, 239.4 umol, 1 eq.), Pd₂(dba)₃ (22 mg, 23.9 umol, 0.1 eq.), t-Bu Xphos (10 mg, 23.9 umol, 0.1 eq.) and KOH (27 mg, 478.9 umol, 2 eq.) in dioxane (2.4 mL) and H₂O (0.9 mL) was degassed and purged with N₂ for 3 times, and then the mixture was stirred at 100° C. for 1 hr under N₂ atmosphere. The mixture was quenched by H₂O (15 mL) and extracted with EtOAc (50 mL*3). The organic layer was washed with brine (10 mL*5), dried over Na₂SO₄, filtered, concentrated and purified by prep-HPLC (TFA condition; column: Phenomenex Luna 80*30 mm*3 μm; mobile phase: [water (0.1% TFA)-ACN]; B %: 20%-45%, 8 min) to yield 1-(7-chloro-6-fluoro-9-hydroxy-5-methyl-3,4-dihydro-1H-pyrido[4,3-b]indol-2-yl)-2-hydroxy-ethanone, 1, (69 mg, 212.4 umol, 88.7% yield, 96.3% purity) as a yellow solid.

Compound 2 (general method A). A mixture of A-4, (50 mg, 120 umol, 1.0 eq.), 5-(4,4,5,5-tetramethyl-1,3,2-dioxaborolan-2-yl) pyridin-2-ol (106 mg, 479 umol, 4.0 eq.), Pd(dppf)Cl₂ (9 mg, 12 umol, 0.1 eq.), K₃PO₄ (102 mg, 479 umol, 4.0 eq.) in H₂O (0.5 mL) and dioxane (1.5 mL) was stirred at 80° C. for 12 hrs under N₂ atmosphere, then diluted with H₂O (2 mL) and extracted with EtOAc (3 mL*3). The organic layer was dried over Na₂SO₄, filtered, concentrated and purified by prep-HPLC (column: Phenomenex Luna 80*30 mm*3 μm; mobile phase: [water (TFA)-ACN]; B %: 20%-50%, 8 mins) to yield compound 2, (6 mg, 13 umol, 11.3% yield, 92.6% purity) as a yellow gum.

1.2. Compound 3 (Scheme 1B). Prepared similarly as described for compound 1, except the starting material A-5 was used, besides the Pd-catalyzed conversion of bromo function in compound A-7 to phenol in A-8.

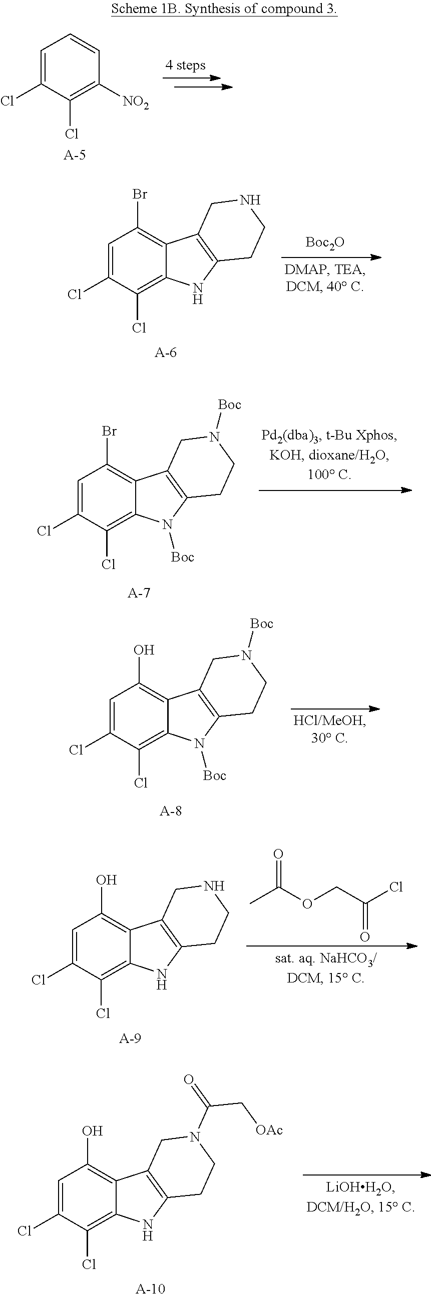

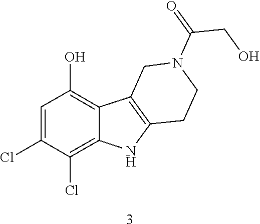 [SMILES: O=C(CO)N1CCc2[nH]c3c(Cl)c(Cl)cc(O)c3c2C1 ; "3"]

Compound A-6. The commercially available compound A-5 was converted to A-6 in 4 steps as described for A-1 to A-2 in Scheme 1A. The title product was obtained as a pale pink solid (5.5 g, crude, HCl. ¹H NMR (400 MHZ, DMSO-d₆) δ=12.14 (s, 1H), 9.22 (br s, 1H), 7.52 (s, 1H), 4.59 (s, 2H), 3.50 (br t, J=6.0 Hz, 2H), 3.05 (br t, J=5.8 Hz, 2H). ESI [M+H]=320.9/318.9.

Compound A-7. To a solution of A-6, (700 mg, 2.2 mmol, 1.0 eq.) and TEA (664 mg, 6.6 mmol, 3.0 eq.) in DCM (10 mL) was added Boc₂O (2.9 g, 13.1 mmol, 6.0 eq.) and DMAP (27 mg, 218.7 umol, 0.1 eq.) and stirred at 40° C. for 12 hrs. Water (10 mL) was added and extracted with DCM (10 mL*3). The combined organic layers were dried over Na₂SO₄, filtered, concentrated and purified by column chromatography (SiO₂, Petroleum ether: Ethyl acetate=20:1 to 15:1) to yield compound A-7, (550 mg, 1.1 mmol, 48.3% yield) as a white solid. 1H NMR (400 MHZ, CDCl₃) δ=7.49 (s, 1H), 4.90 (br s, 2H), 3.77 (br s, 2H), 2.90 (br s, 2H), 1.64 (s, 9H), 1.51 (s, 9H). ESI [M-Boc+H]=421.0/419.0

Compound A-8. A mixture of compound A-7, (480 mg, 922.7 umol, 1.0 eq.), Pd₂ (dba) 3 (85 mg, 92.3 umol, 0.1 eq.), t-Bu Xphos (39 mg, 92.3 umol, 0.1 eq.) and KOH (104 mg, 1.9 mmol, 2.0 eq.) in dioxane (4 mL) and H₂O (1.5 mL) stirred at 100° C. for 1 hr under N₂ atmosphere. The mixture was added H₂O (5 mL), extracted with EtOAc (20 mL*3), dried over Na₂SO₄, filtered, concentrated and purified by prep-HPLC (TFA condition; column: Phenomenex luna C18 100*40 mm*5 μm; mobile phase: [water (0.1% TFA)-ACN]; B %: 40%-80%, 8 min) to yield A-8, (110 mg, 240.5 umol, 26.1% yield) as a white solid. ¹H NMR (400 MHz, DMSO-d₆) δ=6.80 (s, 1H), 4.63 (br s, 2H), 3.76-3.60 (m, 2H), 3.01-2.79 (m, 2H), 1.64-1.56 (m, 9H), 1.45 (s, 9H). ESI [M-Boc+H]=357.1/359.1

Compound A-9 (general method B). A mixture of compound A-8 (80 mg, 174.9 umol, 1.0 eq.) in HCl/MeOH (4 mL) (4 M) was stirred at 30° C. for 18 hrs. The reaction mixture was concentrated and dried in vacuo to yield A-9 (50 mg, crude) as a pale brown solid. ESI [M+H]=257.0/259.0

Compound A-10. To a mixture of A-9 (35 mg, 136.1 umol, 1.0 eq.) in DCM (1 mL) and sat.aq. NaHCO₃ (0.5 mL) was added 2-acetoxyacetyl chloride (149 mg, 1.1 mmol, 8.0 eq.) and the resulting mixture was stirred at 15° C. for 1 hr. Water (2 mL) was added and extracted with DCM (5 mL*3). The combined organic layers were dried over Na₂SO₄, filtered, concentrated and dried in vacuo to yield A-10 (50 mg, crude) as a yellow solid. ESI [M+H]=357.0/359.0

Compound 3 (general method C). To a solution of compound A-10, (50 mg, 140.0 umol, 1.0 eq.) in DCM (2 mL) was added LiOH·H₂O (1 M, 1.4 mL, 10.0 eq.) and the mixture was stirred at 15° C. for 1 hr. The reaction was quenched with HCl (2 mL, 1M), extracted with DCM (5 mL*3), dried over Na₂SO₄, filtered, concentrated and purified by prep-HPLC (TFA condition; column: Phenomenex Luna C18 150*30 mm*5 μm; mobile phase: [water (0.1% TFA)-ACN]; B %: 1%-35%, 8 min) to yield compound 3 (5 mg, 14.0 umol, 10.0% yield, 100.0% purity) as a yellow gum.

1.3. Compounds 4 and 5 (Scheme 1C). The title compounds were prepared using A-6 in 5 steps. First, compound A-6 was Boc-protected giving A-11 and then N-methylated to give A-12. The latter underwent Suzuki coupling with N-methyl pyrazole boronic acid derivative to afford A-13. Subsequent deprotection to A-14, followed by reaction with 2-hydroxy propanoic acid or 2-methyl-2-hydroxy propanoic acid afforded compounds 4 and 5.

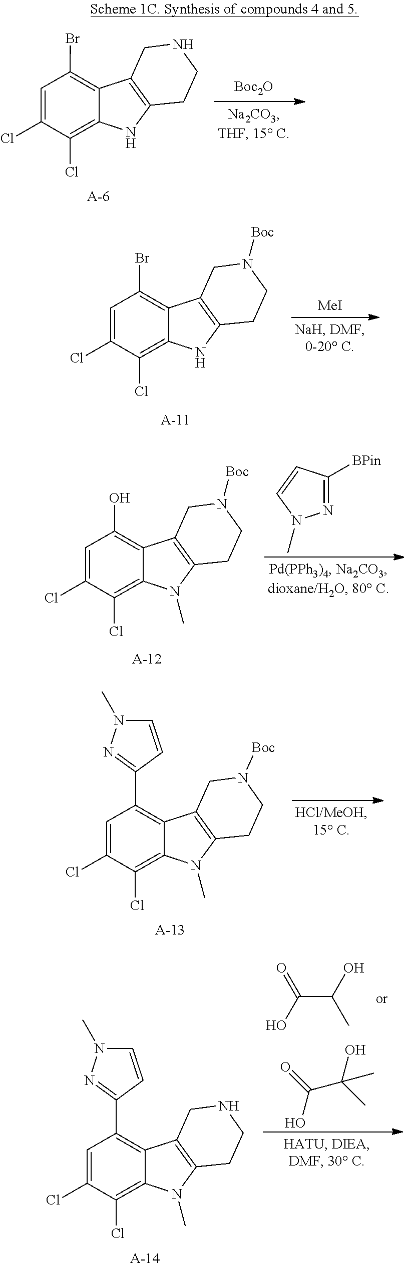

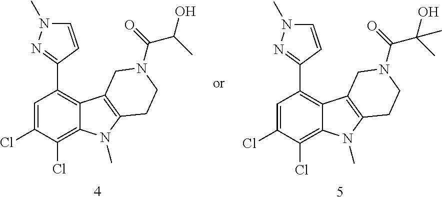

Compound A-11. To a solution of compound A-6, (700 mg, 2.0 mmol, 1.0 eq., HCl) in THF (8 mL) was added sat. aq·Na₂CO₃ (2 mL) and Boc₂O (429 mg, 2.0 mmol, 1.0 eq.). The mixture was stirred at 15° C. for 1 hr. Water (2 mL) was added and extracted with EtOAc (10 mL*3), dried over Na₂SO₄, filtered, concentrated and purified by column chromatography (SiO₂, Petroleum ether: THF, 10:1 to 2:1) to yield A-11 (670 mg, 1.6 mmol, 81.2% yield) as a yellow solid. ¹H NMR (400 MHz, DMSO-d₆) δ=11.87 (s, 1H), 7.44 (s, 1H), 4.83 (br s, 2H), 3.70 (br t, J=5.6 Hz, 2H), 2.81 (br s, 2H), 1.45 (s, 9H). ESI [M-tBu+H]=364.9/362.9

Compound A-12. To a solution of A-11, (580 mg, 1.4 mmol, 1.0 eq.) in DMF (5 mL) was added NaH (110 mg, 2.8 mmol, 60.0% purity, 2.0 eq.) and CH₃I (235 mg, 1.7 mmol, 1.2 eq.) at 0° C. The resulting mixture was stirred at 20° C. for 1 hr. The reaction mixture was quenched by sat. aq. NH₄Cl (10 mL), extracted with EtOAc (10 mL*3), dried over Na₂SO₄, filtered, concentrated and triturated with EtOAc (5 mL) to yield A-12 (520 mg, 1.2 mmol, 84.4% yield, 97.3% purity) as a white solid. ¹H NMR (400 MHZ, DMSO-d₆) δ=7.44 (s, 1H), 4.83 (br s, 2H), 3.94 (s, 3H), 3.70 (t, J=5.8 Hz, 2H), 2.83-2.73 (m, 2H), 1.44 (s, 9H). ESI [M+H]=378.8/376.8

Compound A-13. A mixture of A-12, (250 mg, 575.8 umol, 1.0 eq.), 1-methyl-3-(4,4,5,5-tetramethyl-1,3,2-dioxaborolan-2-yl) pyrazole (144 mg, 691.0 umol, 1.2 eq.), Pd(PPh₃)₄ (67 mg, 57.6 umol, 0.1 eq.) and Na₂CO₃ (183 mg, 1.7 mmol, 3.0 eq.) in dioxane (4.5 mL) and H₂O (1.5 mL) was degassed and purged with N₂ for 3 times, and then stirred at 80° C. for 4 hrs under N₂ atmosphere. The mixture was diluted with H₂O (10 mL) and extracted with EtOAc (10 mL*3), dried over Na₂SO₄, filtered, concentrated and purified by prep-HPLC (TFA condition; column: Phenomenex Luna 80*30 mm*3 μm; mobile phase: [water (0.1% TFA)-ACN]; B %: 50%-85%, 8 min) to yield tert-butyl A-13 (310 mg, crude) as a green solid. ¹H NMR (400 MHZ, DMSO-d₆) δ=7.78 (d, J=2.2 Hz, 1H), 7.15 (s, 1H), 6.50-6.37 (m, 1H), 4.22 (s, 2H), 3.96 (s, 3H), 3.91 (s, 3H), 3.68-3.59 (m, 2H), 2.78 (br t, J=5.6 Hz, 2H), 1.35 (br s, 9H). ESI [M+H]=435.1/437.1

Compound A-14. A solution of A-13 (260 mg, 597.2 umol, 1.0 eq.) in HCl/MeOH (4 M, 5 mL) was stirred at 15° C. for 1 hr. The reaction mixture was concentrated to yield A-14 (192 mg, crude HCl salt) as a brown solid. ESI [M+H]=335.1/337.1.

Compound 4 (general method D). To a solution of A-14, (50 mg, 134.5 umol, 1.0 eq., HCl) and DIEA (70 mg, 538.1 umol, 4.0 eq.) in DMF (1 mL) was added 2-hydroxypropanoic acid (24 mg, 269.0 umol, 2.0 eq.), HATU (77 mg, 201.8 umol, 1.5 eq.) and the mixture was stirred at 30° C. for 1 hr. The mixture was filtered to obtain filtrate and purified by prep-HPLC (TFA condition; column: Phenomenex Luna 80*30 mm*3 μm; mobile phase: [water (TFA)-ACN]; B %: 23%-53%, 8 min) to yield 4 (7 mg, 15.5 umol, 11.5% yield, 93.2% purity) as a yellow solid.

Compound 5. Obtained by method D, as described for 4, using intermediate A-14 and 2-hydroxy-2-methyl propanoic acid.

1.4. Compounds 6-8 (Scheme 1D). The title compounds were prepared using intermediate A-12. First, intermediate A-12 was deprotected under acidic conditions and the resulting amine A-15 was reacted with NBoc glycine to afford A-16. Subsequent Boc deprotection in A-16 afforded compound 6. Separately, Suzuki coupling of A-16 with pyrimidine boronic acid derivative gave compound 7 and was followed by Boc deprotection to afford 8.

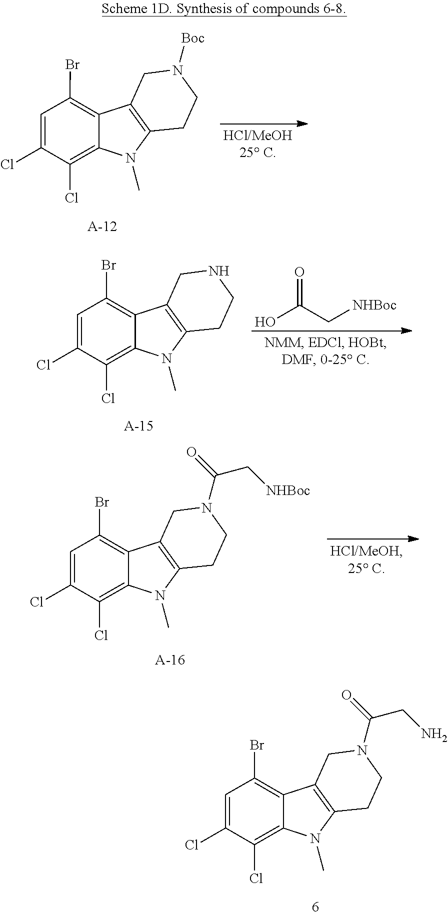

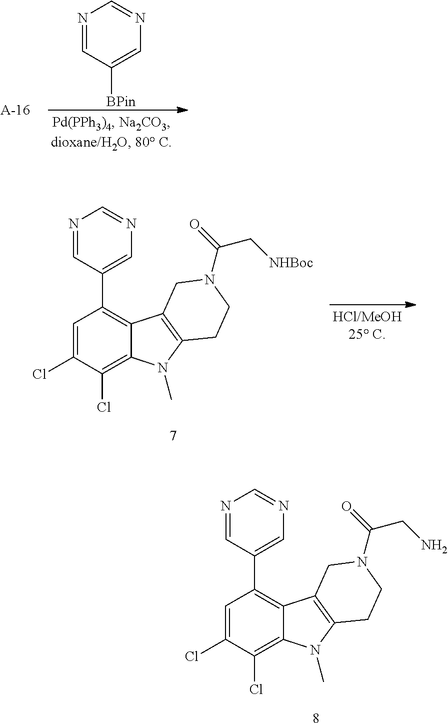

Compound A-15. Boc deprotection in A-12 using methanolic HCl afforded A-15 HCl salt as yellow solid. ESI [M+H]=334.9/332.9.

Compound A-16. To a solution of A-15 (10 mg, 30.0 umol, 1.0 eq.) and NMM (12 mg, 119.8 umol, 4.0 eq.) in DMF (1 mL) was added EDCI (9 mg, 44.9 umol, 1.5 eq.), HOBt (6 mg, 44.9 umol, 1.5 eq.) and N-Boc glycine (11 mg, 59.9 umol, 2.0 eq.) at 0° C. and the mixture was stirred at 20° C. for 1 hr. The reaction mixture was filtered and purified by prep-HPLC (column: Phenomenex Luna 80*30 mm*3 μm; mobile phase: [water (TFA)-ACN]; B %: 45%-85%, 8 min) to yield A-16 (6 mg, 11.9 umol, 39.9% yield, 100.0% purity) as a white solid. 1H NMR (400 MHZ, DMSO-d₆) δ=7.51-7.44 (m, 1H), 6.91-6.77 (m, 1H), 4.97-4.87 (m, 2H), 3.95 (s, 3H), 3.94-3.89 (m, 2H), 3.87-3.76 (m, 2H), 2.93-2.74 (m, 2H), 1.38 (s, 9H). ESI [M-tBu+H]=435.9/433.9.

Compound 6. Prepared by Boc deprotection in A-16 using method B. The title product (6 mg, 13.3 umol, 43.7% yield, 97.0% purity) was obtained as a yellow solid.

Compound 7. A mixture of A-16 (12 mg, 24.4 umol, 1.0 eq.), 5-(4,4,5,5-tetramethyl-1,3,2-dioxaborolan-2-yl)pyrimidine (6 mg, 29.3 umol, 1.2 eq.), Pd(PPh₃)₄ (3 mg, 2.4 umol, 0.1 eq.) and Na₂CO₃ (8 mg, 73.3 umol, 3.0 eq.) in dioxane (0.9 mL) and H₂O (0.3 mL) was degassed and purged with N₂ for 3 times, and then the mixture was stirred at 80° C. for 2 hrs under N₂ atmosphere. The reaction mixture was concentrated and purified by prep-HPLC (column: Phenomenex Luna 80*30 mm*3 μm; mobile phase: [water (TFA)-ACN]; B %: 35%-65%, 8 min) to yield compound 7 (11 mg, 23.0 umol, 94.0% yield, 99.4% purity) as a yellow gum.

Compound 8). Prepared by Boc deprotection in compound 7 using method B to obtain 8 (12 mg, 30.4 umol, 74.4% yield, 98.7% purity) as a white solid.

1.5. Compound 9 (Scheme 1E). Compound 9 was prepared by NaBH₃CN reaction of compound 7, followed by Boc-deprotection under acid conditions.

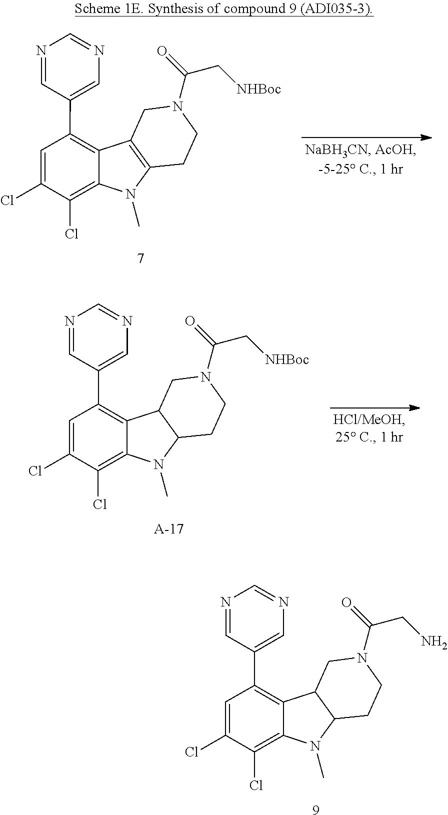

Compound A-17. A mixture of compound 7 (70 mg, 142.8 umol, 1.0 eq.) in AcOH (1 mL) was added NaBH₃CN (11 mg, 171.3 umol, 1.2 eq.) at −5° C. and stirred at 25° C. for 1 hr. The mixture was diluted with sat·aq·Na₂CO₃ (5 mL) and extracted with EtOAc (10 mL*3). The organic layer was washed with brine (3 mL*5), dried over Na₂SO₄, filtered, concentrated and purified by prep-HPLC (column: Waters Xbridge BEH C18 100*30 mm*10 μm; mobile phase: [water (NH₄HCO₃)-ACN]; B %: 30%-60%, 10 min) to yield A-17 (6 mg, 12.2 umol, 8.5% yield) as a white solid. ESI [M+H]=492.2/494.2

Compound 9. Obtained (2 mg, 6.3 umol, 62.0% yield, 100.0% purity) using method B as a yellow gum.

1.6. Compounds 10-12 (Scheme 1F). The key intermediate A-12 underwent Suzuki coupling with N-methylpyrazol boron-pinacol (Pin) affording compound A-18 and the later underwent Boc-deprotection giving free amine A-19, followed by amide formation with the N-Boc glycine giving compound 10, and Boc-deprotection to give free amine 11. Compound 12 was obtained by NaBH₃CN reduction of 11 as described above for compound 7 to 8.

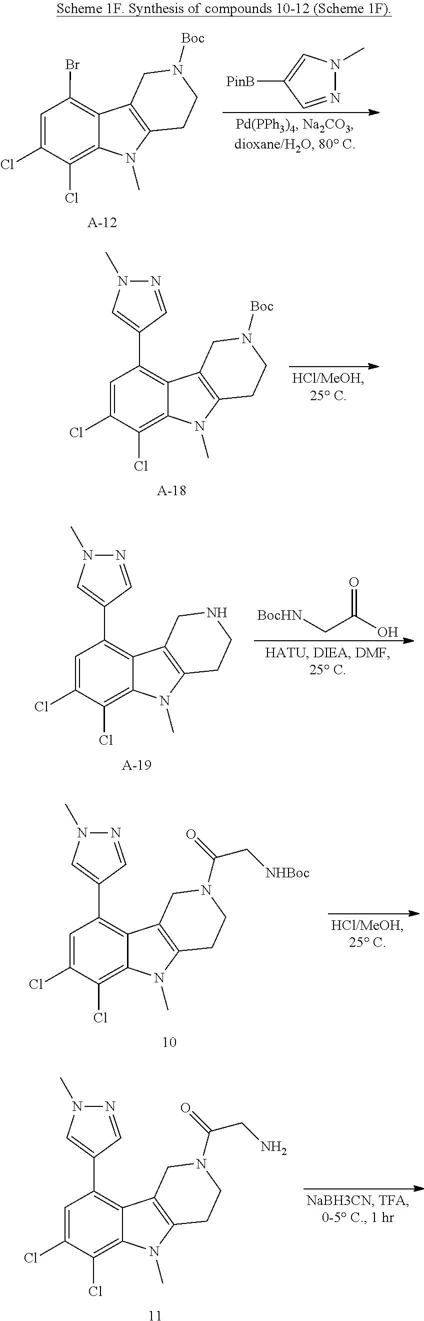

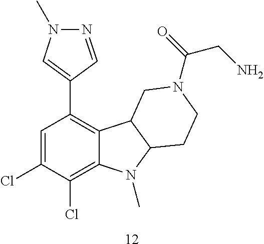

Compound A-18. A mixture of compound A-12, (580 mg, 1.3 mmol, 1.0 eq.), 1-methyl-4-(4,4,5,5-tetramethyl-1,3,2-dioxaborolan-2-yl) pyrazole (334 mg, 1.6 mmol, 1.2 eq.), Pd(PPh₃)₄ (154 mg, 133.4 umol, 0.1 eq.), Na₂CO₃ (425 mg, 4.0 mmol, 3.0 eq.) in dioxane (9 mL) and H₂O (3 mL) was degassed and purged with N₂ for 3 times, and then the mixture was stirred at 80° C. for 2 hrs under N₂ atmosphere. The mixture was diluted with H₂O (10 mL), extracted with EtOAc (10 mL*5), dried over Na₂SO₄, filtered, concentrated and purified by prep-HPLC (column: Phenomenex Luna 80*30 mm*3 μm; mobile phase: [water (TFA)-ACN]; B %: 50%-80%, 8 min) to yield compound A-18 (210 mg, 482.4 umol, 36.1% yield, 100.0% purity) as a yellow solid. ¹H NMR (400 MHZ, MeOH-d4) δ=7.72 (s, 1H), 7.54 (s, 1H), 6.99 (s, 1H), 4.16 (br s, 2H), 4.03-3.96 (m, 6H), 3.76 (brs, 2H), 2.81 (br t, J=5.7 Hz, 2H), 1.49-1.37 (m, 9H). ESI [M+H]=435.1/437.1

Compound A-19. Obtained as a yellow solid (3.44 mg, 9.43 umol, 41.04% yield, 91.861% purity) by performing N-Boc deprotection in A-18 using Method B. ¹H NMR (400 MHz, DMSO-d₆) δ=8.91 (br s, 1H), 7.95 (s, 1H), 7.60 (s, 1H), 7.11 (s, 1H), 4.02 (s, 3H), 3.96-3.88 (m, 5H), 3.47 (br s, 2H), 3.07 (br t, J=5.8 Hz, 2H). ESI [M+H]=335.0/336.9

Compound 10. Prepared by reacting A-24 with N-Boc glycine using method C. The title product 10 (7 mg, 14.1 umol, 47.3% yield, 95.9% purity) was obtained as a yellow solid.

Compound 11. N-Boc deprotection of compound 10 using methanolic HCl (method B) afforded 11 (11 mg, 26.5 umol, 43.4% yield, 97.4% purity) as a yellow solid.

Compound 12. To a solution of 11 (15 mg, 30.5 umol, 1.0 eq.) in TFA (0.5 mL) was added NaBH₃CN (10 mg, 152.3 umol, 5.0 eq.) at 0° C. and the mixture was stirred at 15° C. for 0.5 h. Then the mixture was concentrated, diluted with sat·aq·Na₂CO₃ (10 mL), extracted with EtOAc (10 mL*3), dried over Na₂SO₄, filtered, concentrated and purified by prep-HPLC (column: Waters Xbridge BEH C18 100*30 mm*10 μm; mobile phase: [water (NH₄HCO₃)-ACN]; B %: 25%-55%, 10 min) to yield 12 (1.0 mg, 2.5 umol, 8.2% yield, 97.0% purity) as a yellow gum.

1.7. Compounds 13-18 (Scheme 1G). The title compounds were prepared using indole derivatives A-6 and A-15 in 2-3 steps. First, A-6 and A-15 reacted with Boc-protected proline to afford amides A-20 and A-21, and these compounds underwent appropriate boronic acid derivatives to afford compounds 13-15. Next, these products underwent Boc-deprotection to afford the free amine compounds 16-18.

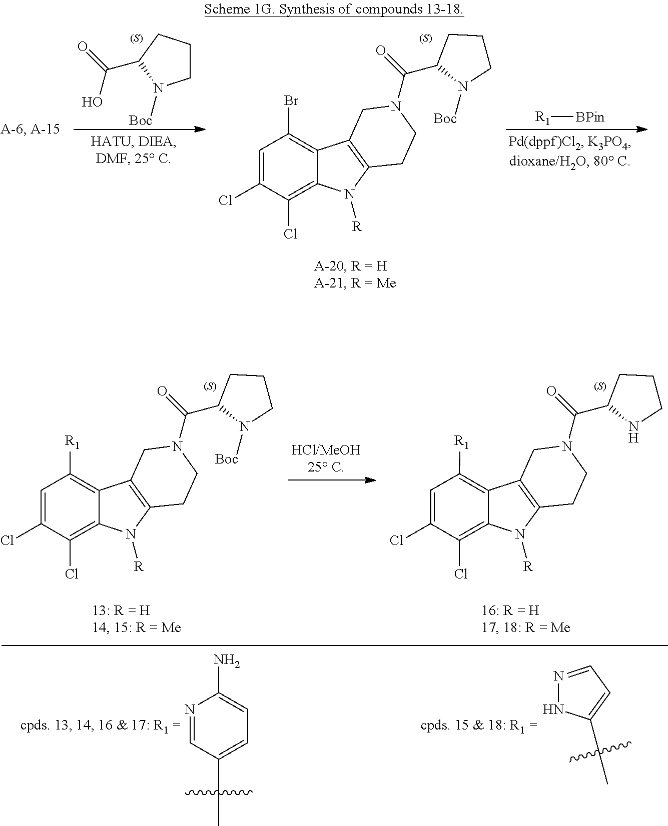

1.8. Compounds 19 and 20 (Scheme 1H). The title compounds were prepared using compound A-6 in three steps. Here, A-6 underwent amide formation with N-Boc protected aminocyclopropyl and amino-iso-propyl carboxylic acids to afford A-22 and A-24, respectively. Suzuki coupling of A-22 with aminopyridine boronic acid and of A-24 with pyrazole boronic acid derivatives afforded compound A-23 and A-25. Finally, the latter products were N-Boc deprotected to give 19 and 20, respectively.

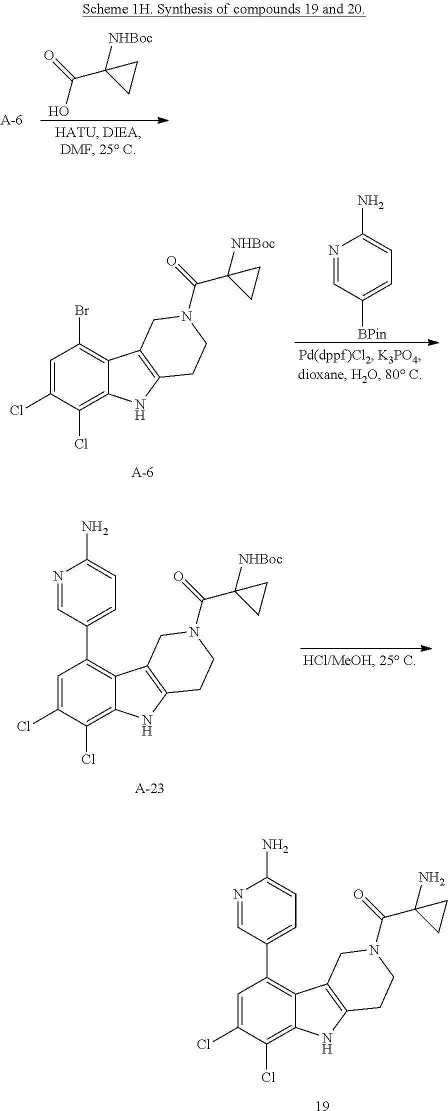

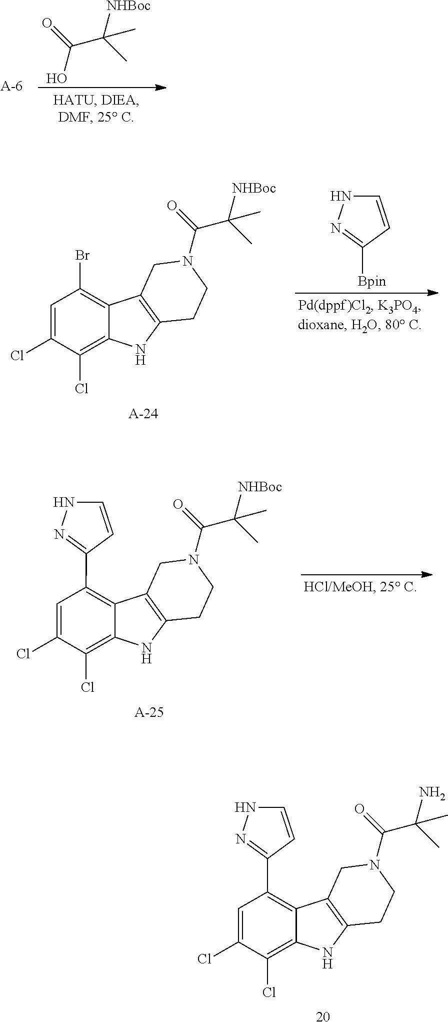

Compound A-22. Compound A-6 was reacted with Boc protected 2-aminocyclopropyl carboxylic acid using method C to afford A-22 as a white solid (140 mg, 278.21 umol, 99.17% yield).

Compound A-23. Compound A-22 was coupled with 2-aminopyridine-5-boronic acid derivative using method A to afford A-23 (8.58 mg, 12.88 umol, 9.30% yield, 94.97% purity, TFA) as a white solid. ¹H NMR (400 MHZ, DMSO-d₆) δ=11.75 (s, 1H), 8.09 (s, 2H), 7.99 (br d, J=8.6 Hz, 2H), 7.79 (br s, 1H), 7.70-7.40 (m, 1H), 7.14 (s, 1H), 7.04 (d, J=9.0 Hz, 1H), 4.12 (br s, 2H), 3.88 (br s, 2H), 2.89 (br s, 2H), 1.29 (br s, 7H), 1.15-1.01 (m, 4H), 0.87-0.79 (m, 2H). ESI [M+H]=516.1/518.1

Compound 19 (ADI-51-9). NBoc deprotection in A-23 using method B afforded compound 19 (1.48 mg, 2.95 umol, 30.58% yield, 90.62% purity) as a HCl salt.

Compound A-24. Compound A-6 was reacted with Boc protected 2-amino-iso-propyl carboxylic acid using method C to afford A-24 (50 mg, crude). ESI [M+H]=506.0/504.0.

Compound A-25. Compound A-24 was coupled with pyrazole-3-boronic acid derivative using method A to afford A-25 (2.78 mg, 5.65 umol, 28.52% yield) as a yellow gum. 1H NMR (400 MHZ, DMSO-d₆) δ=11.55-11.48 (m, 1H), 7.89-7.70 (m, 2H), 7.38-7.13 (m, 2H), 6.54 (br s, 1H), 4.63-4.33 (m, 2H), 4.01-3.86 (m, 2H), 2.86-2.77 (m, 2H), 1.31 (br s, 9H), 1.17-0.94 (m, 6H). ESI [M+H]=492.1/494.1.

Compound 20 (ADI-47-13). NBoc deprotection in A-25 using method B afforded compound 20 (3.39 mg, 8.41 umol, 8.28% yield, 97.31% purity) as a yellow gum.

2. Benzofuran derivatives. We designed, prepared, and evaluated benzofuran derivatives to mimic the indole compounds. Synthesis of various benzofuran derivatives was achieved starting with the commercially available materials as described in sections 2.1-2.x.

2.1. Compound 21 (Scheme 2A). Synthesize of compound 21 started by reacting boronic acid B-1 with N-hydroxyphthalimide to afford compound B-2. The latter underwent removal of phathalic acid by reacting with MeNH₂ giving B-3, which reacted with 4-tetrahydropyridone to afford the key intermediate B-4. Subsequent reaction with 2-acetoxyacetyl chloride and deprotection of the acetate group afforded compounds B-5 and then 21.

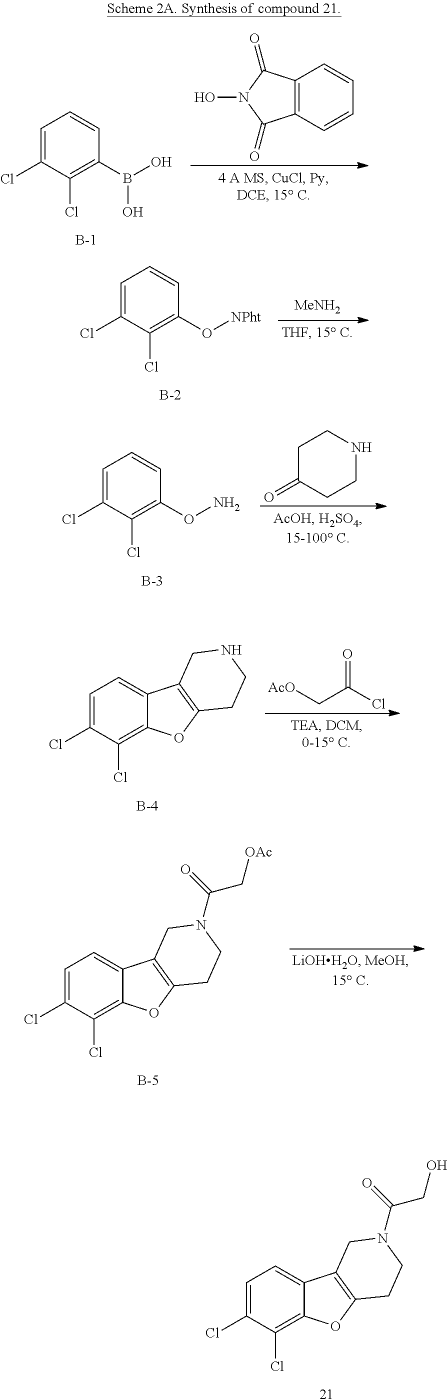

Compound B-2. A mixture of (2,3-dichlorophenyl) boronic acid, B-1, (11.7 g, 61.3 mmol, 2.0 eq.), 2-hydroxyisoindoline-1,3-dione (5.0 g, 30.7 mmol, 1.0 eq.), Py (2.7 g, 33.7 mmol, 1.1 eq.), 4A MS (2.0 g) and CuCl (3.0 g, 30.7 mmol, 1.0 eq.) in DCE (200 mL) was degassed and purged with O₂ for 3 times, and then the mixture was stirred at 40° C. for 48 hrs under O₂ atmosphere. The mixture was diluted with water (50 mL) and extracted with DCM (100 mL*3), dried over Na₂SO₄, filtered, concentrated and purified by prep-HPLC (TFA condition; column: Phenomenex luna C18 250 mm*100 mm*10 μm; mobile phase: [water (0.1% TFA)-ACN]; B %: 40%-70%, 20 min) to yield compound B-2 (550 mg, 1.8 mmol, 5.8% yield) as a brown solid. ¹H NMR (400 MHZ, DMSO-d₆) δ=8.02-7.92 (m, 4H), 7.57 (dd, J=1.2, 8.5 Hz, 1H), 7.49 (dd, J=1.2, 8.1 Hz, 1H), 7.39-7.31 (m, 1H). ESI [M+H]=308/309.9

Compound B-3. A mixture of compound B-2 (550 mg, 1.8 mmol, 1 eq.) and MeNH₂ in EtOH (30.0% MeNH₂ in EtOH, 8 mL) was stirred at 15° C. for 1 hr. The reaction mixture was concentrated to yield compound B-3 (840 mg, crude) as a brown solid. ¹H NMR (400 MHz, DMSO-d₆) δ=7.64 (br d, J=6.7 Hz, 1H), 7.42-7.37 (m, 1H), 7.03-6.97 (m, 1H), 2.34 (s, 2H).

Compound B-4. To a mixture of compound B-3 (840 mg, 4.7 mmol, 1.0 eq.) in AcOH (10 mL) was added H₂SO₄ (1 mL) followed by tert-butyl 4-oxopiperidine-1-carboxylate (1.1 g, 5.7 mmol, 1.2 eq.). The mixture was stirred at 15° C. for 5 mins and then heated to 100° C. for 12 hrs. The reaction mixture was concentrated and purified by prep-HPLC (TFA condition; column: Phenomenex luna C18 250*50 mm*10 μm; mobile phase: [water (0.1% TFA)-ACN]; B %: 15%-45%, 10 min) to yield B-4 (350 mg, 982.8 umol, 20.8% yield, TFA salt) as a brown solid. 1H NMR (400 MHz, DMSO-d₆) δ=7.70-7.65 (m, 1H), 7.61-7.57 (m, 1H), 4.38 (s, 2H), 3.59 (br t, J=6.0 Hz, 2H), 3.20-3.11 (m, 2H). ESI [M+H]=242.0/244.0

Compound B-5. A mixture of compound B-4, (100 mg, 280.8 umol, 1.0 eq., TFA) and TEA (57 mg, 561.6 umol, 2.0 eq.) in DCM (1 mL) was added 2-acetoxyacetyl chloride (58 mg, 421.2 umol, 1.5 eq.) at 0° C. and stirred at 15° C. for 1 hr. The mixture was quenched with MeOH (0.5 mL) and concentrated to yield B-5 (100 mg, crude) as a brown solid. ESI [M+H]=342.0/344.0

Compound 21. A mixture of compound B-5 (100 mg, 292.3 umol, 1.0 eq.) and LiOH·H₂O (25 mg, 584.5 umol, 2.0 eq.) in MeOH (2 mL) was stirred at 15° C. for 1 hr. The mixture was concentrated and purified by rep-HPLC (column: Waters Xbridge BEH C18 100*30 mm*10 μm; mobile phase: [water (10 mM NH₄HCO₃)-ACN]; B %: 35%-65%, 10 min) to yield compound 21 (8 mg, 27.6 umol, 9.4% yield, 100.0% purity) as a white solid.

2.2. Compounds 22-26 and 28-29 (Scheme 2B). To prepare the title compounds, the key benzofuran intermediates B-20 and B-21 were synthesized starting with the commercially available compounds B-6 and B-7, via boronic acids B-12 and B-13, respectively, (Scheme 2B). Here, compounds B-6 and B-7 were reacted with sodium methoxide to give B-8 and B-9, which underwent Pd-catalyzed boronylation with B₂Pin₂ and the resulting products B-10 and B-11 were treated with sodium periodate giving B-12 and B-13. The latter products were reacted with N-hydroxy phthalimide and the resulting products B-14 and B-15 were hydrolyzed using MeNH₂ in ethanol giving compounds B-16 and B-17. Subsequently, B-16 and B-17 underwent coupling with N-Boc-4-tetrahydropyridone and the resulting benzofurans B-18 and B-19 were reacted with BBr₃ to afford key intermediates B-20 and B-21. The latter compounds reacted with 2-acetoxyacetyl chloride in the presence of Na₂CO₃ and the products, B-22 and B-23, were converted to triflates B-24 and B-25 by reacting with triflic anhydride. Next, B-24 and B-25 underwent Suzuki coupling with appropriate heterocyclic boronic acid derivatives to afford compounds B-(26-32) and acetate deprotection to afford the title products 22-26 and 28-29. Methods described here are for compound B-7 and processed similarly for compound B-6.

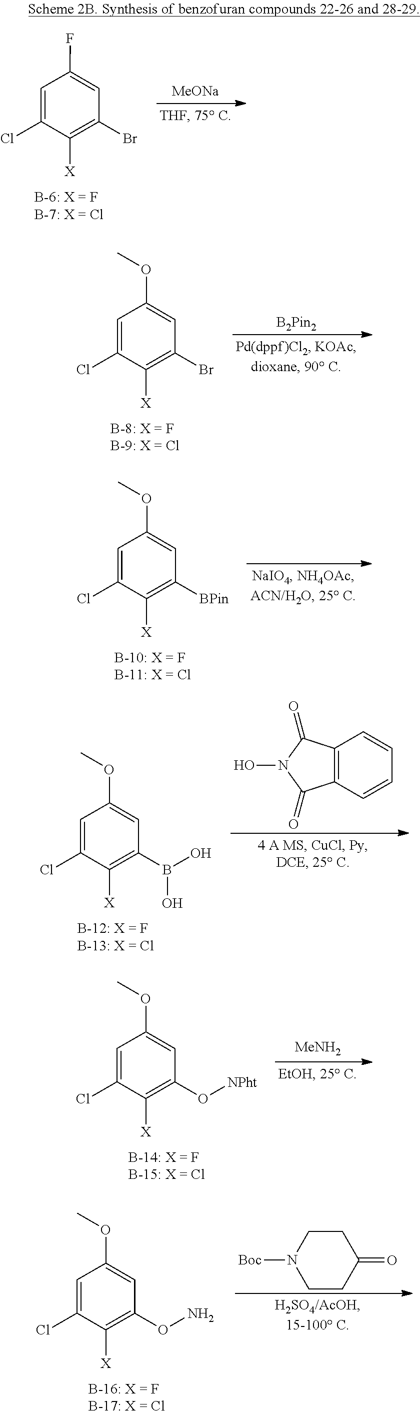

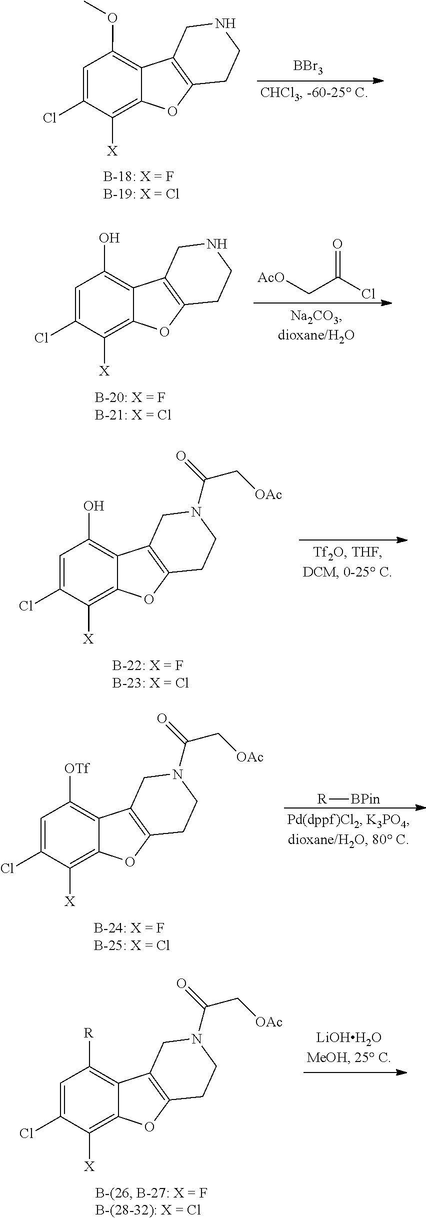

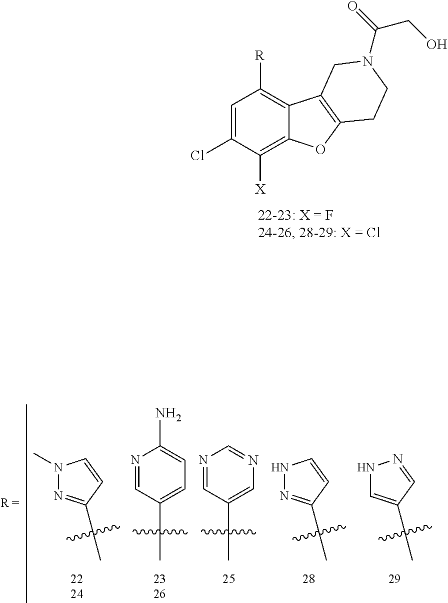

Compound B-9. A mixture of compound B-7 (10.0 g, 41.0 mmol, 1.0 eq.) and NaOMe (8.9 g, 164.0 mmol, 4.0 eq.) in THF (100 mL) was degassed and purged with N₂ for 3 times, and then the mixture was stirred at 75° C. for 4.5 hrs under N₂ atmosphere. The mixture was diluted with H₂O (50 mL), extracted with EtOAc (80 mL*3) and dried over Na₂SO₄, filtered and concentrated under reduced pressure to give a residue. The residue was purified by column chromatography (SiO₂, Petroleum ether: Ethyl acetate=95:5 to 9:1) to yield B-9 (4.6 g, 18.0 mmol, 43.8% yield) as a white solid. ¹H NMR (400 MHZ, CHLOROFORM-d) 8=7.12 (d, J=2.9 Hz, 1H), 7.00 (d, J=2.9 Hz, 1H), 3.80 (s, 3H)

Compound B-11. A mixture of B-9 (4.2 g, 16.4 mmol, 1.0 eq.), Pin₂B₂ (12.5 g, 49.2 mmol, 3.0 eq.), Pd(dppf)Cl₂·CH₂Cl₂ (1.3 g, 1.6 mmol, 0.1 eq.) and KOAc (4.8 g, 49.2 mmol, 3.0 eq.) in dioxane (60 mL) was degassed and purged with N₂ for 3 times, and then the mixture was stirred at 90° C. for 12 hrs under N₂ atmosphere. The residue was diluted with H₂O (50 mL) and extracted with EtOAc (50 mL*3), then the organic layer was dried over Na₂SO₄, filtered, concentrated and purified by column chromatography (SiO₂, Petroleum ether: Ethyl acetate=1:0 to 1:1) to yield B-11 (2.2 g, 7.3 mmol, 44.2% yield) as a green oil. ¹H NMR (400 MHz, DMSO-d₆) δ=7.32 (d, J-3.1 Hz, 1H), 7.07 (d, J=3.1 Hz, 1H), 3.79 (s, 3H), 1.31 (s, 12H).

Compound B-13. A mixture of B-11 (2.2 g, 7.1 mmol, 1.0 eq.), NaIO₄ (6.1 g, 28.4 mmol, 4.0 eq.) and NH₄OAc (2.2 g, 28.4 mmol, 4.0 eq.) in H₂O (10 mL) and ACN (10 mL) was stirred at 25° C. for 1 hr. The reaction mixture was filtered and purified by prep-HPLC (column: Phenomenex luna C18 (250*70 mm, 15 μm); mobile phase: [water (TFA)-ACN]; B %: 15%-45%, 30 min) to yield B-13 (1.2 g, 5.2 mmol, 73.4% yield) as a white solid. ¹H NMR (400 MHZ, CHLOROFORM-d) 8=7.39 (d, J-3.0 Hz, 1H), 7.12 (d, J=3.1 Hz, 1H), 5.36 (s, 2H), 3.83 (s, 3H).

Compound B-15. A mixture of B-13 (740 mg, 3.4 mmol, 1.0 eq.), 2-hydroxyisoindoline-1,3-dione (1.1 g, 6.7 mmol, 2.0 eq.), Py (292 mg, 3.7 mmol, 1.1 eq.), 4A MS (1 g) and CuCl (332 mg, 3.4 mmol, 1.0 eq.) in DCE (30 mL) was degassed and purged with O₂ for 3 times, and then the mixture was stirred at 25° C. for 12 hrs under O₂ atmosphere. The reaction mixture was filtered and then diluted with H₂O (10 mL), extracted with DCM (30 mL*3), dried over Na₂SO₄, filtered, concentrated and purified by column chromatography (SiO₂, Petroleum ether: Ethyl acetate=1:0 to 5:1) to yield B-15 (510 mg, 1.5 mmol, 45.0% yield) as a white solid. ¹H NMR (400 MHZ, DMSO-d₆) δ=8.02-7.83 (m, 4H), 7.17 (d, J-2.8 Hz, 1H), 7.08 (d, J=2.8 Hz, 1H), 3.75 (s, 3H). ESI [M+H]=338.1/340.1

Compound B-17. A mixture of B-15 (500 mg, 1.5 mmol, 1.0 eq.) in MeNH₂ (30.0% purity, 6 mL, in EtOH) was stirred at 25° C. for 1 hr. The reaction mixture was concentrated to yield B-17 (550 mg, crude) as a yellow oil. ¹H NMR (400 MHZ, DMSO-d₆) δ=6.98 (d, J=2.9 Hz, 1H), 6.59 (d, J=2.8 Hz, 1H), 3.72 (s, 3H).

Compound B-19. A mixture of B-17 (500 mg, 2.4 mmol, 1.0 eq.) in AcOH (3 mL) was added H₂SO₄ (0.3 mL), followed by tert-butyl 4-oxopiperidine-1-carboxylate (575 mg, 2.9 mmol, 1.2 eq.) was added. The mixture was stirred at 15° C. for 5 mins and then heated to 100° C. for 12 hrs. The mixture was quenched by sat. aq. Na₂CO₃ (30 mL), until pH>8, extracted with EtOAc (10 mL*3), dried over Na₂SO₄, filtered, concentrated and purified by prep-HPLC (column: Phenomenex C18 80*40 mm*3 μm; mobile phase: [water (NH₄HCO₃)-ACN]; B %: 15%-65%, 8 min) to yield B-19 (80 mg, 294.0 umol, 12.2% yield) as a brown solid. ¹H NMR (400 MHz, DMSO-d₆) δ=6.99 (s, 1H), 3.86-3.80 (m, 5H), 2.98 (t, J=5.7 Hz, 2H), 2.67-2.63 (m, 2H). ESI [M+H]=272.0/274.0

Compound B-21. A mixture of B-19 (10 mg, 36.8 umol, 1.0 eq.) in CHCl₃ (1 mL) was added BBr₃ (558 mg, 220.5 umol, 215 μL, 6.0 eq.) at −60° C. The mixture was stirred at 25° C. for 3 hrs. The mixture was quenched by sat·aq. Na₂CO₃ (30 mL), until pH>8, extracted with EtOAc (10 mL*3), dried over Na₂SO₄, filtered, and concentrated to yield B-21 (15 mg, crude) as a brown oil. ESI [M+H]=258.0/260.0.

Compound B-23. Compound B-21 was reacted with 2-acetoxyacetyl chloride as described for conversion of compound A-2 to A-3 to afford B-23.

Compound B-25. A mixture of B-23 (280 mg, 781.75 umol, 1 eq.) in DCM (4 mL) and TEA (395 mg, 3.91 mmol, 5 eq.) in DCM (1 mL) was added Tf₂O (551.40 mg, 1.95 mmol, 2.5 eq.) in DCM (0.5 mL) at 0° C., stirred at 25° C. for 1 hr under N₂ atmosphere, concentrated, diluted with H₂O (5 mL), extracted with EtOAc (10 mL*3). The organic layer was washed with brine (5 mL*3). The organic layer was dried over Na₂SO₄, filtered, and concentrated to yield B-25 (110 mg, crude) as a brown oil. ESI [M+Na]=511.8/510.0

Compounds B-(28-32). Compound B-25 was coupled with substituted boronic ester derivatives using method A to afford B-(28-32).

Compounds 24-26 and 28-29. Ester hydrolysis in B-(26-32) using method C afforded compound 24-26 and 28-29.

2.3. Compounds 27 and 30-31 (Scheme 2C). Triflate B-25 was converted to a boronic acid derivative B-33, reacted with bromo-heterocycles to afford compounds B-(34-36), and subsequently deprotected to give compounds 27 and 30-31.

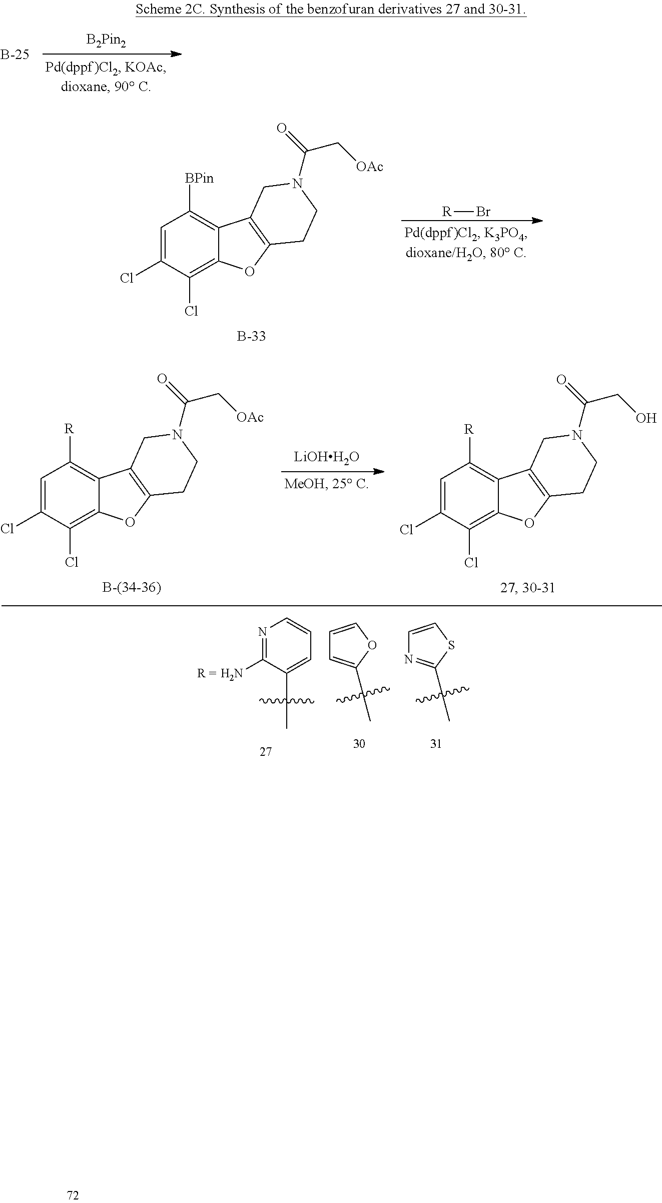

Compound B-33. A mixture of B-25 (130 mg, 265.18 umol, 1 eq.), 4,4,5,5-tetramethyl-2-(4,4,5,5-tetramethyl-1,3,2-dioxaborolan-2-yl)-1,3,2-dioxaborolane (336.69 mg, 1.33 mmol, 5 eq.), Pd(dppf)Cl₂ (19.40 mg, 26.52 umol, 0.1 eq.), KOAc (78.07 mg, 795.54 umol, 3 eq.) in dioxane (6 mL) was degassed and purged with N₂ for 3 times, and then the mixture was stirred at 110° C. for 12 hr under N₂ atmosphere. The residue was diluted with H₂O (10 mL) and extracted with EtOAc (15 mL*3). The combined organic layers were dried over Na₂SO₄, filtered, and concentrated under reduced pressure to give a residue. The residue was purified by column chromatography (SiO₂, Petroleum ether: Ethyl acetate=1:1 to 1:2) to yield B-33 (120 mg, 256.34 umol, 96.67% yield) as a white solid.

Compound B-34-36. Compound B-33 was coupled with substituted bromide derivatives using method A to yield B-34-36 (1.56 mg, 1.95 umol, 3.04% yield, 94% purity) as a pale-yellow solid.

Compound 27, 30 and 31. Ester hydrolysis of compound B-34-36 using method C afforded compound 27, 30 and 31.

2.4. Compounds 32-35 (Scheme 2D). Synthesis of compounds 32-35 was achieved using the above-mentioned intermediate B-21, which underwent peptide coupling with Boc-protected glycine and 2-methylalanine to give compounds B-37 and B-38. The latter products were reacted with triflic anhydride in the presence of Et₃N to give triflates B-39 and B-40, which underwent Suzuki coupling with appropriate bromo-heterocycles to afford B-(41-44) and subsequent Boc-deprotection to give compounds 32-35.

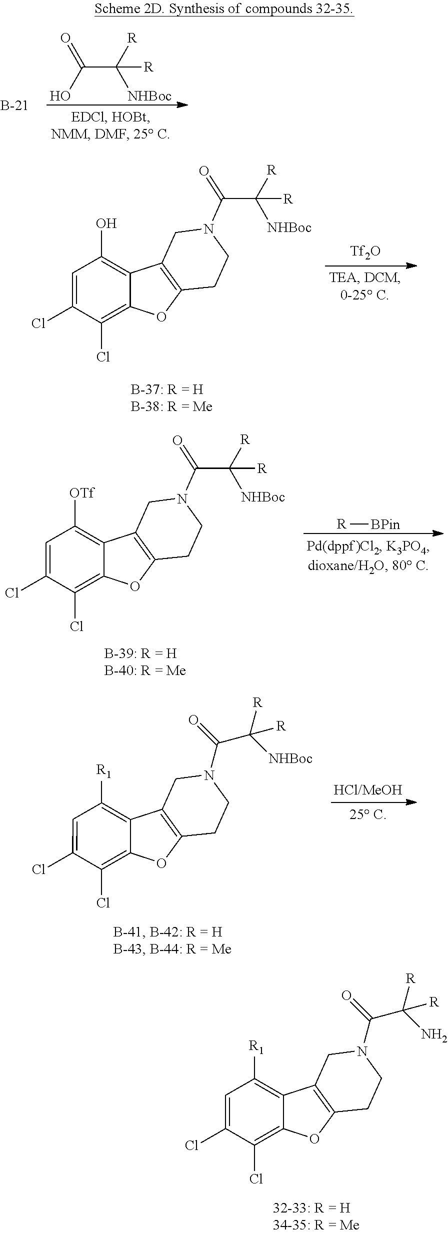

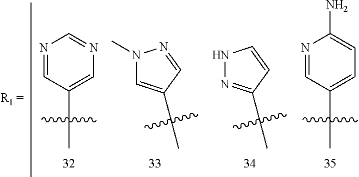

Compound B-37. A mixture of B-21 (45 mg, 152.8 umol, 1.0 eq., HCl), 2-(tert-butoxycarbonyl amino) acetic acid (54 mg, 305.5 umol, 2.0 eq.) and NMM (62 mg, 611.1 umol, 4.0 eq.) in DMF (1 mL) was added EDCI (44 mg, 229.2 umol, 1.5 eq.), HOBt (31 mg, 229.2 umol, 1.5 eq.) and stirred at 25° C. for 1 hr. Then the mixture was quenched with sat·aq·Na₂CO₃ (1 mL) and extracted with EtOAc (5 mL*3). The organic layer was washed with brine (3 mL*5), dried over Na₂SO₄, filtered, concentrated and purified by column chromatography (SiO₂, Petroleum ether: Ethyl acetate=5:1 to 3:1) to yield B-37 (45 mg, 108.4 umol, 70.9% yield) as a white solid. ¹H NMR (400 MHZ, DMSO-d₆) δ=10.79-10.59 (m, 1H), 6.93-6.69 (m, 2H), 4.75-4.60 (m, 2H), 3.97-3.83 (m, 2H), 3.81-3.66 (m, 2H), 2.90 (br s, 1H), 2.87-2.79 (m, 1H), 1.38 (s, 9H). ESI [M−H]=413.0/415.0

Compound B-39. A mixture of B-37 (40 mg, 96.3 umol, 1.0 eq.) and TEA (97 mg, 963.2 umol, 10.0 eq.) in DCM (1 mL) was degassed and purged with N₂ for 3 times, and then Tf₂O (136 mg, 481.6 umol, 5.0 eq.) in DCM (0.2 mL) was added slowly at 0° C. under N₂ atmosphere. Then the mixture was stirred at 25° C. for 1 hr under N₂ atmosphere. Then the mixture was quenched with H₂O (2 mL) and extracted with EtOAc (5 mL*2). The organic layer were dried over Na₂SO₄, filtered, concentrated and purified by prep-TLC (SiO₂, Petroleum ether: Ethyl acetate=2:1) to yield B-39 (48 mg, 87.7 umol, 91.1% yield) as a yellow oil. ESI [M−H]=544.9/546.9.

Compound B-41-42. Compound B-39 was coupled with substituted boronic ester derivatives using method A to yield B-41-42 (1.56 mg, 1.95 umol, 3.04% yield, 94% purity) as a pale-yellow solid.

Compound 32-35. NBoc deprotection in B-41-44 using method B afforded compound 32-35.

2.5. Compound 36-38 (Scheme 2E). Synthesis of compounds 36-38 was achieved using compounds B-16 and B-17, which reacted with 1-Boc-3-piperidone to afford the key benzofuran intermediates B-45 and B-46. For isolation these products were N-Boc protected using Boc₂O giving compound B-47 and B-48, and subsequently treated with BBr₃ to afford amino-phenols B-49 and B-50. The latter products were converted to the next key intermediates B-53 and B-54 by addition of the acetoxyacetyl linker to give amides B-51 and B-52, followed by treatment with triflicanhydride/TEA in dichloromethane.

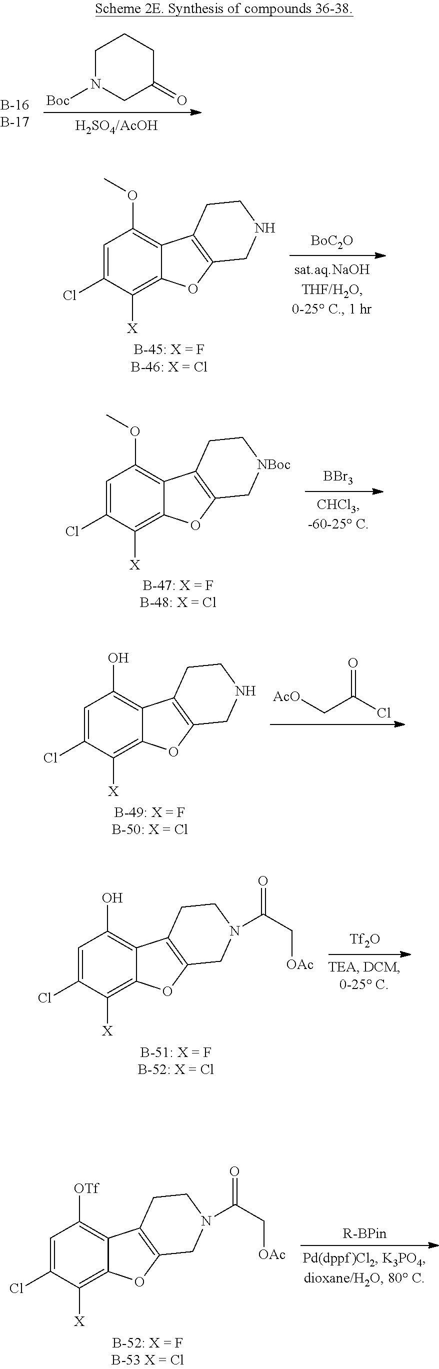

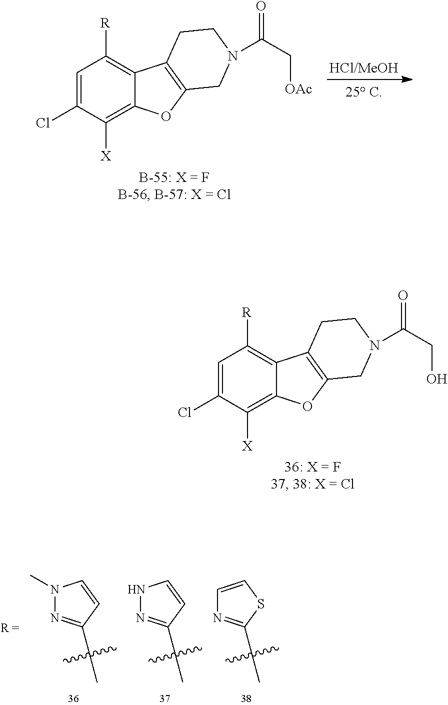

Compound B-46. To a solution of B-17 (1.9 g, 9.13 mmol, 1 eq.), tert-butyl 3-oxopiperidine-1-carboxylate (2.18 g, 10.96 mmol, 1.2 eq.) in AcOH (20 mL) was added H₂SO₄ (4 mL). The mixture was stirred at 90° C. for 1 hr. The reaction mixture diluted with sat·aq·NaOH (30 mL) to adjusted pH=9 at 0° C. The mixture was extracted with 2-Me-THF (20 mL*3). The combined organic layers were dried over Na₂SO₄, filtered, and concentrated under reduced pressure to yield B-46 (2.88 g, crude) as a white solid.

Compound B-48. To a solution of B-46 (2.88 g, 10.58 mmol, 1 eq.) in sat·aq·NaOH (20 mL), THF (30 mL) was added Boc₂O (4.62 g, 21.17 mmol, 4.86 mL, 2 eq.) at 0° C. The mixture was stirred at 25° C. for 1 hr. The mixture was concentrated and extracted with 2-Me-THF (20 mL*3). The combined organic layers were dried over Na₂SO₄, filtered, and concentrated under reduced pressure to give a residue. The residue was purified by column chromatography (SiO₂, Petroleum ether: Ethyl acetate=10:1 to 1:1) to yield B-48 (780 mg, 2.10 mmol, 19.80% yield) as a yellow solid. ESI [M-Boc+H]=272.0/274.0

Compound B-50. To a solution of B-48 (630 mg, 1.69 mmol, 1 eq.) in CHCl₃ (10 mL) was added BBr₃ (4.24 g, 16.92 mmol, 1.63 mL, 10 eq.) at −60° C. The mixture was stirred at 25° C. for 1 hr. The reaction mixture was quenched by Na₂CO₃ (100 mL) at 0° C. The solution was concentrated to remove the CHCl₃ and used to next step directly without further purification.

Compound B-52. To a solution of B-50 (50 mg, 135 umol, 1.0 eq., HCl) in sat·aq·NaHCO₃ (1 mL) and dioxane (1 mL) was added (2-chloro-2-oxo-ethyl) acetate (74 mg, 540 umol, 4.0 eq.) at 0° C. and stirred at 25° C. for 16 hrs, then diluted with H₂O (3 mL) and extracted with EtOAc (5 mL*3). The organic layer was dried over Na₂SO₄, filtered, and concentrated to yield B-52 (60 mg, crude) as a yellow solid.

Compound B-54. A mixture of B-52 (70 mg, 162 umol, 1.0 eq.) and TEA (328 mg, 3 mmol, 20.0 eq.) in DCM (1 mL) was added Tf₂O (229 mg, 811 umol, 5.0 eq.) in DCM (0.5 mL) at 0° C., stirred at 25° C. for 1 hr under N₂ atmosphere, diluted with H₂O (2 mL), extracted with EtOAc (3 mL*3). The organic layer was dried over Na₂SO₄, filtered, concentrated, and purified by prep-TLC (SiO₂, Petroleum ether:EtOAc=1:1) to yield B-54 (66 mg, 117 umol, 72.2% yield) as a yellow solid.

Compound B-55-57. Compound B-54 was coupled with substituted boronic ester derivatives using method A to yield B-55-57 (1.56 mg, 1.95 umol, 3.04% yield, 94% purity) as a pale-yellow solid.

Compound 36-38. Ester hydrolysis of compound B-55-57 using method C afforded compound 32-35.

2.6. Compounds 39-41 (Scheme 2F). Synthesis of compounds 39-41 was achieved using intermediate B-50 and reacting with N-Boc glycine to afford B-58. The latter reacted with Tf₂O to afford triflate B-59, which underwent Suzuki reaction with heterocycles-boronic acid derivatives to afford compounds B-(60-62) and then Boc-deprotection to give the title products 39-41.

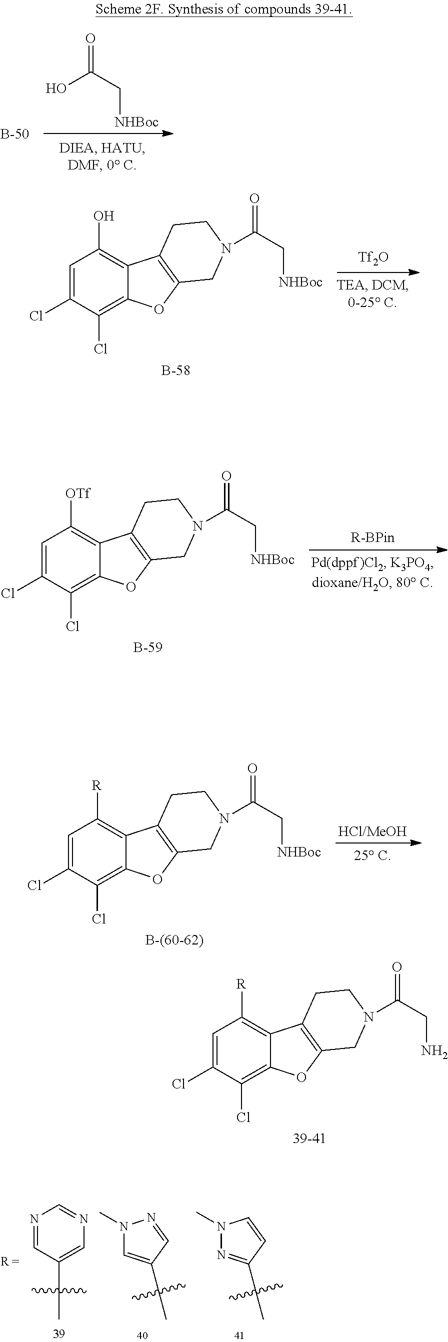

Compound B-58. To a solution of B-50 (476 mg, 1.62 mmol, 1 eq., HCl) in DMF (5 mL) was added 2-(tert-butoxycarbonylamino) acetic acid (849.26 mg, 4.85 mmol, 3 eq.) and DIEA (626.56 mg, 4.85 mmol, 3 eq.), HATU (921.66 mg, 2.42 mmol, 1.5 eq.). The mixture was stirred at 0° C. for 1 hr. The reaction mixture was diluted with H₂O (10 mL) extracted with EtOAc (20 mL*3). The combined organic layers were washed with brine (10 mL*3), dried over Na₂SO₄, filtered and concentrated under reduced pressure to give a residue. The residue was purified by column chromatography (SiO₂, Petroleum ether: Ethyl acetate=1:1 to 0:1) to yield B-58 (380 mg, 915.07 umol, 56.63% yield) was obtained as a yellow solid. ESI [M+H]=414.9/416.9

Compound B-59. A mixture of B-58 (370 mg, 890.99 umol, 1 eq.), TEA (450.80 mg, 4.45 mmol, 5 eq.) and Tf₂O (628.46 mg, 2.23 mmol, 2.5 eq.) in DCM (5 mL) was degassed and purged with N₂ for 3 times at 0° C., and then the mixture was stirred at 25° C. for 1 hr under N2 atmosphere. The reaction mixture was concentrated under reduced pressure to remove solvent. The residue was diluted with H₂O (10 mL) and extracted with EtOAc (20 mL*3). The combined organic layers were dried over Na₂SO₄, filtered, and concentrated under reduced pressure to give a residue. The residue was purified by column chromatography (SiO₂, Petroleum ether: Ethyl acetate=0:1 to 1:1) to yield B-59 (220 mg, 401.95 umol, 45.11% yield) as a yellow oil. ESI [M+Na⁺]=568.9/570.9.

Compounds B-(60-62). Compound B-59 was coupled with substituted boronic ester derivatives using method A to yield B-(60-62).

Compound 39-41. NBoc deprotection in B-(60-62) using method B afforded compound 39-41 as pale-yellow solids.

### 3. Benzothiofuran Derivatives.

3.1. Compounds 42-44 (Scheme 3A). Synthesis of compounds 42-44 started with methylation of phenol C-1 followed by bromination affording bromide C-2. Pd-catalyzed reaction with ethyl 3-mercaptopropionate afforded compound C-3, which reacted with N-Boc-3,3-epoxy-piperidine, followed by oxidation of the resulting alcohol to afford compound C-4. The latter underwent phosphoric acid-mediated cyclization to afford the key intermediate C-5, and subsequently reacted with 2-acetoxyacetyl chloride to afford intermediate C-6, which serves the precursors of compounds 42-44, as well as other similar compounds.

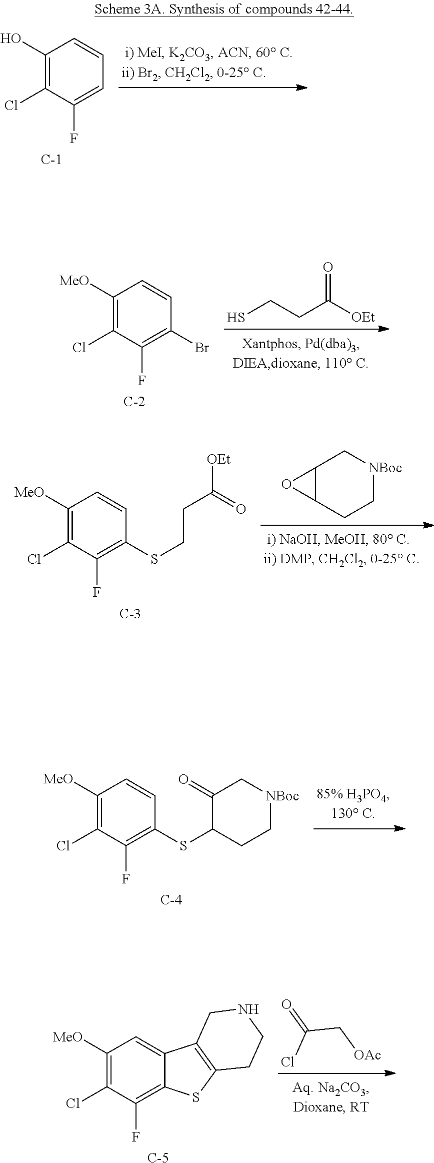

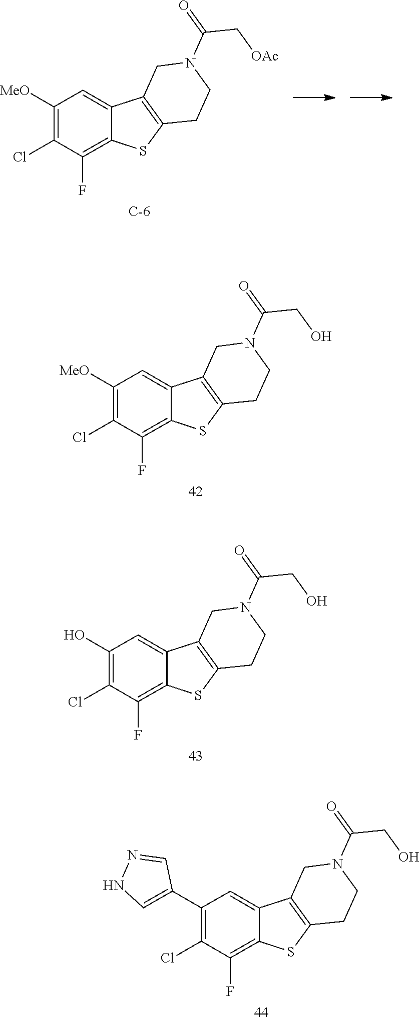

3.2. Compounds 45-52 (Scheme 3B). Synthesis of compounds 45-52 started with compounds C-7 and C-8, which underwent sequence of reactions, as described above for the synthesis of compound C-5, to afford intermediates C-13 and C-14, via alcohols C9/C10 and ketones C11/C12. BBr₃ deprotection of methoxy in compounds C-13 and C-14 afforded aminophenols C-15 and C16, which reacted with 2-acetoxy acetyl chloride to afford compounds C-17 and C-18. Phenol was converted to triflate giving C-19 and C-20, and Pd-catalyzed reaction with the boronic acid derivatives followed by acetate-deprotection under basic conditions afforded the target compounds 45-52 via the acetate precursors C-21 and C-(22-27) (Scheme 3B). Detailed methods are provided below for the conversion of compound C-8 to 46-48 and compound C-7 was converted to 45 similarly.

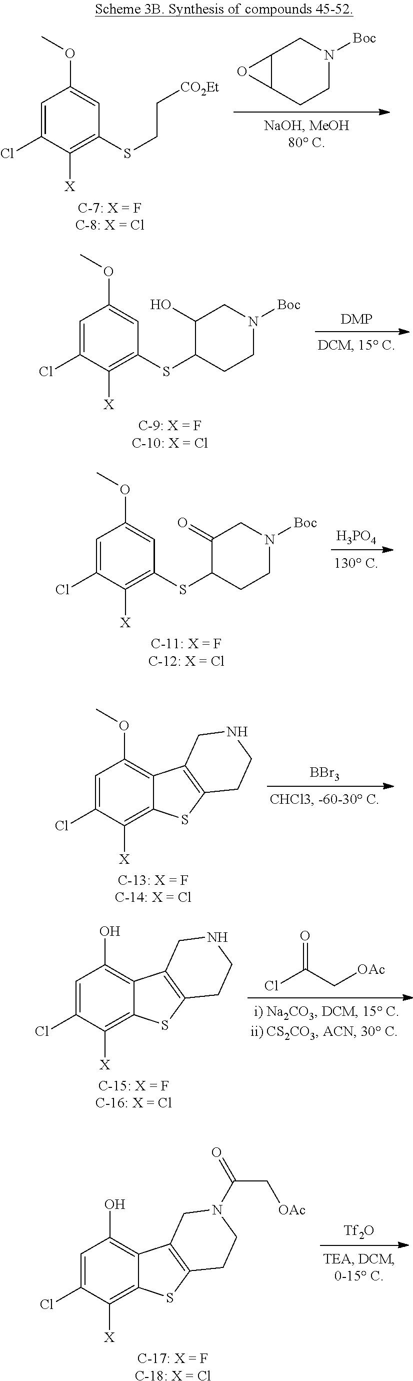

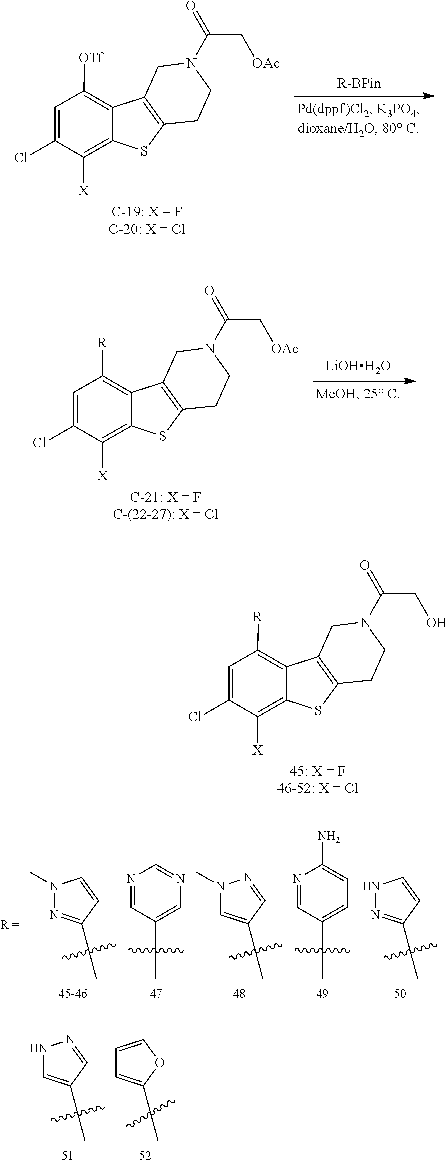

Compound C-10. To a solution of compound C-8 (2.0 g, 7 mmol, 1.0 eq.), tert-butyl 7-oxa-3-azabicyclo[4.1.0]heptane-3-carboxylate (1.4 g, 7 mmol, 1.1 eq.) in MeOH (30 mL) was added NaOH (247 mg, 6 mmol, 0.9 eq.) and stirred at 80° C. for 2 hrs, quenched by H₂O (20 mL) and extracted with DCM (20 mL*5). The organic layer was dried over Na₂SO₄, filtered, concentrated and purified by column chromatography (SiO₂, Petroleum ether:EtOAc=3:2 to 3:2) to yield C-10 (2.4 g, 6 mmol, 86.0% yield) as a colorless oil. ¹H NMR (400 MHZ, DMSO-d₆) δ=7.16-7.04 (m, 2H), 5.53 (d, J=4.9 Hz, 1H), 3.98-3.81 (m, 2H), 3.79 (s, 3H), 3.76-3.67 (m, 1H), 3.46-3.33 (m, 2H), 3.03-2.87 (m, 1H), 2.04-1.99 (m, 1H), 1.39 (s, 9H), 1.34-1.26 (m, 1H).

Compound C-12. To a solution of C-10 (2.4 g, 5.8 mmol, 1.0 eq.) in DCM (20 mL) was added DMP (3.0 g, 7 mmol, 1.2 eq.), stirred at 15° C. for 2 hrs, quenched by sat·aq·NaHCO₃ (30 mL) and sat·aq·Na₂S203 (30 mL) and extracted with DCM (50 mL*5). The organic layer was dried over Na₂SO₄, filtered, concentrated and purified by prep-HPLC (column: Phenomenex Luna 80*30 mm*3 μm; mobile phase: [water (TFA)-ACN]; B %: 45%-80%, 8 mins) to yield C-12 (3.3 g, crude) as a yellow oil. ¹H NMR (400 MHZ, DMSO-d₆) δ=7.21-7.11 (m, 1H), 7.09 (d, J=2.4 Hz, 1H), 4.55 (br d, J=2.3 Hz, 1H), 4.08-3.87 (m, 2H), 3.81 (br s, 3H), 3.54-3.42 (m, 2H), 2.58 (br s, 2H), 1.40 (br d, J=6.0 Hz, 9H). ESI [M-tBu+H]=349.9/351.9.

Compound C-14. To a solution of C-12 (3.0 g, 7 mmol, 1.0 eq.) in H₃PO₄ (30 mL, 85.0% purity) was stirred at 130° C. for 12 hrs, quenched by 5M NaOH (150 mL) to pH=14 and extracted with DCM (150 mL*4). The organic layer was dried over Na₂SO₄, filtered, concentrated and purified by prep-HPLC (column: Phenomenex luna C18 (250*70 mm, 15 μm); mobile phase: [water (TFA)-ACN]; B %: 25%-55%, 20 mins) to yield C-14 (600 mg, 2 mmol, 28.2% yield) as a light brown solid. ¹H NMR (400 MHZ, DMSO-d₆) δ=7.22 (s, 1H), 4.50 (s, 2H), 3.94 (s, 3H), 3.48 (br t, J=5.8 Hz, 2H), 3.14 (br t, J=5.8 Hz, 2H). ESI [M−H]=287.9/289.9.

Compound C-16. To a solution of compound C-14 (530 mg, 2 mmol, 1.0 eq.) in CHCl₃ (6 mL) was added BBr₃ (10.4 g, 42 mmol, 23 eq.) at −60° C., stirred at 30° C. for 48 hrs and quenched by sat·aq·Na₂CO₃ (20 mL) at 0° C. to pH=9 to yield C-16 (504 mg, crude) in CHCl₃ and H₂O as a pale yellow liquid. ESI [M+H]=274.0/276.0

Compound C-18. To a solution of compound C-16 (500 mg, 2 mmol, 1.0 eq.) in DCM (40 mL), Na₂CO₃ (30 mL) and CHCl₃ (30 mL) was added 2-acetoxyacetyl chloride (996 mg, 7 mmol, 4.0 eq.), stirred at 15° C. for 1 hr, diluted with H₂O (20 mL), extracted with DCM (40 mL*3). The organic layer was dried over Na₂SO₄, filtered, concentrated and purified by column chromatography (SiO₂, Petroleum ether:EtOAc=3:7 to 3:7) to yield O-acetyl derivative of C-18, (470 mg, crude) as a pale yellow solid. ESI [M+H]=473.9/475.9

A mixture of the above-mentioned O-acetyl derivative of C-18 (50 mg, 105 umol, 1.0 eq.) and Cs₂CO₃ (137 mg, 422 umol, 4.0 eq.) in ACN (1 mL) was stirred at 30° C. for 1 hr, then filtrated and quenched by 1N HCl (0.1 mL) to pH=4, extracted with EtOAc (30 mL*3). The organic layer was dried over Na₂SO₄, filtered and concentrated to yield compound C-18, (60 mg, crude) as a brown oil. ESI [M+H]=373.9/375.9

Compound C-20. To a solution of C-18, (50 mg, 134 umol, 1.0 eq.) and TEA (54 mg, 534 umol, 4.0 eq.) in DCM (1 mL) was added Tf₂O (75 mg, 267 umol, 2.0 eq.) in DCM (0.5 mL) at 0° C., stirred at 15° C. for 1 hr, quenched by sat. aq. Na₂CO₃ (1 mL) and extracted with DCM (30 mL*3). The organic layer was dried over Na₂SO₄, filtered and concentrated to yield C-20 (80 mg, crude) as a brown oil. ESI [M+H]=505.9/507.9.

Compound C-(21-27). Compound C-14 was coupled with substituted boronic ester derivatives using method A to yield C-(22-27) as brown solids. Compound C-21 was prepared similarly.

Compound 45-52. Ester hydrolysis of compound C-21-27 using method C afforded compounds 45-52.

3.3. Compounds 53-56 (Scheme 3C). Synthesis of compounds 53-56 started with reaction of intermediate C-16 with N-Boc-protected glycine and 2-methyl alanine to afford compounds C-28 and C-29. The latter compounds reacted with triflic anhydride to afford compounds C-30 and C-31, which underwent Pd-catalyzed Suzuki coupling with the heterocycle-boronic acid derivatives to afford compounds C-(32-35), and subsequently Boc deprotection to give compounds 53-56.

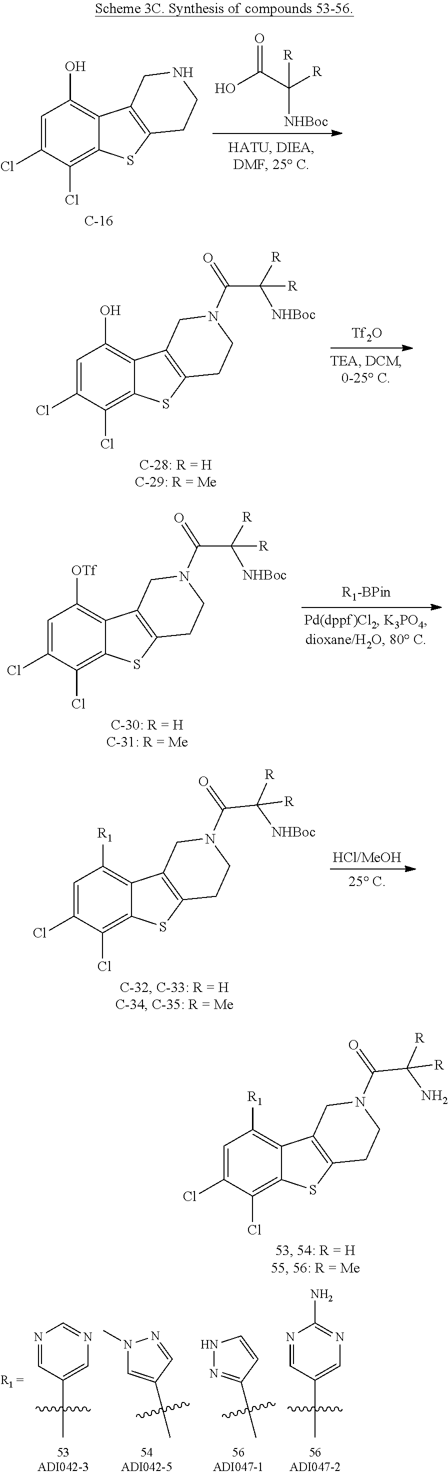

Compound C-28. To a solution of C-16 (484.16 mg, 2.38 mmol, 2 eq.) in DMF (50 mL) was added DIEA (461.83 mg, 3.57 mmol, 622.41 uL, 3 eq.), 6,7-dichloro-1,2,3,4-tetrahydrobenzothiopheno [3,2-c]pyridin-9-ol (370 mg, 1.19 mmol, 1 eq., HCl) and HATU (905.81 mg, 2.38 mmol, 2 eq.). The mixture was stirred at 25° C. for 1 hr, added sat·aq·Na₂CO₃ (10 mL), stirred 12 hrs at 15° C., diluted with H₂O (20 mL), extracted with EtOAc (15 mL*3). The organic layer was washed with brine (5 mL*3). The organic layer was dried over Na₂SO₄, filtered, concentrated and purified by column chromatography (SiO₂, Petroleum ether: Ethyl acetate=1:2) to yield C-28 (500 mg, 1.09 mmol, 91.38% yield) as a yellow oil. ESI [M-Boc+H]=359.1/361.0

Compound C-30. A mixture of C-28 (500 mg, 1.09 mmol, 1 eq.) in DCM (60 mL) and TEA (551 mg, 5 mmol, 5 eq.) in DCM (5 mL) and Tf₂O (768 mg, 2.72 mmol, 2.5 eq.) in DCM (5 mL) at 0° C., stirred at 25° C. for 1 hr under N₂ atmosphere, diluted with H₂O (10 mL), extracted with DCM (30 mL*3). The organic layer was washed with brine (10 mL*3), The organic layer was dried over Na₂SO₄, filtered, concentrated and purified by column chromatography (SiO₂, Petroleum ether: Ethyl acetate=1:1) to yield C-30 (200 mg, 338.15 umol, 31.07% yield) as a white solid. ESI [M+Na]=612.9/614.7

Compound C-(32-35). Compound C-30 was coupled with substituted boronic ester derivatives using method A to afforded C-(32-35) as white solids.

Compound 53-56. NBoc deprotection of C-(32-35) using method B afforded compound 53-56 as white solids.

3.4. Compound 57 (Scheme 3D). Synthesis of 57 started with peptide coupling of amine C-16 with N-Boc protected (R)-proline to afford compound C-36. The phenol reacted with Tf₂O affording triflate C-37 and the latter underwent Suzuki coupling with 2-amino-pyridine boronic acid derivative to afford compound C-38, and subsequently Boc deprotection to give the title product 57 (Scheme 3D).

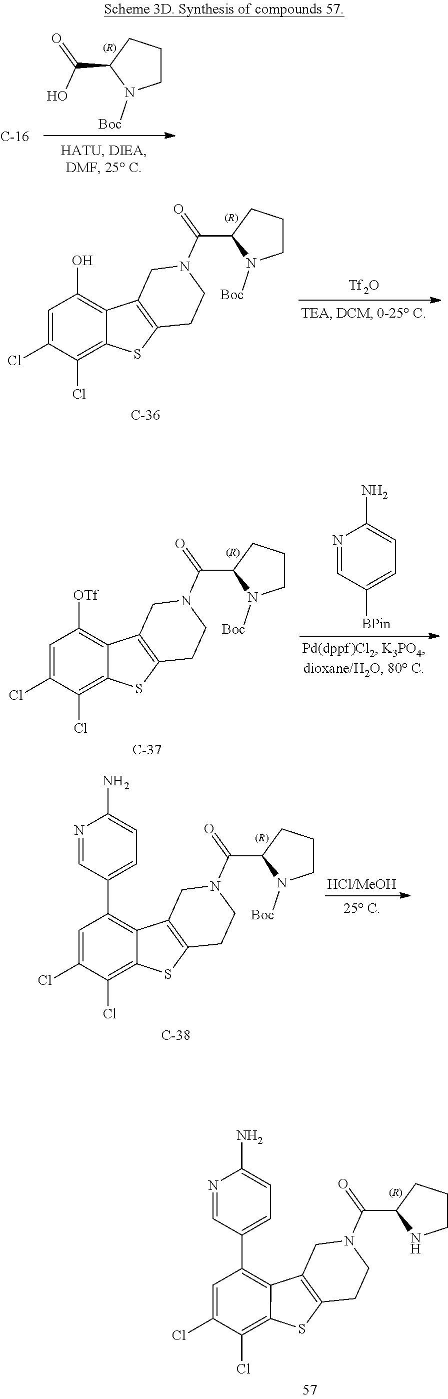

Compound C-36. To a solution of C-16 (150 mg, 482.89 umol, 1 eq., HCl) in DMF (15 mL) was added DIEA (187 mg, 1 mmol, 3 eq.), (2R)-1-tert-butoxycarbonylpyrrolidine-2-carboxylic acid (208 mg, 966 umol, 2 eq.) and HATU (367 mg, 965 umol, 2 eq.), was stirred at 25° C. for 1 hr, added sat·aq·Na₂CO₃ (10 mL), stirred 12 hrs at 15° C., diluted with H₂O (20 mL), extracted with EtOAc (15 mL*3). The organic layer was washed with brine (5 mL*3). The organic layer was dried over Na₂SO₄, filtered, concentrated and purified by column chromatography (SiO₂, Petroleum ether: Ethyl acetate=1:2) to yield C-36 (200 mg, 424.27 umol, 87.86% yield) as a brown solid. ESI [M-Boc+H]=371.0/372.9

Compound C-37. A mixture of C-36 (120 mg, 254.56 umol, 1 eq.) in DCM (4 mL) and TEA (129 mg, 1 mmol, 5 eq.) in DCM (1 mL) and Tf₂O (180 mg, 636 umol, 2.5 eq.) in DCM (1 mL) at 0° C., stirred at 25° C. for 1 hr under N₂ atmosphere, concentrated, diluted with H₂O (5 mL), extracted with EtOAc (5 mL*3). The organic layer was washed with brine (5 mL*3), The organic layer was dried over Na₂SO₄, filtered, concentrated and purified by column chromatography (SiO₂, Petroleum ether: Ethyl acetate=1:1) to yield C-37 (100 mg, 165.71 umol, 65.10% yield) as a white solid. ESI [M-Boc+H]=502.9/504.7

Compound C-38. Compound C-37 was coupled with substituted boronic ester derivatives using method A to yield C-38 (9.74 mg, 17.79 umol, 21.47% yield) as a white solid. 1H NMR (400 MHZ, DMSO-d₆) δ=7.94 (br s, 1H), 7.47-7.32 (m, 2H), 6.59 (br d, J=8.5 Hz, 1H), 5.96 (br s, 2H), 4.71-4.20 (m, 1H), 4.00 (br d, J=1.9 Hz, 2H), 3.92-3.66 (m, 2H), 3.33 (br t, J=6.7 Hz, 2H), 2.98 (br s, 2H), 2.26-1.93 (m, 1H), 1.91-1.66 (m, 3H), 1.36-1.10 (m, 9H). ESI [M+H]=547.1/549.1.

Compound 57. Ester hydrolysis of compound C-38 using method C afforded compound 57 (15 mg, 32 umol, 25.01% yield, HCl) as a white solid.

3.5. Compound 58 (Scheme 3E). Synthesis of compound 58 started with the nucleophilic displacement of bromide in N-Boc-3-bromopyridone with thiol in 2,3-dichlorobenzene thiol affording thioether C-39, which underwent PPA-mediated cyclization to afford benzothiofuran intermediate C-40. The latter was reacted with 2-acetoxyacetyl chloride and the resulting product C-41 underwent basic hydrolysis to afford the target compound 58.

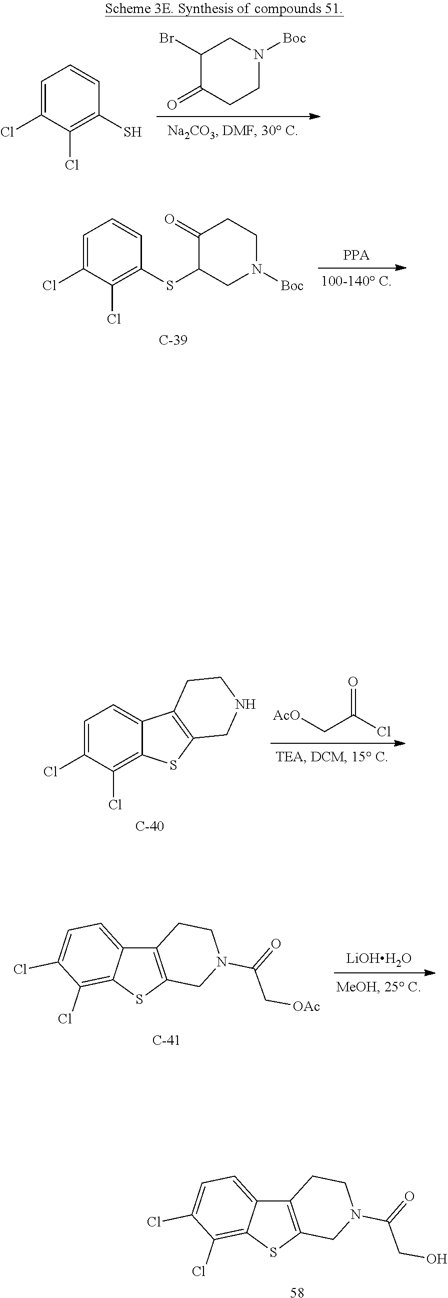

Compound C-39. A mixture of 2,3-dichloro-thiophenol (5.0 g, 27.9 mmol, 1.0 eq.), tert-butyl 3-bromo-4-oxo-piperidine-1-carboxylate (7.8 g, 27.9 mmol, 1.0 eq.) and Na₂CO₃ (5.9 g, 55.9 mmol, 2.0 eq.) in DMF (50 mL) was stirred at 30° C. for 1.5 hrs. The mixture was quenched with H₂O (40 mL), extracted with EtOAc (50 mL*3), washed with brine (20 mL*5), dried over Na₂SO₄, filtered, concentrated and purified by prep-HPLC (neutral condition; column: Phenomenex Titank C18 Bulk 250*100 mm 10u; mobile phase: [water (10 mM NH₄HCO₃)-ACN]; B %: 50%-80%, 20 min) to yield C-39 (7.7 g, 20.5 mmol, 73.3% yield) as a yellow oil. 1H NMR (400 MHZ, DMSO-d₆) δ=7.59-7.48 (m, 2H), 7.42-7.33 (m, 1H), 4.54-4.46 (m, 1H), 4.07 (br s, 1H), 3.94-3.84 (m, 1H), 3.50 (br s, 2H), 2.64-2.54 (m, 2H), 1.41 (br s, 9H). ESI [M-tBu+H]=320.0/322.0

Compound C-40. The solution of PPA (2 mL) was stirred at 100° C. for 30 mins. Then compound C-39 (200 mg, 531.5 umol, 1.0 eq.) was added and stirred at 140° C. for 2 hrs. The mixture was added H₂O (10 mL), adjust pH>8 with aqt·aq·NaOH (10 mL), extracted with EtOAc (50 mL*3), washed with brine (20 mL*5), dried over Na₂SO₄, filtered, concentrated and purified by prep-HPLC (neutral condition; column: Phenomenex Gemini-NX C18 75*30 mm*3 μm; mobile phase: [water (10 mM NH₄HCO₃)-ACN]; B %: 35%-65%, 8 min) to yield C-40 (27 mg, 103.2 umol, 19.4% yield, 100.0% purity) as a yellow solid. ¹H NMR (400 MHZ, DMSO-d₆) δ=7.72-7.59 (m, 2H), 3.99 (br s, 2H), 3.04 (br s, 2H), 2.71 (br s, 2H). ESI [M+H]=258.0/260.0

Compound C-41. To a solution of C-40 (170 mg, 658.5 umol, 1.0 eq.) and TEA (200 mg, 2.0 mmol, 3.0 eq.) in DCM (3 mL) was added 2-acetoxyacetyl chloride (539 mg, 4.0 mmol, 6.0 eq.). The mixture was stirred at 15° C. for 1 hr. The mixture was quenched by H₂O (5 mL), extracted with DCM (10 mL*3), dried over Na₂SO₄, filtered, concentrated and purified by prep-HPLC (TFA condition; column: Phenomenex Luna C18 150*30 mm*5 μm; mobile phase: [water (0.1% TFA)-ACN]; B %: 28%-58%, 8 min) to yield C-41 (130 mg, 361.1 umol, 54.8% yield, 99.5% purity) as a brown solid. ¹H NMR (400 MHz, DMSO-d₆) δ=7.75-7.68 (m, 1H), 7.67-7.62 (m, 1H), 4.96-4.84 (m, 2H), 4.83-4.76 (m, 2H), 3.87-3.72 (m, 2H), 2.97-2.78 (m, 2H), 2.08 (s, 3H). ESI [M+H]=357.9/359.9

Compound 58. To a solution of C-41 (100 mg, 279.1 umol, 1.0 eq.) in MeOH (2 mL) was added LiOH·H₂O (35 mg, 837.4 umol, 3.0 eq.). The mixture was stirred at 25° C. for 12 hrs. The mixture was concentrated, dilute with H₂O (5 mL), extracted with DCM (10 mL*3), dried over Na₂SO₄, filtered, concentrated and purified by prep-HPLC (TFA condition; column: Phenomenex Luna 80*30 mm*3 μm; mobile phase: [water (0.1% TFA)-ACN]; B %: 23%-53%, 8 min) to yield compound 58 (32 mg, 101.2 umol, 36.3% yield, 100.0% purity) as a pale yellow solid.

3.6. Compounds 59-66 (Scheme 3F). To prepare compounds 59-66, we started with the above-described intermediates C-7 and C-8 and reacted these with 1-Boc-3-bromo-4-pryimidinone to afford thioether C-42 and C-43. Oxidation of alcohol afforded ketones in C-44 and C-45, and the phosphoric acid-mediated cyclization gave the key benzothiofuran derivatives C-46 and C-47. Methoxy group was deprotected using BBr₃ in CHCl₃ at a low temperature and the resulting products C-48 and C-49 were converted to triflates C-50 and C-51. Suzuki coupling of the triflates with the appropriate heterocycles-boronic acid derivatives afforded compounds C-52 and C-(53-59), and acetate removal in the latter compounds afforded the target products 59-66 (Scheme 3F).

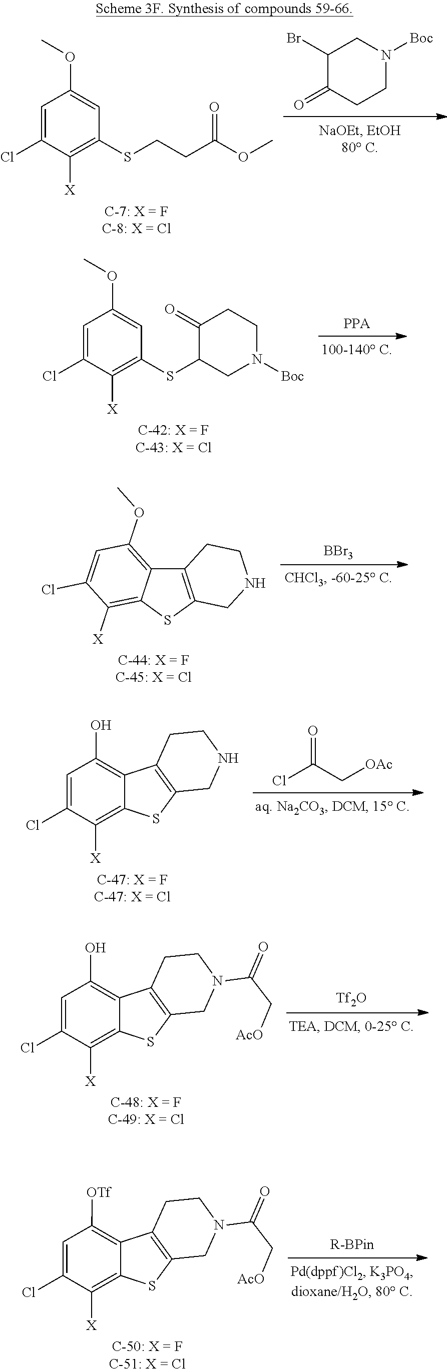

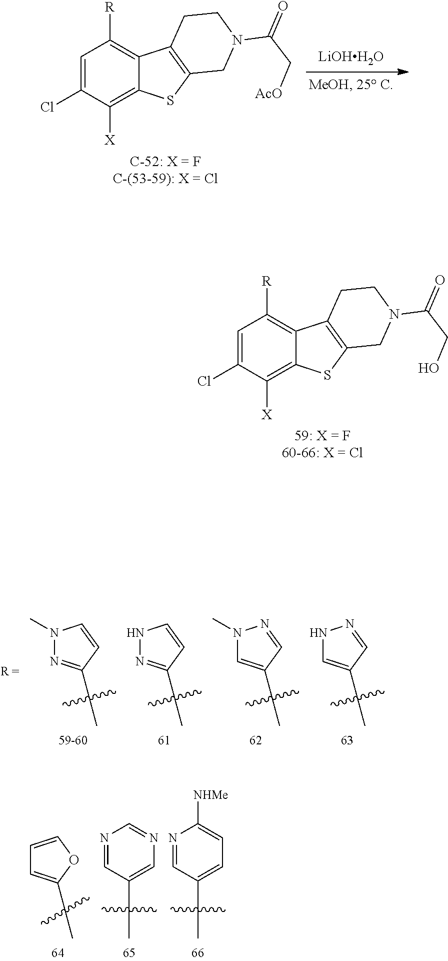

Compound C-43. A mixture of C-8 (100 mg, 338.8 umol, 1.0 eq.) in EtOH (2 mL) was added EtONa (69 mg, 1.0 mmol, 3.0 eq.). The mixture was stirred at 25° C. for 1 hr. The mixture was added tert-butyl 3-bromo-4-oxo-piperidine-1-carboxylate (283 mg, 1.0 mmol, 3.0 eq.) and stirred at 25° C. for 1 hr. The mixture was quenched with H₂O (10 mL), extracted with EtOAc (10 mL*3), dried over Na₂SO₄, filtered, concentrated and purified by prep-TLC (SiO₂, Petroleum ether: Ethyl acetate=5:1) to yield Compound C-43 (100 mg, 246.1 umol, 72.7% yield) as a colorless oil. ¹H NMR (400 MHz, DMSO-d₆) δ=7.19 (br s, 1H), 7.11 (d, J=2.8 Hz, 1H), 4.05 (q, J=7.2 Hz, 2H), 3.83 (br s, 3H), 3.65-3.58 (m, 2H), 3.52 (br dd, J-7.0, 13.7 Hz, 2H), 3.20-3.13 (m, 1H), 1.41 (d, J=5.9 Hz, 9H). ESI [M-tBu+H]=306.0/308.0.

Compound C-45. The solution of PPA (0.5 mL) was stirred at 100° C. for 30 mins. Then C-43 (20 mg, 49.2 umol, 1.0 eq.) was added and stirred at 140° C. for 2 hrs. The reaction mixture was diluted with MeOH (5 mL), purified by prep-HPLC (column: Phenomenex Luna 80*30 mm*3 μm; mobile phase: [water (TFA)-ACN]; B %: 15%-45%, 8 min) to yield C-45 (1 mg, 4.2 umol, 8.4% yield, 98.0% purity) as a pale yellow solid. ¹H NMR (400 MHZ, DMSO-d₆) δ=7.20 (s, 1H), 4.38 (br s, 2H), 3.92 (s, 3H), 3.81 (br s, 2H), 3.17 (br d, J=5.4 Hz, 2H). ESI [M+H]=287.9/289.9.

Compound C-47. A mixture of C-45 (820 mg, 2.9 mmol, 1.0 eq.) in CHCl₃ (10 mL) was added BBr₃ (2.1 g, 8.5 mmol, 3.0 eq.) at −60° C. The mixture was stirred at 25° C. for 12 hrs, then quenched by sat·aq. Na₂CO₃ (20 mL) until pH>8, extracted with DCM (15 mL*3). The organic layer was dried over Na₂SO₄, filtered, concentrated and purified by prep-HPLC (column: Waters Xbridge Prep OBD C18 150*40 mm*10 μm; mobile phase: [water (NH₃H₂O+NH₄HCO₃)-ACN]; B %: 20%-50%, 8 min) to yield C-47 (110 mg, 367.6 umol, 12.9% yield, 91.6% purity) as a brown solid. 1H NMR (400 MHZ, DMSO-d₆) δ=6.89 (s, 1H), 3.96 (s, 2H), 3.02-2.94 (m, 4H). ESI [M+H]=273.9/275.9.

Compound C-49. To a solution of C-47 (390 mg, 1.26 mmol, 1 eq., HCl) in DCM (4 mL) and sat·aq·Na₂CO₃ (4 mL) was added (2-chloro-2-oxo-ethyl) acetate (514.26 mg, 3.77 mmol, 404.93 uL, 3 eq.). The mixture was stirred at 15° C. for 3 hrs. The mixture was diluted with H₂O (20 mL) and extracted with EtOAc (30 mL*3). The organic layer was dried over Na₂SO₄, filtered and concentrated under reduced pressure to give a residue. The residue was purified by column chromatography (SiO₂, DCM:THF=1:0 to 5:1) to yield C-49 (280 mg, 748.19 umol, 59.59% yield) as a brown solid. 1H NMR (400 MHZ, DMSO-d₆) δ=10.70 (s, 1H), 6.89 (s, 1H), 4.98-4.84 (m, 2H), 4.83-4.65 (m, 2H), 3.72-3.56 (m, 2H), 3.21-2.97 (m, 2H), 2.09 (s, 3H). ESI [M−H]=372.0/374.0.

Compound C-51. A mixture of C-49 (40 mg, 106.88 umol, 1 eq.) in DCM (1 mL) and TEA (216.31 mg, 2.14 mmol, 20 eq.) was degassed and purged with N₂ for 3 times, then Tf₂O (90.47 mg, 320.65 umol, 3 eq.) in DCM (0.5 mL) was added at 0° C., and then the mixture was stirred at 25° C. for 12 hrs under N₂ atmosphere. The mixture was diluted with H₂O (10 mL) and extracted with DCM (15 mL*3). The organic layer was dried over Na₂SO₄, filtered, and concentrated to yield C-51 (130 mg, crude) as a brown solid. ESI [M+H]=506.0/508.0

Compounds C-(52-59). Compounds C-50-51 were coupled with substituted boronic ester derivatives using method A to yield C-52-59 as brown solid.

Compound 59-66. Ester hydrolysis of compounds C (52-59) using method C afforded compound 59-66.

3.7. Compounds 67-72 (Scheme 3G). Synthesis of 67-72 was achieved by reacting triflate C-51 and dipinacolato-diborane (B₂Pin₂) together, followed by Suzuki coupling of the resulting boronic acid derivative C-60 with bromoheterocycles to afford compounds C-(61-66) and ester hydrolysis to give the title products.

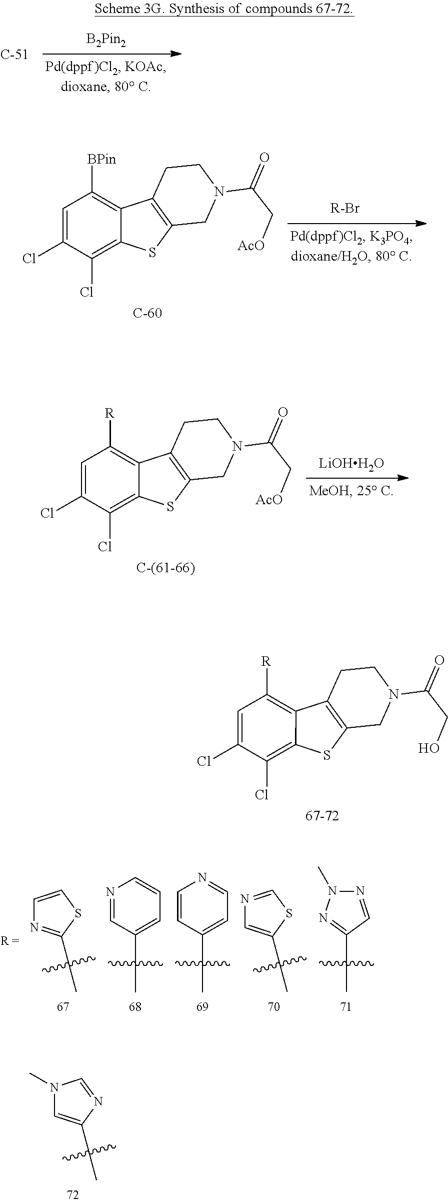

Compound C-60. A mixture of C-51 (660 mg, 1.30 mmol, 1 eq.), 4,4,5,5-tetramethyl-2-(4,4,5,5-tetramethyl-1,3,2-dioxaborolan-2-yl)-1,3,2-dioxaborolane (993.08 mg, 3.91 mmol, 3 eq.), Pd(dppf)Cl₂ (95.38 mg, 130.36 umol, 0.1 eq.), KOAc (383.81 mg, 3.91 mmol, 3 eq.) in dioxane (20 mL) was degassed and purged with N₂ for 3 times, and then the mixture was stirred at 80° C. for 12 hr under N₂ atmosphere. The reaction mixture was concentrated under reduced pressure to remove solvent. The residue was purified by column chromatography (SiO₂, Petroleum ether: Ethyl acetate=1:0 to 0:1) to yield C-60 (430 mg, 888.06 umol, 68.13% yield) as a white solid. ESI [M+H]=483.9/485.7.

Compound C-(61-66). Compound C-60 was coupled with substituted boronic ester derivatives using method A to yield C-(61-66) as a white solid.

Compound 67-72. Ester hydrolysis of compound C-54-56 using method C afforded compound 67-72.

3.8. Compounds 73-84 (Scheme 3H). Synthesis of compounds 73-84 was achieved starting with compound C-47 and reacting the latter with N-Boc protected glycine and 2-methylalanine. The resulting amide-alcohols C-67 and C-68 were converted to triflates C-69 and C-70 by reacting with triflic anhydride and subsequently treated with heterocyle-boronic acid derivatives (R₁BPin) to afford compounds C-(71-83). Finally, C-(71-83) were stirred in methanolic HCl affording 73-85 as HCl salts.

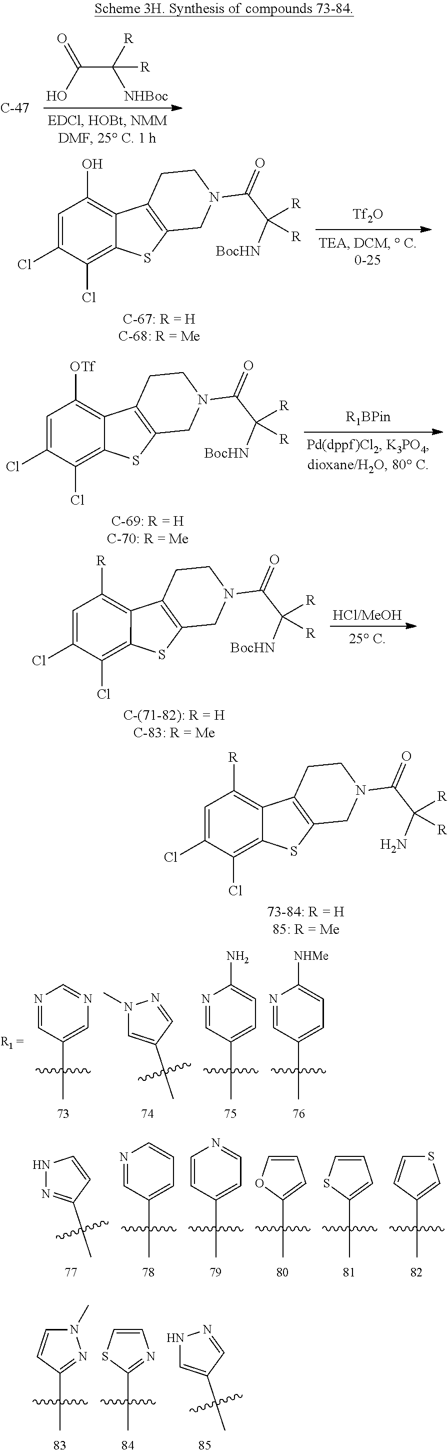

Compound C-67. A mixture of C-49 (10 mg, 36.5 umol, 1.0 eq.), N-boc glycine (19 mg, 109.4 umol, 3.0 eq.) and NMM (15 mg, 145.9 umol, 4.0 eq.) in DMF (1 mL) was added EDCI (10 mg, 54.7 umol, 1.5 eq.) and HOBt (7 mg, 54.7 umol, 1.5 eq.). The mixture was stirred at 25° C. for 1 hr. The reaction mixture was filtered and purified by prep-HPLC (TFA condition; column: Phenomenex Luna 80*30 mm*3 μm; mobile phase: [water (TFA)-ACN]; B %: 40%-75%, 8 min) to yield C-67 (8 mg, 17.8 umol, 48.7% yield, 100.0% purity) as a white solid. 1H NMR (400 MHZ, DMSO-d₆) δ=10.70 (s, 1H), 6.96-6.75 (m, 2H), 4.83-4.67 (m, 2H), 3.98-3.83 (m, 2H), 3.80-3.65 (m, 2H), 3.15-2.97 (m, 2H), 1.39 (s, 9H). ESI [M-tBu+H]=374.9/376.9.

Compound C-69. A mixture of C-67 (75 mg, 173.9 umol, 1.0 eq.) and TEA (352 mg, 3.5 mmol, 20.0 eq.) in DCM (1.0 mL) was degassed and purged with N₂ for 3 times, and Tf₂O (245 mg, 869.4 umol, 5.0 eq.) in DCM (0.5 mL) was added at 0° C., then the mixture was stirred at 25° C. for 1 hr under N₂ atmosphere. The reaction mixture was concentrated and purified by column chromatography (SiO₂, Petroleum ether: Ethyl acetate=3:2 to 3:2) to yield C-69 (93 mg, 165.1 umol, 95% yield) as a brown solid. ESI [M−H]=561.0/563.0

Compound C-(71-83). Compound C-69 was coupled with substituted boronic ester derivatives using method A to yield C-71-82 as a white solid. Compound C-83 was prepared similarly

Compound 73-85. NBoc deprotection of C-(71-83) using method B afforded compound 73-85 as a white solid.

3.9. Compounds 86-93 (Scheme 31). To prepare compounds 86-93, triflate C-69 was converted to boronic acid derivative C-84, and reacted to bromohetercocycles, as needed, giving compounds C-(85-92). The latter underwent N-Boc deprotection using methanolic HCl to afford the title products.

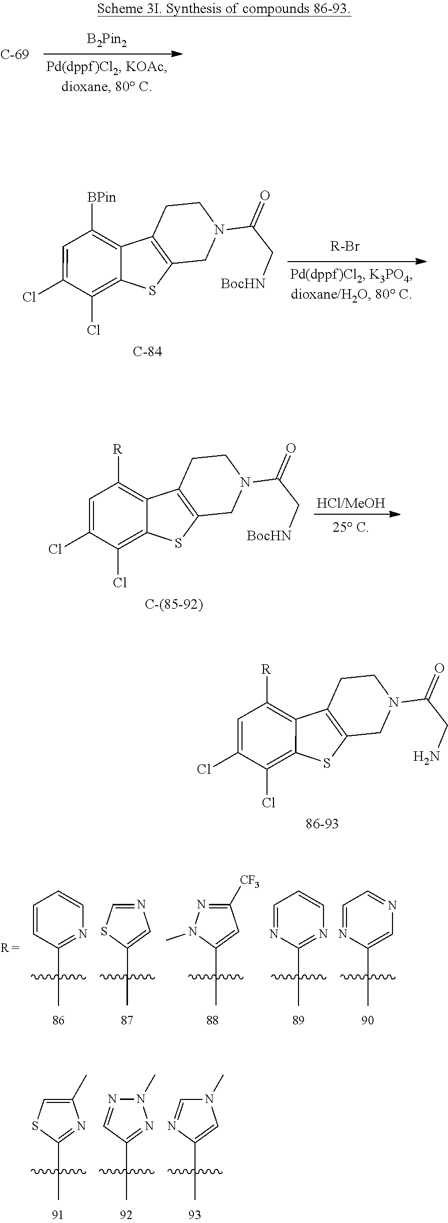

Compound C-84. A mixture of C-69 (1.6 g, 2.84 mmol, 1 eq.), 4,4,5,5-tetramethyl-2-(4,4,5,5-tetramethyl-1,3,2-dioxaborolan-2-yl)-1,3,2-dioxaborolane (1.44 g, 5.68 mmol, 2 eq.), Pd(dppf)Cl₂ (207.80 mg, 283.99 umol, 0.1 eq.), KOAc (836.15 mg, 8.52 mmol, 3 eq.) in dioxane (50 mL) was degassed and purged with N₂ for 3 times, and then the mixture was stirred at 80° C. for 12 hr under N₂ atmosphere. The reaction mixture was concentrated under reduced pressure to remove solvent. The residue was purified by column chromatography (SiO₂, Petroleum ether: Ethyl acetate=1:1 to 1:2) to yield C-84 (1.5 g, 2.77 mmol, 97.58% yield) as a white solid. ¹H NMR (400 MHZ, DMSO-d₆) δ=7.76 (s, 1H), 6.90 (br d, J=4.9 Hz, 1H), 4.94-4.85 (m, 2H), 3.83 (br d, J=3.8 Hz, 2H), 3.69-3.65 (m, 2H), 3.10-2.94 (m, 2H), 1.44 (br s, 12H), 1.22 (s, 4H), 1.13 (s, 5H).

Compound C-(85-92). Compound C-84 was coupled with substituted boronic ester derivatives using method A to yield C-(85-92) as a white solid.

Compound 86-93. NBoc deprotection of C-(85-92) using method B afforded compound 86-93 as white solid. Compounds C-(85-92). Compound C-84 was coupled with substituted boronic ester derivatives using method A to yield C-(85-92) as a white solid.

Compounds 86-93. NBoc deprotection of C (85-92) using method B afforded compound 86-93 as white solid.

### 3.10. Compounds 94-97 (Scheme 3J).

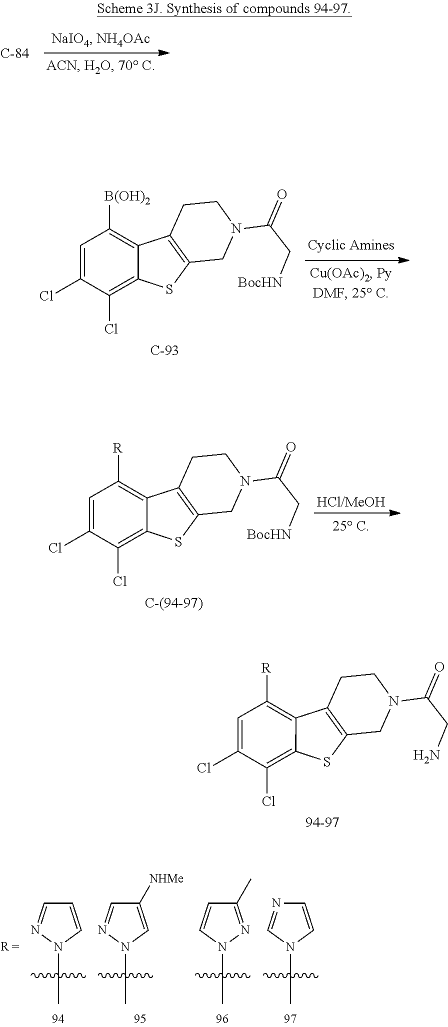

Compound C-93. To a solution of C-84 (1 g, 1.85 mmol, 1 eq.) in MeCN (15 mL) and H₂O (6 mL) was added NaIO₄ (1.58 g, 7.39 mmol, 409.48 uL, 4 eq.) and NH₄OAc (569.62 mg, 7.39 mmol, 4 eq.). The mixture was stirred at 70° C. for 12 hr. The reaction mixture was filtered purified by prep-HPLC (column: Phenomenex luna C18 250*50 mm*10 μm; mobile phase: [water (TFA)-ACN]; B %: 30%-70%, 10 min) to yield C-93 (350 mg, 762.28 umol, 41.26% yield) as a pale yellow solid. LCMS: m/z 458.9 [M+H]⁺.

Compound C-(94-97). A mixture of C-93 (30 mg, 65.34 umol, 1 eq.), substituted cyclic secondary amines (98.01 umol, 1.5 eq.), Cu(OAc)₂ (35.60 mg, 196.02 umol, 3 eq.), Py (25.84 mg, 326.70 umol, 5 eq.) in DMF (2 mL) was degassed and purged with O₂ for 3 times, and then the mixture was stirred at 25° C. for 1 hr under O₂ atmosphere. The reaction mixture diluted with H₂O (2 mL) and extracted with EtOAc (5 mL*3). The combined organic layers were dried over Na₂SO₄, filtered, and concentrated under reduced pressure to give a residue. The residue was purified by prep-HPLC (column: Waters Xbridge Prep OBD C18 150*40 mm*10 μm; mobile phase: [water (NH₄HCO₃)-ACN]; B %: 50%-85%, 8 min) to yield C-(94-97) as a white solid.

Compound 94-97. NBoc deprotection of intermediate C-(94-97) using method B afforded compound 94-97 as white solid.

### 4. Pyridoimidazole Derivatives

4.1. Compounds 98-101 (Scheme 4A).

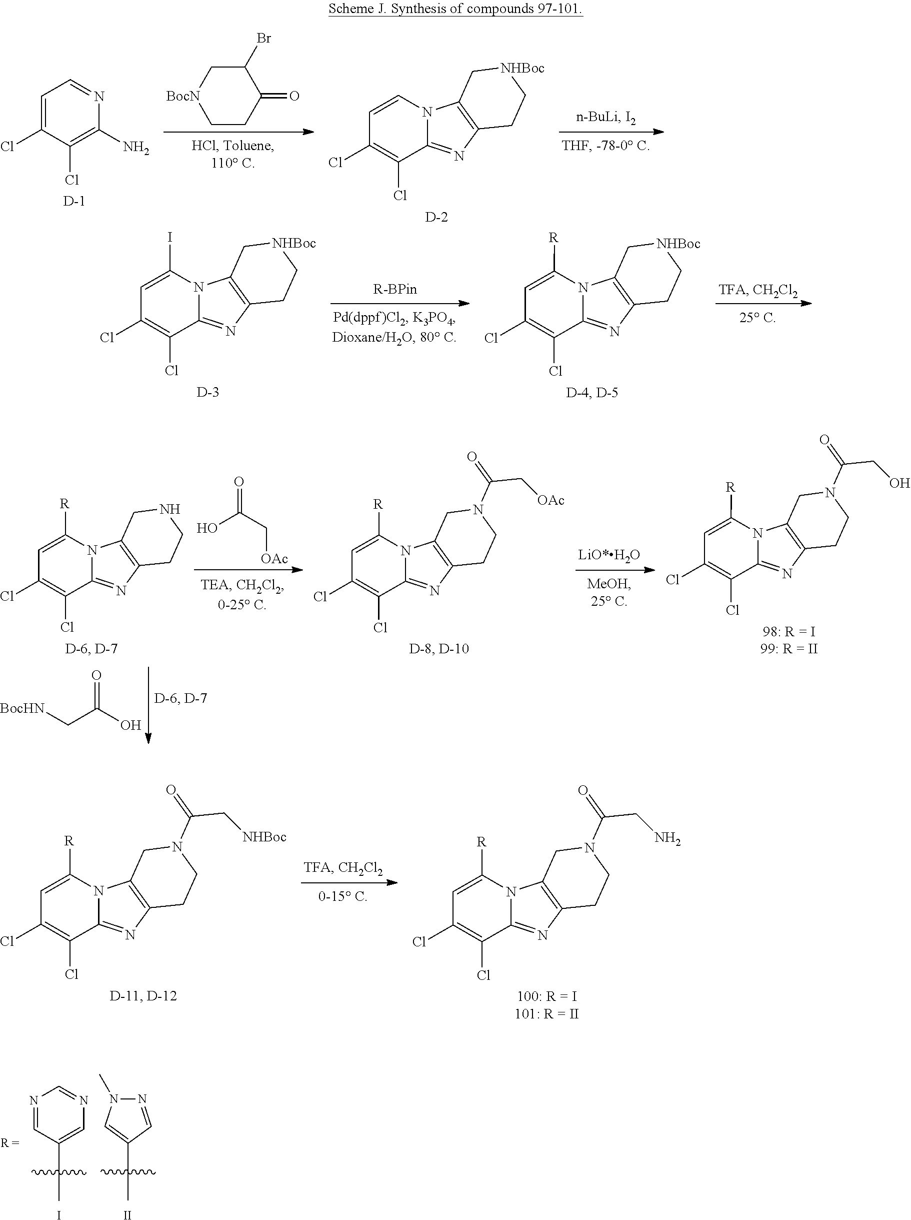

Compound D-2. A mixture of D-1 (980 mg, 6.0 mmol, 1.0 eq.), tert-butyl 3-bromo-4-oxo-piperidine-1-carboxylate (5.0 g, 18.0 mmol, 3.0 eq.) and HCl (1 M, 601 μL, 0.1 eq) in Tol. (10 mL) was stirred at 110° C. for 24 hrs. The mixture was quenched with H₂O (10 mL) and extracted with EtOAc (50 mL*3), dried over Na₂SO₄, filtered and concentrated under reduced pressure to give a residue, purified by column chromatography (SiO₂, Petroleum ether: Ethyl acetate=1:0 to 78:22) to yield D-2 (1.1 g, 3.2 mmol, 53.5% yield) as a white solid. 1H NMR (400 MHZ, DMSO-d₆) δ=8.41 (d, J=7.3 Hz, 1H), 7.16 (d, J=7.3 Hz, 1H), 4.72 (s, 2H), 3.73 (t, J=5.8 Hz, 2H), 2.80 (br t, J=5.6 Hz, 2H), 1.45 (s, 9H). ESI [M+H]=342.1/344.1

Compound D-3. A mixture of D-2 (330 mg, 964.3 umol, 1.0 eq.) in THF (4 mL) was degassed and purged with N₂ for 3 times, added n-BuLi (2.5 M, 578 μL, 1.5 eq.) at −78° C. and stirred for 0.5 hr, and then the mixture was added I₂ (367 mg, 1.4 mmol, 1.5 eq.) and stirred at 0° C. for 1 hr under N₂ atmosphere. The mixture was quenched by sat. aq·NH₄Cl (5 mL) and extracted with EtOAc (20 mL*3), dried over Na₂SO₄, filtered and concentrated under reduced pressure to give a residue, purified by column chromatography (SiO₂, Petroleum ether: Ethyl acetate=6:1 to 4:1) to yield D-3 (350 mg, 748 umol, 77.5% yield) as a yellow solid. ¹H NMR (400 MHZ, DMSO-d₆) δ=7.62 (s, 1H), 5.25 (br s, 2H), 3.67 (t, J=5.7 Hz, 2H), 2.78 (br t, J=5.3 Hz, 2H), 1.43 (s, 9H). ESI [M+H]=467.9/469.8.

Compound D-4 and D-5. Compound D-3 was coupled with substituted boronic ester derivatives using method A to yield D-4 and D-5 as a white solid. Similarly

Tert-butyl 10,11-dichloro-13-pyrimidin-5-yl-1,4,8-triazatricyclo [7.4.0.02,7]trideca-2 (7),8,10,12-tetraene-4-carboxylate, D-4 (70 mg, 166.6 umol, 91.7% yield). ¹H NMR (400 MHZ, DMSO-d₆) δ=9.42 (s, 1H), 9.14 (s, 2H), 7.30 (s, 1H), 3.78-3.72 (m, 2H), 3.60 (br s, 2H), 2.80 (br t, J=5.3 Hz, 2H), 1.41-1.27 (m, 9H). ESI [M+H]=420.2/422.1.

Tert-butyl 10,11-dichloro-13-(1-methylpyrazol-4-yl)-1,4,8-triazatricyclo [7.4.0.02,7]trideca-2 (7), 8,10,12-tetraene-4-carboxylate, D-5 (70 mg, 165.8 umol, 91.3% yield) as a brown oil. ¹H NMR (400 MHZ, DMSO-d₆) δ=8.17 (s, 1H), 7.80 (br d, J=2.2 Hz, 1H), 7.00 (s, 1H), 3.96 (br s, 2H), 3.69-3.60 (m, 5H), 2.86-2.75 (m, 2H), 1.37 (br s, 9H). ESI [M+H]=422.2/424.2

Compound D6 and D7. A mixture of D-4 and D-5 (65 mg, 154.6 umol, 1.0 eq.) in DCM (1.5 mL) was added TFA (0.5 mL) at 0° C. The mixture was stirred at 25° C. for 1 hr. The reaction was concentrated to yield D6 and D7 (70 mg, crude, TFA) as a brown oil, used into the next step without further purification.

Compound D8 and D-10. A mixture of D6 and D7 (60 mg, 138.2 umol, 1.0 eq, TFA) and TEA (70 mg, 690.9 umol, 5 eq.) in DCM (1 mL) was added (2-chloro-2-oxo-ethyl) acetate (28 mg, 207.3 umol, 1.5 eq.) at 0° C. The mixture was stirred at 25° C. for 1 hr. The mixture was quenched with Na₂CO₃ (2 mL) and extracted with EtOAc (3 mL*5), the organic layer was dried over Na₂SO₄, filtered, and concentrated under reduced pressure to yield D8 and D-10 (50 mg, crude) as a brown solid, used into the next step without further purification.

Compound 98 and 99. Ester hydrolysis of compound D8 and D-10 using method C afforded compound 98 and 99.

Compound D11 and D-12. A mixture of D6/D7 (25 mg, 69.7 umol, 1.0 eq., HCl) and 2-(tert-butoxycarbonylamino) acetic acid (24 mg, 139.4 umol, 2.0 eq.) in DMF (1 mL) was added NMM (28 mg, 278.8 umol, 4.0 eq.), EDCI (20 mg, 104.6 umol, 1.5 eq.) and HOBT (14 mg, 104.6 umol, 1.5 eq.). The mixture was stirred at 25° C. for 1 hr. The mixture was quenched with H₂O (3 mL) and extracted with EtOAc (3 mL*5), the organic layer was washed with brine (3 mL*3), dried over Na₂SO₄, filtered and concentrated under reduced pressure to yield D11 and D-1 (25 mg, crude) as a brown solid, used into the next step without further purification.

Compound 100 and 101. A mixture of D11/D-12 (20 mg, 41.9 umol, 1.0 eq.) in DCM (0.9 mL) was added TFA (0.3 mL) at 0° C., then stirred at 25° C. for 1 hr. The reaction was concentrated, diluted with sat·aq·Na₂CO₃ (5 mL) and extracted with EtOAc (5 mL*5), dried over Na₂SO₄, filtered, concentrated and purified by prep-HPLC (column: Waters Xbridge BEH C18 100*30 mm*10 μm; mobile phase: [water (NH₄HCO₃)-ACN]; B %: 15%-45%, 10 min) to yield 100 and 101 (2 mg, 4.3 umol, 10.3% yield, 100.0% purity) as a pale yellow solid.

TABLE 1
Spectral Data

| Cpd. | | |
|---|---|---|
| No. | Structure | Spectral Data |
| 1 | 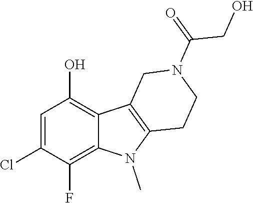 [SMILES: Cn1c2c(c3c(O)cc(Cl)c(F)c31)CN(C(=O)CO)CC2] | ¹H NMR (400 MHZ, DMSO-d6) δ = 9.91-9.82 (m, 1H), 6.34 (d, J = 5.0 Hz, 1H), 4.78-4.64 (m, 2H), 4.26-4.10 (m, 2H), 3.90-3.81 (m, 1H), 3.74 (s, 3H), 3.70 (br t, J = 5.5 Hz, 1H), 2.89-2.73 (m, 2H). ESI [M + H] = 313.1/315.0. |
| 2 | 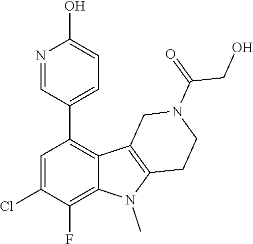 [SMILES: Cn1c2c(c3c(-c4ccc(O)nc4)cc(Cl)c(F)c31)CN(C(=O)CO)CC2] | ¹H NMR (400 MHZ, DMSO-d6) δ = 7.53-7.46 (m, 1H), 7.42 (br s, 1H), 6.95 (d, J = 6.3 Hz, 1H), 6.40 (d, J = 9.4 Hz, 1H), 4.31-4.10 (m, 3H), 4.00-3.89 (m, 1H), 3.82 (s, 3H), 3.75- 3.65 (m, 2H), 2.92-2.78 (m, 2H). LCMS: m/z 390.1 [M + H]⁺. |
| 3 | 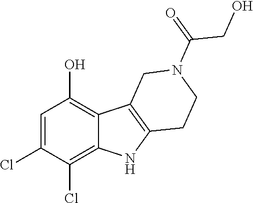 [SMILES: O=C(CO)N1CCc2[nH]c3c(Cl)c(Cl)cc(O)c3c2C1] | ¹H NMR (400 MHZ, DMSO-d6) δ = 11.31-11.19 (m, 1H), 10.10-10.01 (m, 1H), 6.51 (s, 1H), 4.75 (s, 1H), 4.66-4.61 (m, 1H), 4.24-4.13 (m, 2H), 3.85-3.59 (m, 2H), 2.85-2.69 (m, 2H). ESI [M + H] = 315.0/317.0. |
| 4 | 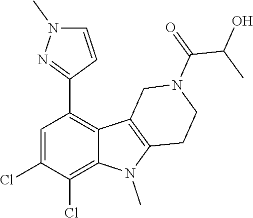 [SMILES: CC(O)C(=O)N1CCc2c(c3c(-c4ccn(C)n4)cc(Cl)c(Cl)c3n2C)C1] | ¹H NMR (400 MHZ, DMSO-d6) δ = 7.83 (s, 1H), 7.23 (d, J = 11.7 Hz, 1H), 6.59-6.49 (m, 1H), 4.57 (s, 1H), 4.54-4.48 (m, 0.5H), 4.42 (br d, J = 5.1 Hz, 1H), 4.30 (q, J = 6.7 Hz, 0.5H), 4.00 (s, 3H), 3.95 (d, J = 9.2 Hz, 3H), 3.91-3.77 (m, 2H), 2.96-2.78 (m, 2H), 1.23-1.11 (m, 3H). ESI [M + H] = 407.0/409.0. |
| 5 | 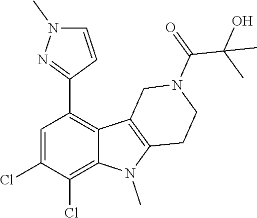 [SMILES: Cn1ccc(-c2cc(Cl)c(Cl)c3c2c2c(n3C)CCN(C(=O)C(C)(C)O)C2)n1] | ¹H NMR (400 MHZ, DMSO-d6) δ = 7.81 (s, 1H), 7.20 (s, 1H), 6.50 (d, J = 2.2 Hz, 1H), 4.89 (br s, 1H), 4.43-4.19 (m, 2H), 4.00 (s, 3H), 3.94 (s, 3H), 3.86 (br s, 2H), 2.97-2.76 (m, 2H), 1.39-1.16 (m, 6H). ESI [M + H] = 421.0/423.0. |
| 6 | 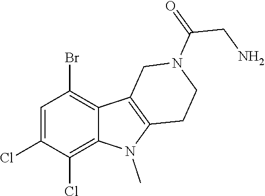 [SMILES: Cn1c2c(c3c(Br)cc(Cl)c(Cl)c31)CN(C(=O)CN)CC2] | ¹H NMR (400 MHZ, DMSO-d6) δ = 8.11 (br s, 2H), 7.49-7.44 (m, 1H), 5.04-4.83 (m, 2H), 4.05 (s, 2H), 4.02-3.98 (m, 0.5H), 3.95 (s, 3H), 3.94-3.89 (m, 0.5H), 3.76 (br t, J = 5.6 Hz, 1H), 3.00-2.77 (m, 2H). ESI [M + H] = 391.9/389.9. |
| 7 | 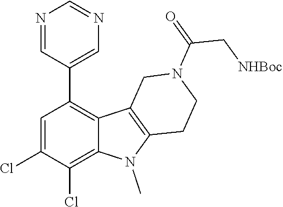 | ¹H NMR (400 MHZ, DMSO-d6) δ = 8.11 (br s, 2H), 7.49-7.44 (m, 1H), 5.04-4.83 (m, 2H), 4.05 (s, 2H), 4.02-3.98 (m, 0.5H), 3.95 (s, 3H), 3.94-3.89 (m, 0.5H), 3.76 (br t, J = 5.6 Hz, 1H), 3.00-2.77 (m, 2H). LCMS: m/z 391.9 [M + H]⁺. |
| 8 | 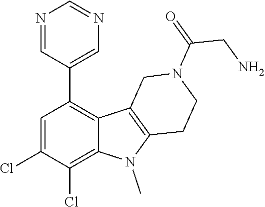 [SMILES: Cn1c2c(c3c(-c4cncnc4)cc(Cl)c(Cl)c31)CN(C(=O)CN)CC2] | ¹H NMR (400 MHZ, DMSO-d6) δ = 9.33 (s, 1H), 8.99-8.88 (m, 2H), 7.30 (s, 1H), 4.02 (s, 4H), 3.90-3.68 (m, 3H), 3.41 (br s, 2H), 2.96-2.80 (m, 2H). LCMS: m/z 390.1 [M + H]⁺. |
| 9 | 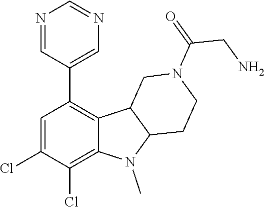 [SMILES: CN1c2c(Cl)c(Cl)cc(-c3cncnc3)c2C2CN(C(=O)CN)CCC21] | ¹H NMR (400M HZ, DMSO-d6) δ = 8.06 (br s, 2H), 7.36-7.23 (m, 1H), 6.34 (s, 1H), 4.74 (s, 1H), 4.59-4.44 (m, 2H), 4.04 (br s, 2H), 3.99 (s, 3H), 3.95 (br s, 1H), 3.79 (br s, 1H), 3.03-2.81 (m, 3H). ESI [M + H] = 392.0/394.0. |
| 10 | 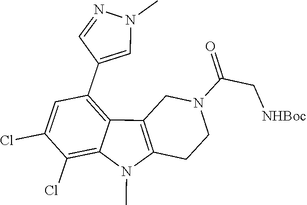 [SMILES: Cn1cc(-c2cc(Cl)c(Cl)c3c2c2c(n3C)CCN(C(=O)CNC(=O)OC(C)(C)C)C2)cn1] | ¹H NMR (400 MHZ, DMSO-d6) δ = 8.00-7.86 (m, 1H), 7.67- 7.49 (m, 1H), 7.02 (s, 1H), 6.83-6.63 (m, 1H), 4.34-4.13 (m, 2H), 3.96 (s, 3H), 3.91 (s, 3H), 3.86-3.71 (m, 4H), 2.91- 2.73 (m, 2H), 1.35 (s, 9H). LCMS: m/z 492.1 [M + H]⁺. |
| 11 | 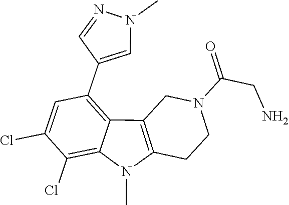 [SMILES: Cn1cc(-c2cc(Cl)c(Cl)c3c2c2c(n3C)CCN(C(=O)CN)C2)cn1] | ¹H NMR (400 MHZ, Methanol-d4) δ = 7.75 (d, J = 8.6 Hz, 1H), 7.56 (br d, J = 10.9 Hz, 1H), 7.01 (d, J = 6.1 Hz, 1H), 4.38 (s, 1H), 4.14 (s, 1H), 4.09 (br d, J = 4.0 Hz, 1H), 4.01-3.98 (m, 6H), 3.95 (br t, J = 6.0 Hz, 1H), 3.78 (br t, J = 5.4 Hz, 1H), 3.60-3.50 (m, 1H), 2.93-2.82 (m, 2H). LCMS: m/z 392.0 [M + H]⁺. |
| 12 | 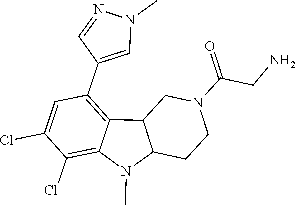 [SMILES: CN1c2c(Cl)c(Cl)cc(-c3cnn(C)c3)c2C2CN(C(=O)CN)CCC21] | ¹H NMR (400 MHZ, DMSO-d₆) δ = 8.27-8.13 (m, 1H), 7.86 (d, J = 10.5 Hz, 1H), 7.11 (d, J = 12.4 Hz, 1H), 4.06 (br dd, J = 5.6, 13.4 Hz, 1H), 3.88 (s, 3H), 3.69-3.69 (m, 1H), 3.79-3.58 (m, 1H), 3.57-3.40 (m, 2H), 3.17-3.09 (m, 1H), 3.06 (d, J = 1.5 Hz, 3H), 2.97-2.85 (m, 1H), 2.78 (br dd, J = 8.8, 13.8 Hz, 1H), 2.11-2.03 (m, 1H), 1.95-1.87 (m, 1H). ESI [M + H] = 394.0/396.0. |
| 13 | 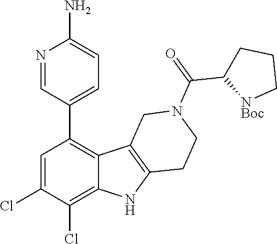 | ¹H NMR (400 MHZ, DMSO-d6) δ = 11.36 (br s, 1H), 7.96 (s, 1H), 7.44 (br d, J = 7.5 Hz, 1H), 6.97 (s, 1H), 6.58 (br d, J = 8.3 Hz, 1H), 5.86 (br s, 2H), 4.65 (br s, 1H), 4.41-4.22 (m, 1H), 4.11 (br s, 1H), 3.92-3.68 (m, 2H), 3.37-3.25 (m, 2H), 2.86 (br s, 2H), 1.77 (br s, 2H), 1.38 (br s, 2H), 1.35-1.01 (m, 9H). LCMS: m/z 530.2 [M + H]⁺. |
| 14 | 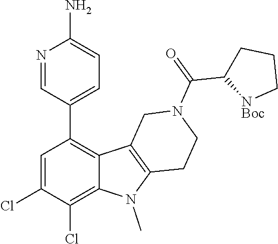 | ¹H NMR (400 MHZ, DMSO-d6) δ = 7.93 (d, J = 1.9 Hz, 1H), 7.41 (dd, J = 2.0, 8.4 Hz, 1H), 7.01 (s, 1H), 6.58 (br d, J = 8.3 Hz, 1H), 5.88 (br s, 2H), 4.09 (br s, 1H), 3.99 (s, 3H), 3.93- 3.74 (m, 2H), 3.36-3.27 (m, 2H), 3.15 (br s, 2H), 2.86 (br s, 2H), 1.78 (br s, 3H), 1.45-1.38 (m, 1H), 1.36-1.09 (m, 9H). LCMS: m/z 544.2 [M + H]⁺. |
| 15 | 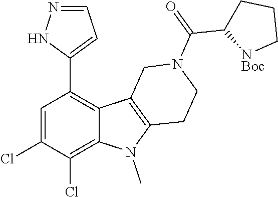 | ¹H NMR (400 MHZ, DMSO-d6) δ = 13.21-12.65 (m, 1H), 7.89-7.59 (m, 1H), 7.28-7.11 (m, 1H), 6.61-6.36 (m, 1H), 4.54-4.13 (m, 2H), 4.10-3.92 (m, 3H), 3.85-3.67 (m, 1H), 3.40-3.24 (m, 2H), 3.22-3.16 (m, 2H), 2.93-2.85 (m, 2H), 1.87-1.66 (m, 3H), 1.47-1.40 (m, 1H), 1.38-1.17 (m, 9H). LCMS: m/z 518.1 [M + H]⁺. |
| 16 | 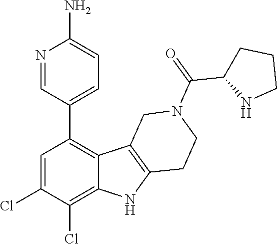 [SMILES: Nc1ccc(-c2cc(Cl)c(Cl)c3[nH]c4c(c23)CN(C(=O)[C@@H]2CCCN2)CC4)cn1] | ¹H NMR (400 MHZ, DMSO-d6) δ = 11.95-11.86 (m, 1H), 10.27-10.11 (m, 1H), 8.42 (br d, J = 4.4 Hz, 3H), 8.14-8.08 (m, 1H), 8.02 (dd, J = 2.0, 9.1 Hz, 1H), 7.20-7.14 (m, 2H), 4.72-4.64 (m, 1H), 4.37-4.30 (m, 1H), 4.22 (br d, J = 15.6 Hz, 1H), 3.98-3.64 (m, 2H), 3.26-3.13 (m, 2H), 3.05-2.95 (m, 1H), 2.94-2.87 (m, 1H), 2.48-2.38 (m, 1H), 2.00-1.67 (m, 3H). LCMS: m/z 430.1 [M + H]⁺. |
| 17 | 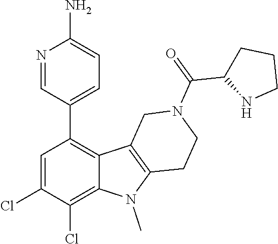 | ¹H NMR (400 MHz, DMSO-d6) δ = 7.91 (br s, 1H), 7.48-7.35 (m, 1H), 7.08-6.98 (m, 1H), 6.59-6.49 (m, 1H), 6.19-6.05 (m, 2H), 4.27-4.01 (m, 3H), 3.98 (d, J = 3.1 Hz, 3H), 3.83 (br d, J = 6.0 Hz, 1H), 3.64-3.57 (m, 1H), 3.02-2.72 (m, 5H), 1.72-1.48 (m, 3H), 1.24 (s, 1H). LCMS: m/z 444.2 [M + H]⁺. |
| 18 | 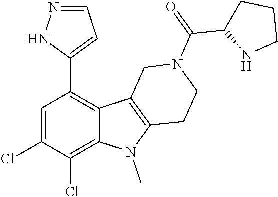 | ¹H NMR (400 MHZ, DMSO-d6) δ = 9.84-9.69 (m, 1H), 8.54- 8.31 (m, 1H), 7.89-7.78 (m, 1H), 7.31-7.19 (m, 1H), 6.63- 6.48 (m, 1H), 4.71-4.62 (m, 1H), 4.51-4.32 (m, 3H), 4.20- 4.09 (m, 1H), 4.00 (d, J = 1.3 Hz, 3H), 3.84 (br t, J = 5.7 Hz, 2H), 3.01-2.83 (m, 3H), 2.04-1.65 (m, 4H). LCMS: m/z 418.1 [M + H]⁺. |
| 19 | 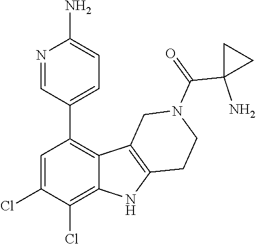 [SMILES: Nc1ccc(-c2cc(Cl)c(Cl)c3[nH]c4c(c23)CN(C(=O)C2(N)CC2)CC4)cn1] | ¹H NMR (400 MHZ, DMSO-d6) δ = 11.85 (s, 1H), 8.12 (br d, J = 1.9 Hz, 2H), 8.02 (br d, J = 8.8 Hz, 2H), 7.71-7.63 (m, 1H), 7.58 (br dd, J = 2.2, 7.2 Hz, 1H), 7.17 (s, 1H), 7.09 (br d, J = 9.1 Hz, 1H), 4.26 (s, 2H), 3.95-3.87 (m, 2H), 2.96 (br s, 2H), 1.30-1.25 (m, 2H), 1.19-1.10 (m, 2H). LCMS: m/z 416.0 [M + H]⁺. |
| 20 | 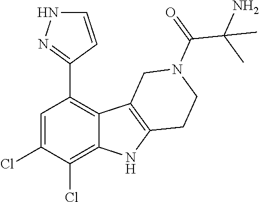 [SMILES: CC(C)(N)C(=O)N1CCc2[nH]c3c(Cl)c(Cl)cc(-c4cc[nH]n4)c3c2C1] | ¹H NMR (400 MHZ, DMSO-d6) δ = 13.04 (br d, J = 2.8 Hz, 1H), 11.72-11.43 (m, 1H), 7.90-7.76 (m, 1H), 7.74-7.28 (m, 2H), 7.21 (s, 1H), 6.57 (br s, 1H), 4.87-4.43 (m, 2H), 4.26- 3.93 (m, 2H), 2.86 (br s, 2H), 1.25 (br s, 6H). LCMS: m/z 392.1 [M + H]⁺. |
| 21 | 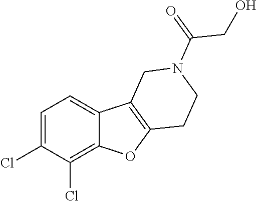 [SMILES: O=C(CO)N1CCc2oc3c(Cl)c(Cl)ccc3c2C1] | ¹H NMR (400MHZ, DMSO-d6) δ = 7.71-7.59 (m, 1H), 7.53 (br d, J = 7.9 Hz, 1H), 4.83-4.71 (m, 1H), 4.70-4.56 (m, 2H), 4.33-4.20 (m, 2H), 3.94 (br s, 1H), 3.79 (br s, 1H), 3.03-2.86 (m, 2H). ESI [M + H] = 300.0/302.0. |
| 24 | 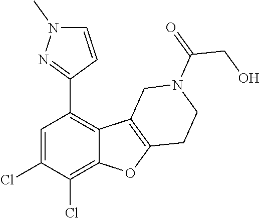 [SMILES: Cn1ccc(-c2cc(Cl)c(Cl)c3oc4c(c23)CN(C(=O)CO)CC4)n1] | ¹H NMR (400 MHz, DMSO-d₆) δ = 7.89-7.80 (m, 1H), 7.74- 7.64 (m, 1H), 6.79 (dd, J = 1.8, 17.2 Hz, 1H), 4.75 (br d, J = 6.3 Hz, 2H), 4.19 (br d, J = 13.9 Hz, 2H), 4.00-3.93 (m, 3H), 3.91-3.72 (m, 2H), 3.01-2.87 (m, 2H). LCMS: m/z 380.1 [M + H]⁺. |
| 25 | 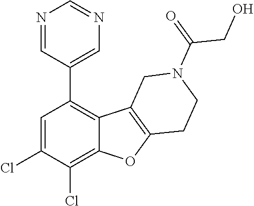 [SMILES: O=C(CO)N1CCc2oc3c(Cl)c(Cl)cc(-c4cncnc4)c3c2C1] | ¹H NMR (400 MHZ, DMSO-d₆) δ = 9.34 (s, 1H), 9.03 (s, 2H), 7.69 (s, 1H), 4.84-4.64 (m, 1H), 4.26-4.06 (m, 3H), 3.98- 3.81 (m, 1H), 3.77-3.65 (m, 1H), 3.08-2.83 (m, 2H). LCMS: m/z 377.9 [M + H]⁺. |
| 26 | 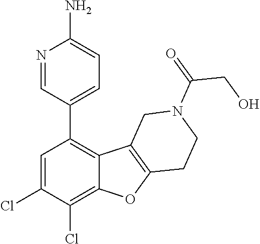 [SMILES: Nc1ccc(-c2cc(Cl)c(Cl)c3oc4c(c23)CN(C(=O)CO)CC4)cn1] | ¹H NMR (400 MHZ, DMSO-d₆) δ = 8.19 (d, J = 1.5 Hz, 1H), 8.05 (br d, J = 8.4 Hz, 1H), 7.54 (s, 1H), 7.08 (d, J = 9.1 Hz, 1H), 4.30 (br s, 1H), 4.20 (br s, 2H), 4.09-4.01 (m, 1H), 3.89- 3.81 (m, 1H), 3.75 (br s, 1H), 3.04-2.90 (m, 2H). LCMS: m/z 392.0 [M + H]⁺. |
| 28 | 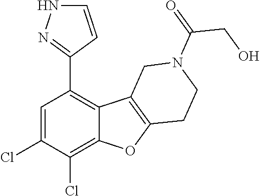 [SMILES: O=C(CO)N1CCc2oc3c(Cl)c(Cl)cc(-c4cc[nH]n4)c3c2C1] | ¹H NMR (400 MHZ, DMSO-d₆) δ = 13.37-13.07 (m, 1H), 7.88 (br s, 1H), 7.74-7.59 (m, 1H), 6.87-6.74 (m, 1H), 4.74-4.65 (m, 2H), 4.23-4.11 (m, 2H), 3.98-3.69 (m, 2H), 3.02-2.92 (m, 2H). LCMS: m/z 366.0 [M + H]⁺. |
| 29 | 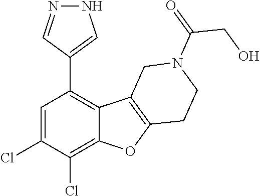 [SMILES: O=C(CO)N1CCc2oc3c(Cl)c(Cl)cc(-c4cn[nH]c4)c3c2C1] | ¹H NMR (400 MHz, DMSO-d₆) δ = 7.88 (br s, 2H), 7.43-7.33 (m, 1H), 4.41 (br s, 2H), 4.15 (br s, 2H), 3.83 (br s, 2H), 2.97 (br s, 2H). LCMS: m/z 366.0 [M + H]⁺. |
| 30 | 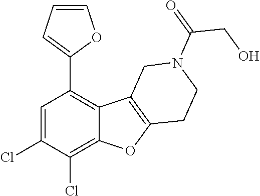 [SMILES: O=C(CO)N1CCc2oc3c(Cl)c(Cl)cc(-c4ccco4)c3c2C1] | ¹H NMR (400 MHZ, DMSO-d₆) δ = 7.98-7.88 (m, 1H), 7.86- 7.75 (m, 1H), 7.07 (br d, J = 2.8 Hz, 1H), 6.77-6.66 (m, 1H), 4.80-4.70 (m, 2H), 4.23 (br dd, J = 5.8, 14.4 Hz, 2H), 3.81- 3.62 (m, 2H), 3.02-2.77 (m, 2H). LCMS: m/z 366.0 [M + H]⁺. |
| 31 | 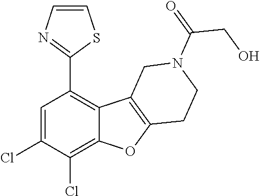 [SMILES: O=C(CO)N1CCc2oc3c(Cl)c(Cl)cc(-c4nccs4)c3c2C1] | ¹H NMR (400 MHZ, DMSO-d₆) δ = 8.18-8.10 (m, 1H), 8.05 (br d, J = 3.1 Hz, 1H), 7.96-7.85 (m, 1H), 4.76-4.71 (m, 2H), 4.31-4.16 (m, 2H), 4.01-3.91 (m, 1H), 3.82 (br t, J = 5.3 Hz, 1H), 3.13-2.97 (m, 2H) LCMS: m/z 383.0 [M + H]⁺. |
| 32 | 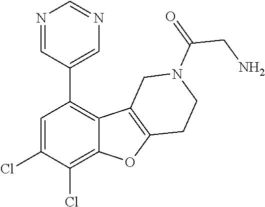 [SMILES: NCC(=O)N1CCc2oc3c(Cl)c(Cl)cc(-c4cncnc4)c3c2C1] | ¹H NMR (400 MHZ, DMSO-d6) δ = 9.29 (s, 1H), 8.98 (s, 2H), 8.06 (br s, 2H), 7.72-7.65 (m, 1H), 4.85-4.78 (m, 2H), 4.00 (br d, J = 1.4 Hz, 2H), 3.71 (br t, J = 5.6 Hz, 1H), 3.55 (br t, J = 5.3 Hz, 1H), 2.39 (br s, 1.5H), 2.28 (br s, 0.5H). ESI [M − H] = 377.0/379.0. |
| 33 | 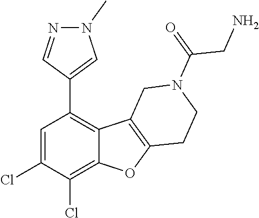 [SMILES: Cn1cc(-c2cc(Cl)c(Cl)c3oc4c(c23)CN(C(=O)CN)CC4)cn1] | ¹H NMR (400 MHZ, DMSO-d₆) δ = 8.05 (br d, J = 7.8 Hz, 1H), 7.78-7.67 (m, 1H), 7.43-7.35 (m, 1H), 4.75 (br s, 1H), 4.51-4.31 (m, 1H), 3.91 (d, J = 9.7 Hz, 3H), 3.79-3.67 (m, 1H), 3.59 (br s, 1H), 3.50-3.43 (m, 2H), 3.02-2.85 (m, 2H), 2.68-2.53 (m, 2H). LCMS: m/z 379.0 [M + H]⁺. |
| 34 | 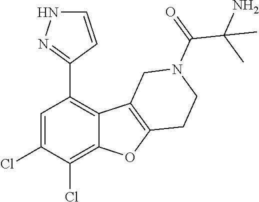 [SMILES: CC(C)(N)C(=O)N1CCc2oc3c(Cl)c(Cl)cc(-c4cc[nH]n4)c3c2C1] | ¹H NMR (400 MHZ, DMSO-d₆) δ = 8.29 (br s, 2H), 7.85 (d, J = 2.1 Hz, 1H), 7.66 (s, 1H), 6.74 (d, J = 2.3 Hz, 1H), 4.87 (br s, 2H), 3.84 (br t, J = 5.3 Hz, 2H), 2.81 (br s, 2H), 1.63 (s, 6H) ESI [M + H] = 393.1/395.1 |
| 35 | 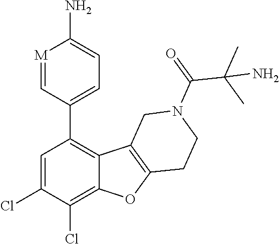 | ¹H NMR (400 MHZ, DMSO-d₆) δ = 8.24 (br s, 4H), 8.17 (d, J = 1.5 Hz, 1H), 7.98 (br d, J = 8.0 Hz, 1H), 7.59-7.52 (m, 1H), 7.06-7.03 (m, 1H), 4.33 (s, 2H), 3.97 (br t, J = 5.5 Hz, 2H), 3.02 (br s, 2H), 1.55 (br s, 6H). LCMS: m/z 419.1 [M + H]⁺. |
| 39 | 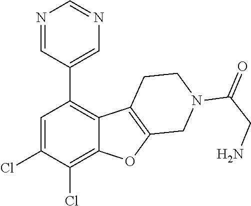 [SMILES: NCC(=O)N1CCc2c(oc3c(Cl)c(Cl)cc(-c4cncnc4)c23)C1] | ¹H NMR (400 MHZ, DMSO-d₆) δ = 9.37 (s, 1H), 9.06 (s, 2H), 8.30 (br d, J = 5.3 Hz, 2H), 7.82-7.74 (m, 1H), 4.90 (s, 2H), 4.12-4.04 (m, 2H), 3.83-3.75 (m, 1H), 3.69-3.61 (m, 1H), 2.48 (br s, 1H), 2.40-2.34 (m, 1H). ESI [M + H] = 377.0/379.0 |
| 40 | 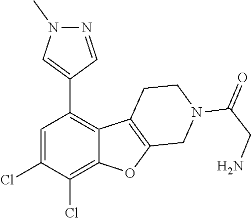 [SMILES: Cn1cc(-c2cc(Cl)c(Cl)c3oc4c(c23)CCN(C(=O)CN)C4)cn1] | ¹H NMR (400 MHZ, DMSO-d₆) δ = 8.20 (br s, 2H), 8.05 (d, J = 1.6 Hz, 1H), 7.70 (d, J = 6.4 Hz, 1H), 7.42 (d, J = 4.4 Hz, 1H), 4.81 (br d, J = 5.4 Hz, 2H), 4.02 (br dd, J = 5.6, 9.4 Hz, 2H), 3.91 (s, 3H), 3.77 (br t, J = 5.3 Hz, 1H), 3.62 (br t, J = 5.4 Hz, 1H), 2.70 (br s, 1H), 2.60 (br s, 1H). ESI [M + H] = 379.0/381.0 |
| 41 | 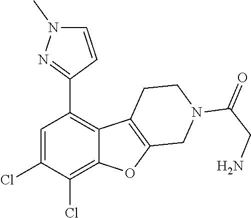 [SMILES: Cn1ccc(-c2cc(Cl)c(Cl)c3oc4c(c23)CCN(C(=O)CN)C4)n1] | ¹H NMR (400 MHZ, DMSO-d₆) δ = 8.17 (br s, 2H), 7.82 (dd, J = 2.1, 5.2 Hz, 1H), 7.64 (d, J = 5.1 Hz, 1H), 6.70 (dd, J = 2.2, 5.7 Hz, 1H), 4.81 (br d, J = 7.4 Hz, 2H), 4.03 (br dd, J = 5.8, 11.9 Hz, 2H), 3.93 (d, J = 5.0 Hz, 3H), 3.80-3.77 (m, 1H), 3.63 (br t, J = 5.5 Hz, 1H), 2.90 (br s, 1H), 2.80 (br s, 1H). ESI [M + H] = 379.0/381.0. |
| 46 | 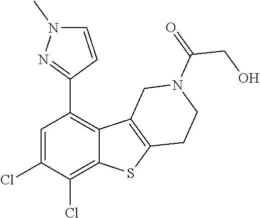 [SMILES: Cn1ccc(-c2cc(Cl)c(Cl)c3sc4c(c23)CN(C(=O)CO)CC4)n1] | ¹H NMR (400 MHZ, DMSO-d6) δ = 7.86 (br s, 1H), 7.50 (s, 1H), 6.50 (d, J = 1.9 Hz, 1H), 4.22 (s, 1H), 4.16-4.11 (m, 1H), 4.10-4.04 (m, 1H), 3.97-3.92 (m, 3H), 3.89-3.81 (m, 1H), 3.78 (br t, J = 5.4 Hz, 1H), 3.67-3.63 (m, 1H), 3.07-2.88 (m, 2H). LCMS: m/z 396 [M + H]⁺. |
| 47 | 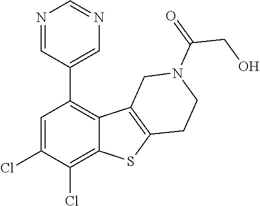 [SMILES: O=C(CO)N1CCc2sc3c(Cl)c(Cl)cc(-c4cncnc4)c3c2C1] | ¹H NMR (400 MHZ, DMSO-d6) δ = 9.34 (s, 1H), 8.97 (s, 2H), 7.67 (s, 1H), 4.67-4.54 (m, 1H), 4.12 (br d, J = 4.9 Hz, 1H), 3.90 (br s, 1H), 3.80-3.61 (m, 4H), 3.05-2.93 (m, 2H). LCMS: m/z 394.0 [M + H]⁺. |
| 48 | 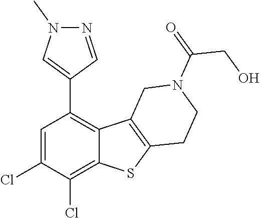 [SMILES: Cn1cc(-c2cc(Cl)c(Cl)c3sc4c(c23)CN(C(=O)CO)CC4)cn1] | ¹H NMR (400 MHZ, DMSO-d6) δ = 7.98-7.91 (m, 1H), 7.60 (s, 1H), 7.40 (s, 1H), 4.22 (br s, 1H), 4.14 (s, 1H), 4.01 (br s, 1H), 3.92 (s, 3H), 3.84 (br s, 1H), 3.79-3.62 (m, 2H), 3.06-2.88 (m, 2H). LCMS: m/z 396.1 [M + H]⁺. |
| 49 | 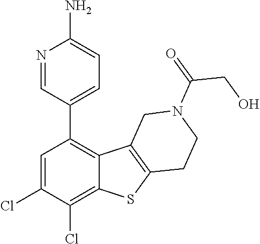 [SMILES: Nc1ccc(-c2cc(Cl)c(Cl)c3sc4c(c23)CN(C(=O)CO)CC4)cn1] | ¹H NMR (400 MHZ, DMSO-d6) δ = 7.89 (br s, 1H), 7.45-7.33 (m, 2H), 6.50 (d, J = 8.4 Hz, 1H), 6.16 (s, 2H), 4.82-4.76 (m, 2H), 4.74 (br t, J = 5.7 Hz, 1H), 4.14 (br d, J = 5.1 Hz, 2H), 3.48 (br s, 1H), 2.31-2.10 (m, 2H). LCMS: m/z 408.1 [M + H]⁺. |
| 50 | 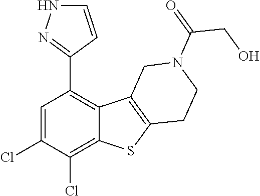 [SMILES: O=C(CO)N1CCc2sc3c(Cl)c(Cl)cc(-c4cc[nH]n4)c3c2C1] | ¹H NMR (400 MHZ, DMSO-d6) δ = 7.97-7.82 (m, 1H), 7.57-7.41 (m, 1H), 6.60-6.47 (m, 1H), 4.17-4.09 (m, 2H), 4.06-3.47 (m, 4H), 3.05-2.91 (m, 2H). LCMS: m/z 382.0 [M + H]⁺. |
| 51 | 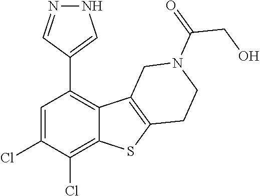 [SMILES: O=C(CO)N1CCc2sc3c(Cl)c(Cl)cc(-c4cn[nH]c4)c3c2C1] | ¹H NMR (400 MHZ, DMSO-d6) δ = 13.15 (br s, 1H), 7.99 (br s, 1H), 7.72-7.63 (m, 1H), 7.40 (s, 1H), 4.64-4.55 (m, 1H), 4.18 (br s, 1H), 4.13 (br d, J = 5.5 Hz, 1H), 3.95 (br s, 1H), 3.82-3.63 (m, 3H), 3.04-2.88 (m, 2H). LCMS: m/z 382.0 [M + H]⁺. |
| 52 | 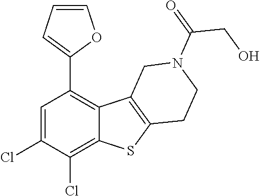 [SMILES: O=C(CO)N1CCc2sc3c(Cl)c(Cl)cc(-c4ccco4)c3c2C1] | ¹H NMR (400 MHZ, DMSO-d6) δ = 7.96-7.85 (m, 1H), 7.71-7.60 (m, 1H), 6.85-6.67 (m, 2H), 4.67-4.56 (m, 1H), 4.28-4.25 (m, 1H), 4.19-4.04 (m, 2H), 3.86 (br d, J = 5.3 Hz, 1H), 3.83-3.64 (m, 2H), 3.08-2.93 (m, 2H). LCMS: m/z 382.0 [M + H]⁺. |
| 53 | 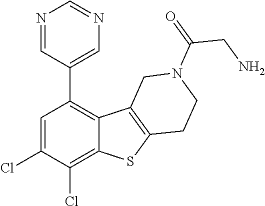 [SMILES: NCC(=O)N1CCc2sc3c(Cl)c(Cl)cc(-c4cncnc4)c3c2C1] | ¹H NMR (400 MHZ, DMSO-d6) δ = 9.30 (s, 1H), 8.92 (s, 2H), 7.69-7.54 (m, 1H), 4.82 (br s, 2H), 3.63-3.43 (m, 2H), 3.37 (br s, 2H), 2.16-1.97 (m, 2H). LCMS: m/z 393.1 [M + H]⁺. |
| 54 | 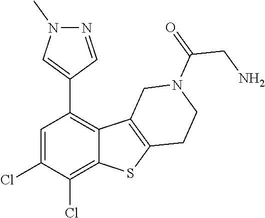 [SMILES: Cn1cc(-c2cc(Cl)c(Cl)c3sc4c(c23)CN(C(=O)CN)CC4)cn1] | ¹H NMR (400 MHZ, DMSO-d6) δ = 7.95 (s, 1H), 7.74-7.53 (m, 1H), 7.45-7.32 (m, 1H), 4.22 (s, 1H), 4.01 (s, 1H), 3.97-3.87 (m, 3H), 3.80-3.58 (m, 2H), 3.41-3.37 (m, 1H), 3.05 (br s, 1H), 3.03-2.85 (m, 2H). LCMS: m/z 395.0 [M + H]⁺. |
| 55 | 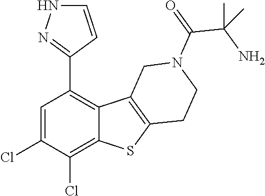 [SMILES: CC(C)(N)C(=O)N1CCc2sc3c(Cl)c(Cl)cc(-c4cc[nH]n4)c3c2C1] | ¹H NMR (400 MHZ, DMSO-d6) δ = 13.26-13.05 (m, 1H), 7.90 (br s, 1H), 7.49 (s, 1H), 6.51 (d, J = 2.0 Hz, 1H), 4.65- 3.88 (m, 4H), 2.97 (br s, 2H), 1.31 (s, 1H), 1.17 (br s, 5H). LCMS: m/z 409.1 [M + H]⁺. |
| 56 | 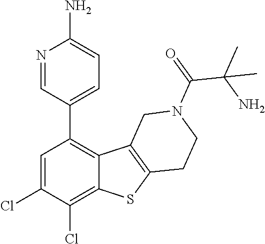 [SMILES: CC(C)(N)C(=O)N1CCc2sc3c(Cl)c(Cl)cc(-c4ccc(N)nc4)c3c2C1] | ¹H NMR (400 MHZ, DMSO-d6) δ = 8.25 (br s, 4H), 8.13 (s, 1H), 7.98 (br d, J = 8.0 Hz, 1H), 7.58 (s, 1H), 7.09 (br d, J = 8.8 Hz, 1H), 4.18 (s, 2H), 3.90 (br d, J = 5.0 Hz, 2H), 3.04 (br s, 2H), 1.61-1.41 (m, 6H). LCMS: m/z 435.0 [M + H]⁺. |
| 57 | 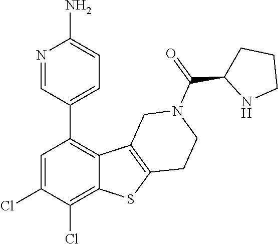 [SMILES: Nc1ccc(-c2cc(Cl)c(Cl)c3sc4c(c23)CN(C(=O)[C@H]2CCCN2)CC4)cn1] | ¹H NMR (400 MHZ, DMSO-d6) δ = 10.22 (br d, J = 4.5 Hz, 1H), 8.58 (br s, 2H), 8.27-8.16 (m, 1H), 8.08 (br d, J = 7.6 Hz, 1H), 7.68-7.59 (m, 1H), 7.24 (d, J = 9.1 Hz, 1H), 4.81-4.56 (m, 1H), 4.44-4.25 (m, 1H), 4.23-4.13 (m, 1H), 3.97-3.76 (m, 2H), 3.31-3.07 (m, 4H), 2.53-2.40 (m, 1H), 2.11-1.76 (m, 3H). LCMS: m/z 447.1 [M + H]⁺. |
| 58 | 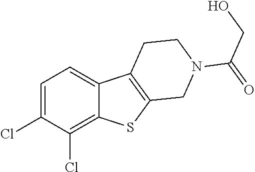 [SMILES: O=C(CO)N1CCc2c(sc3c(Cl)c(Cl)ccc23)C1] | ¹H NMR (400 MHZ, DMSO-d6) δ = 7.76-7.71 (m, 1H), 7.70-7.65 (m, 1H), 4.89-4.75 (m, 3H), 4.30-4.17 (m, 2H), 3.91-3.71 (m, 2H), 2.96-2.80 (m, 2H). ESI [M + H] = 315.9/318.0. |
| 60 | 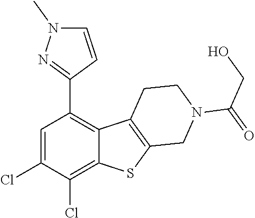 [SMILES: Cn1ccc(-c2cc(Cl)c(Cl)c3sc4c(c23)CCN(C(=O)CO)C4)n1] | ¹H NMR (400 MHZ, DMSO-d6) δ = 7.82 (d, J = 2.1 Hz, 1H), 7.47 (s, 1H), 6.45 (d, J = 2.0 Hz, 1H), 4.86-4.75 (m, 2H), 4.16 (s, 2H), 3.92 (s, 3H), 3.58 (br s, 2H), 2.38-2.21 (m, 2H). LCMS: m/z 396.0 [M + H]⁺. |
| 61 | 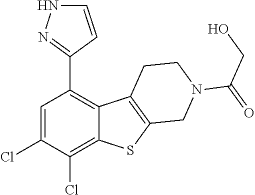 [SMILES: O=C(CO)N1CCc2c(sc3c(Cl)c(Cl)cc(-c4cc[nH]n4)c23)C1] | ¹H NMR (400 MHZ, DMSO-d6) δ = 13.18 (br s, 1H), 7.94 (br s, 1H), 7.60-7.50 (m, 1H), 6.55 (s, 1H), 4.86 (br d, J = 12.5 Hz, 2H), 4.21 (br s, 2H), 3.65-3.53 (m, 2H), 2.38-2.23 (m, 2H). LCMS: m/z 382.0 [M + H]⁺. |
| 62 | 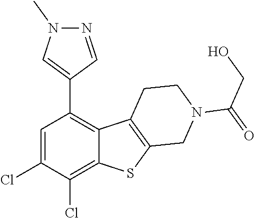 [SMILES: Cn1cc(-c2cc(Cl)c(Cl)c3sc4c(c23)CCN(C(=O)CO)C4)cn1] | ¹H NMR (400 MHZ, DMSO-d6) δ = 7.91 (br s, 1H), 7.60-7.53 (m, 1H), 7.38 (br s, 1H), 4.81-4.68 (m, 2H), 4.17 (br s, 2H), 3.91 (br s, 3H), 3.67-3.44 (m, 2H), 2.45-2.32 (m, 2H). LCMS: m/z 396.1 [M + H]⁺. |
| 63 | 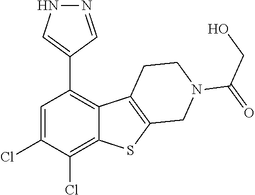 [SMILES: O=C(CO)N1CCc2c(sc3c(Cl)c(Cl)cc(-c4cn[nH]c4)c23)C1] | ¹H NMR (400 MHZ, DMSO-d6) δ = 13.17-13.02 (m, 1H), 7.97 (br d, J = 3.9 Hz, 1H), 7.74-7.60 (m, 1H), 7.41 (s, 1H), 4.85-4.69 (m, 3H), 4.17 (d, J = 5.5 Hz, 2H), 3.64-3.56 (m, 1H), 3.51-3.45 (m, 1H), 2.39 (br s, 1H), 2.29 (br s, 1H). LCMS: m/z 382.1 [M + H]⁺. |
| 64 | 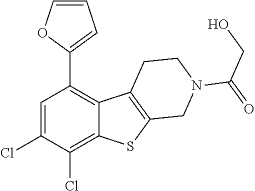 [SMILES: O=C(CO)N1CCc2c(sc3c(Cl)c(Cl)cc(-c4ccco4)c23)C1] | ¹H NMR (400 MHZ, DMSO-d6) δ = 7.90-7.83 (m, 1H), 7.64 (s, 1H), 6.76 (d, J = 3.2 Hz, 1H), 6.69 (br d, J = 1.8 Hz, 1H), 4.82 (br d, J = 10.5 Hz, 2H), 4.18 (br s, 2H), 3.65-3.49 (m, 2H), 2.44-2.29 (m, 2H). ESI [M + H] = 381.9/383.9 |
| 65 | 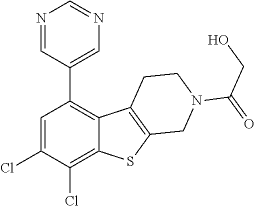 [SMILES: O=C(CO)N1CCc2c(sc3c(Cl)c(Cl)cc(-c4cncnc4)c23)C1] | ¹H NMR (400 MHZ, DMSO-d6) δ = 9.30 (s, 1H), 8.92 (s, 2H), 7.76-7.54 (m, 1H), 4.82 (br s, 1H), 4.81-4.68 (m, 2H), 4.13 (br d, J = 5.6 Hz, 2H), 3.61-3.39 (m, 2H), 2.21-1.96 (m, 2H). LCMS: m/z 393.9 [M + H]⁺. |
| 66 | 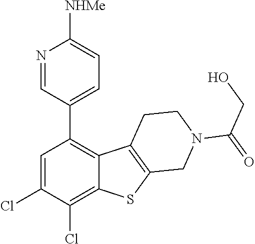 [SMILES: CNc1ccc(-c2cc(Cl)c(Cl)c3sc4c(c23)CCN(C(=O)CO)C4)cn1] | ¹H NMR (400 MHZ, DMSO-d6) δ = 7.99 (br s, 1H), 7.45-7.36 (m, 1H), 6.80-6.74 (m, 1H), 6.52 (br d, J = 8.8 Hz, 1H), 4.87-4.71 (m, 2H), 4.24-4.03 (m, 2H), 2.99-2.95 (m, 3H), 2.83 (br d, J = 3.4 Hz, 2H), 2.35-2.06 (m, 2H). ESI [M + H] = 422.0/424.0 |
| 67 | 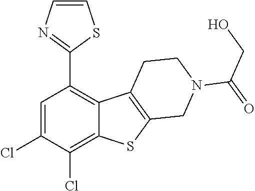 [SMILES: O=C(CO)N1CCc2c(sc3c(Cl)c(Cl)cc(-c4nccs4)c23)C1] | ¹H NMR (400 MHZ, DMSO-d6) δ = 8.06-8.01 (m, 2H), 7.75 (s, 1H), 4.86-4.78 (m, 2H), 4.15 (br s, 2H), 3.58-3.49 (m, 2H), 2.26-2.11 (m, 2H). LCMS: m/z 398.9 [M + H]⁺. |
| 68 | 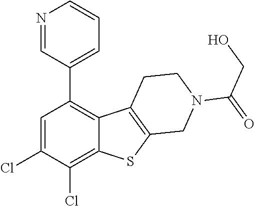 [SMILES: O=C(CO)N1CCc2c(sc3c(Cl)c(Cl)cc(-c4cccnc4)c23)C1] | ¹H NMR (400 MHZ, DMSO-d6) δ = 8.91 (br s, 2H), 8.29 (br s, 1H), 7.87 (br s, 1H), 7.67 (br s, 1H), 4.94-4.85 (m, 2H), 4.27-4.14 (m, 2H), 3.74-3.41 (m, 2H), 2.23-2.02 (m, 2H). ESI [M + H] = 393.0/395.0 |
| 69 | 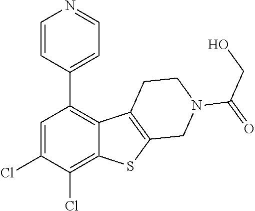 [SMILES: O=C(CO)N1CCc2c(sc3c(Cl)c(Cl)cc(-c4ccncc4)c23)C1] | ¹H NMR (400 MHZ, DMSO-d6) δ = 8.95 (d, J = 6.0 Hz, 2H), 8.00 (d, J = 5.9 Hz, 2H), 7.63 (s, 1H), 4.90-4.78 (m, 2H), 4.15 (br s, 2H), 3.55 (br s, 1H), 3.44 (br s, 1H), 2.14 (br s, 1H), 2.10-2.05 (m, 1H). ESI [M + H] = 392.9/394.9. |
| 70 | 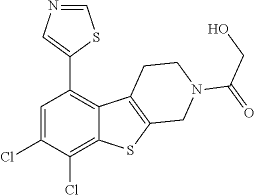 [SMILES: O=C(CO)N1CCc2c(sc3c(Cl)c(Cl)cc(-c4cncs4)c23)C1] | ¹H NMR (400 MHZ, DMSO-d6) δ = 9.28 (s, 1H), 8.00 (s, 1H), 7.64 (s, 1H), 4.83-4.77 (m, 2H), 4.15 (br d, J = 5.3 Hz, 2H), 3.62-3.55 (m, 1H), 3.52-3.47 (m, 1H), 2.29-2.23 (m, 1H), 2.20-2.12 (m, 1H). ESI [M + H] = 398.9/400.9 |
| 73 | 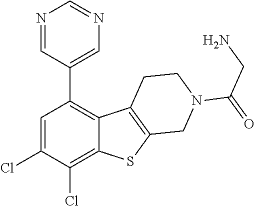 [SMILES: NCC(=O)N1CCc2c(sc3c(Cl)c(Cl)cc(-c4cncnc4)c23)C1] | ¹H NMR (400 MHZ, DMSO-d6) δ = 9.31 (s, 1H), 9.00-8.81 (m, 2H), 8.08 (br s, 2H), 7.73-7.62 (m, 1H), 4.90-4.82 (m, 2H), 3.95 (s, 2H), 3.65-3.45 (m, 2H), 2.22-1.95 (m, 2H). LCMS: m/z 392.9 [M + H]⁺. |
| 74 | 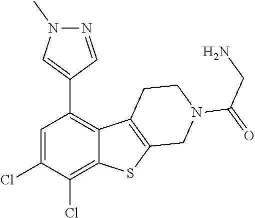 [SMILES: Cn1cc(-c2cc(Cl)c(Cl)c3sc4c(c23)CCN(C(=O)CN)C4)cn1] | ¹H NMR (400 MHZ, DMSO-d6) δ = 7.91 (s, 1H), 7.62-7.55 (m, 1H), 7.38 (s, 1H), 4.85-4.75 (m, 2H), 3.91 (s, 3H), 3.64-3.48 (m, 2H), 3.44 (br s, 2H), 2.45-2.30 (m, 2H), 2.07 (s, 2H). LCMS: m/z 395.1 [M + H]⁺. |
| 75 | 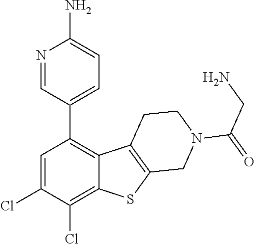 [SMILES: NCC(=O)N1CCc2c(sc3c(Cl)c(Cl)cc(-c4ccc(N)nc4)c23)C1] | ¹H NMR (400 MHZ, DMSO-d6) δ = 8.59-8.33 (m, 2H), 8.29 (br s, 2H), 8.10-8.04 (m, 1H), 7.98-7.91 (m, 1H), 7.59-7.54 (m, 1H), 7.17-7.09 (m, 1H), 5.07-4.69 (m, 2H), 3.95 (br s, 2H), 3.85-3.57 (m, 2H), 2.45-2.38 (m, 1H), 2.30 (br s, 1H). LCMS: m/z 407.0 [M + H]⁺. |
| 76 | 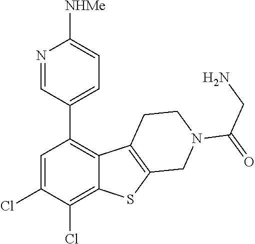 [SMILES: CNc1ccc(-c2cc(Cl)c(Cl)c3sc4c(c23)CCN(C(=O)CN)C4)cn1] | ¹H NMR (400 MHZ, DMSO-d6) δ = 10.11-9.14 (m, 1H), 8.33 (br s, 2H), 8.05-7.82 (m, 2H), 7.59-7.52 (m, 1H), 7.28-7.18 (m, 1H), 5.02-4.72 (m, 2H), 3.95 (br s, 2H), 3.75-3.57 (m, 2H), 3.06 (br s, 3H), 2.48-2.36 (m, 1H), 2.29 (br s, 1H). LCMS: m/z 421.0 [M + H]⁺. |
| 77 | 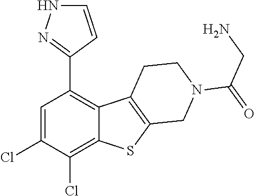 [SMILES: NCC(=O)N1CCc2c(sc3c(Cl)c(Cl)cc(-c4cc[nH]n4)c23)C1] | ¹H NMR (400 MHZ, DMSO-d6) δ = 13.30-12.88 (m, 1H), 7.91-7.72 (m, 1H), 7.49 (br s, 1H), 6.49 (br s, 1H), 4.86-4.75 (m, 2H), 3.56 (br d, J = 4.6 Hz, 1H), 3.49 (br s, 2H), 3.15 (br s, 3H), 2.29 (br s, 1H), 2.18 (br s, 1H). LCMS: m/z 381.1 [M + H]⁺. |
| 78 | 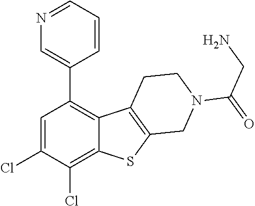 [SMILES: NCC(=O)N1CCc2c(sc3c(Cl)c(Cl)cc(-c4cccnc4)c23)C1] | ¹H NMR (400 MHZ, DMSO-d6) δ = 8.69 (dd, J = 1.5, 4.8 Hz, 1H), 8.63 (br s, 1H), 7.87 (br d, J = 7.5 Hz, 1H), 7.56-7.49 (m, 2H), 4.82 (br s, 2H), 3.45 (br t, J = 5.2 Hz, 1H), 3.40 (br s, 1H), 3.37 (s, 1H), 3.33 (br s, 1H), 2.51 (br s, 2H), 2.07 (br s, 1H), 1.96 (br s, 1H). LCMS: m/z 392.1 [M + H]⁺. |
| 79 | 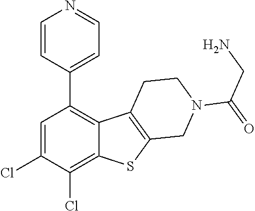 [SMILES: NCC(=O)N1CCc2c(sc3c(Cl)c(Cl)cc(-c4ccncc4)c23)C1] | ¹H NMR (400 MHz, DMSO-d6) δ = 8.94-8.82 (m, 2H), 8.26-8.17 (m, 2H), 7.81 (br d, J = 5.4 Hz, 2H), 7.64-7.57 (m, 1H), 4.93-4.84 (m, 2H), 3.94 (br t, J = 5.6 Hz, 2H), 3.60 (br t, J = 5.6 Hz, 1H), 3.48 (br t, J = 5.4 Hz, 1H), 2.18 (br s, 1H), 2.08 (br s, 1H). LCMS: m/z 392.0 [M + H]⁺. |
| 80 | 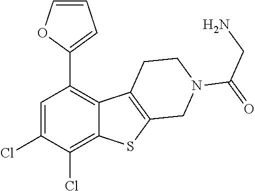 [SMILES: NCC(=O)N1CCc2c(sc3c(Cl)c(Cl)cc(-c4ccco4)c23)C1] | ¹H NMR (400 MHZ, DMSO-d6) δ = 7.90-7.88 (m, 1H), 7.99-7.79 (m, 1H), 6.84-6.60 (m, 2H), 4.95-4.73 (m, 2H), 3.68-3.39 (m, 4H), 3.32 (br s, 2H), 2.41 (br s, 1H), 2.31 (br s, 1H). LCMS: m/z 381.0 [M + H]⁺. |
| 81 | 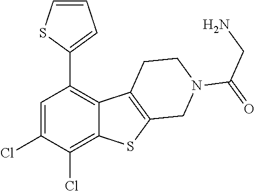 [SMILES: NCC(=O)N1CCc2c(sc3c(Cl)c(Cl)cc(-c4cccs4)c23)C1] | ¹H NMR (400 MHZ, DMSO-d6) δ = 7.73 (br d, J = 5.8 Hz, 1H), 7.59-7.51 (m, 1H), 7.23-7.15 (m, 2H), 4.83 (br s, 2H), 3.61-3.37 (m, 4H), 3.32 (s, 2H), 2.28 (br s, 1H), 2.18 (br d, J = 2.1 Hz, 1H). LCMS: m/z 397.0 [M + H]⁺. |
| 82 | 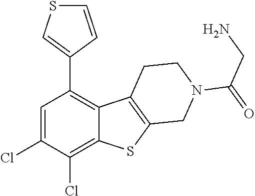 [SMILES: NCC(=O)N1CCc2c(sc3c(Cl)c(Cl)cc(-c4ccsc4)c23)C1] | ¹H NMR (400 MHZ, DMSO-d6) δ = 7.68 (dd, J = 2.9, 4.8 Hz, 1H), 7.63 (br s, 1H), 7.49-7.42 (m, 1H), 7.24-7.23 (m, 1H), 4.85-4.75 (m, 2H), 3.61-3.52 (m, 1H), 3.52-3.39 (m, 3H), 3.36-3.32 (m, 2H), 2.23 (br s, 1H), 2.12 (br d, J = 1.4 Hz, 1H). LCMS: m/z 397.0 [M + H]⁺. |
| 83 | 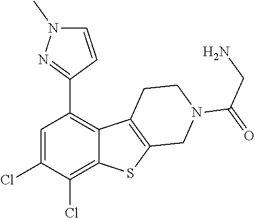 [SMILES: Cn1ccc(-c2cc(Cl)c(Cl)c3sc4c(c23)CCN(C(=O)CN)C4)n1] | ¹H NMR (400 MHZ, DMSO-d6) δ = 7.83 (d, J = 1.9 Hz, 1H), 7.48 (s, 1H), 6.47 (br s, 1H), 4.88-4.77 (m, 2H), 3.93 (s, 3H), 3.63-3.55 (m, 1H), 3.50 (br t, J = 5.1 Hz, 1H), 3.42 (s, 2H), 3.33 (br s, 2H), 2.36 (br s, 1H), 2.26 (br s, 1H). LCMS: m/z 395.1 [M + H]⁺. |
| 84 | 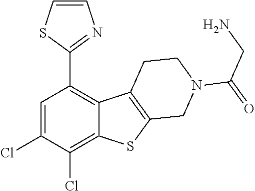 [SMILES: NCC(=O)N1CCc2c(sc3c(Cl)c(Cl)cc(-c4nccs4)c23)C1] | ¹H NMR (400 MHZ, DMSO-d6) δ = 8.23-8.09 (m, 2H), 8.04 (s, 2H), 7.80-7.75 (m, 1H), 4.93-4.83 (m, 2H), 3.98-3.93 (m, 2H), 3.67-3.59 (m, 1H), 3.53 (br t, J = 5.6 Hz, 1H), 2.29 (br s, 1H), 2.21-2.10 (m, 1H). LCMS: m/z 398.0 [M + H]⁺. |
| 85 | 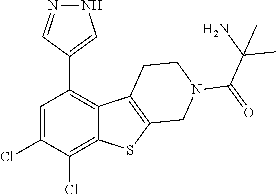 [SMILES: CC(C)(N)C(=O)N1CCc2c(sc3c(Cl)c(Cl)cc(-c4cn[nH]c4)c23)C1] | ¹H NMR (400 MHZ, DMSO-d6) δ = 8.19-8.14 (m, 2H), 8.02-7.60 (m, 2H), 7.42 (s, 1H), 4.92 (br s, 2H), 3.79-3.63 (m, 2H), 2.42-2.34 (m, 2H), 1.57 (s, 6H). LCMS: m/z 409.0 [M + H]⁺. |
| 86 | 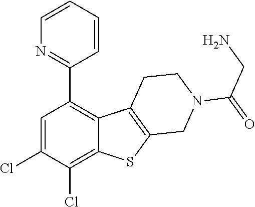 [SMILES: NCC(=O)N1CCc2c(sc3c(Cl)c(Cl)cc(-c4ccccn4)c23)C1] | ¹H NMR (400 MHz, DMSO-d6) δ = 8.92-8.83 (m, 1H), 8.44-8.32 (m, 3H), 7.97-7.84 (m, 2H), 7.79-7.73 (m, 1H), 4.89 (br s, 2H), 4.00-3.88 (m, 2H), 3.62-3.55 (m, 1H), 3.48 (br t, J = 5.4 Hz, 1H), 2.14 (br s, 1H), 2.01 (br s, 1H). LCMS: m/z 392.0 [M + H]⁺. |
| 87 | 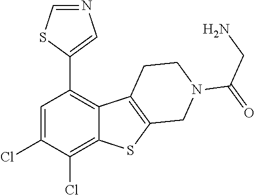 [SMILES: NCC(=O)N1CCc2c(sc3c(Cl)c(Cl)cc(-c4cncs4)c23)C1] | ¹H NMR (400 MHZ, DMSO-d6) δ = 9.33-9.28 (m, 1H), 8.21-8.11 (m, 2H), 7.98 (s, 1H), 7.69-7.62 (m, 1H), 4.90-4.84 (m, 2H), 3.95 (br d, J = 5.4 Hz, 3H), 2.31 (br s, 2H), 2.18 (br s, 1H). LCMS: m/z 397.9 [M + H]⁺. |
| 88 | 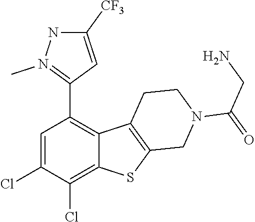 | ¹H NMR (400 MHZ, DMSO-d6) δ = 7.79-7.71 (m, 1H), 6.96 (s, 1H), 4.97-4.87 (m, 1H), 4.84-4.71 (m, 1H), 3.66 (s, 3H), 3.66-3.52 (m, 2H), 3.39 (br s, 4H), 2.17-2.00 (m, 2H). LCMS: m/z 463.1 [M + H]⁺. |
| 89 | 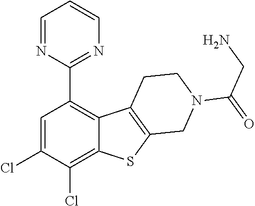 [SMILES: NCC(=O)N1CCc2c(sc3c(Cl)c(Cl)cc(-c4ncccn4)c23)C1] | ¹H NMR (400 MHZ, DMSO-d6) δ = 8.99 (d, J = 5.0 Hz, 2H), 8.21 (br s, 2H), 7.77-7.73 (m, 1H), 7.64 (q, J = 5.1 Hz, 1H), 4.91-4.87 (m, 2H), 3.99-3.91 (m, 2H), 3.60 (br t, J = 5.6 Hz, 1H), 3.48 (br t, J = 5.6 Hz, 1H), 2.25 (br s, 1H), 2.14 (br t, J = 5.3 Hz, 1H). LCMS: m/z 393.0 [M + H]⁺. |
| 90 | 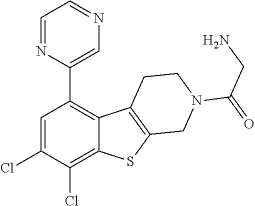 [SMILES: NCC(=O)N1CCc2c(sc3c(Cl)c(Cl)cc(-c4cnccn4)c23)C1] | ¹H NMR (400 MHZ, DMSO-d6) δ = 8.93-8.89 (m, 1H), 8.81-8.77 (m, 2H), 8.30-8.17 (m, 2H), 7.76-7.72 (m, 1H), 4.92-4.85 (m, 2H), 3.99-3.91 (m, 2H), 3.55-3.41 (m, 2H), 2.14 (br s, 1H), 2.06-1.98 (m, 1H). LCMS: m/z 393.0 [M + H]⁺ |
| 91 | 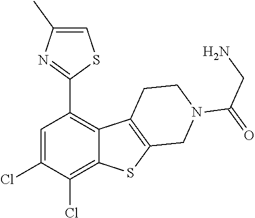 [SMILES: Cc1csc(-c2cc(Cl)c(Cl)c3sc4c(c23)CCN(C(=O)CN)C4)n1] | ¹H NMR (400 MHz, DMSO-d6) δ = 7.71 (s, 1H), 7.54 (s, 1H), 4.90-4.78 (m, 2H), 3.59 (br d, J = 5.8 Hz, 1H), 3.54-3.49 (m, 1H), 3.42 (br s, 2H), 3.38-3.34 (m, 2H), 2.49-2.45 (m, 3H), 2.26 (br s, 1H), 2.17 (br s, 1H). LCMS: m/z 412.0 [M + H]⁺. |
| 92 | 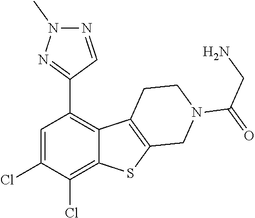 [SMILES: Cn1ncc(-c2cc(Cl)c(Cl)c3sc4c(c23)CCN(C(=O)CN)C4)n1] | ¹H NMR (400 MHZ, DMSO-d6) δ = 8.02 (s, 1H), 7.59 (s, 1H), 4.87-4.78 (m, 2H), 4.26 (s, 3H), 3.61 (br t, J = 5.1 Hz, 1H), 3.51 (br t, J = 5.4 Hz, 1H), 3.43 (br s, 4H), 2.30 (br s, 1H), 2.22 (br s, 1H). LCMS: m/z 396.1 [M + H]⁺. |
| 93 | 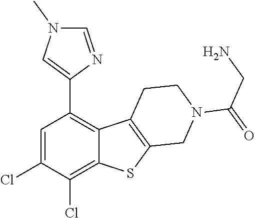 [SMILES: Cn1cnc(-c2cc(Cl)c(Cl)c3sc4c(c23)CCN(C(=O)CN)C4)c1] | ¹H NMR (400 MHZ, DMSO-d6) δ = 9.19 (br d, J = 12.1 Hz, 1H), 8.29 (br d, J = 5.1 Hz, 3H), 7.91 (d, J = 5.9 Hz, 1H), 7.71 (d, J = 3.4 Hz, 1H), 4.91 (br d, J = 5.0 Hz, 2H), 3.98-3.93 (m, 4H), 3.70 (br t, J = 5.6 Hz, 1H), 3.59-3.55 (m, 2H), 2.47 (br s, 1H), 2.38-2.32 (m, 1H). LCMS: m/z 395.0 [M + H]⁺. |
| 94 | 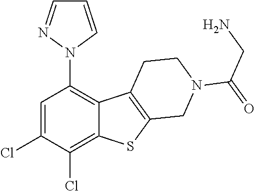 [SMILES: NCC(=O)N1CCc2c(sc3c(Cl)c(Cl)cc(-n4cccn4)c23)C1] | ¹H NMR (400 MHZ, DMSO-d6) δ = 8.18-8.04 (m, 3H), 7.80 (d, J = 1.1 Hz, 1H), 7.77-7.73 (m, 1H), 6.59 (t, J = 2.1 Hz, 1H), 4.93-4.82 (m, 2H), 3.95 (br d, J = 2.4 Hz, 2H), 3.66-3.59 (m, 1H), 3.51 (br t, J = 5.8 Hz, 1H), 2.14-2.10 (m, 1H), 2.04-1.98 (m, 1H). LCMS: m/z 381.0 [M + H]⁺. |
| 95 | 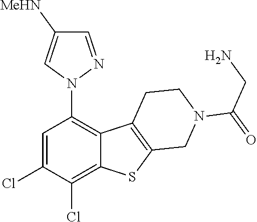 [SMILES: CNc1cnn(-c2cc(Cl)c(Cl)c3sc4c(c23)CCN(C(=O)CN)C4)c1] | ¹H NMR (400 MHZ, DMSO-d6) δ = 8.30-8.19 (m, 1H), 8.11 (br d, J = 4.9 Hz, 3H), 7.87 (br d, J = 8.1 Hz, 1H), 7.81-7.75 (m, 1H), 4.93-4.82 (m, 2H), 4.02-3.91 (m, 2H), 3.64 (br t, J = 5.6 Hz, 2H), 2.88 (s, 3H), 2.20 (br s, 1H), 2.06 (br s, 1H). LCMS: m/z 410.0 [M + H]⁺. |
| 96 | 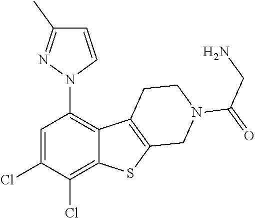 [SMILES: Cc1ccn(-c2cc(Cl)c(Cl)c3sc4c(c23)CCN(C(=O)CN)C4)n1] | ¹H NMR (400 MHZ, DMSO-d6) δ = 8.10 (br s, 2H), 7.99 (d, J = 2.1 Hz, 1H), 7.74-7.65 (m, 1H), 6.36 (d, J = 2.1 Hz, 1H), 4.91-4.82 (m, 2H), 3.96 (br s, 2H), 3.64 (br t, J = 5.7 Hz, 1H), 3.53 (br t, J = 5.8 Hz, 1H), 2.31-2.26 (m, 3H), 2.21 (br s, 1H), 2.13-2.07 (m, 1H). LCMS: m/z 395.0 [M + H]⁺. |
| 97 | 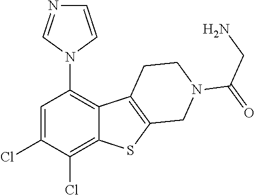 [SMILES: NCC(=O)N1CCc2c(sc3c(Cl)c(Cl)cc(-n4ccnc4)c23)C1] | ¹H NMR (400 MHz, DMSO-d6) δ = 9.64-9.24 (m, 1H), 8.35-8.27 (m, 2H), 8.13-8.01 (m, 2H), 7.49-7.37 (m, 1H), 4.91 (br s, 2H), 3.95 (br d, J = 4.6 Hz, 3H), 3.62-3.59 (m, 1H), 2.21 (br s, 1H), 2.09 (br d, J = 3.9 Hz, 1H). LCMS: m/z 381.0 [M + H]⁺. |
| 98 | 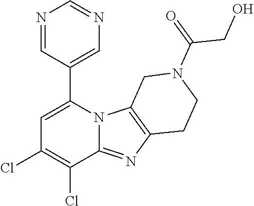 [SMILES: O=C(CO)N1CCc2nc3c(Cl)c(Cl)cc(-c4cncnc4)n3c2C1] | ¹H NMR (400 MHZ, DMSO-d₆) δ = 9.43 (s, 1H), 9.19-9.08 (m, 2H), 7.31 (s, 1H), 4.69-4.59 (m, 1H), 4.13 (br d, J = 5.4 Hz, 1H), 3.94 (s, 2H), 3.86-3.72 (m, 2H), 3.63 (br t, J = 5.3 Hz, 1H), 2.90-2.78 (m, 2H). ESI [M + H] = 378.2/380.2 |
| 99 | 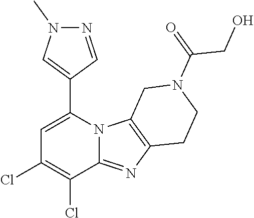 [SMILES: Cn1cc(-c2cc(Cl)c(Cl)c3nc4c(n23)CN(C(=O)CO)CC4)cn1] | ¹H NMR (400 MHZ, DMSO-d₆) δ = 8.23-8.14 (m, 1H), 7.86-7.74 (m, 1H), 7.08-6.89 (m, 1H), 4.68 (br s, 1H), 4.19 (br d, J = 19.9 Hz, 2H), 4.10-3.81 (m, 5H), 3.80-3.61 (m, 2H), 2.95-2.73 (m, 2H). ESI [M + H] = 380.0/382.0 |
| 100 | 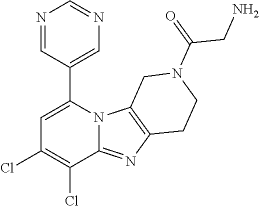 [SMILES: NCC(=O)N1CCc2nc3c(Cl)c(Cl)cc(-c4cncnc4)n3c2C1] | ¹H NMR (400 MHZ, METHANOL-d₄) δ = 9.48-9.35 (m, 1H), 9.10 (s, 2H), 7.28-7.12 (m, 1H), 4.64 (br d, J = 1.8 Hz, 2H), 4.11 (s, 1H), 3.97-3.88 (m, 1H), 3.76 (br t, J = 5.6 Hz, 1H), 3.57 (s, 1H), 3.00-2.88 (m, 2H). ESI [M + H] = 376.9/379.0. |
| 101 | 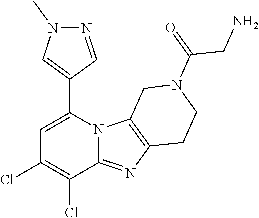 [SMILES: Cn1cc(-c2cc(Cl)c(Cl)c3nc4c(n23)CN(C(=O)CN)CC4)cn1] | ¹H NMR (400 MHZ, METHANOL-d₄) δ = 8.03 (s, 1H), 7.85-7.76 (m, 1H), 7.00-6.92 (m, 1H), 4.33 (s, 1H), 4.09-4.00 (m, 4H), 3.92 (br t, J = 5.6 Hz, 1H), 3.87-3.67 (m, 2H), 3.61 (br s, 1H), 2.99-2.85 (m, 2H). ESI [M + H] = 379.2/381.2 |

Cell-free mouse and human cGAS enzyme inhibition assay (Table 2). Activity of all compounds (in Table 1) against m-cGAS (30 nM) and or h-cGAS, (100 nM) was determined by measuring the conversion of ATP and GTP (100 μM each) to cGAMP in the presence of dsDNA (5 ug/mL) in a reaction buffer composed of Tris-HCl (20 mM, pH7.4), NaCl (150 mM), MnCl₂ (0.2 mM, h-cGAS) or MgCl₂ (5 mM, m-cGAS) and Tween-20 (0.01%), and the remaining ATP concentration using Kinase-Glo®Max Luminescent Kinase Assay (Promega, Madison, WI), as described (Lama, et. al., 2019). Briefly, 10 μl of a master mix of 0.4 mM ATP, 0.4 mM GTP, 0.02 mg/mL dsDNA in the reaction buffer supplemented with 2 mM DTT was added to reaction wells containing TDI-6570 or TDI-8246 (2× of the desired concentration) in the same buffer (20 uL) using a Thermo 8-channel Multidrop Combi dispenser. Next, 10 μl of a 4×m-cGAS (0.120 μM) or h-cGAS (0.4 μM) solution in the reaction buffer supplemented with 2 mM DTT was added to appropriate wells. Similar sets of reactions without cGAS or the inhibitor was set by adding the buffer alone. The reactions with the plates sealed were incubated at 37° C. (1 hour for m-cGAS and 3 hours for h-cGAS) and stopped by addition of 40 μl of the Kinase-Glo®Max. The luminescence was recorded in relative light units (RLUs) using a Biotek Synergy H1 Hybrid plate reader (BioTek, Winooski, VT). The ATP depletion was normalized against the positive control (no cGAS) and negative control (yes cGAS) as follows: % inhibition=100×(RLU sample-RLU average negative control)/(RLU average positive control-RLU average negative control). All reactions were run in triplicates and calculated using GraphPad Prism 9. The values represent mean IC₅₀±S.D.

TABLE 2
Activity of cGAS inhibitors in cell-free Enzyme assay.

| Cpd. | h-cGAS, conc. | | m-cGAS, conc. | |
|---|---|---|---|---|
| No. | 10 μM | 1 μM | 10 μM | 1 μM |
| 1 | +++ | − | +++ | n.d. |
| 2 | − | n.d. | − | n.d. |
| 3 | +++ | ++ | +++ | +++ |
| 4 | +++ | + | − | n.d. |
| 5 | +++ | + | − | n.d. |
| 6 | +++ | − | +++ | n.d. |
| 7 | +++ | ++ | − | n.d. |
| 8 | +++ | ++ | − | n.d. |
| 9 | +++ | ++ | − | n.d. |
| 10 | +++ | n.d. | − | n.d. |
| 11 | +++ | +++ | + | n.d. |
| 12 | +++ | − | − | n.d. |
| 13 | − | n.d. | − | n.d. |
| 14 | − | n.d. | − | n.d. |
| 15 | − | n.d. | − | n.d. |
| 16 | +++ | n.d. | − | n.d. |
| 17 | +++ | + | − | n.d. |
| 18 | − | n.d. | − | n.d. |
| 19 | − | n.d. | − | n.d. |
| 20 | +++ | + | − | n.d. |
| 21 | + | n.d. | +++ | n.d. |
| 24 | +++ | ++ | ++ | − |
| 25 | ++ | + | ++ | − |
| 26 | +++ | ++ | + | n.d. |
| 28 | ++ | + | n.d. | n.d. |
| 29 | +++ | + | n.d. | n.d. |
| 30 | ++ | − | n.d. | n.d. |
| 31 | +++ | ++ | | |
| 32 | ++ | + | +++ | ++ |
| 33 | +++ | ++ | +++ | +++ |
| 34 | ++ | + | n.d. | n.d. |
| 35 | +++ | + | n.d. | n.d. |
| 39 | +++ | − | n.d. | n.d. |
| 40 | +++ | ++ | n.d. | n.d. |
| 41 | +++ | ++ | n.d. | n.d. |
| 46 | +++ | ++ | − | n.d. |
| 47 | ++ | − | ++ | − |
| 48 | ++ | − | − | n.d. |
| 49 | − | n.d. | + | n.d. |
| 50 | − | n.d. | − | n.d. |
| 51 | + | − | − | n.d. |
| 52 | ++ | − | − | n.d. |
| 53 | ++ | − | +++ | − |
| 54 | +++ | ++ | ++ | − |
| 55 | − | n.d. | − | n.d. |
| 56 | ++ | n.d. | − | n.d. |
| 57 | ++ | n.d. | − | n.d. |
| 58 | ++ | − | +++ | n.d. |
| 60 | + | n.d. | ++ | − |
| 61 | + | n.d. | n.d. | n.d. |
| 62 | + | n.d. | +++ | + |
| 63 | + | n.d. | ++ | + |
| 64 | + | − | n.d. | n.d. |
| 65 | + | n.d. | +++ | − |
| 66 | − | n.d. | n.d. | n.d. |
| 67 | + | n.d. | n.d. | n.d. |
| 68 | − | n.d. | n.d. | n.d. |
| 69 | − | n.d. | n.d. | n.d |
| 70 | − | n.d. | n.d. | n.d. |
| 73 | ++ | − | ++ | − |
| 74 | + | − | +++ | + |
| 75 | ++ | − | +++ | n.d. |
| 76 | ++ | − | +++ | n.d. |
| 77 | ++ | + | +++ | + |
| 78 | ++ | − | +++ | + |
| 79 | + | n.d. | +++ | + |
| 80 | +++ | + | +++ | + |
| 81 | ++ | − | ++ | − |
| 82 | ++ | − | ++ | − |
| 83 | ++ | + | +++ | + |
| 84 | +++ | + | ++ | − |
| 85 | ++ | − | n.d. | n.d. |
| 86 | ++ | − | + | − |
| 87 | ++ | − | +++ | + |
| 88 | − | n.d. | − | n.d. |
| 89 | ++ | − | + | − |
| 90 | ++ | − | ++ | + |
| 91 | ++ | + | ++ | − |
| 92 | ++ | + | +++ | + |
| 93 | ++ | − | ++ | − |
| 94 | ++ | + | ++ | − |
| 95 | ++ | n.d. | ++ | n.d. |
| 96 | + | − | + | n.d. |
| 97 | + | n.d. | ++ | n.d. |
| 98 | − | n.d. | − | n.d. |
| 99 | − | n.d. | − | n.d. |
| 100 | − | n.d. | − | n.d. |
| 101 | − | n.d. | − | n.d. |

Note:
0-24% Activity in cell free is expressed as ″−″; 25-49% as +; 50-74% as ++; and 75-100% as +++.

Evaluation of active cGAS inhibitors in THP1 cells (Table 3). Cellular activity of all compounds (in Table 3) was determined using THP1-Dual™ cells as described (Lama et. al., 2019). THP1-Dual™ cells (Invivogen) (50000 cells in 100 μl media) were seeded into each well of a 96-well plate and treated with compounds at various dilution for 4 hours. Subsequently, cells were transfected with DNA (2 ug/ml) and lipofectamine and incubate the cells were incubated overnight. Ifnb expression was measured in cell supernatant using Quantiluc, nfkb expression was determined using Quantiblue, and cell viability using Promega® CellTiter-Glo®.

TABLE 3
Example of biological evaluation in THP1 assay.

| | Activity-inhibition and cell viability | | | | | |
|---|---|---|---|---|---|---|
| Cpd. | IFNB | | NFkB-SEAP | | Cell viability | |
| No. | 10 μM | 30 μM | 10 μM | 30 μM | 10 μM | 30 μM |
| 1 | − | − | − | − | +++ | +++ |
| 3 | − | n.d. | − | − | +++ | +++ |
| 4 | − | ++ | − | + | +++ | +++ |
| 5 | − | − | − | − | +++ | +++ |
| 6 | − | − | − | + | +++ | +++ |
| 7 | + | +++ | − | + | +++ | +++ |
| 8 | ++ | +++ | + | ++ | +++ | − |
| 9 | − | − | − | − | +++ | +++ |
| 10 | − | +++ | − | ++ | +++ | +++ |
| 11 | ++ | +++ | − | ++ | ++ | − |
| 12 | − | n.d. | − | n.d. | +++ | n.d. |
| 16 | +++ | +++ | +++ | +++ | +++ | + |
| 17 | + | +++ | + | +++ | +++ | ++ |
| 20 | ++ | +++ | +++ | +++ | +++ | +++ |
| 21 | n.d. | − | n.d. | − | n.d. | +++ |
| 24 | ++ | +++ | − | n.d. | +++ | +++ |
| 25 | n.d. | − | − | n.d. | +++ | n.d. |
| 26 | ++ | +++ | n.d. | n.d. | +++ | +++ |
| 28 | ++ | ++ | n.d. | n.d. | +++ | +++ |
| 29 | ++ | +++ | n.d. | n.d. | +++ | +++ |
| 30 | − | + | n.d. | n.d. | +++ | +++ |
| 31 | ++ | ++ | n.d. | n.d. | +++ | +++ |
| 32 | − | − | − | − | +++ | +++ |
| 33 | + | +++ | + | +++ | +++ | + |
| 34 | +++ | +++ | n.d. | n.d. | +++ | − |
| 35 | +++ | +++ | n.d. | n.d. | +++ | +++ |
| 39 | + | +++ | n.d. | n.d. | +++ | +++ |
| 40 | +++ | +++ | n.d. | n.d. | +++ | − |
| 41 | ++ | +++ | n.d. | n.d. | +++ | − |
| 46 | + | +++ | n.d. | n.d. | +++ | ++ |
| 48 | + | ++ | − | ++ | +++ | +++ |
| 51 | + | +++ | n.d. | n.d. | +++ | +++ |
| 52 | + | +++ | n.d. | n.d. | +++ | +++ |
| 53 | − | +++ | + | ++ | +++ | − |
| 54 | +++ | +++ | +++ | +++ | ++ | − |
| 56 | +++ | +++ | n.d. | n.d. | + | − |
| 57 | +++ | +++ | n.d. | n.d. | − | − |
| 62 | ++ | n.d. | − | n.d. | +++ | n.d. |
| 63 | ++ | +++ | + | +++ | +++ | +++ |
| 73 | − | +++ | − | +++ | +++ | − |
| 74 | +++ | +++ | + | ++ | +++ | − |
| 75 | +++ | +++ | − | +++ | +++ | +++ |
| 76 | +++ | +++ | − | +++ | +++ | +++ |
| 77 | + | +++ | ++ | +++ | +++ | − |
| 78 | +++ | +++ | ++ | +++ | + | − |
| 79 | +++ | +++ | ++ | +++ | + | − |
| 80 | ++ | +++ | ++ | +++ | +++ | − |
| 81 | ++ | +++ | +++ | +++ | +++ | − |
| 82 | ++ | +++ | +++ | +++ | +++ | + |
| 83 | ++ | +++ | +++ | +++ | ++ | − |
| 84 | + | +++ | − | ++ | +++ | − |
| 85 | +++ | +++ | n.d. | n.d. | + | − |
| 86 | − | +++ | − | ++ | +++ | − |
| 87 | +++ | +++ | ++ | +++ | − | − |
| 88 | + | +++ | ++ | +++ | +++ | − |
| 89 | − | ++ | − | ++ | +++ | ++ |
| 90 | + | ++ | + | +++ | +++ | − |
| 91 | ++ | n.d. | + | n.d. | ++ | − |
| 92 | +++ | +++ | +++ | +++ | ++ | − |
| 93 | − | +++ | − | ++ | +++ | − |
| 94 | − | n.d. | − | n.d. | +++ | n.d. |
| 95 | − | + | + | + | +++ | +++ |
| 96 | − | − | − | − | +++ | +++ |
| 97 | ++ | +++ | + | +++ | +++ | +++ |
| Positive ctrl | ++ | +++ | +++ | +++ | +++ | +++ |

Note:
Cell viability <24%, in THP1 cells is expressed as “−”similarly, +, ++, and +++ mean the compound respectively shows 25-49%; 50-74%; or 75-100% viability to cells.

Pharmacokinetic studies, off-target effects, protein binding studies, and metabolic studies data for selected compounds are provided in Table 4-7.

TABLE 4
Summary of Plasma (upper) and Brain (Lower) PK parameters

| PK parameters | | Compounds | | |
|---|---|---|---|---|
| (plasma) | Unit | 48 | 63 | TDI-8246 |
| Dose/route | mg/Kg | 25, I.P. | 25, I.P. | 25, I.P. |
| T_{1/2} | h | 4.53 | 4.79 | 0.729 |
| Tₘₐₓ | h | 1.00 | 2.00 | 1.00 |
| Cₘₐₓ | ng/mL | 6323 | 5073 | 3603 |
| AUCₗₐₛₜ | h*ng/ml | 33477 | 34242 | 6326 |
| AUC_{Inf} | h*ng/ml | 33490 | 34261 | 6331 |
| AUC_%Extrap_obs | % | 0.0394 | 0.0536 | 0.0766 |
| MRT_{Inf}_obs | h | 4.95 | 4.76 | 1.57 |
| AUCₗₐₛₜ/D | h*mg/mL | 1339 | 1370 | 253 |
| F | % | NA | NA | NA |
| PK parameters | | Compounds | | |
| (Brain) | Unit | 48 | 63 | TDI-8246 |
| Dose/route | mg/Kg | 25, I.P. | 25, I.P. | 25, I.P. |
| T_{1/2} | h | 3.45 | 3.86 | 0.512 |
| Tₘₐₓ | h | 2.00 | 4.00 | 1.00 |
| Cₘₐₓ | ng/ml | 19853 | 3721 | 1655 |
| AUCₗₐₛₜ | h*ng/ml | 127025 | 33856 | 2425 |
| AUC_{Inf} | h*ng/ml | 128066 | 34413 | 2447 |
| AUC_%Extrap_obs | % | 0.813 | 1.62 | 0.884 |
| MRT_{Inf}_obs | h | 4.69 | 6.51 | 1.37 |
| AUCₗₐₛₜ/D | h*mg/mL | 5081 | 1354 | 97.0 |
| Cmax Ratio | | 3.14 | 0.734 | 0.459 |
| (brain/plasma) | | | | |
| AUClast Ratio | | 3.79 | 0.989 | 0.383 |
| (brain/plasma) | | | | |

TABLE 5
Off-target effects of compounds 48 and 63

| | | | | Agonist Mode | | Antagonist Mode | |
|---|---|---|---|---|---|---|---|
| | The list of Safety | | | Mean percentage | | Mean percentage | |
| | Panel Screening | | | activation (%) at 10 μM | | inhibition (%) at 10 μM | |
| Class | Family | Target | Assay format | 48 | 63 | 48 | 63 |
| GPCR | Dopamine | D1 | cAMP assay | 4.36 | 6.19 | 6.62 | 3.08 |
| | 5- | 5HT2B | Ca2+ mobilization | −0.48 | 22.36 | 34.49 | 49.43 |
| | Hydroxytryptamine | | | | | | |
| hERG | Potassium channel | hERG | Automated patch | NA | NA | 62.98 | 49.70 |
| | | | clamp | | | | |
| Transporter | Serotonin | SET | Neurotransmitter | NA | NA | 46.25 | 15.15 |
| | Transporter | | uptake | | | | |
| Enzyme | Phosphodiesterase | PDE4D2 | IMAP | NA | NA | 56.43 | 73.16 |

TABLE 6
Protein binding results of cGAS inhibitors fractions in mouse brain homogenates

| | | | | | | % |
|---|---|---|---|---|---|---|
| | | | | | % | Remaining |
| Cpd. # | Species | % Bound_{Brain} | % Unbound_{Brain} | LogK | Recovery | at 6 hr |
| Telmisartan | Mouse | 98.12 | 1.88 | 1.72 | 99.29 | 103.38 |
| (ctrl comp) | | | | | | |
| 33¹ | Mouse | >99.996 | <0.004 | >4.39 | <82.40 | 90.87 |
| 48¹ | Mouse | >99.99 | <0.01 | >4.17 | <91.07 | 92.46 |
| 63¹ | Mouse | >99.998 | <0.002 | >4.72 | <88.44 | 108.27 |
| 74 | Mouse | 92.94 | 7.06 | 1.12 | 92.81 | 98.24 |
| 99 | Mouse | 81.03 | 18.97 | 0.64 | 90.09 | 99.35 |
| TDI-6570³ | Mouse | 99.58 | 0.42 | 2.38 | 89.39 | 94.51 |
| TDI-8264 | Mouse | 98.56 | 1.44 | 1.84 | 82.78 | 95.58 |

¹The test compound could not be detected in buffer side (below the limit of detection, BLOD), and LOD was used as peak area to calculate the cut-off value.
2 The compound was unstable, and the value of % remaining was lower than 50%.
³TDI-6570 was used as a mouse-selective cGAS inhibitor as a contriol

TABLE 7
Metabolic stability of test compounds

| | | | | | | |
|---|---|---|---|---|---|---|
| | | T½ | CLint(mic) | CLint(liver) | Remaining | Remaining |
| Cpd. # | R2 | (min) | (μL/min/mg) | (mL/min/kg) | (T = 60 min) | (NCF = 60 min) |

human Liver Microsomes

| | | | | | | |
|---|---|---|---|---|---|---|
| 11 | 0.4256 | >145 | <9.6 | <8.6 | 88.2% | 116.1% |
| 33 | 0.6510 | >145 | <9.6 | <8.6 | 87.6% | 109.6% |
| 54 | 0.5346 | >145 | <9.6 | <8.6 | 85.4% | 129.8% |
| 74 | 0.1584 | >145 | <9.6 | <8.6 | 93.6% | 89.2% |
| TDI-6570 | 0.9798 | 18.3 | 75.8 | 68.2 | 11.3% | 120.6% |
| TDI-8246 | 0.9634 | 80.6 | 17.2 | 15.5 | 59.5% | 134.1% |
| Testosterone | 0.9887 | 12.7 | 109.1 | 98.2 | 4.0% | 109.9% |
| Diclofenac | 0.9998 | 4.3 | 323.6 | 291.2 | 0.0% | 114.8% |
| Propafenone | 0.9355 | 5.4 | 255.0 | 229.5 | 0.0% | 117.3% |

Mouse Liver Microsomes

| | | | | | | |
|---|---|---|---|---|---|---|
| 11 | 0.8004 | >145 | <9.6 | <38.0 | 80.1% | 97.7% |
| 24 | 0.8749 | >184.78 | <7.50 | <23.07 | 6.3% | 116.2% |
| 33 | 0.8553 | 142.5 | 9.7 | 38.5 | 72.5% | 90.0% |
| 48 | 0.6968 | 2.0 | 679.2 | 87.2 | 88.1% | 94.7% |
| 54 | 0.9172 | >145 | <9.6 | <38.0 | 82.0% | 94.5% |
| 74 | 0.8676 | >145 | <9.6 | <38.0 | 84.8% | 97.8% |
| TDI-6570 | 0.9860 | 5.5 | 251.9 | 997.5 | 0.0% | 100.0% |
| TDI-8246 | 0.9902 | 17.0 | 81.7 | 323.4 | 8.8% | 99.8% |
| Verapamil | NA | 16.4 | 84.5 | 71.6 | 0.0% | 104.0% |
| Testosterone | 1.0000 | 3.7 | 375.9 | 1488.6 | 0.0% | 88.7% |
| Diclofenac | 0.9971 | 38.0 | 36.5 | 144.6 | 33.1% | 100.4% |
| Propafenone | 0.9905 | 1.8 | 776.9 | 3076.4 | 0.0% | 103.5% |

Notes:
NCF: no co-factor. No NADPH is added to NCF samples (replaced by buffer) during the 60 minute incubation. If the NCF remaining is less than 60%, then possibly non-NADPH dependent metabolism occurs
R²: correlation coefficient of the linear regression for the determination of kinetic constant (see raw data worksheet)
T_{1/2}: half life
CL_{int(mic)}: intrinsic clearance
CL_{int(mic)} = 0.693/T_{1/2}/mg microsome protein per mL
CLᵢₙₜ₍ₗᵢᵥₑᵣ₎ = CL_{int(mic)} * mg microsomal protein/g liver weight * g liver weight/kg body weight

### INCORPORATION BY REFERENCE

All of the U.S. patents and U.S. patent application publications cited herein are hereby incorporated by reference.

### EQUIVALENTS

Those skilled in the art will recognize or be able to ascertain using no more than routine experimentation, many equivalents to the various embodiments of the disclosure described herein. Such equivalents are encompassed by the following claims.

We claim:

1. A compound of Formula (I), or (II): 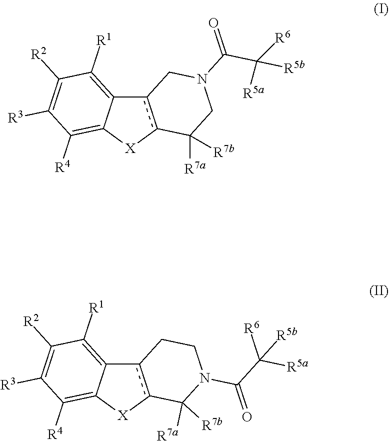
wherein:
R¹ is heteroaryl, halogen, aryl, cyclic amine, hydroxy, —OC(O)alkyl, —NH₂, —N(H)CO— alkyl, or alkoxy;
R² is H, alkyl, —CHF₂, —CF₃, —CN, —OR^{c}, halogen, or heterocyclyl;
R³ and R⁴ are independently H, halogen, —CHF₂, —CF₃, —CN, —OR^{c}, or —OCF₃;
R^{5a} and R^{5b} are independently H, alkyl, aryl, or cycloalkyl;
R⁶ is N(H)R^{a}, O-alkyl, OH, —CO₂R^{d};
or R^{5a} and R⁶ are taken together to form a 5-6 membered heterocyclyl;
R^{7a} and R^{7b} are independently H or alkyl, or, taken together with the carbon to which they are attached form a 3-membered aliphatic carbocyclic ring;
R^{a} is H, alkyl, —COR^{b}, —CON(H)R^{b}, or —CO₂R^{e};
R^{b} is alkyl or aryl;
R^{c} is H, alkyl, or —C(O)-alkyl;
R^{d} is H or alkyl;
R^{e} is alkyl, or aryl;
X is NH, NMe, NEt, O, or S; and
is a single or double bond; or a pharmaceutically acceptable salt thereof.

2-3. (canceled)

4. The compound of claim 1, wherein R¹ is

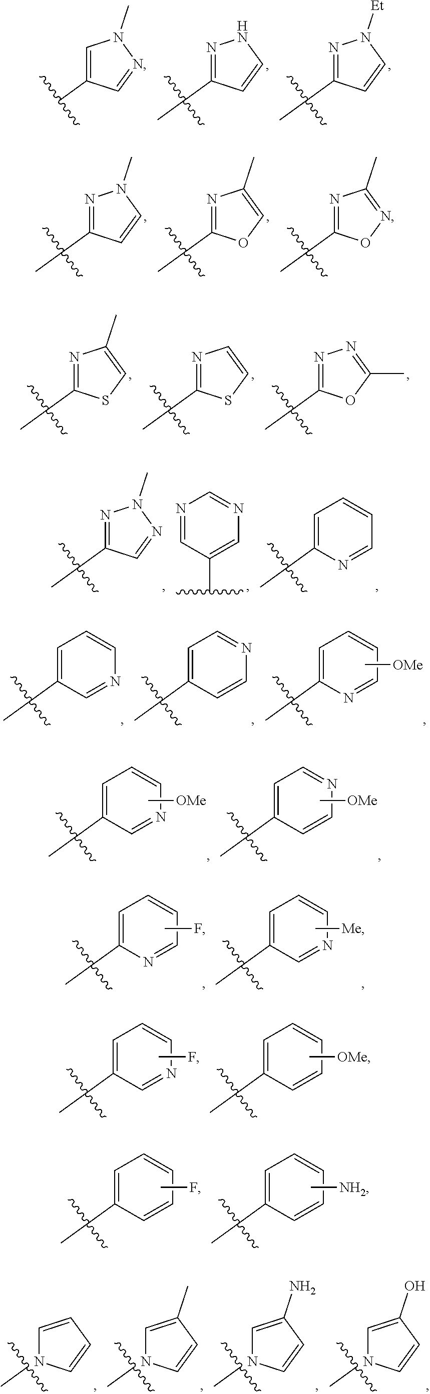

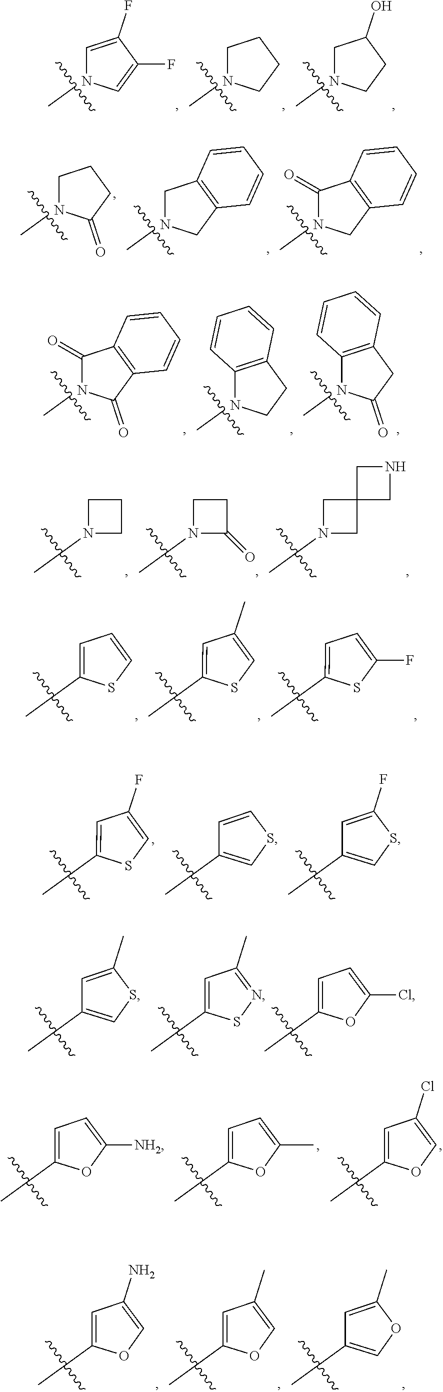

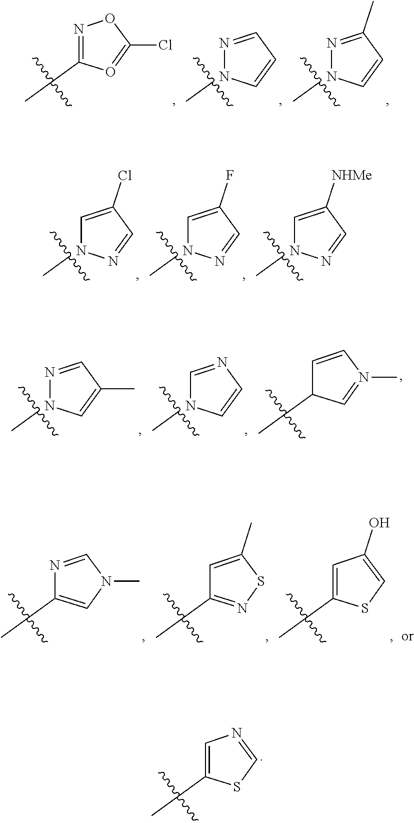

5-12. (canceled)

13. The compound of claim 1, wherein R² is H or halogen.

14. The compound of claim 1, wherein R³ and R⁴ are the same.

15. The compound of claim 14, wherein R³ and R⁴ are Cl.

16. The compound of claim 1, wherein at least one of R^{5a} and R^{5b} is H.

17. The compound of claim 1, wherein R^{5a} is alkyl.

18-19. (canceled)

20. The compound of claim 1, wherein R^{5a} and R^{5b} are methyl.

21. The compound of claim 1, wherein R⁶ is N(H)R^{a}.

22. The compound of claim 21, wherein R⁶ is NH₂ or N(H)Boc.

23-24. (canceled)

25. The compound of claim 1, wherein R⁶ is OH, CO₂H, or CO₂ (C₁₋₄-alkyl).

26-27. (canceled)

28. The compound of claim 1, wherein R^{7a} and R^{7b} are both hydrogen.

29. The compound of claim 1, wherein at least one of R^{7a} and R^{7b} is hydrogen.

30-31. (canceled)

32. The compound of claim 1, wherein X is NH or NMe.

33. (canceled)

34. The compound of claim 1, wherein X is O or S.

35. (canceled)

36. The compound of claim 1, wherein the compound is selected from: 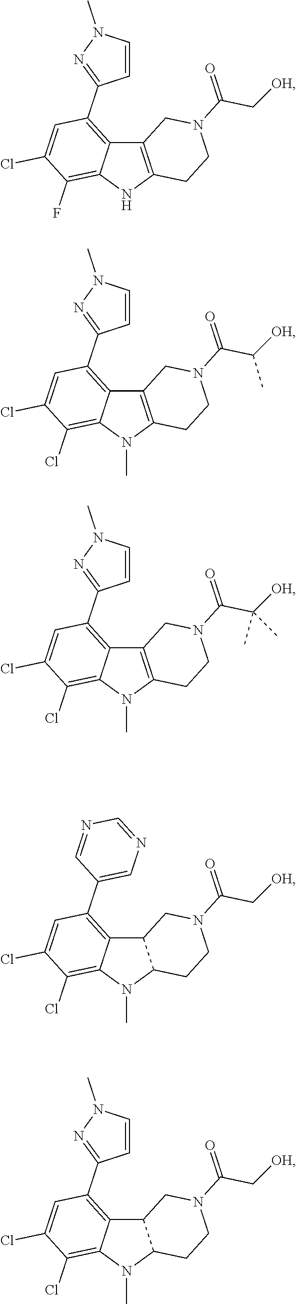 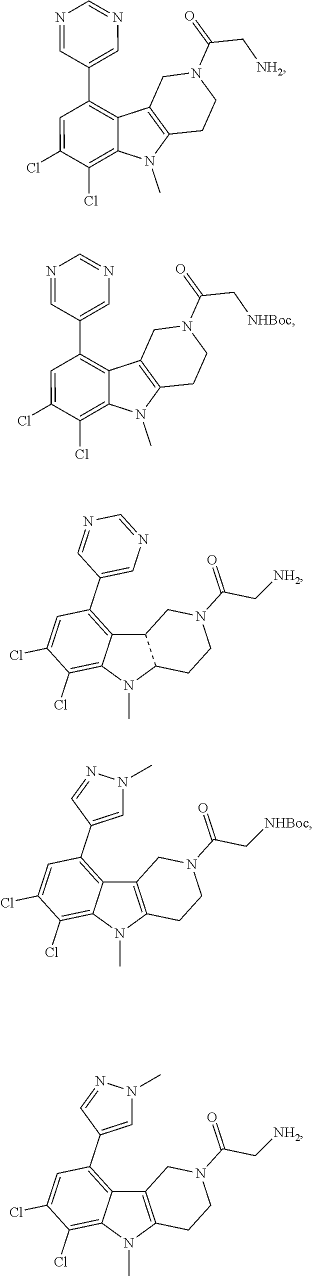 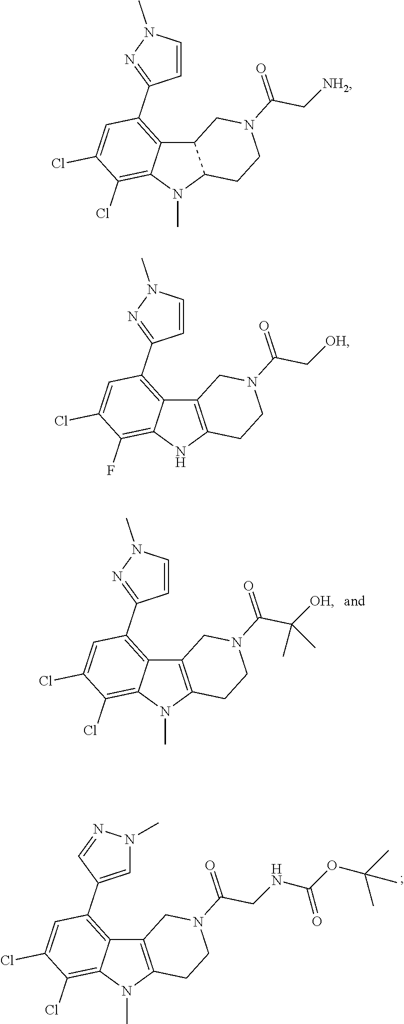
or selected from: 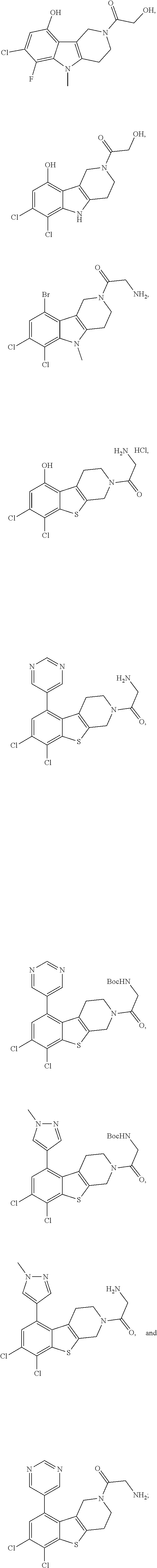
or selected from

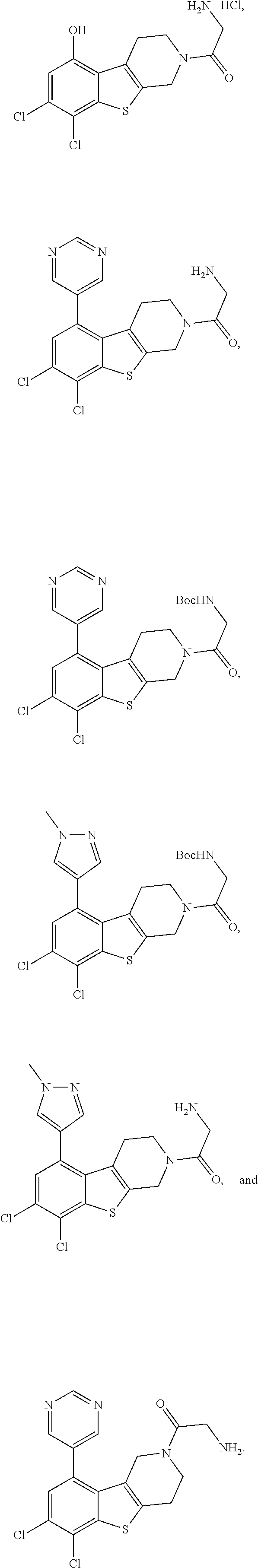

37.-38. (canceled)

39. The compound of claim 1, wherein the compound is selected from a compound of Table 1.

40. A pharmaceutical composition comprising a compound of claim 1 or salt thereof, and a pharmaceutical acceptable carrier.

41. A method of inhibiting inflammation or dsDNA-triggered interferon expression comprising administering to a subject in need thereof a compound of claim 1.

42. (canceled)

43. A method of treating a neurodegenerative disease or disorder, epilepsy, viral infection-associated dementia, or neurological disorders associated with COVID, comprising administering to a subject in need thereof a compound of any claim 1.

44-49. (canceled)
